(19) United States
(12) Patent Application Publication
(10) Pub. No.: US 2025/0322963 A1
(43) Pub. Date: Oct. 16, 2025

(54) MEDICAL MODELING ARCHITECTURE, INTELLIGENCE AND METHODS

(71) Applicant: Gemini Corp., San Francisco, CA (US)

(72) Inventor: Neal Solomon, Oakland, CA (US)

(73) Assignee: Gemini Corp., San Francisco, CA (US)

(21) Appl. No.: 19/094,887

(22) Filed: Mar. 29, 2025

Related U.S. Application Data
(60) Provisional application No. 63/631,056, filed on Apr. 8, 2024.
(60) Provisional application No. 63/680,852, filed on Aug. 8, 2024.

(51) Int. Cl. G16H 50/50; G16B 25/10; G16H 10/60; G16H 50/20; G16H 70/40; G16H 70/60

(52) U.S. Cl. CPC G16H 50/50; G16B 25/10; G16H 10/60; G16H 50/20; G16H 70/40; G16H 70/60

(57) ABSTRACT

Systems and methods for computer modeling in medicine. A sort of period table of medical models is described for personalized diagnostics, prognostics and therapeutics, including at least 80 major categories of medical models. Generative artificial intelligence and geometric deep learning techniques, and algorithms including 2D and 3D graph machine learning and GenAI algorithms, are described, tailored and applied to diagnostic disease description, prognostic prediction and therapeutic development and management, including generation of novel synthetic drugs. The AI and machine learning techniques and algorithms are applied to understand each individual's genetic, RNA and protein anomalies that represent the source of many unique patient diseases. AI-enabled software agents assist physicians and researchers in building patient medical models. Several personalized medicine applications of individualized medical modeling include cardiovascular disease, cancer, neurological disorders, immune system disorders and genetic diseases.

### FIELD OF THE INVENTION

The invention pertains to biological, biochemical, biomedical and medical modeling systems. Individualized medical modeling are computational duplicates of biomedical objects that delineate object structures, functions and interactions. Medical models apply computational chemistry and computational biology to represent, assess and test biological molecular and cellular dynamics. In addition to digital representation of microbiochemical models, medical models are also applied to organ, tissue, biosystem, body and population models. Individualized medical modeling is applied to medical diagnostics, prognostics, pharmacogenomics, in silico pharmacology and therapeutics. Medical modeling are an essential component of drug discovery, personalized medicine and precision medicine technologies.

The invention pertains to computer modeling of biological and biomedical phenomena. The invention involves analysis of combinatorial biology, combinatorial chemistry, biomedical anatomy, biomedical physiology and biophysics. The invention involves medical modeling of biomolecular and cellular phenomena. The invention applies artificial intelligence, machine learning and deep learning to computational biology, digital biology, biomedical systems, medical diagnostics, medical prognostics and medical therapeutics.

### BACKGROUND OF THE INVENTION

Digital twins represent computer modeling of physical entities. Historically, digital twins originated with NASA seeking to analyze industrial components in a computer model. This approach to industrial digital twins has grown to include numerous industrial applications.

Scientists have developed different categories of digital twins. These include a “static twin” in which a simple digital replica of a fixed physical entity or system is represented. A “mirror twin” or “function twin” is a static twin with dynamic behaviors, such as a mechanical device that changes positions. A “shadow twin” or self-adaptive twin is a functional twin with the ability to track real-time data updates; this dynamical representation requires data tracking its evolution over time. An “intelligent twin” is a self-adaptive twin that includes artificial intelligence and autonomy; this type of digital twin accounts for two-way dynamic information exchange of virtual and physical domains.

Digital twins have emerged in the biomedical field. Virtual You (Princeton, 2023), by Coveney and Highfield, describes academic work in medical digital twins. Most medical digital twins are generic representations of reference biological systems. For example, the classic illustration is the construction of a digital twin of a generic human heart. In this case, a generalized heart muscle is configured in a computer model. These generic DTs are useful for baseline reference, but are not personalized to an individual, much as the earliest decoding of the human genome involved an aggregation of numerous individual's DNA. As such, medical DTs so far have generally relied on academic use of supercomputers to construct models of generalized patients. These generic DTs are not tuned to an individual patient. That is, these medical digital twins do not represent or model a specific patient and their unique medical conditions. These primarily academic medical DTs focus on specific generic organ modeling—heart, liver, kidneys and brain—and modeling of body systems, such as the immune system. To the degree that prior medical DTs deal with patients, they are restricted to merely automating symptom-based diagnostics and simple existing drug selection processes.

Nvidia has developed a generative artificial intelligence (GenAI) platform for drug discovery called BioNeMo. This platform applies a pre-trained large language model (LLM) of biology foundation models, particularly the BERT biological model. BioNeMo has 3B parameters, which is fairly small when compared to very large 2T parameter LLMs. This platform enables biological researchers to apply the LLM for drug discovery and development. In contrast to this specialized biological LLM, much larger general LLMs such as Open AI's Chat GPT 4, 4o or 5, have trillions of parameters. There are scores of specialized biology LLMs for gene, protein and biological molecules, typically with 650M-10B parameters. DeepMind 3, introduced in May, 2024, is an example of this specialized LLM, which is programmed to predict protein structure representations and interaction data from gene sequence data. However, all of these biological LLMs generate generic biological data. For example, they will generate a specific generic protein molecule from gene or RNA sequence information. Biological LLMs can be programmed to identify a protein target, to generate drug candidates and to screen drug candidates, thereby accelerating drug discovery. One challenge of these LLMs is that it takes sometimes over a year or two to gestate these massive models, thereby making the information on which they rely inherently obsolete. Also, these LLMs have a tendency to hallucinate, that is, to generate false information. While these LLMs are a form of model, they represent relatively limited domains. Furthermore, they represent generic data about idealized healthy biomedical phenomena. The protein representations that are generated by bio LLMs focus on perfect optimized versions that provide a reference to which to compare unhealthy proteins.

The 2020s experienced a revolution in modern medicine that some describe as medicine 4.0. According to this view, the first generation of modern medicine occurred with the discovery by Watson and Crick in 1953 of the DNA double helix molecule. The second generation of modern medicine occurred in 2000 with the development of the human genome. The third generation of modern medicine is represented by the convergence of biology and engineering for integration of medicine and medical devices. Finally, the present era, medicine 4.0, is represented by computer modeling, AI and machine learning. However, while medicine 4.0 is a goal, there are still a number of important elements missing in order to realize the prospect of personalized medicine that applies advanced AI and modeling technologies to bioinformatics and individual patient pathologies in order to develop precision diagnostics and effective drug therapies. This latest era of the fourth generation of modern medicine—digital medicine—represents the hope of a truly personalized medicine in which quality and efficiency are optimized while costs are minimized. In this sense, most complex medical problems involve computational analysis and bioinformatics in order to strive for diagnostic and therapeutic solutions.

### SUMMARY OF THE INVENTION

### Problems that Individualized Medical Modeling Solves

There is a set of problems in biomedical modeling that individualized medical modeling (IMM) can solve. First, it is important to correctly diagnose each individual patient's disease, not an idealized textbook disease. Second, it is important to diagnose the specific source of each unique patient disease. This diagnosis typically requires an analysis of molecular and cellular conditions that describes the disease of each patient. Third, it is important to predict an individual patient's specific disease progress over multiple scenarios, particularly in scenarios without therapeutic intervention. Fourth, it is important to identify therapeutic solution options to the precise patient disease. Fifth, it is important to predict the therapy success of different therapy options in different situations.

Only the application of IMMs and AI can solve complex medical problems in a personalized way. IMMs optimize personalized medicine by precisely identifying a disease diagnosis, providing prognostic predictions of the disease progress and supplying therapeutic options and adaptations. IMMs are applied to solve complex medical challenges. For example, IMMs are applied to solving complex and difficult pathologies, including cardiovascular disease, neurogenerative disease and cancer. IMMs are applicable to orphan, genetic and rare diseases as well. IMMs are applied to optimize drug clinical trials in order to make them more effective and time and cost efficient. In addition, IMMs are applied to preemptive medicine in order to develop a personalized approach to anticipating chronic diseases. Moreover, IMMs are applied to autoimmune diseases by solving individualized chronic disease challenges involving dysregulation of the immune system. Finally, IMMs are applied to one of the most challenging problems in medicine, viz., the complex problem of metastatic cancer.

The present invention consists of a medical modeling architecture comprised of thirteen levels and about 80 major categories, including IMM categories representing diagnostic levels, therapeutic levels, prognostic levels and general medicine levels. In addition, the invention reveals connections regarding the functional dynamics between the IMM categories.

The invention discloses the mechanics of the IMM system, including software components, AI and ML components, personal health assistants (PHAs) and an integrated health record platform (IHRP). The invention shows the application of ML and GenAI to IMMs for medical diagnostics, prognostics and therapeutics. The invention discloses novel 3D geometric deep learning (GDL) and novel generative 3D GDL techniques and algorithms applied to IMMs with applications to medical diagnostics, prognostics and therapeutics.

IMMs are shown applied to medical diagnostics. IMMs are applied to biomarker analysis as well as identification of novel biomarkers. The invention discloses how to apply in silico experiments in IMMs for diagnostics, including with applications of ML and GenAI. IMMs are shown applied to cardiovascular, neurodegenerative and oncology pathology applications.

IMMs are also shown applied to diagnostic prognostics, including biomarker analysis for prognostics, in silico experiments for prognostics and applications of ML and GenAI to IMMs for diagnostic prognostics.

The invention discloses the application of IMMs to therapeutics. IMMs are shown with applications to drug discovery, including drug discovery modeling and experiments, with applications of ML and GenAI.

IMMs are shown with applications to novel synthetic drug design, including with applications of ML and GenAI.

IMMs are shown applied to therapeutic prognostics. For example, models indicating biomarkers for therapeutics prediction with feedback are shown as well as applications of ML and GenAI to IMMs for therapeutic prognostics.

IMMs are shown applied to drug clinical trials, preemptive medicine, autoimmune disorders and metastatic cancer. These applications illustrate the utility of IMMs to personalized medicine with a goal to identify and solve complex diseases.

### Novelties of the Invention

The present invention presents many novelties. The present invention presents a novel medical modeling architecture that consists of scores of IMM categories configured into several differentiated biomedical levels. The connections and data flows between the IMM categories are novel. This original medical modeling architecture for precision individualized medicine represents the connective tissue of digitalization for personalized medicine. Consequently, the present system delineating a medical modeling architecture supply clinicians with integrated medical solutions for complex molecular, cellular and macro medical challenges.

The invention is configured to collect medical data on each patient. These patient medical data—including DNA, RNA and protein biomarker data—are identified and analyzed by applying AI and ML techniques. Some of these AI, ML and GenAI algorithms are novel. The IMMs are built and analyzed by applying personalized health assistants (PHAs), software agents that collect, aggregate and analyze patient biomedical data. In addition, a novel digital medical record system, which tracks patient medical information, is shown applied to IMMs.

The invention shows a novel approach to applying IMMs to diagnostics, viz., with biomarker identification and analysis. The present MMs generate disease diagnostics with AI and ML analyses, which is useful for clinicians to identify patient pathologies on an individualized basis.

The invention provides IMMs to model 3D protein and cell structures by developing simulations that revolutionize medical diagnostics. In addition, the MMs of the invention develop simulations of healthy protein pathways and dysfunctional protein pathways, thereby showing precisely the source of individual diseases. Furthermore, the MMs are applied to develop 4D simulations of protein-protein interactions of dysfunctional proteins, illustrating how individual diseases operate.

The invention describes a novel approach for diagnostic prognostics by applying IMMs. The system is configured to track and analyze patient biomarkers, which enable pathology prognosis scenario development, particularly without therapeutic intervention. In addition, the system is useful for enabling preemptive prediction of disease development.

The invention shows how IMMs are applied to generate therapy solution options to match patient disease diagnoses. The system applies MMs for drug development in order to promote personalized medicine for targeting a molecular (gene or protein) target. MMs are also applied to generate novel synthetic drug design to fit a unique target.

The invention describes novel approaches for therapeutic prognostics. The IMMs are applied to predict the application of patient reactions to drugs. These therapeutic prognostics are useful to adapt therapy with the latest data on drug effects.

The invention describes the application of software agents to IMMs. The system applies novel AI techniques to MMs. In addition, a novel AI method—namely, 3D geometric deep learning (3D GDL)—with applications to several AI techniques is described. This original AI approach is shown in the context of specific MM applications, particularly involving therapeutic drug design.

The invention shows novel applications of IMMs to drug clinical trials. MMs enable precision drug clinical trials with AI and ML analyses. Particularly in the context of precision medicine in which specific drugs are configured to treat specific genetic disorders or specific abnormal proteins in dysfunctional cells, it is shown how the invention applies MMs to optimize personalized medicine. It is also shown how to construct an original social network connecting physicians of patients with orphan diseases, on the one hand, and bio or pharma companies, on the other hand, for aggregating clinical trials worldwide.

The invention applies the novel medical modeling system to preemptive medicine in order to enable clinicians to identify and track disease before they manifest, thereby saving patients years of time and quality of life.

The invention also applies the novel IMM system to autoimmune and inflammatory diseases. Moreover, the invention applies the novel IMM system to the medical challenge of metastatic cancer.

While throughout the description of the invention, several interesting classes of medical challenges are discussed as examples of application of the IMM system, including cardiovascular disease, neurology and psychiatry, numerous prominent cancers and autoimmune diseases, the invention is not limited to the diagnoses, prognostics or therapeutics of a particular type of disease.

### Advantages of the Invention

The present invention has many advantages. One prominent advantage of the present system refers to AI-based MM ability to target precise disease diagnoses, thereby saving clinicians and patients time and money. The AI-enabled MM modeling system shows a drug development process that is targeted and precise, thereby finding medical solutions faster, saving time and money. The AI-enabled MM system for personalized clinical trials also saves time and money.

The present invention applies AI-enabled MM to prediction of disease evolution, which helps to establish realistic expectations. The AI-enabled MM system also provides ways to predict therapy reactions, which saves time and money, and enables the adaptation and optimization of individualized therapies.

The present invention utilizes medical databases, biomarker analyses and AI to construct IMMs to solve difficult medical challenges with greater accuracy, thereby promoting personalized medicine. The system enables medical researchers to find precise solutions to hard medical challenges by applying the tools of AI and in silico experimentation integrated in the individualized medical modeling system.

The present invention applies IMMs for personalized medicine to solve individual patient medical challenges. The system enables clinicians to model patient diseases, which facilitates accurate diagnoses and identification of precision therapies, including drug discovery and novel drug design.

Consequently, this revolutionary technology furthers the paradigm of medicine 4.0, according to which medicine is digitized and integrated with artificial intelligence, to identify and solve complex medical challenges.

Reference to the remaining portions of the specification, including the drawings and claims, will realize other features and advantages of the present invention. Further features and advantages of the present invention, as well as the structure and operation of various embodiments of the present invention, are described in detail below with respect to accompanying drawings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

Overview of Individualized Medical Modeling System Architecture
1. Individualized Medical Modeling Logic
2. Biological System Analysis
3. Main Medical Modeling Map
   a. Level 1: General Patient Model
   b. Level 2: Diagnostics, Bioinformatics, Organ and Body System Analyses
   c. Level 3: Molecular and Cellular Description and Analysis
   d. Level 4: Structural Genetic Variant Combination Pathology Identification
   e. Level 5: Functional Molecular and Cellular Pathology Diagnosis
   f Level 6: Diagnostic Prognosis Simulations
   g. Level 7: General Therapy Solutions
   h. Level 8: Unique Therapy Solution Genesis
   i. Level 9: Therapy Option Testing and Simulations
   j. Level 10: Therapy Prediction Scenarios
   k. Level 11: Unified Patient Model
   l. Level 12: Human Population Model
   m. Level Ø: Master Individualized Medical Model
4. Functional Dynamics Between MM Categories

Mechanics of Individualized Medical Modeling System
1. Software for IMMs
2. AI and ML Applied to IMMs
   a. ML Applied to IMMs
   b. GenAI Applied to IMMs
   c. Geometric Deep Learning (GDL) Applied to IMMs
   d. Generative GDL Applied to IMMs
   e. Novel 3D GDL Techniques Applied to IMMs
   f. Novel Generative 3D GDL Techniques Applied to IMMs
3. Personal Health Assistants (PHAs) as Multifunctional Intelligent Software Agents Applied to IMMs
   a. PHA Mechanics
   b. PHAs for Modeling Functions
   c. PHA Typology
4. Integrated Health Record Platform: Integrating IMMs, Health Data Management, Medical Data Security and Patient Relationship Management
   a. IHRP and IMMs
   b. Patient Data Security Management in IHRP
   c. Patient Relationship Management

Individualized Medical Modeling for Diagnostics
1. IMMs for Personalized Medicine (PM) Diagnostics
2. Biomarker Analysis in IMMs for Diagnostics
3. Identification of Novel Biomarkers in IMMs
4. In Silico Experiments for Diagnostics in IMMs
5. Applications of ML and GenAI to Diagnostics in IMMs
6. IMMs Applied to Analyzing Diagnostics in Critical Diseases
   a. Cardiovascular Applications
   b. Neurological and Psychiatric Applications
   c. Oncology Applications
7. Individualized Medical Modeling for Diagnostic Prognostics
   a. Biomarker Analysis in IMMs for Prognostics
   b. In Silico Experiments for Diagnostic Prognostics in IMMs
   c. Applications of ML and GenAI to Diagnostic Prognostics in IMMs

Individualized Medical Modeling for Therapeutics
1. Drug Discovery with IMMs
   a. Personalized Medicine with IMMs Applied to Drug Discovery
   b. IMMs and In Silico Laboratory: Drug Discovery Modeling and Experiments
   c. Applications of ML and GenAI to Therapeutics Drug Discovery with IMMs
2. Novel Synthetic Drug Design with Individualized Medical Modeling
   a. Applications of ML and GenAI to Novel Synthetic Drug Design with IMMs
3. Individualized Medical Modeling for Therapeutic Prognostics
   a. Biomarkers in Therapeutics Prediction with Feedback
   b. Applications of ML and GenAI to Therapeutic Prognostics in IMMs

Applications of Individualized Medical Modeling System
1. Drug Clinical Trials with IMMs
2. Pre-emptive Medicine with IMMs
3. Autoimmune and Inflammatory Disease Analyses with IMMs
4. Metastatic Cancer Analysis with IMMs

### List of Acronyms

AI: Artificial intelligence
API Application programming interface
ASIC: Application specific integrated circuit
CAR T: Chimeric antigen receptor T cells
CPLD: Complex programmable logic device
CRBM: Conditional restricted Boltzmann machine
DL: Deep learning
DNA: Deoxyribonucleic acid
EGGNet: Equivariant graph of graphs neural network
EHR: Electronic health record
EMR: Electronic medical record
FPGA: Field programmable gate array
GAE: Graphical autoencoder
GAN: Generative adversarial network
GAT: Graph attention network
GenAI: Generative AI
GCN: Graph convolutional neural network
GCNN: Generative convolutional neural network
GDL: Geometric deep learning
GNN: Graph neural network
GPT: Generative pre-trained transformer
GPU: Graphic processing unit
HBM: High bandwidth memory
IHRP: Integrated Health Record Platform
IMM: Individualized medical model
LLM: Large language model
MAS: Multi-agent system
ML: Machine Learning
MM: Medical model
MSA: Multi sequence alignment
MVN: Manifold valued neural network
NGS: Next generation sequencing
NLP: Natural language processing
PDE: Partial differential equations
PDSM: Patient data security management
PHA: Personal Health Assistant
PLM: Protein language model
PM: Personalized Medicine
PRM: Patient relationship management
RAG: Retrieval augmented generation
RBM: Restricted Boltzmann machine
RNA: Ribonucleic acid
SCN: Spherical convolutional neural network
SNP: Single nucleotide polymorphism
SoC: System on a chip
VAE: Variational autoencoder

### BRIEF DESCRIPTION OF THE DRAWINGS

FIG. 1 is a table describing medical modeling architecture and modeling typology categories.

FIG. 2 is a table showing artificial intelligence categories applied to biomedical modeling technologies.

FIG. 3 is a table showing an RNA typology.

FIG. 4 is a table showing biomarkers of disease types.

FIG. 5 is a table showing a protein object structure classification system and neural network type matching.

FIG. 6 is a block diagram of the general medical modeling system architecture.

FIG. 7 is a diagram illustrating a comparison of healthy protein structure and unhealthy protein structure models.

8 is a diagram showing dysfunctional protein structural functionality.

FIG. 9 is a diagram showing dysfunctional protein outcome probabilities.

10 is a diagram showing 3D and 4D models of abnormal protein structure and function.

FIG. 11 is a diagram showing IMMs analyzing abnormal protein structure and configuring solutions.

FIG. 12 is a diagram showing IMM analysis of biomarkers to identify patient pathology.

FIG. 13 is a flow chart showing the process of disease discovery utilizing IMMs.

FIG. 14 is a flow chart showing the process of moving from diagnostics to therapeutics by utilizing IMMs.

FIG. 15 is a diagram showing IMMs applied to personalized medicine to assess a patient's disease diagnosis and prognosis.

FIG. 16 is a diagram showing a database table describing abnormal protein expression on a spectrum.

FIG. 17 is a flow chart showing the process of applying IMMs to identify therapeutic solutions to unique pathologies.

FIG. 18 is a diagram showing IMM categories.

FIG. 19 is a diagram showing the medical modeling architecture outline with data pipelines.

FIG. 20 is a diagram showing IMMs as active models.

FIG. 21 is a diagram illustrating databases inputting data into an IMM that generates models.

FIG. 22 is a diagram showing PHAs performing functions in IMMs.

FIG. 23 is a diagram showing data flows between layers of the IMM system.

FIG. 24 is a diagram showing IMM inter-layer dynamics of layers within each level.

FIG. 25 is a diagram showing inter-level data sharing within the IMM system.

FIG. 26 is a diagram showing dynamics of relations between layers of different levels.

FIG. 27 is a diagram showing PHAs facilitating two or more simultaneous data exchanges between layers.

FIG. 28 is a diagram showing the simultaneous processing of two or more MMs or simulations in the IMM system.

FIG. 29 is a diagram showing the application of APIs between levels and PHAs between layers of some levels connecting MM types in the IMM system.

FIG. 30 is a diagram showing two or more models on two or more layers communicating data to other models on different layers in the IMM system.

FIG. 31 is a diagram showing GDL techniques applied to analyze protein and cellular geometric properties.

FIG. 32 is a diagram showing GDL techniques applied to identify abnormal gene, RNA and protein geometric properties.

FIG. 33 is a diagram showing GDL techniques applied to compare abnormal proteins to optimal proteins.

FIG. 34 is a diagram showing GDL techniques applied to predict anomalous protein structure and function.

FIG. 35 is a diagram showing graph neural network general architecture.

FIG. 36 is a diagram showing graph representation of an input object in a GNN.

FIG. 37 is a diagram showing a 3D graph representation of a 3D graph neural network input.

FIG. 38 is a diagram showing a 3D GNN analysis of a 3D object and prediction of node connections.

FIG. 39 is a diagram showing 3D GNN with convolutional layers to output probabilistic options, with convolution layers applying different filters.

FIG. 40 is a diagram showing a 3D object converted to a 3D matrix and layer sampling for conversion to a 3D graph.

FIG. 41 is a diagram showing a 3D object converted to a 3D graph and nodes weighted in preparation for 3D GAT NN.

FIG. 42 is a diagram showing connections between 3D object nodes that are weighted and messages sent between nodes in a 3D GAT NN.

FIG. 43 is a diagram showing attention scores aggregated for nodes and connections for presentation to 3D GAT NN.

FIG. 44 is a diagram showing a 3D GNN with convolutional and GAT hybrid configuration to predict protein interaction.

FIG. 45 is a diagram showing a 3D graph of graph NN inputting two types of node and connection analyses.

FIG. 46 is a diagram showing two types of vectors analyzed in a 3D GoGNN.

FIG. 47 is a diagram showing a 3D autoencoder GNN model.

FIG. 48 is a diagram showing a 3D MV-GNN of a 3D abnormal protein with curved surfaces.

FIG. 49 is a diagram showing a protein LLM comparing healthy protein structure data to abnormal protein structure data.

FIG. 50 is a diagram showing a 3D GNN analyzing an abnormal protein structure.

FIG. 51 is a diagram showing a 3D GNN analyzing an abnormal protein structure to generate solution options.

FIG. 52 is a diagram showing an MM analyzing abnormal biomarkers and comparing the abnormal biomarkers to healthy DNA, RNA, proteins and antibodies, with an MM applying 3D GDL types to construct a novel synthetic drug to match to the drug target.

FIG. 53 is a diagram showing synthesis of an LLM and 3D GDL to identify, generate and test a novel synthetic protein.

FIG. 54 is a diagram showing the synthesis of an LLM and 3D GDL to identify, generate and test a novel synthetic antibody.

FIG. 55 is a flow chart showing the synthesis of an LLM and GDL to identify, generate and test a novel synthetic gene and transcription process.

FIG. 56 is a flow chart showing the synthesis of an LLM and 3D GDL to identify, generate and test novel synthetic RNA and translation process.

FIG. 57 is a flow chart showing the synthesis of an LLM and 3D GDL to identify, generate and test a novel synthetic small molecule.

FIG. 58 is a flow chart showing the synthesis of an LLM and 3D GDL to identify, generate and test a novel synthetic DNA, RNA, protein or antibody to modify stem cells.

FIG. 59 is a diagram showing a 3D GNN as descriptive of an abnormal protein and predictive of abnormal protein interactions.

FIG. 60 is a diagram showing an LLM-GNN hybrid model.

FIG. 61 is a diagram showing 3D GNNs connected to a 3D database management system.

FIG. 62 is a diagram showing APIs in the MM system.

FIG. 63 is a diagram showing the process of novel synthetic drug design.

FIG. 64 is a diagram showing an intelligent medical modeling system.

FIG. 65 is a diagram showing MM data interaction.

FIG. 66 is a diagram showing PHAs generating medical summaries from medical articles, databases or LLMs.

FIG. 67 is a diagram showing PHAs accessing patient medical test data and EMR, EHR and IHR data to build an IMM.

FIG. 68 is a diagram showing specialized PHAs in a multi-agent system applying skills to perform functions and communicate with each other.

FIG. 69 is a diagram showing a PHA combining two or more AI techniques or algorithms into a hybrid AI technique or algorithm and applied to an MM.

FIG. 70 is a diagram showing PHAs supplying different AI techniques or algorithms to different types of MMs.

FIG. 71 is a diagram showing PHAs acting as interfaces with doctors, a patient MM and patient tasks.

FIG. 72 is a diagram showing PHAs collecting and analyzing health data to develop diagnostic, prognostic or therapeutic solutions.

FIG. 73 is a diagram showing PHAs generating MMs, analyzing incomplete data and solving MM problems over time.

FIG. 74 is a diagram showing PHAs conducting in silico experiments to compare dysfunctional proteins to reference genes, RNA and proteins.

FIG. 75 is a diagram showing PHAs enabling an MM to supply diagnostic, prognostic and therapeutic solutions.

FIG. 76 is a block diagram showing PHA system dynamics.

FIG. 77 is a list of IHRP levels.

FIG. 78 is a diagram showing a natural language processing program analyzing health data that are input to the IHRP and MMs.

FIG. 79 is a diagram showing an IHRP interacting with MMs and generating patient health records.

FIG. 80 is a diagram showing the PDSM system layers.

FIG. 81 is a diagram showing the PDSM filtering patient security for MMs.

FIG. 82 is a diagram showing patient abnormal proteins analyzed and compared to healthy proteins to assess a patient disease in an IMM.

FIG. 83 is a diagram showing a MiR database of biomarker types that indicate the presence of a disease.

FIG. 84 is a flow chart showing how multiple biomarkers are analyzed to assess the sources of diseases.

FIG. 85 is a diagram showing protein abnormalities ranked on a scale based on geometrical configuration distortion degree.

FIG. 86 is a flow chart showing MMs performing biomarker analyses.

FIG. 87 is a diagram showing an MM analyzing many biomarkers to identify several critical biomarkers as a source of disease and as drug targets.

FIG. 88 is a flow chart showing the reverse engineering process for identifying novel biomarkers.

FIG. 89 is a flow chart showing the process of pathology analysis from a gene mutation to tracking abnormal protein pathways.

FIG. 90 is a diagram of different biomarkers associated with different phases of disease progress.

FIG. 91 is a diagram of AI and ML algorithms applied in an IMM to patient pathology biomarker data to evaluate protein and cellular dynamics.

FIG. 92 is a flow chart showing an MM generating in silico experiments to test and analyze patient biomarkers to identify the source of disease.

FIG. 93 is a diagram showing an IMM performing in silico experiments to assess patient abnormal proteins and propose a diagnosis.

FIG. 94 is a diagram showing an IMM analyzing biomarkers to identify genetic variant combinations that reveal disease targets.

FIG. 95 is a diagram showing an IMM performing in silico experiments of protein and drug interaction processes and building simulations.

FIG. 96 is a diagram showing an IMM performing in silico simulations of DNA, RNA, protein and cellular processes.

FIG. 97 is a diagram showing a healthy reference model compared to a patient pathology model in order to assess the evolution of a disease.

FIG. 98 is a diagram showing protein and cellular interaction processes simulated in IMMs.

FIG. 99 is a diagram showing diagnostic prognosis identifying and tracking DNA, RNA and protein degradation and evolution.

FIG. 100 is a diagram showing an MM comparing patient disease analysis and aggregate patients' diseases and their evolution to develop a prognosis of patient disease.

FIG. 101 is a diagram showing different patient disease progress scenarios mapped and rated.

FIG. 102 is a diagram showing MMs receiving and analyzing quality and quantify biomarker data in order to predict a pathology evolution.

FIG. 103 is a diagram showing biomarker data analyzed in MMs to predict disease prognosis and assign a prognosis score.

FIG. 104 is a diagram showing biomarker data analyzed in MMs in snapshots over four phases with different probable scenario outcomes over time.

FIG. 105 is a diagram showing an MM analyzing biomarker data to assess the evolution of patient disease outcomes.

FIG. 106 is a flow chart showing biomarker analysis in MMs to identify a pathology evolution and drug targets.

FIG. 107 is a diagram showing MMs applying in silico experiments to analyze biomarker data, develop 3D and 4D simulations and map probable pathology scenarios.

FIG. 108 is a diagram showing a micro-prognostics analysis applied in silico experiments in MMs to compare healthy and dysfunctional proteins and predict disease progress.

FIG. 109 is a diagram showing an MM applying in silico experiments to identify a drug target and drug-target fit and making drug-disease predictions.

FIG. 110 is a diagram showing a process to identify patient pathology on a molecular level.

FIG. 111 is a diagram showing MMs applying ML and AI to analyze biomarker data to diagnose a patient disease and to develop therapeutic drug options.

FIG. 112 is a flow chart showing MMs identifying and testing drug solutions for a drug target.

FIG. 113 is a diagram showing abnormal protein and antibody targets and application of mRNA solutions.

FIG. 114 is a flow chart showing an MM applying CADD to construct and test different hypothesis to solve a drug target.

FIG. 115 is a flow chart showing MMs identifying, evaluating and updating drug therapy options to solve patient pathology.

FIG. 116 is a flow chart showing MMs applied to describe the precise molecular geometry of a dysfunctional protein and to custom design a novel synthetic drug therapy.

FIG. 117 is a diagram showing GenAI and GDL algorithms applied to a protein language model to develop a novel protein or small molecule to solve a dysfunctional drug target.

FIG. 118 is a flow chart showing a GDL applied to describe dysfunctional protein and GenAI applied to custom design a drug solution.

FIG. 119 is a flow chart showing MMs designing and testing novel drugs to match a dysfunctional protein target by applying AI and ML techniques.

FIG. 120 is a diagram showing 3D GDL applied to describe dysfunctional protein and 2D GenGDL and 3D GenGDL applied to design novel drug therapies.

FIG. 121 is a diagram showing antibody specific protein LLMs, GenAI and GenGDL applied to MMs to construct a novel antibody.

FIG. 122 is a flow chart showing GenAI, 2D GenGDL or 3D GenGDL applied to MMs to design novel siRNA code, novel ligands and novel enzymes.

FIG. 123 is a diagram showing an MM designing a novel synthetic drug to optimize structural properties to fit a drug target.

FIG. 124 is a diagram showing MMs designing several classes of novel customized synthetic biologics.

FIG. 125 is a diagram showing AI-endowed PHAs collecting biological data for MM analysis of diagnostics, prognostics and therapeutics.

FIG. 126 is a diagram showing therapeutic prognostics describing drug options on disease progress and predicting a drug's effect on a disease.

FIG. 127 is a diagram showing different drugs providing effects on disease evolution and assigning drug reaction probability scores.

FIG. 128 is a flow chart showing an MM analyzing and comparing a patient's disease progress with and without drug therapy.

FIG. 129 is a diagram showing an MM analyzing and comparing effectiveness of two drugs on a patient disease progress.

FIG. 130 is a diagram showing biomarker measurements applied to compare disease progress with and without intervention.

FIG. 131 is a diagram showing drug therapy intervention applied, assessed (via biomarkers) and modified to show pathology improvement. [0175]132 is a diagram showing an MM analyzing patient biomarkers to assess pathology progress and recommending a modified therapy that shows major improvement.

FIG. 133 is a diagram showing an MM evaluating small molecule therapy biomarker feedback and recommending a novel synthetic drug which shows positive effect.

FIG. 134 is a diagram showing an MM analyzing a protein target, identifying a drug candidate and assessing the drug candidate effects on the protein target.

FIG. 135 is a diagram showing an MM assessing disease progress with and without therapy intervention.

FIG. 136 is a diagram showing an MM evaluating two drug therapy options in relation to no therapy control and ranking two therapy effects.

FIG. 137 is a diagram showing an MM identifying, predicting and recommending various drug therapy options to solve a patient pathology.

FIG. 138 is a diagram showing an MM analyzing biomarkers to predict or select treatment options.

FIG. 139 is a diagram showing an MM analyzing patient biomarkers on a scale and recommending different drug treatments at different times in evaluation of disease progress.

FIG. 140 is a diagram showing an MM tracing a control arm of drug clinical trials.

FIG. 141 is a diagram showing an MM applied to drug clinical trials for precision diagnosis and emulation of virtual patients.

FIG. 142 is a flow chart showing MMs analyzing and aggregating patient data in the active arm of clinical trials.

FIG. 143 is a diagram showing MMs applied to track active arm patient progress and compare to control arm patient progress.

FIG. 144 is a diagram showing an MM comparing and aggregating control and active arms data.

FIG. 145 is a diagram showing an MM applied to analyze biomarker data feedback of a drug to target a specific protein and analyze molecular interactions to show drug effectiveness.

FIG. 146 is a diagram showing an MM diagnosing a precise disease (and identifying abnormal protein targets) and identifying drug candidate options to match to the protein targets.

FIG. 147 is a diagram showing an MM analyzing aggregated control arm and active arm data.

FIG. 148 is diagram showing an MM analyzing hybrid control arm (including virtual patients) diagnostic prognostics data and active arm therapeutic prognostics data.

FIG. 149 is a diagram showing an MM analyzing therapeutic prognostics biomarker data from application of a drug candidate and modifying the drug to optimize effectiveness.

FIG. 150 is a diagram showing an MM predicting drug performance and modifying the drug when actual performance lags.

FIG. 151 is a diagram showing MMs analyzing biomarker data from clinical trial phases I and II to assess a drug candidate and modifying or replacing the drug in phase III.

FIG. 152 is a flow chart showing MMs analyzing different patient genetic, RNA or protein abnormalities in stratified sub-types to apply drug candidates to different patient clusters.

FIG. 153 is a diagram showing an MM applied to analyze the source of a genetic disease and to identification of a drug to treat the disease.

FIG. 154 is a diagram showing a patient relationship management program coordinating clinical trials with MMs and PHAs.

FIG. 155 is a flow chart showing drug companies generating a doctor network to coordinate clinical trials on targeted patients.

FIG. 156 is a diagram showing an MM generating virtual control arm data from diagnostic prognostics data to compare to therapeutic prognostics data of the active arm.

FIG. 157 is a flow chart showing MMs applied to generate synthetic patient data to use as virtual patient data of a hybrid control arm of drug clinical trials.

FIG. 158 is a diagram showing an MM assessing patient genetic and hereditary data to diagnose, predict and treat patient diseases that may develop in the future.

FIG. 159 is a flow chart showing MMs analyzing patient biomarker data to develop pre-emptive pre-diagnostic prediction of a probable future patient disease.

FIG. 160 is a diagram showing an MM analyzing biomarker data to assess probable scenarios of neuro-degenerative disease development trajectories over time.

FIG. 161 is a diagram showing an MM applying AI and ML to analyze biomarker and biomedical database data to identify future disease progression scenarios.

FIG. 162 is a diagram showing an MM developing and testing therapeutic options after pre-diagnosis of probable disease progression.

FIG. 163 is a diagram showing an MM applying, assessing and modifying drug therapy options in pre-emptive personalized medicine.

FIG. 164 is a diagram showing an MM stratify autoimmune disease subgroups by analyzing different classes of molecular biomarkers.

FIG. 165 is a diagram showing an MM developing a diagnosis and diagnostic prognosis of an autoimmune disease.

FIG. 166 is a diagram showing MMs building models that design novel synthetic proteins and novel synthetic antibodies.

FIG. 167 is a flow chart showing an MM designing novel synthetic therapies to solve abnormal autoimmune behaviors and optimizing a patient's immunity.

FIG. 168 is a diagram showing an MM identifying and reprogramming B cell receptors to bind an antibody to a specific antibody target.

FIG. 169 is a diagram showing an MM designing T cells to attack over-active B cells that overproduce autoantibodies that attack autoantigens in CAAR T therapy.

FIG. 170 is a flow chart showing an MM applying therapeutic modalities of SMC stem cells and RNA editing to modify T cells and limit generation of autoantibodies.

FIG. 171 is a list showing metastatic cascade stages.

FIG. 172 is a diagram showing a modified therapeutic modality applied to treat a secondary tumor.

FIG. 173 is a diagram showing therapies applied to address CTCs at stages 3, 4 and 5 after unique stage biomarkers are identified.

FIG. 174 is a diagram showing ctDNA of CTCs at secondary tumor site enabling the identification of a primary tumor site.

FIG. 175 is a diagram showing metastasized cancer cells, with modified genetic profiles, reprogrammed in their new tissues.

FIG. 176 is a diagram showing CSC's reprogramming protein pathways to resist drugs and immunity and reprogramming a secondary tumor site micro-environment.

FIG. 177 is a diagram showing detection of mRNA and ctDNA biomarkers predicting drug and immunity resistance at secondary tumor sites.

FIG. 178 is a diagram showing an MM analyzing biomarkers to diagnose, predict and treat cancer at each stage of development.

FIG. 179 is a diagram showing an MM applying different AI and ML techniques for cancer diagnostics, prognostics and therapeutics.

FIG. 180 is a diagram showing MMs analyzing biomarkers at different stages of cancer metastasis, with MMs developing novel drug therapies at each state.

### DETAILED DESCRIPTION OF THE INVENTION

### Overview of Individualized Medical Modeling System Architecture

### Individualized Medical Modeling Logic

The field of medicine is experiencing the convergence of several dramatic technological revolutions. First, medical databases have developed systematic libraries of genes, RNA and proteins. Second, next-generation sequencing (NGS) technologies have developed methods of rapidly deciphering data on individual patient DNA, RNA, proteins and lipids. Third, biomarker data are rapidly being identified as markers of disease and prognosis. Fourth, artificial intelligence and machine learning technologies have developed rapidly, particularly involving neural networks and large language models, which have the ability to predict 3D protein structures from DNA sequence data. These advanced models have been accelerated with the advent, fifth, of next-generation graphic processing units (GPUs) and system on chip (SoC) circuits. Sixth, the combination of these technologies has enabled biological modeling technologies. Seventh, these computer, bioinformatics and medical data technologies together enable a personalized medicine (PM) revolution that reveals accurate diagnostics of unique and complex patient pathologies. Eighth, the PM revolution supplies researchers with tools to identify targeted therapies to treat complex patient diseases. Finally, PM enables a new generation of precision drug clinical trials.

Since the decoding of the human genome, we have discovered about 20,000 genes in chromosomes that inhabit every human cell. DNA comprises a quaternary system of biological organization that consists of nucleic acids. DNA encodes for four nucleotides, from which about twenty useful amino acids are constructed of three nucleotides each. Each nucleotide is comprised of three parts—a sugar, a phosphate and a nitrogenous base. The sugar molecule is deoxyribose in DNA and ribose in RNA. One-dimensional strings of amino acids are constructed from nucleic acids, with about twenty useful amino acids comprising the building blocks of proteins. Proteins consist of a few dozen to thousands of amino acids.

Since protein structure data are inferred from DNA, RNA and amino acid sequence data, correctly configuring complex three-dimensional protein structure data from one-dimensional sequence data is challenging. The prediction of protein structure from genetic sequence data is referred to as the “protein folding problem” (PFP) and has eluded scientists until recently. Research teams have cracked the code. Researchers at Meta have developed ESM-2 and ESMFold, a 15B parameter LLM and protein structure prediction tool, which can generate novel synthetic protein structures. In addition, DeepMind's AlphaFold 3 uses a multiple sequence alignment (MSA) process and a diffusion model to predict 3D protein structure and protein interactions from one-dimensional amino acid sequence data. Salesforce's ProGen LLM, with 1.2B parameters, also develops protein structure prediction. These approaches employ protein LLMs according to which protein sequence data are converted to tokens and protein patterns are analyzed. In addition to predicting 3D protein structure from amino acid sequence data, AlphaFold 3 can also generate novel synthetic proteins by applying diffusion model neural networks that convert “text” sequences to images. These LLMs operate by training massive data sets, employing massive computer circuit capacity and steadily increasing the LLM parameter size to optimize scaling improvements.

However, the protein folding problem only supplies a reference benchmark for healthy or optimized proteins. Solutions to the PFP are useful for filling in the blanks of protein libraries in order to describe accurate 3D protein folding of optimized proteins. But these models are silent regarding dysfunctional proteins, which comprise the main universe of the source of diseases. These LLMs do not address the problem of variant genes and RNA and the abnormal protein structures that are constructed from these variants. Since abnormal proteins are at the root of diseases, these LLMs are not useful for helping to predict these unhealthy protein structures. Even beyond the abnormal protein structures, understanding the mechanisms of abnormal protein functions are particularly important to understanding the operation of diseases, an important feature about which these LLMs are also silent. Therefore, these LLMs are not seen as a solution to decipher the causes of disease. However, the idealized and perfected protein structures generated from the LLMs are useful to show the benchmark to which dysfunctional DNA, RNA and proteins can be compared. In this sense, these LLMs fill in gaps of the human genome database by accurately inferring protein structures from genetic sequence information. Finally, while these LLMs are useful for general biological research, they are not applied yet to personalized medicine.

While it is useful to have a reference benchmark of healthy DNA, RNA and proteins, what is needed is a modelling system that can decipher DNA and RNA variants, abnormal protein structures and dysfunctional protein operations. While the complexity of the protein folding problem is daunting, the complexity of deciphering the challenge of abnormal protein configurations and dysfunctional protein operation in cellular protein network pathways is magnitudes more complex. Consequently, tracking the network pathway of a mutated gene through transcription into a variant RNA sequence through translation into an abnormal protein structure (that substantially varies from a healthy protein structure) and into a dysfunctional protein operation in a cellular network presents a biological grand challenge. Predicting the abnormal protein structure of a mutated gene is particularly complex since there are numerous modes of mutation that may present which make prediction of abnormal protein structure a probabilistic challenge. Such a model needs to view pathology as a spectrum from healthy to the most extreme pathological situations. For example, a minor gene mutation may lead to only a minor (i.e., a single peptide) protein structure abnormal configuration which may have limited pathology consequences. On the other hand, a major gene mutation, or the combinations of multiple major gene mutations, may lead to a dramatic abnormal protein structure configuration which may have profound adverse consequences. The problem becomes particularly complicated when considering that many diseases have multiple genetic mutation and protein dysfunction sources which need to be considered in combination in order to identify the source of a disease. Finally, it is ideal to identify these complex sources of a patient's disease on an individual basis, that is, in the context of personalized medicine. How can we find solutions to these important biochemical challenges if we do not have a clear idea of the problems themselves of identifying gene and RNA variants and protein abnormalities and their pathology consequences?

While LLMs have utility in identifying healthy reference gene expression and protein structures, we need new modeling tools to solve these complex problems. We can develop solutions to these complex biological molecular challenges of identifying the multivariate sources of pathologies by applying individualized medical modeling. IMMs are useful to model abnormal protein structure configurations as well as to model abnormal protein interactions with healthy and unhealthy proteins. These 3D models describe the geometrical configurations of abnormal protein structures. In addition, IMMs develop 4D simulations to describe the operational processes of abnormal proteins as well as abnormal protein interactions and dysfunctional protein expression in intracellular protein pathways. How can we begin to find accurate therapeutic solutions if we cannot first identify and model the precise configuration and mechanics of abnormal proteins? As an analogy, this approach to modeling biomolecular structures and processes enables a lock and key model in which we can solve a pathology if we can find a precise configuration of a lock (i.e., a dysfunctional protein). Nevertheless, the ultimate goal of medicine is to construct a key for the unique lock that is represented in the abnormal protein(s). In this sense, we endeavor to develop models that precisely reverse engineer a protein or peptide solution. For example, a novel drug may consist of development of a synthetic protein or peptide (or RNA instructions to encode for a novel protein) to custom fit a protein target and correct for a protein abnormality. Only IMMs can solve these complex pathology challenges on a fine-grained individualized basis.

Individualized medical models are comprised of concrete and detailed patient medical data, general medical reference data and AI-based analytics. Medical models (MMs) are excellent at identifying and describing patient pathologies on a fine-grained basis. In addition, MMs are excellent at making useful and accurate predictions of disease progress. MMs are also excellent at making therapeutic recommendations based on existing treatment protocols. But among the interesting aspects of MMs are their ability to develop novel therapeutic solutions to unique complex patient pathologies. In some cases, MMs are instrumental to developing a novel synthetic drug design to fit a unique patient pathology.

Computer models are abstract mathematical representations of real objects, phenomena or systems. Models are representational systems generated to imitate features of the real world. Biological modeling enables researchers to apply computers to simulate or study biological, biochemical or biophysical objects or complex systems using mathematical, physical, biological or computer sciences and techniques. Computer simulations are the process of applying mathematical modeling, performed on a computer, in order to predict the behavior, including the causes and effects, of physical phenomena or systems. Computer simulation modeling is useful in designing, generating, evaluating and predicting complex systems by replicating a real or proposed representation of phenomena by applying computer software when changes to an actual system are particularly hard, expensive or impractical. Computer models are created to imitate aspects of the world, to predict events and to test hypotheses. In some cases, computer models can not only describe objects or solve problems, but also generate novel synthetic entities. In the context of biology, modeling can be applied to diagnostics in order to identify pathologies or describe biomolecular phenomena, to prognostics in order to predict pathology progressive events, with and without therapeutic intervention, and to therapeutics in order to identify effective drugs or to design novel pathology solutions.

Individualized medical modeling applies computer models, representations and simulations in order to describe, predict, project and prescribe biological, biochemical and biophysical phenomena. MMs are applied to molecular phenomena, including structural molecular entities such as gene, RNA, protein, peptide, lipid, antibody, ligand and small molecule objects. In addition, MMs are applied to describe actual and potential protein-protein interactions as well as potential drug-target interactions. MMs are applied to functional molecular entities that involve physiological and operative processes, events and interactions. MMs are applied to describing and predicting cellular anatomy, phenomena, events, signaling and interactions. MMs are applied to describing and predicting organ and tissue anatomy, physiology, events and interactions. MMs are applied to describing and predicting biosystems anatomy and functions. MMs are applied to describing and predicting patient anatomy, physiology, pathologies, activities and event progression. MMs build graphs, tables, maps, simulations and representations of biomedical data pertaining to the aforementioned molecular, cellular, organ, tissue, biosystem and general patient ontologies. MMs describe, predict and analyze specific patient diseases. Consequently, the IMM system models specific patient diagnostic, prognostic and therapeutic biological phenomena in order to identify precise patient pathologies and to develop or design effective drug therapies accurately targeting these precise diagnoses. The terms “individualized medical models” (IMMs) and “medical models” (MMs) are often used interchangeably to describe medical models that are applied to identifying and solving medical challenges involving individual patients. On a practical level, MMs require or involve computer hardware and software in order to operate. Additionally, MMs increasingly require sophisticated software, including database software, software agents, AI and ML.

### Biological System Analysis

IMMs tell a story. In many cases, IMMs represent the accumulation of multiple episodes illustrating the discovery of disease pathology over the life of a patient. This approach views IMMs as similar to chapters in a book in which each disease event is registered in the IMM schema for each patient. But in addition to recording the basic events of an individual's medical experiences, the IMM system is also able to assist physicians by offering therapeutic recommendations to diseases and even developing original therapeutic solutions. These complex novel drugs developed for individual patient's unique pathologies are the result of a combination of accurately identifying a patient's disease and surveying and applying a vast range of medical research information. The patient's unique pathology is analyzed by applying AI-based techniques in the IMM system, while the IMM system also surveys and analyzes prior medical research data in order to develop novel therapies for each patient pathology. The IMM system also supplies accurate predictions of disease evolution based on comparison of a patient's pathology condition with an analysis of the IMM's accessible vast medical library.

One major tool in an IMM's assessment of a patient pathology involves biomarkers. While biomarkers can be biological, imaging or digital, the main tool applied by IMMs involve biological biomarkers such as DNA, RNA or protein analyses. In a sense, a biomarker assessment represents a snapshot of a patient condition in time. It is precisely an analysis of the 3D structures of the abnormal protein and RNA biomarkers that provides the essential data for building an accurate diagnostic picture of a patient pathology. By combining multiple 3D biological biomarker snapshot data, an IMM can build a 4D simulation of dysfunctional protein interactions and abnormal cell dynamics to accurately describe the biomolecular anatomy and physiology that forms the basis for a patient pathology. These biomarker guideposts enable the IMM system to track and predict the phases of a patient's disease progress over time.

The process of disease discovery can be described by utilizing IMMs. Once an IMM obtains gene and RNA sequencing data, a model identifies genetic mutations. Biomarker data are analyzed in order to ascertain abnormal protein structures. The IMM generates a list of gene and RNA mutations and dysfunctional DNA, RNA or protein biomarkers. The IMM generates a table to compare the biomarker data to healthy protein or biomolecular data. The IMM compares the healthy biomarker data to abnormal protein structure and function data. The IMM analyzes dysfunctional protein interactions and protein pathway mechanics from the biomarker analyses. The IMM then identifies and validates specific protein target(s) as a source of a patient's disease. Finally, the IMM tracks disease progress by tracking biomarkers over time and updating the model delineating patient pathology.

The process of moving from diagnostics to therapeutics can be described by utilizing IMMs. First, an IMM identifies DNA and RNA variant data from next-generation sequencing (NGS) analyses. The IMM accesses biological LLMs in order to identify healthy protein structure prediction models. The IMM then identifies abnormal protein structure model and/or abnormal protein function model data. The IMM identifies and assesses a patient's RNA, protein and small molecule biomarkers. The model then compares healthy versus abnormal protein interactions, including protein-protein, protein-lipid, protein-small molecule, protein-disease and protein-drug interactions. The IMM accurately identifies and describes the abnormal protein(s) that cause the patient pathology. The IMM applies GenAI techniques to develop a novel synthetic protein to design a drug to solve the abnormal protein pathology.

IMMs are applied to personalized medicine. PM requires a fine-grained analysis of a patient disease in order to develop an understanding of the disease, the disease progress and possible therapeutic modalities. In order to facilitate the realization of PM by IMMs, it is necessary to apply various biomedical and computational tools. First, IMMs require access to data from biological and medical databases in order to describe healthy molecular and cellular structures and functions. These medical databases and medical research articles represent the foundation of medical experience. Second, IMMs require bio LLM data, which predict healthy molecular structures. Third, DNA, RNA and protein sequence data on each individual is essential in order for IMMs to develop a map of individual pathology. Fourth, these biological sequence data are utilized by the IMMs to identify biomarker data over time of individual patient pathologies. The IMMs apply analytical tools to compare individual patient unique gene and biomarker data to biomedical databases and bio LLMs. Fifth, the IMMs analyze each patient's biomarker data in order to track disease progression and make analytical predictions about a disease evolution.

In order to examine the qualities and attributes of abnormal protein expression, MMs employ database tables. The tables of abnormal protein expression reveal a spectrum of dysfunctional protein manifestations. The IMM system may engage AI techniques in order to predict abnormal protein expression features from gene or RNA specific mutation types. The IMM system accesses tables of gene and RNA mutations in order to develop an analysis of abnormal protein expression by comparing table fields.

IMMs are applied to finding therapeutic solutions to unique pathologies. After an IMM identifies a unique gene, RNA and/or protein dysfunction as a source of disease, the IMM accesses medical or biological databases to obtain a reference for optimum molecular health for comparison of patient disease. Once the IMM identifies optimal existing drug options to solve a patient disease, the IMM ranks the drug options and selects an optimal drug therapy. If a drug candidate is applied and is unsuccessful, the IMM generates a novel synthetic drug design by applying AI techniques and by applying in silico experiments to identify a unique drug solution, seeking to identify a key with a good fit to a unique lock. After a drug is applied to a patient, the IMM identifies biomarkers to track the therapeutic prognosis. The IMM predicts a specific drug's effects on the patient's disease.

While there are different ways of categorizing IMMs, one straightforward way of typing MMs is by seeing IMMs as Object MMs, Process MMs and System MMs. Object MMs involve construction of 3D models that describe an object's structure or multiple objects' structures. For example, a protein, cell, organ or drug chemical structure can be elucidated in an IMM. Process MMs are simulations. These 4D sims provide a functional description of biomedical processes. As examples, a disease evolution can be described by showing abnormal protein evolution and interactions in protein pathways or a DNA or protein degradation process can be shown. Process MMs are akin to a video when compared to an object MM snapshot. System MMs are models that combine biomedical components into unified models to enable an understanding of all elements of a pathology. The system approach transcends a single object or process and embraces multiple views of a medical problem.

The present system represents a sort of periodic table of individual medical models that represents a spectrum from healthy patients to a broad range of patient pathologies. The IMM system architecture consists of a set of MM categories that reference specific medical categories configured in a typology of functional medical levels and layers. The foundational MM system is designed to connect the multiple MM categories in an integrated digital fabric. Data pipelines are organized to connect to the various MM categories in order to supply connections to physical and virtual objects. AI is integrated into the MM system architecture. There are dozens of distinct AI techniques and algorithms that are applied to computational analysis of various MM categories.

Overall, there are thirteen distinct levels of the IMM system. Each level consists of multiple layers. Each data category is configured in the context of this IMM system architecture.

IMM system mechanics require hardware and software to operate. The hardware includes computer logic and memory circuits, while the software includes databases, data sets, modeling software, analytics software and AI algorithms. The system also includes biological and chemistry design software.

In one mode, a physician may begin the process of building a patient IMM with an aggregated patient model. This generalized patient model can be typified by different major categories such as a baby model, a toddler model, an age-relative child model (3-18), an adult female model, an adult male model, or a geriatric male or female model. These healthy base IMMs are useful as a reference point. From these reference models physicians can construct specific patient models to which to compare patient pathologies.

Beyond the whole patient IMM, the physician can utilize the healthy base model for biomolecular diagnostic analyses too. The basic MM provides reference data for gene, RNA, protein structure, protein function, cell, cell network, tissue, organ and body system data types. The individual patient pathology model can be compared to reference models of healthy biomedical structural and functional data.

While traditional medical models are relatively primitive because they strive mainly to automate twentieth century medical diagnostics, the goal of which is to identify a symptom based disease, and twentieth century medical therapeutics, the goal of which is to identify an existing off-the-shelf drug or treatment to fit the simplistic diagnosis, the present system endeavors to realize the potential of personalized medicine by developing sophisticated models of accurate biomolecular descriptions of pathology as well as novel therapeutic solutions to each unique patient pathology.

The present system views IMMs as active models. IMMs accumulate patient data in databases received from multiple sources. In some cases, the models conduct analytical experiments to solve problems. In other instances, the models seek out medical reference data from biological or medical databases or from bio LLMs in order to complete models akin to completing a puzzle. Each of these data points are accumulated and analyzed. The IMMs map patient medical temporal events, including molecular events, cellular events and drug events. The MMs receive patient biomarker data in order to update its models on patient condition. These temporal elements of IMMs map multiple patient health reports, medical tests and medical events in order to build a picture of the patient medical condition.

The present system includes software agents, which are involved in different aspects of the IMM system. Personal Health Assistants (PHAs) collect generic medical data and specific patient medical data, build IMMs and perform IMM analytics. The PHAs may include different levels of autonomy. For instance, on some levels of the IMM system, the PHAs may collect generic or patient data in order to complete a data table. In other cases, the PHAs may analyze patient data in order to identify a pathology diagnosis or predict a pathology progression. PHAs endow IMMs with autonomy and self-awareness. In this sense, an IMM may realize that it has insufficient information in order to solve a diagnostic, prognostic or therapeutic problem; as such, it may request specific information in order to accomplish a task, such as requesting a particular type of medical test so as to gain insight into a dimension of a patient pathology.

MM categories in the present IMM system can be viewed as a complex lens system applied to assess patient diseases and therapeutics solutions. A microscopic lens can be applied to view molecular and cellular phenomena, which is typically the source of complex or chronic diseases. On the other hand, a wide-angle lens can be seen as a broader view of a body or body systems level. The MM analyses can be viewed as a sort of zoom lens which changes focal length from a wide-angle view to a microscopic view. This analogy of lens system scaling from microscopic to broad views is useful for understanding the IMM system category interactions.

The present IMM system can be applied to different medical specialties to focus on different elements of patient anatomy and physiology. Cardiologists will focus primarily on heart and cardiovascular system MMs. Hematologists will focus mainly on blood cells. Immunologists will focus on the immune system and on immune cells. Oncologists will focus on neoplasms of various cell types. Neurologists will focus on brain and the nervous system. Each specialist will focus on their preferred organ, tissue types and cell types.

### Main Medical Modeling Map

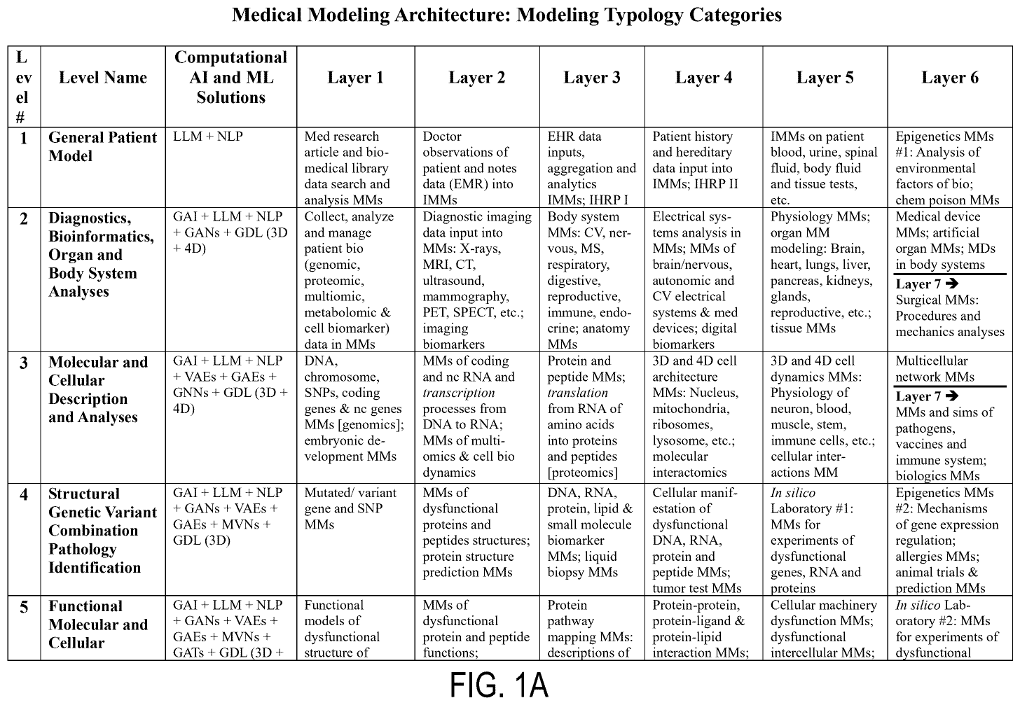

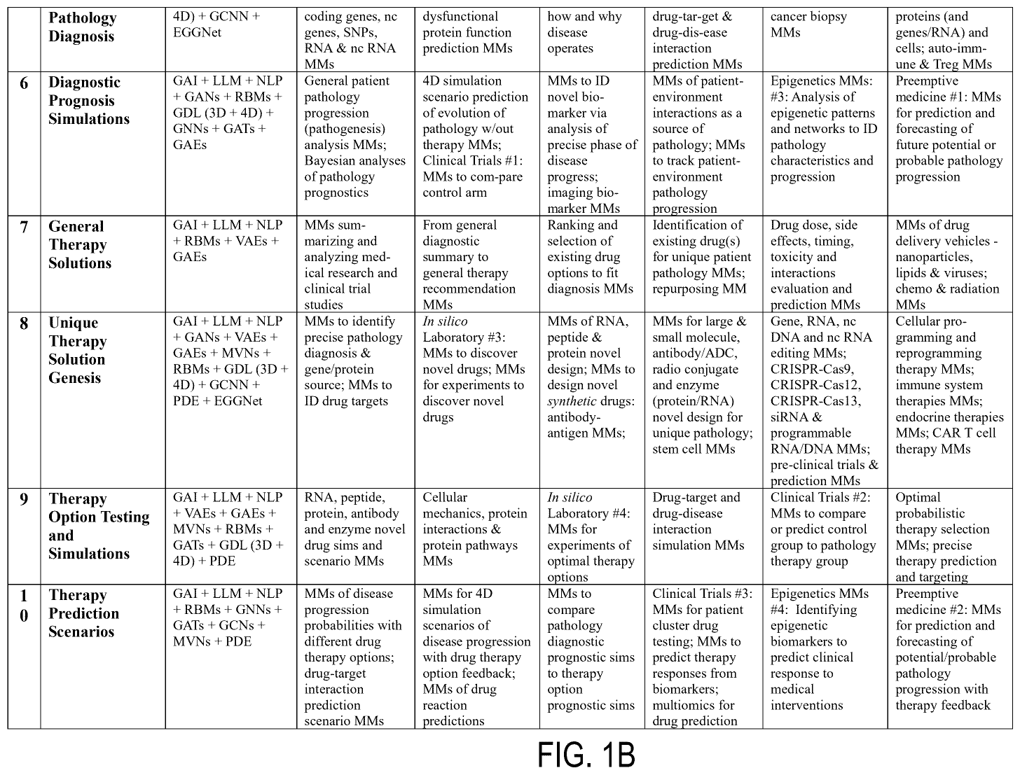

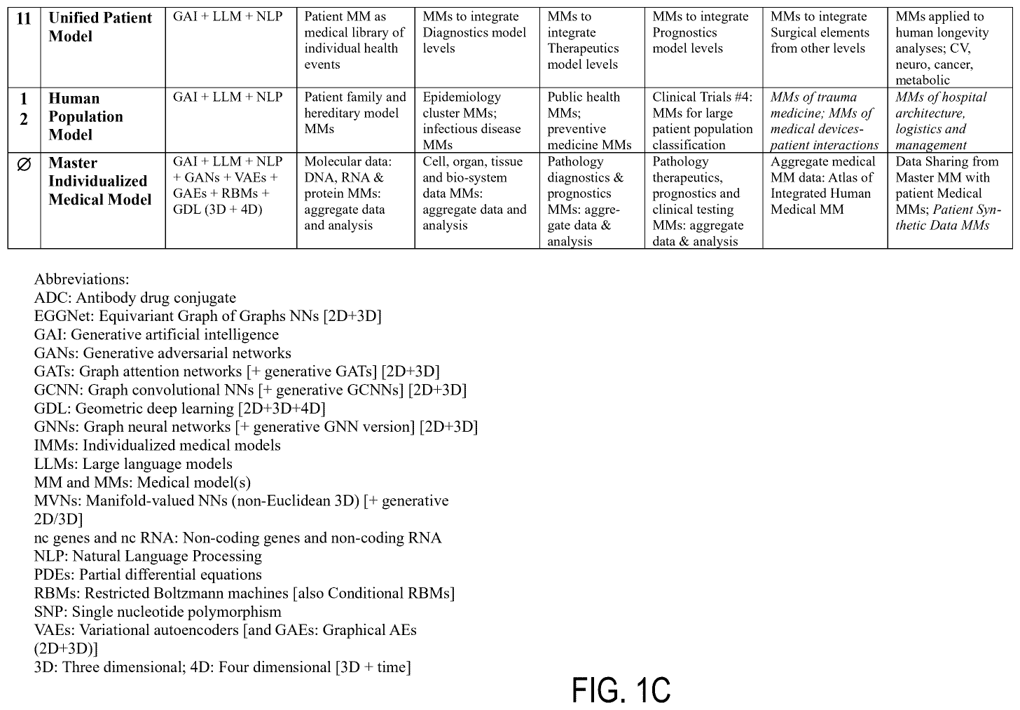

The individualized medical modeling system architecture consists of thirteen levels delineating diagnostic, prognostic, therapeutic and general medical types. Within the IMM modeling typology described in the thirteen levels are several layers within each level. These layers further describe detailed components of the categories—precise and varied biological ontological object references—in each level that represent a differentiated grouping of biomedical objects, events, systems, predictions or therapies. Please refer to the table in FIG. 1 for a consolidated description of the IMM architecture. In addition to disclosing the main categories of the IMM system, the architecture describes a set of AI technologies that are applied to the categories of each level. The table in FIG. 1 shows these AI technology applications to the various biological entities and systems.

### Level 1: General Patient Model

Level 1 of the individualized medical model (IMM) architecture refers to the general patient model. This level consists of MM categories in six layers. Layer 1 refers medical research article and biomedical library data search and analysis MMs. Vast databases of biological and medical libraries, including medical research articles and biodata consisting of DNA, RNA, protein, cell and disease data, are available for search and analysis by researchers. These data on generic medical information are available in MMs. The MM models apply LLMs and NLP to summarize the medical articles and biological data for use by clinicians and researchers.

Layer 2 consists of doctor observations of patient and notes data that are imported into MMs. Most medical doctors use electronic medical records (EMR) in order to track information about patients. These direct physician medical notations about patient visits, tests and events are recorded in EMR and input into MMs. This layer also includes the integrated health record platform. The MMs apply LLMs and NLP to summarize the patient medical records.

Layer 3 consists of electronic health record (EHR) data inputs, aggregation and analytics MMs. The EHR records include multiple EMR records from at least one physician that are aggregated into the IMMs. These EHR records are useful by feeding specific patient medical information into MMs. The MMs apply LLMs and NLP to summarize, organize and manage the patient medical records. This layer also includes the integrated health record platform.

Layer 4 consists of patient history and hereditary data input into MMs. The patient history is useful to provide a background to the present patient medical events. The patient hereditary data is useful to supply medical data that may explain a patient medical situation. These historical and hereditary data supply critical explanatory data for understanding the context for some chronic diseases.

Layer 5 consists of MMs on patient medical tests, including blood, urine, spinal fluid, body fluid and tissue tests. Since collecting and analyzing blood and fluid tests are common, these data should be easily accessible. The analysis of these tests is supplied to the IMMs in order to show the present condition of a patient. In most cases these data supply a baseline reference for the MMs, while in other cases some data are identified that suggest a unique abnormality that may require further testing and analysis.

Layer 6 consists of Epigenetic MMs #1. In this case, epigenetics refers to an analysis of environmental factors of biology. Further, this layer includes chemical poison MMs that illustrate external environmental chemical pathogens that have an adverse reaction on a patient. An example of an environmental factor of disease generation may be a patient smoking cigarettes or being exposed to a toxin.

### Level 2: Diagnostics, Bioinformatics, Organ and Body System Analyses

Level 2 consists of seven layers that address MMs showing diagnostics, anatomy, physiology and bioinformatics of patient biological data including imaging data, body system data, electrical system data and organ data.

Layer 1 consists of MMs that collect, analyze and manage patient biological data, including genomic, proteomic, multiomic, metabolomic and cell biomarker data. GenAI, LLMs, NLP, GANs and GDL may be applied to analyze these patient biological data.

Layer 2 consists of diagnostic imaging data input into MMs. These imaging data types include X-rays, MRI, CT, ultrasound, mammography, PET, SPECT and so forth. In addition, imaging biomarkers data are recorded in these MMs. GenAI, LLMs, NLP, GANs and GDL may be applied to analyze these patient diagnostic imaging data.

Layer 3 consists of body system MMs. These include MMs that collect information on, and analyze, patient data on the cardiovascular system, neuro- and nervous system, pulmonary system, muscular-skeletal system, respiratory system, gastro-intestinal system, reproductive system, immune system, lymph system, endocrine system and so on. GenAI, LLMs, NLP, GANs and GDL may be applied to analyze these patient body systems data.

Layer 4 consists of electrical systems analysis in MMs. MMs of brain, nervous and autonomic system electrical activity are analyzed on this layer. In addition, MMs of cardiovascular system electrical activity are analyzed on this layer. Further, medical devices related to body electrical systems, such as pacemakers or defibrillators are addressed in this layer. Implantable brain stimulation devices (neural implants) are also addressed in this layer. This layer also deals with digital biomarkers, including sensor-derived electrical or temporal data on cardiac or brain activity or condition. GenAI, LLMs, NLP, GANs and GDL may be applied to analyze these patient electrical systems data.

Layer 5 consists of MMs that examine organ and tissue data including patient data on brain, heart, lungs, liver, pancreas, kidneys, glands, reproductive organs, skin, eyes, etc. This layer also deals with MMs that examine tissue consisting of clusters of cells in, and connective tissue between, the organs and body systems. This layer also deals with physiology MMs that deals with operational mechanics of organs and tissue. GenAI, LLMs, NLP, GANs and GDL may be applied to analyze these patient organs and tissue data.

Layer 6 consists of MMs that address medical devices, including medical devices in body systems. MMs that deal with medical devices draw on modeling software techniques from industrial design to configure the anatomy and mechanics of medical devices. These medical devices may be implantable devices or external devices. As the internet of things (IoT) presents intelligent devices that include sensors, intelligent circuits and communications components, these IoT devices are increasingly integrated into medical technologies that interface with medical patients. These medical devices track, analyze and manage patient diseases, activities for which IMMs are well suited to analyze and organize. For implantable devices, MMs are well suited to model, track and manage data for these devices. Layer 6 is also useful for modeling, tracking and managing artificial organs. GenAI, LLMs, NLP, GANs and GDL may be applied to analyze these patient medical device and artificial organ data.

Layer 7 consists of surgical MMs. Surgeons use MMs for understanding each patient's unique surgical requirements. For instance, an orthopedic surgeon may use MMs to analyze a patient's diagnostic imaging information on a knee prior to knee replacement surgery. The precise contours and configuration of a patient's unique artificial knee are constructed by applying the patient IMMs. GenAI, LLMs, NLP, GANs and GDL may be applied to analyze these patient surgical data. Surgical procedures are analyzed prior to intervention by applying the MMs. The mechanics of the surgical procedures can be emulated by applying simulations of projected surgeries. In an embodiment of the invention, a surgical MM can be combined with a virtual reality or an augmented reality program in order to enable a surgeon to study a patient's surgical procedure to prepare for the actual event. In another embodiment, the MMs can be useful during a surgical procedure by advising the surgeon of potential procedural steps at different stages in order to optimize a surgical solution. GenAI, LLMs, NLP, GANs and GDL may be applied to analyze these surgical MMs.

### Level 3: Molecular and Cellular Description and Analyses

Level 3 consists of seven layers that address MMs illustrating cellular and molecular elements of medicine.

Layer 1 consists of MMs delineating DNA, chromosome, SNPs, coding genes and non-coding genes data, collectively embraced in genomics. The models describe a map of chromosomes with specific coding and non-coding genes as represented by unique addresses and zip codes.

In addition to genomic data, layer 1 also features MMs of embryonic development. Thousands of genes are utilized in the embryonic process that are emulated and analyzed in model representations of fetal growth.

Layer 2 consists of MMs of coding and non-coding RNA and describes transcription processes from DNA to RNA. Coding and non-coding DNA are transcribed into coding and non-coding RNA, the processes of which are illuminated in MMs. This layer examines MMs of multiomics from DNA to RNA and to proteins. In addition, this layer describes general cell biology anatomy and dynamics.

Layer 3 consists of MMs of protein and peptide MMs. The translation from RNA of amino acids into peptides and proteins are described. This layer examines MMs of proteomics.

Layer 4 consists of MMs showing 3D and 4D cell architecture, including model representations nucleus, mitochondria, ribosomes, lysosomes and so on. This layer features molecular interactomics.

Layer 5 consists of MMs of 3D and 4D cell dynamics, including model representations of physiology of cell types including neuron, blood, muscle, stem cells, immune cells and so on. This layer features cellular interaction MMs as well.

Layer 6 consists of multicellular network MMs. This layer features descriptions of tissues, cellular network interactions and intercellular communications.

Layer 7 consists of MMs and simulations showing pathogens, vaccines and the immune system. This layer also features MMs of biologics.

Level 3 features GenAI, LLM, NLP, VAEs, GAEs, GNNs and GDL categories of AI applied to molecular and cellular descriptions of objects, events and systems.

### Level 4: Structural Genetic Variant Combination Pathology Identification

Level 4 describes the sources of pathology in the form of combinations of structural genetic variants. Since many diseases develop from mutated genes, the combination of these gene variants presents as aggregations of dysfunctional protein structures and pathways. This level develops models that transcend traditional protein folding models because we are concerned here with mutated genes and abnormal proteins that are generated from the gene variants. In fact, models at this layer are compared to optimized reference protein structure models in order to best describe the genesis of pathologies.

Layer 1 consists of MMs of mutated and variant genes and single nucleotide polymorphisms (SNPs). Each SNP represents a variation in a single DNA nucleotide. As an example, a gene may replace guanine with adenine, creating an error in the DNA segment. If SNPs occur in one in every 1,000 nucleotides, there are about four million SNPs in each individual's genome, including non-coding genes. When SNPs are integrated in coding genes, they may play a role in diseases because they may generate abnormal proteins; when SNPs are located between genes, that is, in the non-coding regions of a chromosome, they may be useful as markers of genes that may generate disease. Since SNPs may be inherited, they may be useful to tracking inherited genes.

Layer 2 consists of MMs of dysfunctional proteins and peptide structures. This layer is also useful for generating MMs for protein structure prediction. Specifically, this layer includes MMs that describe a particular patient's dysfunctional 3D protein structures. These dysfunctional protein structures are compared to reference libraries of 3D protein structures in order to reveal protein folding and structural abnormalities that are inherent in a specific patient's disease.

Layer 3 consists of MMs of DNA, RNA, protein, lipid and small molecule biomarker MMs. Biomarkers—literally biological markers—represent biomolecular information that can be used to identify, predict, validate and monitor a disease. Gene, RNA and protein biomarkers are structural biochemical components that are useful in identifying the presence of a biomedical dysfunction. Lipid and small molecule (metabolomic) biomarkers represent additional biochemical components that supply information on the presence of a disease. Biomarkers are also useful to identify the reaction of drugs, to predict the progress of a disease or to predict a response to a drug therapy. Layer 3 also contains MMs of liquid biopsies for cancer detection.

Layer 4 consists of MMs delineating the cellular manifestation of dysfunctional DNA, RNA, proteins and peptides. While mutated gene and RNA sequences present as abnormal 3D protein and peptide structures, their representation can be shown in the context of a living cell. These MMs show the operation of abnormal proteins in protein pathways. In addition, this layer contains MMs that show tumor tests. Tumors manifest a combination of multiple gene mutations, which are described in models.

Layer 5 delineates in silico laboratory in which MMs can be used to elaborate experiments of dysfunctional genes, RNA and proteins. In silico laboratory experiments of gene and RNA variants and abnormal proteins enable the MMs to computationally apply hypotheses to test assumptions and to provide predictions of aspects of abnormal genomic and proteomic data.

Layer 6 describes epigenetics MMs, specifically, models showing mechanisms of gene expression regulation. In addition, this layer shows allergy MMs. Finally, this layer describes animal and pre-clinal trial MMs.

Level 4 applies AI technologies to the categories in its layers, including GenAI, LLMs, NLP, GANs, VAEs, GAEs, MVNs and GDL.

### Level 5: Functional Molecular and Cellular Pathology Diagnoses

While level 4 is concerned with MMs describing structural biomolecular phenomena, level 5 consists of MMs that describe functional molecular and cellular phenomena that lie at the root of pathologies.

Layer 1 contains functional (dynamic) models of dysfunctional coding and non-coding genes, SNPs and coding and non-coding RNA. In addition to delineating 3D structures of dysfunctional genes and RNA, this layer also describes 4D processes of transcription of DNA to RNA.

Layer 2 consists of MMs of dysfunctional protein and peptide functions, including dysfunctional protein function prediction MMs. This layer shows MMs that describe the functional operation of the process of translation from RNA to abnormal proteins and peptides.

Layer 3 consists of MMs describing protein pathway mapping MMs. In this layer are descriptions of how and why disease operates, including the interactions of abnormal proteins with both normal proteins, lipids and small molecules as well as abnormal proteins, lipids and small molecules. In one embodiment, this layer shows MMs that compare abnormal protein pathway and interaction operation with normal protein pathways and operations.

Layer 4 consists of MMs showing protein-protein, protein-ligand and protein-lipid interactions, including both functional and dysfunctional protein configurations. This layer also shows drug-target and drug-disease interaction prediction MMs. Dysfunctional proteins are targets of drugs, the interactions of which MMs are useful for describing; MMs are also used to predict abnormal protein interactions with drug candidates. Similarly, multiple dysfunctional proteins that represent disease are mapped in MMs as they interact with drugs; drug candidate behavior is predicted in MMs as the drug candidates interact with multiple abnormal proteins.

Layer 5 consists of MMs showing cellular machinery dysfunctions. Since proteins operate as the molecular machinery of cells, abnormal proteins are described by MMs in the context of cellular mechanics. In addition to intracellular cellular machinery dysfunctional operation, MMs are useful in this layer to show dysfunctional intercellular operations, including cell signaling and intercellular communications. Cancer biopsies are also modeled in layer 5. Since tumors represent the manifestation of multiple dysfunctional genes and abnormal proteins, cancer biopsies can be modeled at layer 5.

Layer 6 consists of MMs showing an in-silico laboratory in which MMs describe experiments involving dysfunctional genes, RNA, proteins and cells. In addition, auto-immune and regulatory T-cell (Treg) MMs are modeled at this layer.

Level 5 applies AI technologies to the categories in its layers, including GenAI, LLMs, NLP, GANs, VAEs, GAEs, MVNs, GATs, GDL and GCNN.

### Level 6: Diagnostic Prognostic Simulations

Level 6 delineates MMs showing the progress of disease without intervention. Diagnostic prognosis MMs show actual disease progress as well as predictions of disease progress over time.

Layer 1 consists of MMs analyzing general patient pathology progression (pathogenesis). Bayesian analyses are applied to pathology prognostics in this layer to assess patient pathology progression scenario probabilities.

Layer 2 consists of MMs that represent 4D simulation scenario prediction of pathology evolution without therapy. In a sense, these MMs represent a control aspect of a patient's disease progress without treating the disease. Also in this layer are MMs on the control arm of drug clinical trials in order to show patients' pathology progress without treatment.

Layer 3 consists of MMs to identify novel biomarker(s) via analysis of the precise phases of disease progress. As a patient's disease evolves, different biomarkers are generated at different phases of the pathology progression. These differentiated biomarkers represent unique guideposts that point to the specific points of change in the evolution of the patient's disease. Imaging biomarker MMs are also found at this layer. As a patient's pathology progresses, these imaging data represent key guideposts for identifying the phases of the evolution of the disease. As an example, the imaging biomarkers identified in the multi-phasal development of some solid tumors represents a diagnostic prognostics tool.

Layer 4 consists of MMs of models depicting patient-environment interactions as a source of pathology. For example, an MM can track a patient's excessive eating, drinking and smoking as a source of disease. This layer also includes MMs to track patient-environment pathology progression. As an example, if a patient contracts lung cancer but maintains smoking cigarettes, the progression of the disease may be different than if the patient quits smoking.

Layer 5 consists of epigenetics MMs. These MMs analyze epigenetic patterns and networks to ID pathology characteristics and progression. For example, some diseases involve a complex interchange between a patient and her environment; some food allergies may generate from eating (or not eating) foods at different times in childhood.

Layer 6 consists of preemptive medicine MMs. These MMs predict or forecast future potential or probable pathology progression. An assessment of biomarkers for cardiovascular disease risk may result in recognition of future probable onset of the disease that is highly predictable.

Level 6 applies AI technologies to the categories in its layers, including GenAI, LLMs, NLP, GANs, RBMs, GDL, GNNs, GATs and GAEs.

### Level 7: General Therapy Solutions

MMs are shown for general therapies that may already be known and tested. Once a diagnosis of a disease is made, the first step is to identify general therapeutic solutions of existing drug therapies.

Layer 1 consists of MMs for summarizing and analyzing medical research and clinal trial studies. The accumulated sum of prior medical research articles supplies a robust knowledge pool for physicians to use to identify and solve clinical medical challenges.

Layer 2 consists of MMs that include general therapy recommendations of general diagnostic summaries. These general therapy suggestions refer to existing remedies that are likely to solve specific disease diagnoses.

Layer 3 consists of MMs that rank and select existing drug options to fit disease diagnoses.

Layer 4 consists of MMs for the identification of existing drug(s) for unique patient pathologies. Whereas prior layers suggest a common disease type, this layer focuses on identifying an existing drug therapy for a novel patient disease.

Layer 5 consists of MMs exploring, evaluating and predicting the precise dose, side effects, timing, toxicity and interactions of drugs for a patient.

Layer 6 consists of MMs showing options for drug delivery vehicles, including nanoparticles, lipids and viruses. In addition, this layer includes MMs showing applications of chemotherapy and radiation as therapeutics.

Level 7 applies AI technologies to the categories in its layers, including GenAI, LLMs, NLP, RBMs, VAEs and GAEs.

### Level 8: Unique Therapy Solution Genesis

Many diseases have a genetic source. In some cases, genetic mutations generate abnormal proteins that cause a disease. In other cases, epigenetic expression creates abnormal proteins, lipids or small molecules that cause disease. Most chronic diseases, from cardiovascular disease to neuro-degenerative diseases to cancer and autoimmune diseases have genetic or epigenetic sources. In these cases, it is essential to precisely diagnose a patient's pathology. However, once the molecular mechanisms of a disease are identified, it is clear that a uniquely tailored drug or therapy may be required to cure or manage the disease. The modeling categories of level 8 are intended to identify and select unique therapy solutions.

Layer 1 consists of MMs to identify precise pathology diagnosis and gene and protein sources of a patient's disease, which are critical preparatory data in order to prepare to address a patient's disease. MMs to identify drug targets are also included in layer 1.

Layer 2 consists of in silico laboratory MMs to discover novel drugs. In this layer, MMs are configured to conduct experiments to discover novel drugs. The in silico laboratory is configured to test, rank and select different chemical compounds that may comport to a specific drug target.

Layer 3 consists of MMs of RNA, peptide and protein (drug) novel design to solve a unique pathology. MMs are configured to apply GenAI techniques in order to generate novel synthetic drugs in order to fit a particular dysfunctional protein. In addition, antibody-antigen MMs are included in this layer.

Layer 4 consists of MMs for large and small molecule novel design for a unique pathology. In addition, antibody/ADC radioconjugate novel drugs for unique pathology are included at this layer. Enzyme (protein or RNA) novel designs for unique pathology are included at this layer. Finally, stem cell MMs are included at this layer.

Layer 5 consists of MMs for gene, RNA, non-coding DNA and non-coding RNA editing. CRISPR-Cas9, CRISPR-Cas12, CRISPR-Cas13 (gene silencing) and programmable RNA/DNAMMs are included at this layer. Pre-clinical trials and drug prediction MMs are also included at this layer.

Layer 6 consists of MMs illustrating cellular programming and reprogramming therapy. In addition, immune system therapy MMs are featured at this layer. For example, CAR T cell therapy MMs are included. Finally, endocrine therapy MMs are included at this layer.

Level 8 applies AI technologies to the categories in its layers, including GenAI, LLMs, NLP, GANs, VAEs, GAEs, MVNs, RBMs, GDL, GCNN and PDE.

### Level 9: Therapy Option Testing and Simulations

Level 9 features testing and simulations of various therapy options, from chemical to biochemical and biologic.

Layer 1 consists of MMs for RNA, peptide, protein, antibody and enzyme novel drug simulations and scenarios.

Layer 2 consists of MMs of cellular mechanics, protein interactions and protein pathways.

Layer 3 consists of MMs of in silico laboratory MMs for experiments of optimal therapy options. In some cases, the MMs evaluate various therapy options, rank the options and select the optimal therapeutic option for a specific set of conditions.

Layer 4 consists of MMs for drug-target and drug-disease interaction simulations.

Layer 5 consists of MMs illustrating clinical trials in order to compare or predict a control group to a pathology therapy group.

Layer 6 consists of MMs showing optimal probabilistic therapy selection MMs. These MMs describe precise therapy (drug) prediction and targeting.

Level 9 applies AI technologies to the categories in its layers, including GenAI, LLMs, NLP, VAEs, GAEs, MVNs, RBMs, GATs, GDL and PDE.

### Level 10: Therapy Prediction Scenarios

Diagnostic prognostics include predictions made after a disease is identified and tracked without therapeutic intervention. When a disease is treated, on the other hand, with a drug, changes in the disease evolution can be tracked with therapeutic prognostics. The disease scenarios with treatment (pharmacodynamics) are compared to disease (prediction) scenarios without treatment in order to assess the effectiveness of a drug or therapeutic regimen.

Layer 1 consists of MMs of disease progression probabilities with different drug therapy options. In addition, this layer includes MMs of drug-target interaction prediction scenarios.

Layer 2 consists of MMs for 4D simulation scenarios of disease progression with drug therapy option feedback. This layer includes MMs of drug reaction predictions.

Layer 3 consists of MMs for comparing pathology diagnostic prognostic simulations to therapy option prognostic simulations.

Layer 4 consists of MMs of clinical trials for patient cluster drug testing. These clinical trials tailor drug testing to include a focus on pathologies in which patients share specific genetic, RNA or protein biomarkers of specific diseases. In addition, this layer includes MMs to predict therapy responses from biomarkers. MMs in this layer also address multiomics for drug prediction.

Layer 5 consists of MMs for epigenetics in which epigenetic biomarkers are identified to predict clinical responses to medical interventions.

Layer 6 consists of MMs for preemptive medicine in which the MMs are configured to predict or forecast potential or probable pathology progression with therapy feedback.

Level 10 applies AI technologies to the categories in its layers, including GenAI, LLMs, NLP, RBMs, GNNs, GATs, GCNs, MVNs and PDE.

### Level 11: Unified Patient Model

In this level, the various levels are united into an integrated patient MM.

Layer 1 consists of MMs that view a patient as a medical library of individual health events.

Layer 2 consists of MMs that integrate diagnostics model levels.

Layer 3 consists of MMs that integrate therapeutics model levels.

Layer 4 consists of MMs that integrate prognostics model levels.

Layer 5 consists of MMs that integrate surgical elements from other levels.

Layer 6 consists of MMs applied to human longevity analyses, including cardiovascular disease, neurodegenerative diseases, cancer and metabolic diseases.

Level 11 applies AI technologies to the categories in its layers, including GenAI, LLMs and NLP.

### Level 12: Human Population Model

This level is useful for addressing public health and epidemiological models as well as models involves aspects of hospital system management.

Layer 1 consists of MMs modeling a patient's family and hereditary data.

Layer 2 consists of MMs of epidemiology clusters and MMs of infectious diseases.

Layer 3 consists of MMs for public health data analysis. This layer includes preventive medicine MMs.

Layer 4 consists of MMs featuring clinical trials that classify large patient populations.

Layer 5 consists of MMs of trauma medicine detailing simulations of emergency events. This layer also features MMs showing interactions between patients and medical devices.

Layer 6 consists of MMs of hospital architecture, logistics and management. This layer develops models to optimize the structure and function of hospitals and clinics.

Level 12 applies AI technologies to the categories in its layers, including GenAI, LLMs and NLP.

### Level Ø: Master Individualized Medical Model

Level Ø unifies different elements of the MMs to create aggregated reference models.

Layer 1 consists of molecular data MMs in which DNA, RNA and protein data are aggregated and analyzed.

Layer 2 consists of MMs modeling cell, organ, tissue and biosystem data in which these data types are aggregated and analyzed.

Layer 3 consists of MMs of aggregated pathology diagnosis and prognostics data and analyses.

Layer 4 consists of MMs of aggregated pathology therapeutics, prognostics and clinical testing data and analysis.

Layer 5 consists of MMs of an Atlas of Integrated Human Medical modeling. This layer includes MMs of aggregated medical data that can be used as a reference.

Layer 6 consists of models that share data from the Master MMs with patient MMs. This layer also includes models of patient synthetic data.

Whereas many of the Levels and categories are configured to analyze specialized biomedical problems, Level Ø is unique in its ability to compare actual patient pathologies to a model-generated optimal patient scenario in which the patient's best possible health—without addictions, unhealthy habits, lack-of-exercise, etc.—can be described, along with best-case prognostics under conditions contingent on the patient maintaining excellent health. The optimized version of the patient can be used as an idealized possible reference scenario.

Level Ø applies AI technologies to the categories in its layers, including GenAI, LLMs, NLP, GANs, VAEs, GAEs, RBMs and GDL.

### Functional Dynamics Between MM Categories

Different levels in the IMM architecture system interact. First, diagnostic levels exchange data between molecular, cellular, organ, tissue and body system categories. Molecular and cellular categories exchange data, i.e., Level 3 layers 1-3 may exchange data with Level 3 layers 4 and 5. Layers on each level can exchange data in order to complete MMs on each respective level.

Level 3 layers exchange data with Level 4 layers. Similarly, Level 4 layers share data with Level 5 layers.

Diagnostic Levels 1 and 2 share data with each other. Levels 1 and 2 share data with diagnostic Levels 3-5 and with diagnostic prognostic Level 6 and therapeutic prognostic Level 10.

Diagnostic Levels 3-5 share data with diagnostic prognostics Level 6.

Therapeutics Levels 7, 8 and 9 share data as well. Levels 7 and 8 share data with Level 9.

Therapeutics Levels 7-9 share data with therapeutic prognostics Level 10.

Levels 1-10 share data with Level 11 as well as with Level Ø.

The sharing of data between MM categories enables the IMM system to solve complex problems by gathering more precise information about a patient's precise diagnosis and therapeutic options from various biological or biochemical data sources.

As an example of the data exchange between categories on the same or on different levels, an MM can build simulations on different scales, that is, between molecular scales, cellular scales and tissue, organ and biological system scales. This approach enables the IMM system to process different dimensions of biomedical discovery simultaneously.

In another example, while diagnostics level models may generally be developed before therapeutic level models, the system may develop interactive dynamics between these diagnostics and therapeutics categories in order to generate complementary models simultaneously. For instance, an early diagnostic model may lead to an initial Level 7 therapeutic model, while feedback from this treatment recommendation may lead to deeper analysis of MMs in diagnostics Levels 4 and 5, which then lead to analysis of MMs of therapeutics Level 8 and therapeutic prognostics Level 10. These models may be interactive and iterative.

In an embodiment of the invention, software agents (PHAs) facilitate the exchange of data between MM categories and between MM layers on the same level and between levels.

In another embodiment, the software agents can simultaneously process data exchanges between two or more MM categories between MM layers on the same level and between levels.

The simultaneous processing of multiple MMs resembles the multiprocessing of multiple microprocessor or system-on-chip cores. The multiple simultaneous processing of two or more MMs or simulations enable solving multiple biological problems at the same time.

As more information is gathered in early diagnostic Levels, the data are able to complete complex diagnostic MMs that precisely identify a pathology source. From this realization of a disease cause, therapeutic model MMs can develop drug therapy candidates to target specific abnormal genes or proteins.

In a sense, the categories in layers of some levels represent a reduction from macro biological phenomena (bio systems and organs) to micro biological phenomena (cells, proteins and genes).

In an embodiment of the invention, software agents apply application programming interfaces (APIs) to intra-level and inter-layer MM analyses. The APIs operate to process data on one or more levels and to connect the levels together with communications software. In addition, the APIs can connect layers, layer categories and layer sub-categories, within levels.

Since each layer can have its own MMs, numerous MMs can operate at the same time, sharing data with other MMs and communicating with other MMs. Data can be aggregated among two or more categories or layers simultaneously. Similarly, data from two or more categories or layers may be combined together in order to construct two or more models simultaneously, mainly by sharing data between the two or more categories or layers. In one interpretation, models from one or more category can be conceived as analyzing a specific episode of a patient phenomenon (molecular, cellular, organ, etc.), while combining data from two or more categories or layers enables the modeling system to identify complex patterns among the sequence of events. By combining two or more MMs from two or more categories, the modeling system can identify the signal from the noise in order to articulate a long-term patient health story.

As an example, one or more software agents can engage in diagnostic problem solving by activating MMs in different layers of diagnostic Levels 3-5. Once diagnostics models are constructed that precisely illustrate a patient's disease, the software agents engage in therapeutic problem solving by activating MMs in different layers of therapeutic Levels 7-9. The software agents can activate MMs in the diagnostic prognostics Level 6 and the therapeutic prognostics Level 10 in order to make predictions about pathology development with and without drug interventions.

Note that there are sub-categories in each layer indicating that there may be hundreds of potential MM categories available in the IMM system.

Once the MM category models are activated, with problems solved in multiple layers, the system will generate summaries of analyses, options and predictions.

The software agents may navigate this maze of MM categories with an aim to streamline the disease discovery and therapy problem solving processes.

The different categories of MMs focus on different biomedical phenomena, such as molecular, cellular, organ or biosystem objects or events. Each category of biomedical phenomena may require a different class of AI or ML in order to optimize the descriptive, predictive or prescriptive capabilities of each category of the IMM system. Different MMs will select a different particular AI or ML algorithm in order to apply to different types of biomedical phenomena or to solve different categories of problems. Each MM will match a different AI or ML algorithm or technique from the AI/ML toolkit in order to apply to the different categories of biomedical phenomena. When PHAs build MMs, the PHAs select different AI or ML algorithms (or configure hybrid AI or ML algorithms) in order to describe biomedical categories or solve key biomedical category problems. AI and ML tools can be applied to two or more categories for simultaneous problem solving.

### Mechanics of Individualized Medical Modeling System

Computer modeling involves processing program code (software) on computer hardware that consists of logic and memory circuits. The logic circuits that process computer models consist of multi-core system on chip (SoC) circuits, graphics processing units (GPUs), tensor circuits, complex programmable logic devices (CPLDs), field programmable gate arrays (FPGAs), application specific integrated circuits (ASICs) and neuromorphic circuits. Many of these circuits are fabricated in specialized integrated circuit manufacturing facilities in 10 nm, 7 nm, 5 nm, 4 nm, 3 nm, 2 nm and 1.8 nm nodes. While logic circuits have traditionally been configured in planar formats, increasingly these complex logic circuits are being integrated in three dimensional packages akin to a multi-story building. Intel's Foveros Direct, Samsung's SAINT and TSMCs SoIC-X and SoW 3D chip packaging technologies represent the cutting edge of integrated circuit fabrication. Although there are circuits today consisting of over 200B transistors, these technologies are projected to produce single circuit packages in the near future consisting of trillions of transistors.

In addition to logic circuits, memory circuits are required for the operation of computer modeling. In the main, advanced modeling programs require DRAM technologies that work with complex logic circuits. The most advanced DRAM technology, high bandwidth memory (HBM), is stacked in layers in 3D packages. The HBM packages are bundled with advanced logic circuits, including both SoCs and GPUs, to create high performance computing hardware.

In recent years, the advanced GPUs from Nvidia and AMD have powered the large language models consisting of trillions of parameters. These GPUs require many HBM memory modules and are typically configured in clusters of dozens or hundreds of circuits. Much of the GenAI revolution depends on the performance of these GPUs to process massive data sets. GPUs are particularly adept at processing modeling data, for example, for advanced simulations. While GPUs are a primary tool for training LLMs, GPUs and FPGAs are often applied to inference tuning of LLMs. SoCs are involved in all aspects of the training and inference of LLMs and often work together with GPUs.

### Software for IMMs

There are number of general LLMs for GenAI applications. These include OpenAI's Chat GTP 4 (1.75T parameters) GTP 4o (200B parameters) and Chat GPT 5 (50T), Meta's Llama 3 (405B parameters), Anthropic's Claude and many others. These LLMs are trained in several phases. In a preparatory phase, data are collected and input into the model. In the next phase, the model is trained with self-supervised learning in which deep learning frameworks are configured and parameters are defined. In the third phase, once the data parameters have been cleaned and refined, the model is trained with supervised learning for instruction tuning. This phase redistributes parts of the data for parallel processing in GPUs. In the next phase, the model is trained in reinforcement learning to encourage positive conduct and to fine tune the data sets. Finally, once the LLM has been trained, it must be applied to a particular field with inference engines. In most cases, the LLMs are tuned with text for natural language processing. Since the synthetic data that are generated from the LLMs are dependent on data inputs and training analyses, it is important to have quality data imported into the models at the outset in order to support quality data outputs.

There are a number of significant biological LLMs, though they rarely feature more than 10B parameters and more frequently feature a billion parameters or less, suggesting their limits. The main biomedical LLMs include SciBert, BioBert, BioNLP, BioMegatron, PubMedBERT, ScholarBERT, DARE, BioGPT and BIoGPT-JSL, Galactica, BioMistral, MedLM, Meditron, Hippocratic AI, AntGLM-Med-10B, PaLM-2 and Med-PaLM-2. BERT, an acronym for bidirectional encoder representations from transformers, was introduced in a 2018 article on pre-trained bidirectional transformers applied to natural language processing, and utilized in LLMs in biology, medicine and data science.

LLMs have blind spots. First, since it takes months or years for an LLM to train data before they are prepared to generate novel data, the data are inherently obsolete. The larger the model, the greater the data set, the longer the training period, the more obsolete the data. Second, the data generated from the LLMs have a high rate of unreliability. This is typically called the problem of hallucination in which the LLM generates blatantly false results. Finally, there are varying degrees of precision, completeness and specialization in LLMs. If an LLM has trillions of parameters, it is likely not specialized, while if it has fewer than half a billion parameters it is likely not providing a complete answer.

Computer models require database software for data storage and data management. Database management systems feature structured and unstructured data. Database types include relational databases and object-relational databases. In particular, LLMs can work with SQL databases for managing and querying relational databases. Major databases are produced by IBM, Microsoft, Oracle and open-source companies.

In order to solve problems of LLM data obsolescence, vector databases are increasingly used for advanced LLMs. Encoded data are embedded into vectors. Storing data in vectors allows capturing meanings and context of similar data. Vector databases store, manage and search embedded vectors in order to optimize retrieval of familiar or relevant data in similarity searches, thereby accelerating processing efficiency. Vector databases are useful for processing data updates in LLMs, like a patch that supplements the original, aged, data sets. Consequently, vector databases are faster and more responsive than traditional relational databases. Vector databases work with retrieval augmented generation (RAG) approaches to apply contextual information to data processing in LLMs. While vector databases supply an alternative to traditional relational databases, their application is specialized.

Another advanced database type that is applied to machine learning is three-dimensional (3D) databases or multi-dimensional databases. In addition to the typical two X and Y dimensions of a relational database that stores data in tables, a three-dimensional database stores data in tables with the three dimensions of X, Y and Z. Data are stored by attribute. 3D databases may require a longer set-up period, but typically process data faster, while it is also easier to tune than on a traditional relational database. Consequently, 3D databases are optimized for data with complex object attributes. Additional dimensions can be theoretically developed in advanced multi-dimensional databases.

Application program interfaces (APIs) are software programs that act as a connective tissue that link computational elements. APIs are a set of rules or protocols to allow software apps to communicate or exchange data. APIs enable only the exchange of essential data between separate apps so as to maximize network efficiency and security. For example, APIs may be applied to IMM system categories in which each category represents a model application. In one case, intra-level MM categories can communicate with each other via APIs, which represent a sort of short-cut to share data or supply a particular analysis. Similarly, inter-level MM categories can communicate with each other via APIs in order to share data or institute a particular analysis. In this sense, APIs are a sort of enzyme for MM inter-category communication and operation that supplies an accelerated short-cut for processing data within, or between, specified categories. In addition to interfacing with MM categories, APIs interface with software agents, databases, LLMs, electronic medical records, electronic health records, integrated health records and patient relationship management software.

In an embodiment of the invention, computer models can be operated, accessed and activated with voice control. For instance, some functions of model building can be operated via voice activation or speech recognition.

Computer models process data in one or more computer platforms. These platforms include desktop and laptop computers, smart phones, edge (tablet) computers, workstations, cloud data centers and IoT devices. While most personal computers, tablet computers and smart phones include SoC logic circuits, cloud computers include SoCs, GPUs, FPGAs, ASICs, tensor circuits, neuromorphic circuits and neural circuits. The desktop/laptop computer interaction with cloud computing will likely be a major paradigm for processing data in computer models.

Among the major AI programming languages used by advanced computer modeling programs are Python, C++, Java (particularly Java Script) and SQL.

Rosetta and MatLab provide software programming tools for computer software programs.

The IMM system involves processing medical data types. These medical data types include medical diagnostic imaging data, genomic data, RNA data, proteomics data, multiomics data, metabolomics data, cellular data, biomarker data, patient EHR data, scientific articles literature, biological data as a translation to text and alphanumeric data. The data sources for MMs include data involving medical research articles, gene, RNA and protein databases, personal patient EMR, generalized EHR data, integrated health record platform data, patient medical tests and biomarkers, animal and preclinical drug research data and clinical research data.

### AI and ML Applied to IMMs

On Nov. 30, 2022, OpenAI introduced Chat GPT 3, an event that initiated a revolution in AI technology. While the subsequent development of generative AI has precipitated phenomenal growth in attention to novel types of machine learning and neural networks technologies, the ultimate goals of AI researchers lie in development of artificial general intelligence (AGI). The application of AGI to MMs presents interesting opportunities. In particular, the opportunity exists to build intelligent software agents that work with physicians and medical researchers as co-pilots in order to work together to build models to solve patient pathology problems.

AI is applied to MMs. In particular, machine learning (ML) and deep learning (DL) are applied to model building and to analytics and simulations. Generative AI (GenAI) is a component of ML. GenAI includes numerous categories of technologies that are applicable to MMs, including generative adversarial networks (GANs), variational autoencoders (VAEs), autoregressive models, recurrent neural networks (RNNs), transformer-based models, reinforcement learning, diffusion models, flow models and neural radiance fields. Within GenAI, several algorithms are applicable to MMs, including SGD, Adam, AdaGrad, grid search and Bayesian optimization. GenAI applies to multimodal data, including text, images and sound. For example, image-based GenAI is applied to diagnostic imaging analysis and to building organ MMs.

### ML Applied to IMMs

Machine learning is afield of artificial intelligence that studies the application of probabilistic algorithms that learn to generalize from data. ML applies probabilities and statistics to data analysis to make predictions and perform computer functions without pre-determined training. It performs these tasks by creating its own rules or criteria for data analysis and pattern recognition. As it learns from more data, new data sets in an ML model enables better results; consequently, updating data leads to adaptation and improved predictions. ML models can generalize from training data to unseen data and can make forecasts based on data they have not seen (but extrapolate).

ML models are well suited for analysis of medical data. For instance, ML models are applied to medical diagnostics since they can identify and analyze a medical pathology. ML models are well suited to diagnostic prognostics as well since they provide predictions of disease progress. ML models also identify solutions to medical problems, providing therapeutic candidates to treat a disease.

Deep learning (DL) is a branch of machine learning referring to learning with utilization of neural networks. Learning can be supervised, unsupervised or semi-supervised. DL systems include recurrent neural networks, convolutional neural networks and transformers. Early neural networks were modeled on the human brain, but are now far more advanced. Generally, neural networks are configured with multiple layers. The “deep” part of DL refers to the large number of layers in a NN in which data are processed.

The present system applies geometric deep learning (GDL) for analysis and description of biomedical objects, systems and events. GDL consists of a set of techniques for modeling biomedical phenomena that are discussed below.

### GenAI Applied to IMMs

Generative AI (GenAI) refers to AI technologies capable of generating text, images, sound or video in generative models. These models create novel data from prompts by learning from patterns of their training data. In recent years, large language models (LLMs) have input massive data sets, such as information on the internet, in order to train the data with complex AI algorithms. Some LLMs range from 500M parameters to trillions of parameters.

The field of GenAI consists of numerous specific types of algorithms with applications to biology and medicine. Generative Adversarial Networks (GANs) uses a generator NN to develop synthetic data samples and a discriminator NN to make distinctions between natural and synthetic samples. GANs can be applied to the design of novel molecules. For instance, GANs can configure novel protein and peptide designs.

Restricted Boltzmann Machines (RBMs) and Conditional RBMs are GenAI models trained on contrastive divergence and learning algorithms. RBMs are applied to forecast drug-disease relations, predict drug-target interactions and identify repositioning tasks in drug-disease relation networks. RBMs are optimized to identify drug repositioning tasks in drug-disease relation networks.

Variational Autoencoders (VAEs) are probabilistic generative models that encode and decode samples in a search space. VAEs are comprised of two neural networks. The first neural network is an encoder that maps input data into a latent search space. The second neural network is a decoder that reconstructs the original input data from samples derived from the latent search distribution. The decoder NN generates a reconstructed sample resembling the input data. VAEs generate a chemical compound search space to show compound library diversity. VAEs also identify gene expression stimulated by a chemical compound and predict cell states from attributes of compounds. VAEs can be applied to predicting disease progression and to production of individualized therapeutics programs.

Natural Language Processing (NLP) provides an analysis of structure and content of a language. NLPs supply an analysis of “translational” language of amino acids sequences and relations, a forecast and classification of drug-target interactions and an identification of chemical “cell line” interactions. NLP techniques also design de novo drug compounds that are target specific.

Large Language Models (LLMs) learn probability relations between words. LLMs identify relations between genes, targets and diseases and summarize and analyze medical and biology research articles.

Diffusion models learn through a three-part process of forward, reverse and sampling phases. Diffusion models are effective in image generation and can generate an image from text prompts. In biological applications, diffusion models generate protein structure patterns from gene, RNA or amino acid sequence data and identify and predict potential protein-protein interactions from gene, RNA or amino acid sequence data.

Generative Pre-trained Transformers (GPTs) are AI algorithms that operate in an LLM by analyzing text structures to make predictions. In the biological context, GPTs make protein structure predictions from pretrained protein sequence language models. GPTs can also design proteins with targeted properties.

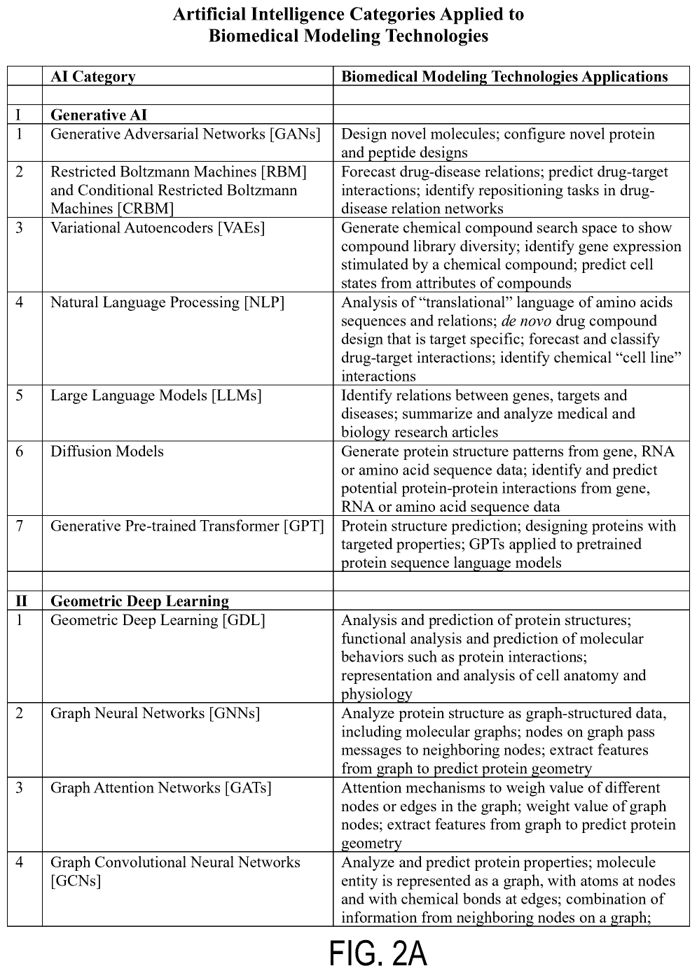

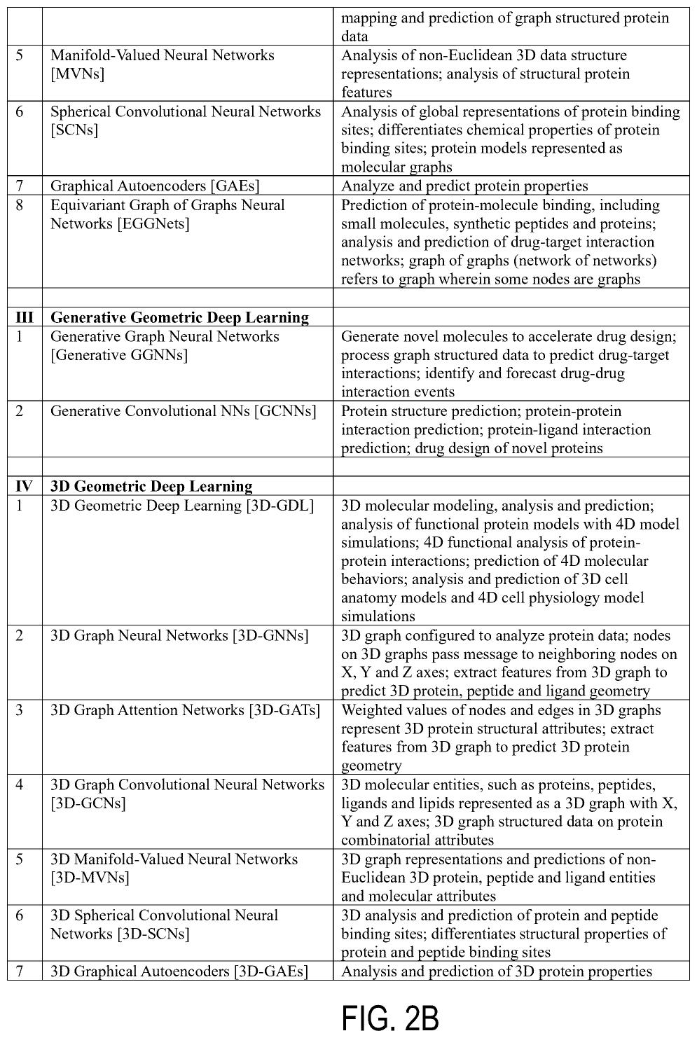

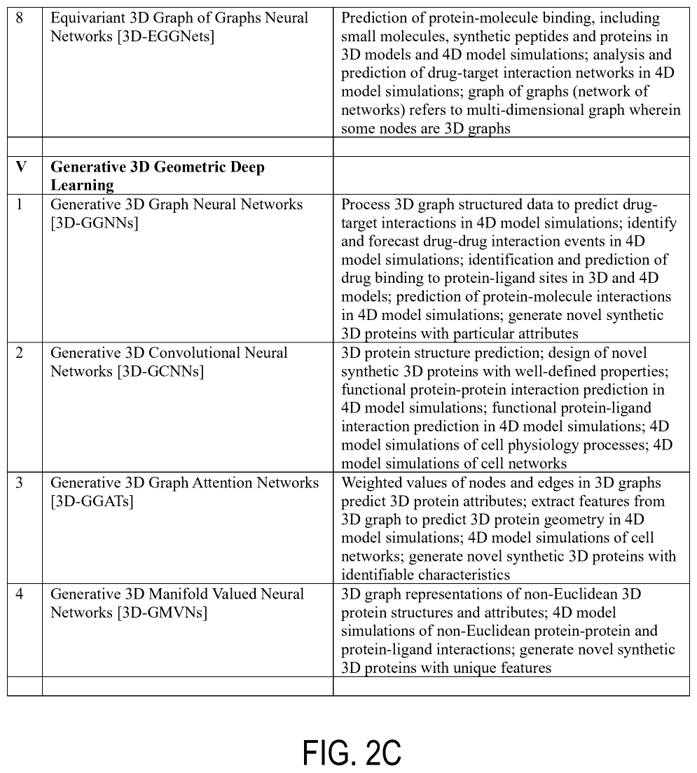

Refer to FIG. 2 for a review of GenAI techniques.

Collectively, these generative AI models are useful for providing the identification, and forecasting the behavior, of biological entities, such as DNA, RNA, proteins, peptides and ligands. In addition, these GenAI models are able to generate novel synthetic protein structures. Whereas these GenAI techniques have been applied mainly to describe or predict healthy molecular and cellular interactions, they are also applicable to comparative modeling and predicting unique patient dysfunctional molecular and cellular phenomena and behaviors that are at the root of pathologies. Consequently, these GenAI algorithms and techniques have a role in developing models of individualized patient pathologies as well as in generating novel solution options, viz., therapeutic solution options for these unique patient pathologies.

GenAI techniques are useful for MMs. These AI techniques have applications to diagnostics, prognostics and therapeutics. For diagnostics, these techniques identify and analyze molecular and cellular structures. In the main, GenAI provides a reference against which to measure biomarkers in a patient pathology model. For prognostics, GenAI supplies techniques for prediction of protein and cellular behaviors that are useful to track a patient's disease. For therapeutics, GenAI is applied to identifying and designing novel protein structures that may become a drug candidate. In some cases, GenAI may be applied to drug clinical trials by emulating synthetic patients in a control group.

But GenAI models have limits too. Their massive data sets require such a long time to train that they are virtually obsolete by the time they are activated months and years later. Also, their data may be prone to hallucinations if the data are impure or their AI training techniques too broad. Finally, they tend to be applicable only to optimized biological structures with algorithms that produce perfected models of healthy gene, RNA, protein, peptide, ligand, lipid and cell architectures and interactions. In this sense, these GenAI models are useful references against which actual patient pathological medical situations can be measured.

### Geometric Deep Learning (GDL) Applied to IMMs

Geometric Deep Learning (GDL) refers to a branch of neural networks (deep learning) that builds models referring to complex spatial or spatio-temporal objects. GDL typically uses graphs that consist of nodes, representing an object, that are connected with edges, which refers to relationships between object features. GDL is applied to modeling the structure of molecular entities such as proteins as well the operation and interaction of molecular networks. For the most part, the graphs to which molecular phenomena are mapped are structured two dimensionally with X and Y axes.

In the biomedical context, GDL is applied to analysis and prediction of protein structures, to functional analysis and prediction of molecular behaviors, such as protein interactions, and to the representation and analysis of cell anatomy and physiology. There are numerous sub-categories of GDL that refer to separate AI techniques for learning in the biomedical context; most of these are represented by different types of graph neural networks.

Graph Neural Networks (GNNs) analyze protein structure as graph-structured data. Nodes on a graph pass messages to neighboring nodes. GNNs extract features from a graph to predict protein geometry.

Graph Attention Networks (GATs) apply attention mechanisms to weight value of different nodes or edges in a graph. Features are extracted from a graph to predict protein geometry. GATs are a subclass of GNNs.

Graph Convolutional Neural Networks (GCNs) analyze and predict protein properties.

Molecular entities are represented as a graph, with atoms as nodes and with chemical bonds as edges. GCNs map and predict graph structure protein data. GCNs are a subclass of GNNs.

Manifold-Valued Neural Networks (MVNs) supply analyses of non-Euclidean 3D data structure representations from two dimensional graphs. MVNs supply an analysis of structural protein features.

Spherical Convolutional Neural Networks (SCNs) provide an analysis of global representations of protein binding sites and differentiates chemical properties of protein binding sites. SCNs supply protein models represented as molecular graphs. SCNs are a subclass of MVNs.

Graphical Autoencoders (GAEs) analyze and predict protein structures.

Equivariant Graph of Graphs Neural Networks (EGGNets) conducts two main classes of protein interaction analyses, comprising (a) prediction of protein-molecule binding, including small molecules, synthetic peptides and proteins and (b) analysis and prediction of drug-target interaction networks. Graph of graphs (network of networks) refers to a graph in which some nodes are graphs.

Refer to FIG. 2 for a review of GDL techniques.

The graph neural networks embodied in geometric deep learning approaches can store data in graph databases, which organize nodes, edges, properties and relations of geometric data. GDL models analyze topographic data in graph databases including molecular surfaces and curvature aspects of protein phenomena.

For the most part, GDL techniques are applied to describe the geometrical properties of objects. In addition, GDL techniques are applied to predict the geometrical behaviors of objects.

Geometric deep learning techniques are applicable to MMs. GDL models analyze RNA to protein translation, protein structures, protein pathway mapping, protein-protein interactions, protein-ligand interactions, protein-lipid interactions, protein-small molecule interactions and cellular component mapping. GDL is also useful in modeling mutated gene and RNA, non-coding DNA and RNA phenomena as well as dysfunctional proteins and peptides. For instance, GDL techniques identify a dysfunctional component of a protein and an optimal geometric configuration of a protein component; the GDL algorithms then apply probabilities analyses to compare a dysfunctional protein geometric configuration to an optimized protein configuration. In another example, GDL can predict a gene mutation that generates an anomalous protein structure configuration and predict the dysfunctional protein function. Identifying ML techniques to represent these abnormal genes and dysfunctional proteins supplies MMs the tools to identify unique patient diagnostics, prognostics and therapeutics.

### Generative GDL Applied to IMMs

Generative GDL techniques refer to NNs with generative graph learning functions.

Generative Graph Neural Networks [Generative GNNs] enables learning by allowing each molecular pattern to be learned from each step in a process so as to generate optimal molecular structures. GGNNs generate novel molecules to accelerate drug design, process graph structured data to predict drug-target interactions and identify and forecast drug-drug interaction events.

Generative Convolutional NNs (GCNNs), a type of geometrical deep learning technique, generalize a set of problems to make predictions from prior analyses. GCNNs make protein structure predictions, protein-protein interaction predictions and protein-ligand interaction predictions. In addition, GCNNs can design novel synthetic proteins and drugs.

Generative GDL algorithms are applied to IMMs mainly for therapeutics. Generative GDL techniques are useful for modeling novel synthetic drug designs.

### Novel 3D GDL Techniques Applied to IMMs

While GDL addresses analysis and prediction of three-dimensional molecular structures by utilizing two dimensional graphs on the X and Y axes, the limitation of two dimensional geometrical molecular representations to analyze three-dimensional objects constrains the traditional GDL modeling approaches.

The present invention discloses 3D GDL and families of techniques embodying 3D GDL. In general, 3D GDL provides 3D molecular modeling, analysis and prediction by applying 3D graph architectures. In addition, 3D GDL provides 4D molecular modeling, including analysis of functional protein models with 4D model simulation, 4D functional analysis of protein-protein interactions, prediction of 4D molecular behaviors, analysis and prediction of 3D cell anatomy models and 4D cell physiology model simulations. Numerous subclasses of 3D GDL apply specific techniques to 3D molecular modeling.

3D GDL includes a set of techniques that are applied to 3D electronic graphic displays. Data, particularly, molecular object data or cellular phenomena data, are placed in a 3D matrix graph grid. A 3D matrix architecture is applied to 3D graph architecture.

Examples of molecular objects that are described and analyzed in 3D GDL systems include healthy protein structures, dysfunctional protein structures, healthy and dysfunctional small peptide structures, small molecules, protein-protein interactions, protein-lipid interactions and protein-ligand interactions. Furthermore, functional molecular objects can be described in 3D GDL simulations, including healthy and dysfunctional protein dynamics and protein-protein pathway interaction dynamics.

3D Graph Neural Networks (3D-GNNs) embody 3D graphs configured to analyze protein data, featuring nodes on 3D graphs that pass messages to neighboring nodes on X, Y and Z axes. 3D-GNNs extract features from 3D graphs to predict 3D protein, peptide and ligand geometries.

3D Graph Attention Networks (3D-GATs) feature weighted values of nodes and edges in 3D graphs that represent 3D protein structural attributes. 3D-GATs extract features from 3D graphs to predict 3D protein geometry.

3D Graph Convolutional Neural Networks (3D-GCNs) analyze and predict 3D molecular entities, such as proteins, peptides, ligands and lipids represented as a 3D graph with X, Y and Z axes. 3D GCNs map 3D graph structured data on protein combinatorial attributes.

3D Manifold-Valued Neural Networks (3D-MVNs) analyze 3D graph representations and predictions of non-Euclidean 3D protein, peptide and ligand entities and molecular attributes.

3D Spherical Convolutional Neural Networks (3D-SCNs) apply 3D analysis and prediction of protein and peptide binding sites. 3D-SCNs differentiate between structural properties of protein and peptide binding sites. Because they involve spherical objects, these models apply non-Euclidean and topological analyses of 3D object features.

3D Graphical Autoencoders (3D-GAEs) analyze and predict 3D protein properties.

Equivariant 3D Graph of Graphs Neural Networks (3D-EGGNets) predict protein-molecule binding, including small molecules, synthetic peptides and proteins in 3D models and 4D model simulations. 3D-EGGNets analyze and predict drug-target interaction networks in 4D model simulations. A 3D graph of graphs refers to multi-dimensional graph wherein some nodes are 3D graphs.

In general, 3D data sets in 3D graphs are utilized in 3D databases in order to optimize analytical efficiency. 3D graphs and 3D databases enable increased precision over models in prior (2D) modelling approaches. This is akin to representing a snowflake in 3D vs 2D.

In one embodiment, 3D data from these models are analyzed in the 3D databases. 3D object features are analyzed as key 3D representations of multi-dimensional phenomena. In another embodiment, multi-dimensional databases are applied to 3D GDL models. In still another embodiment, a 3D model and 3D database can apply (3D) voxels beyond the typical 2D pixel representation.

3D GDL modeling and techniques are useful for MMs. In the case of 3D protein and cell structures, these techniques supply enhanced modeling texture by utilizing 3D graphs. 3D models of 3D protein structures, protein folding prediction from gene and RNA sequence data, transcription and translation process modeling are optimized by utilizing 3D GDL. In the case of 4D modeling, 3D GDL modeling analyze and predict 4D protein function and protein-protein interaction events. While these techniques are useful for modeling 3D structural phenomena and 4D events of healthy optimized genes, RNA, proteins and peptides, these techniques are particularly suited for modeling complex model simulations of gene and RNA variants and dysfunctional protein and peptide structures and operations.

The application of 3D GDL models to MMs can be analogized to a 3D snapshot representation of biomedical phenomena. Taking this one step further, 3D GDL models are applied to 4D simulations, which are akin to video representations of biomedical phenomena. 3D GDL models are useful for diagnostic, prognostic and therapeutics modeling. In particular, 3D GDL algorithms are applied to precision diagnostics and therapeutics in order to identify the properties of protein targets and the attributes of novel synthetic drug designs.

Building 4D simulations are a crucial aspect of understanding complex molecular, cellular and biomedical phenomena. While the application of 3D GDL to structural representations of biological objects is useful, describing object event and object-interaction processes supplies a useful time series analysis of object behaviors. For instance, a 4D depiction of a moving picture of phases of a protein's or a cell's operations dramatically assists in understanding these events.

### Novel Generative 3D GDL Techniques Applied to IMMs

Generative AI is applied to 3D GDL to create novel advanced models. While GDL, and 3D GDL, models are primarily descriptive, Gen 3D GDL (3D GenGDL) models enable synthesis of novel objects. These new techniques enable these models to design synthetic objects such as new proteins that do not exist in nature.

Generative 3D Graph Neural Networks (3D-GGNNs) process 3D graph structured data to predict drug-target interactions in 4D model simulations. These models identify and forecast drug-drug interaction events in 4D model simulations as well as drug binding to protein-ligand sites in 3D and 4D models. 3D-GGNNs predict protein-molecule interactions in 4D model simulations. These models generate novel synthetic 3D proteins with particular attributes.

Generative 3D Convolutional Neural Networks (3D-GCNNs) make 3D protein structure predictions and generate novel synthetic 3D proteins with well-defined properties. In addition, these models predict functional protein-protein interaction in 4D model simulations as well as functional protein-ligand interaction in 4D model simulations. 3D-GCNNs also supply 4D model simulations of cell physiology processes and of cell networks.

Generative 3D Graph Attention Networks (3D-GGATs) analyze weighted values of nodes and edges in 3D graphs to predict 3D protein attributes. These models extract features from 3D graphs to predict 3D protein geometry in 4D model simulations. 3D-GGATs also develop 4D model simulations of 4D cell networks. In addition, these models generate novel synthetic 3D proteins with identifiable characteristics.

Generative 3D Manifold Valued Neural Networks (3D-GMVNs) develop and analyze 3D graph representations of non-Euclidean 3D protein structures and attributes. In addition, these models develop 4D model simulations of non-Euclidean protein-protein and protein-ligand interactions. 3D-GMVNs also generate novel synthetic 3D proteins with unique features.

Refer to FIG. 2 for a review of these generative 3D GDL techniques.

These advanced generative 3D GDL modeling techniques are applied to IMMs. These enhanced (GenAI+3D GDL) models combine the best elements of GenAI with the accurate descriptive attributes of 3D graph-based geometric deep learning. In some cases, LLMs are trained with biomed data in order to supply structured pre-trained data to 3D GDL models. The synthesis of GenAI and 3D GDL techniques or algorithms enables MMs to generate novel artificial protein or biologic designs to solve therapeutic challenges. In addition to developing novel structural objects, such as novel protein designs, these techniques can build models to understand the complex interactions of functional molecular objects. For example, these models can design novel proteins with specific attributes that solve a problem of dysfunctional proteins while minimizing non-target binding and, hence, side effects. In another example, once a protein target is identified, the models can reverse engineer a novel solution by inventing a synthetic protein structure. While the development of AI models for the prediction of healthy protein structures from healthy DNA and RNA sequence data are useful as reference simulations, the combination of GenAI and 3D GDL supplies the tools for the analysis and prediction of dysfunctional proteins and peptides, which enables the opportunity to develop optimized therapeutic solutions. The combination of these technologies in MMs enable the realization of personalized medicine.

### Personal Health Assistants (PHAs) as Multifunctional Intelligent Software Agents Applied to IMMs.

PHAs are intelligent software agents that perform multiple tasks in a computational or network environment. PHAs are applied in the present invention to IMMs. By automating many tasks involving modeling for health challenges, PHAs save time and accelerate the process of finding solutions to complex medical problems.

PHAs perform tasks such as data collection, data management, analytics, AI selection, AI synthesis and communication in a computer network. PHAs operate in a multi-agent system (MAS) in order to perform tasks, including generating specialized agents; PHAs can either cooperate or compete in a network. Applying AI techniques are a central feature of PHAs as they are related to MMs. These issues of PHA mechanics are reviewed and novel elements are articulated.

PHAs are critical components of a IMM system. PHAs build models and simulations, perform experiments to solve diagnostic and therapeutic problems, and make predictions.

### PHA Mechanics

PHAs search for, collect, analyze, synthesize and manage data. As such, PHAs interact with database management systems. PHAs are useful to locate, collect and package medical data. In the context of IMMs, PHAs collect and summarize data from medical articles, medical reference databases and medical and biological LLMs. For example, PHAs search for, analyze, aggregate, and summarize medical research data. In some cases, PHAs apply in-database analytics in order to generate medical research summaries.

In addition to general medical information, PHAs collect data on individual patient medical tests and records, including EMRs, EHRs and integrated health records (IHRs). These data sets are useful for enabling PHAs to build medical MMs on specific patients.

PHAs are optimized to be personalized to specific physicians or medical researchers. Consequently, PHAs can be tailored for specific medical or biological specialties. As such, these software agents can possess immense information on a particular specialized medical field in order to enable a specialist physician to have the most advanced research information. For example, cardiologists can have cardiology-PHAs which possess expertise on cardiovascular systems. Further, the PHAs can fine-tune their customization to individual physician practices or medical researchers. In this sense, PHAs are physician or medical research co-pilots which can provide general medical information, advice and predictions to physicians or researchers in real time.

PHAs are integrated in a multi-agent system (MAS). Different PHAs have different specializations in order to promote or optimize different tasks. When a physician requests information on a particular medical pathology, several agents may simultaneously apply their specialized skills to gather data, analyze data, summarize data and provide recommendations. Multiple agents send messages to each other in order update their programming and task status.

Different PHAs have different tools, in particular, AI tools, to accomplish their computational tasks. For instance, one category of PHA may be applied to diagnostics in which they have a specific toolkit optimized for diagnostics, while other categories of PHA may be applied to therapeutics in which they have a different toolkit optimized for therapeutics.

In an embodiment, a main PHA launches many specialized sub-agents in order to complete computational tasks. Similarly, many different PHAs can cooperate in order to achieve a goal, such as finding the source of a disease or identifying and selecting optimal therapeutic options for a particular patient.

Operationally, PHAs integrate AI algorithms into their program code. These AI algorithms include GenAI techniques, machine learning techniques and deep learning techniques. These AI techniques and algorithms enable the functionality of the PHAs. In addition, AI techniques and algorithms are applied by the PHAs to specific applications such as MMs and simulation sub-types.

AI is a central feature of PHAs. PHAs have access to AI technique and algorithm libraries and select the best algorithms for a particular task. PHAs select different AI techniques to apply to different MM functions. For instance, when a PHA needs to identify a patient pathology, it may access specific specialized types of AI techniques for that task.

However, in the present system, PHAs may also synthesize novel AI techniques by combining different AI approaches—either sequentially or in parallel—for specific MM applications. In effect, the system enables PHAs to combine multiple AI techniques into a custom algorithm for specific MM simulation applications. For example, the PHAs may combine two or more of the AI techniques listed in FIG. 2. Note also the list of AI techniques on each MM Level in the IMM system shown in FIG. 1. In addition, PHAs may use two or more AI techniques in a sequence to solve a problem. In an embodiment, A PHA may reverse engineer a unique synthetic AI technique from the requirements of a task to the AI combinations required for the task.

PHAs can be applied by physicians or medical researchers for different applications. For doctors, PHAs analyze patient medical data, package medical data, provide diagnostics guidance, provide drug trial guidance and provide therapeutic selection guidance.

PHAs can assist in constructing different types of specialized medical MMs. Because they are endowed with AI techniques, PHAs supply diagnostic MM insights, prognostic MM predictions and therapeutic MM solutions.

In addition to assisting physicians and medical researchers, PHAs are useful in assisting patients too. PHAs can assist patients in completing forms, updating patients about diagnostic test status and educating patients on their medical conditions and prognoses. PHAs are also useful for interactions between patients and doctors. In the case of patient oriented PHAs, there is a bi-directional connection between the patient and the patient's MM enabling dynamic feedback between the patient and the MMs. In an embodiment, PHAs act as intermediaries between patient MMs and patient relationship management (PRM) software.

### PHAs for Modeling Functions

PHAs perform model building of MMs and simulations. PHAs collect data from different sources, including medical databases and libraries, patient medical diagnostic tests and patient EHRs and IHRs. In addition to collecting, assimilating and aggregating patient medical data, MMs also analyze the data in order to search for diagnostic solutions and analyze and synthesize the data in order to search for therapeutics solutions. PHAs also assist in building MMs themselves, including identification of a useful technique for each MM level in the IMM system.

PHAs can build two or more MMs simultaneously by applying multiple AI techniques that fit each respective model type. In addition to building MMs, PHAs are applied to building model simulations and in silico experiments too.

PHAs are applied to building diagnostic MMs of individual patient diseases in order to identify the sources of a patient pathology. The PHAs apply various AI techniques in order to identify data, identify areas in which the data are incomplete, and interpret and analyze the limited available data. PHAs not only build the MMs and simulations, but also enable the physician or medical researcher to interact with the MMs to update data, interrogate the model or refocus the model.

PHAs enable medical MMs to provide in silico experiments and analyses. By employing PHAs to activate an in silico experiment, dysfunctional proteins can be identified, compared to healthy reference genes, RNA and proteins, specific attributes recognized and functional consequences of dysfunctional proteins in protein and cellular pathways specified.

PHAs may be configured to design in silico experiments in an MM. PHAs design experiments with a goal and specified methods of operation. PHAs can be tuned for problem solving, classification, sorting and ranking, and supplying scenario outcome options.

PHAs are applied to MM applications in order to supply accurate diagnostics, prognostics and therapeutics. In the case of diagnostic MMs, PHAs build a model of patient pathologies, which includes information collection features and functional problem finding features in order to map a patient pathology. Further, PHAs are configured to provide analyses of biomarker data in order to identify diagnostic solutions.

PHAs are applied to prognostics in order to make predictions. Once a MM has identified a patient diagnosis, PHAs apply AI techniques in order to forecast the evolution of a patient disease. In addition to the application of PHAs to diagnostic prognostics, PHAs are applied to therapeutic prognostics by tracking the patient disease in the context of drug or therapy feedback. In this context, PHAs supply probabilistic scenarios of outcomes from application of different drugs or therapies for each patient. PHAs are also applied to preemptive medicine by predicting disease emergence before the onset of symptoms by analyzing biomarkers and patient data that indicate a probable disease onset in different scenarios.

PHAs are applied to therapeutics in order to select an optimum therapeutic remedy, viz., a therapeutic drug candidate, for a patient pathology, or to construct a novel synthetic drug for a unique patient pathology.

PHAs are the workhorses of the IMM system much as proteins are the workhorses of the human cell.

### PHA Typology

The present invention specifies a set of differentiated PHAs.
1. PHA-m: Model Builders
2. PHA-a: Analyzers
3. PHA-s: Searchers
4. PHA-c: Combiners
5. PHA-i: Interrogators
6. PHA-mes: Messengers
7. PHA-b: Brokers
8. PHA-sec: Security
9. PHA-p: Predictors
10. PHA-sims: Simulators

PHA-m's are model builders that perform tasks associated with building MMs, such as combining data into tables, graphs and models and representing data in models or simulations.

PHA-a's are analyzers that perform tasks involving analysis or synthesis of elements in MMs.

PHA-s's are searchers that seek out data from databases.

PHA-c's are combiners that combine two or more AI techniques or algorithms into a hybrid synthesis for application to a particular issue involved in a MM.

PHA-i's are (adversarial) interrogators that actively interrogate data in order to build or optimize a model.

PHA-mes's are messengers or communicators that pass messages between models and other agents.

PHA-b's are brokers that intermediate between MMs and LLMs or medical databases.

PHA-sec's are security agents that enable different levels of security in MMs.

PHA-p's are predictors that forecast or predict event scenarios based on MM data.

PHA-sims are simulators that construct simulations from MMs.

The various agents work together with a division of labor to accomplish MM goals. The agents can be assigned to a set of tasks such as outlining a MM, seeking data from a medical database, interfacing between two or more MMs, completing two or more MMs, interrogating MM data, storing MM data, making predictions, generating simulations, combining AI algorithms into useful specialized hybrids, analyzing MM data, adversarially interrogating MM data or passing messages between MMs.

In one main mode, PHAs passively build MMs by collecting, sorting and completing data sets. In another mode, PHAs are active, dynamically initiating interactive experiments to test a hypothesis.

PHAs can also launch minor PHAs in order to add capabilities to more quickly and efficiently execute a task.

### Integrated Health Record Platform: Integrating IMMs, Health Data Management, Medical Data Security and Patient Relationship Management

For centuries, physicians took copious paper notes to track and manage patient conditions. This worked well until the computer and cloud revolutions. In the 2000s, medical practices have begun using electronic medical records (EMRs) to collect and manage information about patients. These EMR patient data, however, were limited to a specific doctor's office or in-house clinical practice, mainly because of strict government privacy and security regulations. For the most part, like a patient chart, each individual EMR can be seen as a single record about a single event. While the goal of an EMR was to replace the patient chart, the outcome is less spectacular. Constraints in the quality, cost, standardization and security of maintaining EMRs put pressure on physicians to find alternative solutions.

Electronic Health Records (EHRs) represent a step in the evolution of medical records management. EHRs comprise an electronic version of a patient's medical history. EHRs are accessible to medical care workers in different organizations, enabling the tracking and sharing of medical records among physicians across clinical domains. In some ways, EHRs offer an extension into healthcare from enterprise data management technologies, thus enabling the storage of health records by physicians of medical histories, blood and imaging diagnostic test results, immunization status and billing and insurance information. When used by hospitals, EHR data can be aggregated and analyzed, in order to guide data management practices.

EHRs have challenges too. There are concerns about safety, medical errors or inaccuracies, privacy, security, legal liability and standardization involving EHRs, among other things. So far, physician productivity has not been promoted by EHRs, mainly because of the major time commitment required to develop and manage EHR record keeping. While, in time, it may be possible for GenAI to act as copilots for clinicians in order to efficiently manage patient records, so far this goal is only a dream.

In contrast to EMRs and EHRs, a personal health record (PHR) is an electronic record with medical data controlled by a patient, the content of which the patient determines whether to make available to a health provider.

The advent of novel digital technologies may have facilitated the next step in the evolution of electronic patient records, thereby enabling a next generation of patient record organization that will overcome past challenges.

### Integrated Health Record Platform (IHRP) and IMMs

The present invention specifies an IHRP. The IHRP is a health record platform that integrates different levels of patient healthcare data. The different levels include:
Private health data (doctor)
Multiple doctors sharing private patient health data
Health data with patient permissions
Privileged patient health data (private health data)
Generalized patient health data
Anonymized patient health data
Insurance private patient health data

Previous electronic health record systems, such as EMR or EHR, simply replace written doctor notes with electronic doctor notes. These prior elemental systems provide useful baseline data for the IHRP system as patient data are input. However, the IHRP also includes medical research article data, pre-clinical data, clinical trials data and genomic, proteomic and multiomic data sources as well. All of these data sources supply general medical data and specific patient medical data to the IHRP.

The IHRP can be used on relational, object-relational or other database management systems. The IHRP can also be applied in the cloud (pubic, private or hybrid) as well as at the edge in computers, phones or devices. These data structures enable automatic updating of data.

The IHRP applies natural language processing (NLP) technologies and algorithms to analyze health records. By applying NLP, the IHRP can survey, analyze and summarize medical articles or patient charts and translate medical articles into different languages. By using vector databases, GenAI algorithms can overcome a problem of obsolescence of too-old data in LLM training data. GenAI technologies also enable the transfer of data from lower-level patient electronic records into MMs. Alternatively, GenAI technologies can be programmed to summarize MMs in general patient electronic records. By enabling interaction between MMs and IHRP patient data, patient data can be constantly updated, with updates that feed patient MMs; as the MMs adapt with the newest data, they can make more accurate predictions and recommendations.

PHAs conduct many functions in the IHRP system in order to process patient data, including searching for data, storing data, adding data to MMs, analyzing data, securitizing private health data, generalizing patient health data, anonymizing patient health data and intermediating between a patient's doctor and patient's insurance company.

At an early level, patient medical data are organized, analyzed and summarized. Early level patient data are input into MMs. For instance, traditional EMR and EHR patient data are input into the IHRP.

The MMs apply different data types. The MMs access vast medical research, genomic, proteomic and multiomic databases in order to identify reference health data. But the MMs also receive data inputs from physicians or medical researchers for individual patient data on specific diseases. The HRP integrates these data types. The MMs draw on the generalized medical databases and the individual patient data input by electronic records from the IHRP. But the MMs also export model data to the IRP, particularly model data, analysis and summaries.

Electronic patient data are routed to different MM components from the HRP. For instance, an electronic patient record can feed data to the HRP and then to a patient's medical MM for a diagnosis. Electronic patient records can be structured or unstructured. However, the IHRP can structure the data by imposing a system of standardization on the records.

Electronic patient records accumulate patient data by including patient test results and patient condition assessment. These data are input into the IHRP and then to a patient's MM. In an embodiment, digital health data are translated to a MM by applying NLP. In addition, PHAs can automate data translation from electronic patient records to MMs.

The IHRP system and the IMM system can cooperate by sharing data to solve a problem. For example, an IHRP can aggregate patients' data from multiple patient MMs so as to identify drug targets, predict drug outcomes with different variables or track disease progression under different conditions. Aggregate patient data analysis of multiple patient electronic records can be anonymized (de-identifying patients) and secured. For instance, in the case of clinical trials, patient medical records can be anonymized by removing patient identification from record analysis.

Different stakeholders can have access to the patients' HRP data, including internists, specialists, a hospital or clinic, drug clinical trials and vaccination administrators.

### Patient Data Security Management in IHRP

Patient medical data require different levels of security. Each process of patient data collection, storage, analysis and transmission requires data privacy and security. The patient data security management (PDSM) system is configured to provide several levels of data security management.

The PDSM system is designed to receive patient consent and permissions at different points in the process depending on the recipient of the information. Though general practitioners, specialist physicians and surgeons, registered nurses, physician assistants, clinical trials and insurance companies each require access to patient data, there is a need for patients to supply authorization of data access to particular patient information. As sensitive patient data lie in different healthcare databases, it is necessary to prioritize the security clearance thresholds for these data sets.

The present PDSM system establishes a set of several layers of security for patient healthcare data in order to maintain patient privacy and security. On the top layer are highly confidential patient medical data that is privy to specific physicians such as the patient's primary doctor and/or to the patient's specialist doctor(s). In these instances, access to patient data on a specific patient event or disease is highly restricted and must be approved for transmission to specific physicians and healthcare providers. The patient may provide limited consent to these unique data sets in order to grant access. These data are color-coded as red.

On the next layer are generally confidential patient medical data on a specific patient condition, such as a particular patient pathology, or on a general or chronic patient health condition. The patient must agree to consent on the distribution of patient medical data on these specific pathology to specific physicians. These data are color coded as orange.

On the third layer are general patient information that can be disclosed or transmitted to physicians or other clinicians, such as doctors in a hospital setting, or within a specific clinic or hospital group. For these data, patient consent for transmission will be requested before a hospital stay. These data are color coded as green.

When a patient is admitted on an emergency basis to a hospital or clinic, the administration must request to a consent of information for acute care so that high priority patient health information can be transmitted in their healthcare system. These data are color coded as blue.

In the case of clinical trials, patient data can be anonymized and provided to the clinical trials administrators. These patient data can be supplied to administrators once they are confirmed to be scrubbed of specific identifiable patient data. These data are color coded as purple.

As the PDSM system is integrated into the IHRP, patients are able to provide selective access of private patient health data to doctors with general or specific consent and permissions. As the patient health pathology is investigated, the patient may conclude that the data are essential for other specialists and may thereby provide general consent to data transmission. But some patients may decide that the data must remain private and may only provide consent for access to the patient data to one or two physicians.

While patient data are input into the IHRP, the data are sorted into different levels of security. When the data are transferred from the IHRP to the MMs, the data are also organized according to levels of security priority. There will be times when the MMs will require access to more patient data; at these times, the MMs will request permission to access confidential patient information in order to complete its tasks. Without sufficient information, MMs cannot perform adequate analyses, so it benefits the patient to supply consent to enable access to these data. In an embodiment, the MMs can import and analyze the confidential patient information while keeping the information private. Also, the summary of the MM outputs can include different levels of security to enable a general or specialized private description of a patient disease.

In order to limit access to patient data, the data files are encrypted. When the patient enables selective or general disclosure of their data, the data are decrypted for a specified purpose.

PHAs may be applied to secure patient consent, primarily by requesting consent and explaining to the patient the need for specific data in order to complete a particular task.

In an embodiment of the invention, the IHRP system and MMs are configured on the blockchain. One advantage of the blockchain is the combination of data structured on a distributed ledger with data security. In addition to traditional blockchain-enabled encryption, data on the blockchain can be tokenized, which supplies an additional layer of data security.

### Patient Relationship Management

Patient relationship management (PRM) consists of analytical, operational, patient management and collaboration components. While customer relationship management software for healthcare is tailored mainly for application to administration of hospitals or clinics, PRM focuses on supplying tools directly to patients and doctors in order to optimize the patient healthcare experience. PHAs are applied to actively perform various functions in the PRM domain.

The analytical function of PRM software is supplied, on one level, to interpret and analyze data in health records, interpret patient medical tests and make therapy recommendations. On another, higher, level, the analytical function is primarily to build MMs from general medical inputs and specific patient healthcare data.

The operational function of PRM software is applied mainly to administrative healthcare processes. For instance, a medical doctor office administration, insurance claims processing, doctor employee hiring, training and management, administrative and nursing staff scheduling, patient-nurse/staff communications and doctor-nurse/staff communications are tracked in the operational function.

The patient management function of PRM is applied to tasks such as patient tracking, patient-doctor communications, medicine tracking, treatment feedback and doctor and testing appointment scheduling. The patient management function includes personalizing and automating the patient healthcare experience. These functions include personalized data collection for medical tests, coordination with doctors to interpret tests, coordination of a patient diagnosis with a physician, coordination of therapy options with physicians and facilitation of therapy option assessment and therapy updates. GenAI can be applied to these processes in order to learn about and personalize each patient process. The patient management function of PRM is substantial after interaction of a patient and their doctor. The system enables the automation of medical records management, health benefits (e.g., insurance), digital pharmacies, care giving and coordination, chronic condition management and medical management. PRM software provides interfaces between patients and their care givers.

The collaboration function of PRM enables collaborative interdepartmental data flows. When patients enroll in drug clinical trials, PRM software can connect patients to specific studies according to the unique criteria of the trials in order to match patient eligibility. The collaborative function builds relationships between specialist physicians and drug or biotech companies in order to find matches for clinical trial program eligibility. Since drug companies are seeking very specific patient profiles, such as patients with a unique genetic mutation, they will likely wish to work with many specialists who interface with patients that feature these sorts of genetic mutations.

The advent of digital technologies provides the ability to search for eligible patients worldwide and for any patient to find a clinical study worldwide too. So, the PRM software system can facilitate a process that resembles a sort of broker for information to connect patients and drug companies. In fact, the MMs of patients can be accessed by clinical trial program administrators. Aggregated anonymized patient MMs enable clustering patients with similar pathology situations. Doctor specialists can register patients with unique disease genetics. Similar patients with shared diseases can be in trials and treatments together, but perhaps distributed over a longer distance; for instance, patients can be identified with similar biomarkers and MM diagnostic analyses. This approach leads to space-transcendent distributed studies, trials and treatment programs. The PRM collaborative system enables a social medical diagnostics and treatment network.

MMs are a nexus for PRM since patient data are stored and analyzed in patient's IMMs. From these data, therapeutic solutions may become possible by networking with drug companies and drug clinical trials worldwide.

### Individualized Medical Modeling for Diagnostics

For centuries, physicians evaluated patient symptoms in order to make diagnoses. Even today, many diagnostic systems simply automate the symptom-based process of identifying a disease. But limiting a diagnostic evaluation to patient symptoms alone is insufficient to identify the source of many diseases because similar symptoms tend to mimic different diseases; such an approach is imprecise and incomplete. The discovery of genomics has revealed a far more complex molecular network that underlies the source of many diseases, particularly chronic or hereditary diseases.

In its most advanced implementation, computer analyses can be applied to biomolecular phenomena in order to discover the sources of patient diseases. At the center of these diagnostic investigations lie individualized medical modeling (IMM), a novel modeling system that applies AI to identify each individual's unique disease attributes and sources. While progress has been recently made in identifying the structure of healthy proteins from amino acid, RNA or gene sequences, these data merely show a reference model against which pathologies can be measured. MMs compare the reference medical and healthy protein structure database or LLM protein prediction information to actual patient pathology data in order to develop a precise understanding of each patient's condition. This application of IMMs to personalized medicine applies AI, ML and DL techniques to analyze each patient's gene, RNA and protein biomarkers in order to assess the individuals' disease attributes. Consequently, MMs solve the problem of medical diagnostics at the granular level so we can understand the causes and dynamics of each patient's disease. This precision-oriented perspective of medical diagnostics is generations beyond the traditional symptom-based model of yesteryear.

### IMMs for Personalized Medicine (PM) Diagnostics

One of the challenges of modern medicine is the bifurcation of diagnostics and therapeutics. On the one hand, the internist will diagnose a pathology and then typically refer the patient to a separate specialist for therapy. If the first level diagnosis is imprecise or incomplete, particularly because it is focused on a symptom-based diagnostics paradigm, the therapy may miss the mark. This is a problem with AI applied to the traditional symptom-based diagnostics approach, which merely accelerates an obsolete diagnostics model. Until recently, patient diagnostic test data were limited to blood and imaging diagnostics. The challenge lies in overcoming incomplete information about a patient pathology since it lacks diagnostic precision.

In some ways, medical diagnostics is a sort of puzzle. With limited information, a physician is tasked with finding a solution, viz., an accurate diagnosis of a patient disease. For some simple medical matters, it may be sufficient to analyze incomplete data on a patient condition that is more or less straightforward. But in many cases, a more complete battery of diagnostic testing will be required in order to understand the contours of the patient's disease. Unfortunately, in many cases, doctors are only able to describe the manifestation (i.e., symptoms) of a disease and lack the tools to look deeper.

MMs combine computer modeling and advanced AI and ML techniques in order to analyze biomedical data. Whereas in the past, we were limited to generalized medical information, in the past two decades a revolution has occurred with the discovery and illumination of the human genome. The revelation of understanding the precise genetic sources of our diseases has led to corresponding revolutions in RNA and proteomics, which are the agents and products of our genes. If about 90% of many diseases have as their source a genetic cause, then understanding genetic and proteomic attributes, dynamics and relationships is crucial to advancing medicine and medical diagnostics.

Not only have we deciphered the human genome, we have identified chromosomal maps that locate specific genes at specific addresses and zip codes on the chromosomes. Human genetic data are stored in 23 pairs of chromosomes, with chromosome 1 being the largest and other chromosomes being progressively smaller. The 23^{rd} pair are distinctive chromosomes, X and Y, that allocate sex; females have a pair of X chromosomes while males have an X and Y chromosome. Genes are the constituent components of chromosomes. Because chromosomes are unusually long molecules, they are tightly wrapped around proteins. A centromere is a thin band that separates each chromosome into a long arm (q) and a short arm (p). The genes in each chromosome are classified in a numbering system illustrating cytogenic mapping. For example, a hemoglobin beta gene (HBB) is located on chromosome 11p15.4, which designates that the gene is on the short arm of chromosome 11 and positioned at address 15.4. The largest chromosome, viz., number 1, contains over 3000 genes, number 2 contains over 2500 genes and so forth. By identifying the specific genes and their precise addresses on chromosomes, we can identify risks of disease development by recognizing and analyzing individual gene mutations.

We are all born with genes inherited from our parents. However, genes can decay over time, describing somatic gene mutations that occur as we age. Such somatic gene mutations reflect ordinary wear and tear as well pathologies that accelerate genetic degradation. Epigenetics can influence some features of this genetic degradation. When different cell lines in different biosystems degrade at different rates, the asymmetric somatic gene mutations reflect the gene alternations which are responsible for some chronic pathologies.

Molecular MMs are useful for ascertaining and precisely pinpointing the genetic variants that encode abnormal RNA and proteins that generate specific pathologies. MMs are applied to molecular biomedical data in order to track the sources of pathologies and predict disease progression.

In addition to molecular genetic, RNA and protein maps, MMs are also applied to cellular biology. While proteins are the workhorses of the cell, the internal operations of dysfunctional protein interactions and pathways that generate many intracellular pathologies need to be mapped. Cells, too, contain components that are mapped according to specific cellular addresses and zip codes. For instance, the nucleus of a cell may have a zip code, while the multiple mitochondria may have different addresses. Internal cellular components are mapped in MMs in order to distinguish healthy cellular behavior from pathological cellular behavior. Each major cell type, generally corresponding to the different main organ types, may have a slightly different basic architecture that necessitates a differentiated map that refers to different structural and functional elements. For example, nerve cells embody a different architecture than red blood cells, each different type of which requires a unique map or model. Thus, MMs are applied to modeling intracellular and intercellular anatomy and mechanics.

Organs and organ systems also contain addresses and zip codes that enable the clear mapping of these biological objects. For example, the vasculature has zip codes that specify the different locations of different regions of the system. A map of a patient's heart will be essential to understanding the patient's unique cardiological anatomy and dysfunctions. Similarly, a map of a patient's brain and nervous system will be essential to understanding the patient's unique neurological anatomy or neurodegenerative dysfunctions. Zip codes and addresses are applied to the various human biosystems, from the cardiovascular system and nervous system to the endocrine system and skeletal-muscular system, thereby enabling individualized medical models in order to understand pathologies unique to each patient.

### Biomarker Analysis in IMMs for Diagnostics

According to Wikipedia, a biomarker “is a measurable indicator of some biological state or condition.” Biomarkers can be applied to diagnostics to identify a disease condition or to prognostics to identify the progression of a disease. Biomarker types include blood drawn protein, imaging and digital biomarkers.

There are a number of biomarker categories, including diagnostic biomarkers, prognostic biomarkers, predictive biomarkers, pharmacodynamic biomarkers and risk biomarkers. Diagnostic biomarkers detect the presence of a disease. Prognostic biomarkers predict the probability of a disease progression. Predictive biomarkers are applied to identify genetic features that enable a patient to more likely to respond to a targeted medicine. Pharmacodynamic biomarkers, also called therapeutic prognostics biomarkers, track drug reactions in patients. Risk biomarkers predict the probability of, or predisposition for, a patient to contract a disease.

An example of a typical protein biomarker includes a prostate specific antigen (PSA), which signifies the presence of prostate cancer and cardio reactive protein (CRP) which signifies the presence of inflammation and, in some cases, coronary artery disease. Imaging biomarkers include CT or PET scans of solid tumors. Digital biomarkers apply electronic devices to detect electrical signals such as heart rate or brain electrical signals.

Since the main workhorses in cells are proteins generated from genetic transcription and translation, by discovering ways to accurately identify abnormal proteins we can advance an understanding of disease. Recent developments in assay methods of processing DNA, RNA and proteins provides us with insights into these molecular biomarkers.

While these prior biomarker types have been used in traditional medical diagnostics, the genomics revolution has enabled new classes of molecular biomarkers that include genes, RNA, proteins, peptides and small molecules that supply detailed information about the presence, evolution and origins of diseases. This new generation of molecular biomarkers present enormous opportunities to advance personalized medicine by substantially increasing the precision of medical diagnostics.

Identifying a healthy gene, RNA or protein is insufficient to properly diagnose a disease. Rather, it is necessary to identify gene mutations, RNA variants and abnormal proteins in order to perform a diagnosis. The ability to identify and quantify these abnormal gene, RNA and protein molecules provides a revolutionary opportunity to identify, and understand the sources of, disease. In recent years, there has been an explosion of molecular biomarkers that indicate the presence of disease. The miR database (miRbase) of miRNA biomarkers consists of about 2500 examples. Though the FDA has approved only about 100 biomarkers, there are likely tens of thousands of potential molecular biomarkers that roughly mirror protein coding genes, non-coding genes, protein coding RNA, non-coding RNA, proteins, peptides and metabolite small molecules.

One of the critical enabling technologies for the industrial processing of biomarkers is next generation sequencing (NGS) that rapidly sequences molecular data in massively parallel arrays. For example, RNA-seq devices identify novel RNA variants.

Cancer applications are the paradigm for molecular biomarker study. Since all cancers have as their source genetic mutations, abnormal gene, RNA and protein biomarkers are critical tools for identifying the presence of the disease.

Many diseases have multiple biomarkers. Consequently, a macro-biomarker analytical methodology is required in a biomarker group selection process. ML is applied to analyze a set of biomarkers. The biomarkers are weighted in importance to a specific disease. The biomarker candidates are sorted according to active and passive status. Active biomarkers are ranked. The ML algorithm applies cluster analysis or regression analysis techniques to sort and rank the biomarkers. The result is to separate out the most likely biomarkers that may identify particular diseases among the hundreds of candidates.

Another factor to consider is the quality of a protein's abnormality. Protein abnormalities, like gene mutations, are not fixed, but rather exist on a spectrum. Identifying the specific geometrical configuration of a proteomic abnormality provides important information on the nature of specific pathology. Therefore, identifying the unique abnormality of each biomarker is critical to understanding the nature of a disease.

The existence of hundreds of abnormal molecular biomarkers in a patient may indicate the existence of a disease the precise dimensions of which would be unique to the patient. It is typical for a patient to embody hundreds of abnormal molecular biomarkers without yet exhibiting disease symptoms. However, a smaller subset of specific abnormal molecular biomarkers may reveal the source of a patient's genetic disease.

MMs are a central forum for application of biomarker analyses. Biomarker data are fed into the MM system, which then applies ML tools to analyze the biomarker information. In order to build a model of a disease, the MM needs to evaluate the patient's biomarker data. The MM compares the abnormal patient biomarker data to libraries and databases of healthy DNA, RNA, proteins and metabolites in order to evaluate the biomarker information. The MMs also build 3D models of molecular and cellular structures and attributes.

Physicians utilize MMs by inputting each patient's biomarker data in MMs in order to perform analyses of the data. ML tools apply AI techniques and algorithms to analyze the DNA, RNA and protein biomarker data. As an example, after 150 relevant biomarkers are discovered to identify a patient disease, the MM analysis reveals that five biomarkers are critical to indicate a particular form of the disease, thereby enabling an accurate diagnosis of the patient's underlying condition. Once identified, these critical biomarkers can then be used as drug targets. While MMs are a central tool for analysis of patient pathologies, molecular biomarkers represent the essential data inputs.

Molecular and cellular biomarker data provide clues to functional proteomic processes that indicate the presence of disease. Protein-protein interactions, protein-ligand interactions and protein-lipid interactions are examples of functional proteomic processes that are important to understanding the operation of disease. The MM system compares patient abnormal biomarker data to healthy biomarker data in DNA, RNA and protein databases and libraries in order to understand distinctive differences that differentiate a patient's pathology from a healthy patient.

In an embodiment of the invention, the system enables MMs to collect data from liquid biopsies in which blood tests reveal biomarkers that are then analyzed for the presence of cancer.

### Identification of Novel Biomarkers in IMMs

It is clear that gene variants [single nucleotide polymorphisms (SNPs)], RNA abnormalities and dysfunctional protein structures represent useful biomarkers for diagnostics. Whereas there are thousands of potential known biomarkers, how can we detect novel biomarkers? Novel biomarkers are useful in detecting the presence of, in monitoring, and in predicting the progress of, individualized diseases. RNA-seq is a key method to identify novel biomarkers, particularly RNA biomarkers.

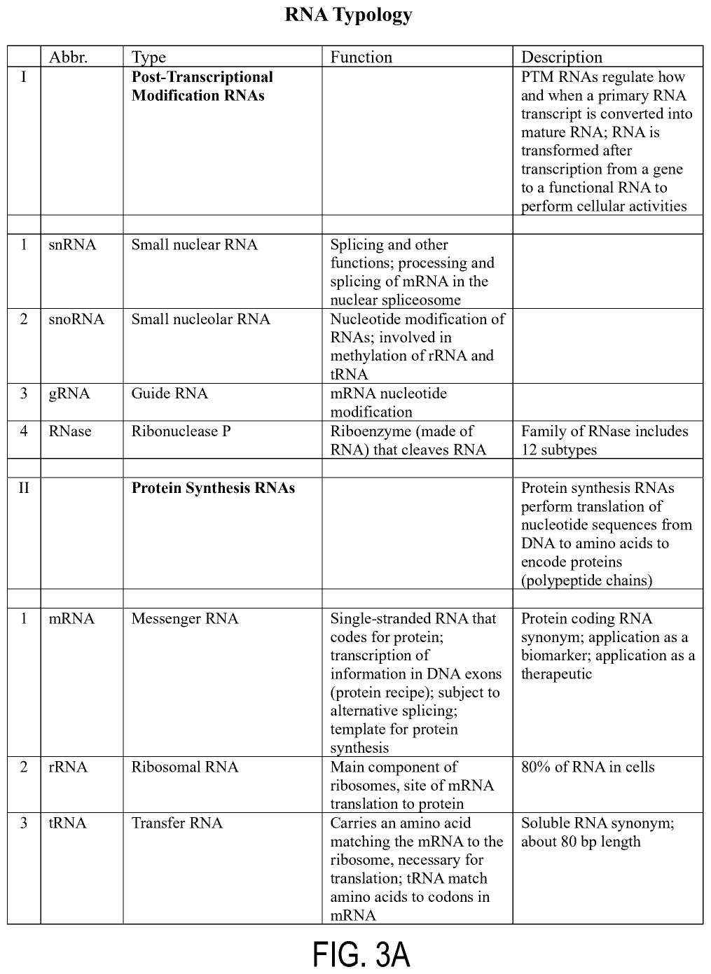

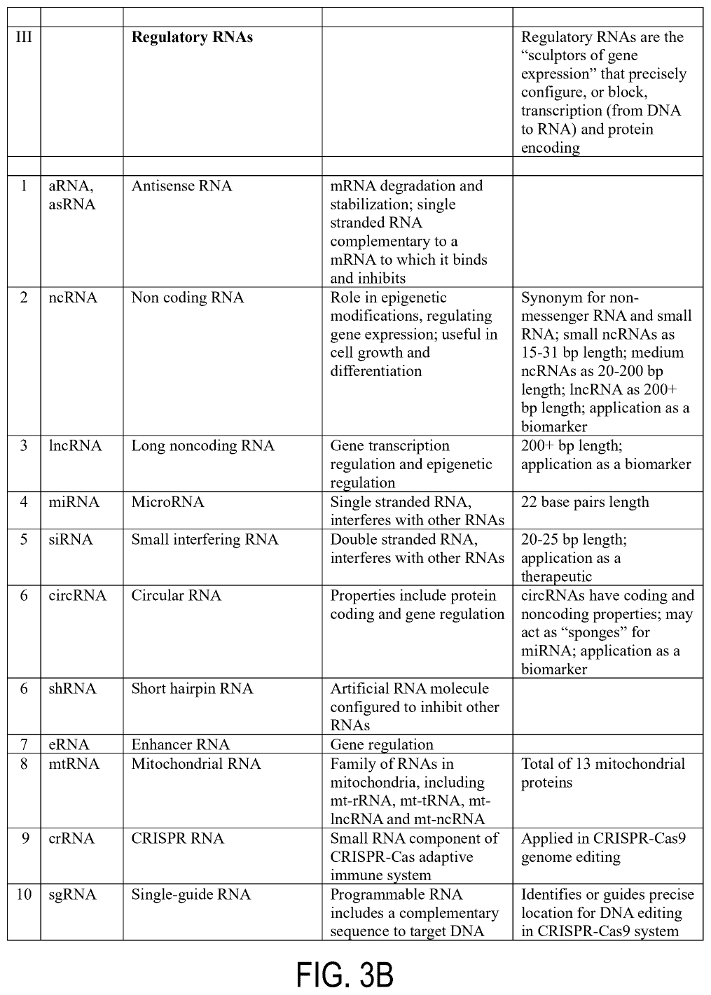

RNA analysis has emerged as a key analytical component for novel biomarker discovery. RNA represents a family of post-transcriptional molecules that convert DNA into proteins. Three main RNA types include post-transcriptional modification RNAs, protein synthesis RNAs and regulatory RNAs. Post-transcriptional modification RNAs include small nuclear RNA (snRNA), which splice mRNA, small nucleolar RNA (snoRNA), which are involved in methylation of rRNA and tRNA, guide RNA (gRNA), which modify mRNA, and ribonuclease (RNase), a family of RNase's which cleaves RNA. Protein synthesis RNAs include messenger RNA (mRNA), a single-stranded RNA that codes for protein, transfer RNA (tRNA), which carries an amino acid matching the mRNA to the ribosome to enable protein translation, and ribosomal RNA (rRNA), the main component of ribosomes and about 80% of the composition of RNA in a cell. Regulatory RNAs are considered the “sculptors of gene expression” that precisely configure, or block, transcription and protein encoding. Regulatory RNAs include antisense RNA (aRNA), a single stranded RNA complementary to mRNA to which it binds and inhibits, non-coding RNA (ncRNA), including small ncRNA (15-31 bp length), medium ncRNA (20-200 bp length) and long ncRNA (lncRNA) (200+bp length), which play roles in epigenetic modifications by regulating gene expression, micro RNA (miRNA), single stranded RNA (about 22 base pairs length) that interferes with other RNAs, small interfering RNA (siRNA), double stranded RNA (about 20-25 bp length) that interferes with other RNAs and circular RNA (circRNA), which possess properties that include protein coding and gene regulation. Among these major RNA types, three types are particularly useful as biomarkers to detect disease or disease progress. The three main RNA types useful as biomarkers are mRNA, lncRNA and miRNA. Refer to FIG. 3 for an RNA typology.

A reverse-engineering process for identifying RNA biomarkers from a specific disease is to take blood, fluid or tumor samples from a patient and initiate an RNA-seq testing process. These raw RNA data, which are typically mRNA, lncRNA or miRNA, are first plotted on a graph and then compared to RNA database reference data of healthy RNA examples. The sample RNAs reveal the expression levels of the patient disease relative to the RNA database reference data. These comparisons reveal substantial differences between the sample RNA examples and the reference RNA benchmark data, which are represented on a graph. The MM then weights the sample RNA examples to give priority to those with the strongest readings. For example, the RNA samples with the most quantity or more significant aberrations or abnormalities are weighted higher than more benign RNA samples. The MM then applies ML techniques to categorize the RNA samples according to functional utility whereby the RNAs are analyzed for their protein pathway utility. The most likely RNA biomarker candidates to signify a correlation with a specific disease are selected and ranked. In the next step, the RNA candidates are validated based on the highest likelihood of prediction of success in identifying a particular disease. Out of an initial set of over 1,000 RNA biomarker candidates, the final outcome may have fewer than twenty validated biomarker candidates. Depending on the quality of the RNA selection criteria, the list of validated biomarker candidates can be further restricted to a handful. At least one PHA may assist the MM in analyzing the RNA biomarker information.

Another method analyzes a pathology and reverse engineers the protein translation process in order to discover biomarker candidates. The biomarkers are analyzed in the context of mapping protein pathways in cellular networks. The identification of novel biomarkers is important in order to identify unique pathologies such as orphan diseases. Since there are at least 8000 orphan diseases, there are likely tens of thousands of novel biomarkers, many of which have yet to be discovered but which are crucial to understanding the origins and evolution of each genetic disease.

Similar exercises can be applied to DNA, protein, lipid and small molecule biomarker candidates. While the FDA has about 100 approved biomarkers, in the coming decades we may see thousands or tens of thousands of useful molecular biomarkers that enable researchers to identify and understand the evolution of many diseases with great precision.

Novel biomarkers are useful in analysis of the identification, and monitoring, of a precise phase of a disease progression. As a disease evolves from phase to phase, novel biomarkers can track the presence of the disease into each new phase. Analysis of patient biomarker data enables stratification of the patient disease according to a risk-based examination.

### In Silico Experiments for Diagnostics in IMMs

In silico experimentation is an important element of MMs. Rather than depend on in vivo or in vitro testing, in silico testing enables the application of a broad range of computational analytical tools that furthers our understanding of biomedicine. An argument can be made that DNA, RNA and protein aberration data alone are insufficient to identify a disease because this information simply says that a patient has a disease but does not understand the functional dynamics—molecular and cellular pathways, proteomic interactions and multiomics mechanics—necessary to understand the disease. For this reason, MMs, in combination with AI and ML techniques, provide a critical advantage in progress towards personalized medicine.

In silico experiments enable MMs to test different assumptions under different hypotheses in order to isolate biochemical, molecular, cellular or biological system problems. Particularly in the case of multivariate biological problems, the in silico experiment enables MMs to identify solutions to complex challenges involving diagnostics.

There are several main types of in silico experiments. First, there are diagnostic experiments to analyze a patient's disease. Second, there are diagnostic prognostics experiments to analyze the progress or evolution of a patient's disease without any treatment. Third, there are therapeutics experiments to analyze the best options to apply an existing therapy to the identified disease. Fourth, there are advanced therapeutics experiments to analyze the best options to design a novel synthetic drug for the identified disease. Finally, there are therapeutic prognostics to analyze the dynamics of a disease progress with application of a drug or drugs. Theoretically, it is possible to analyze a patient's pathology from diagnosis and prognosis through therapeutics and therapeutic prognostics entirely in MMs with in silico experimental analytics.

One example of application of in silico experiments with MMs involves the process of identifying a patient's pathology target. Identifying a mutated gene or an abnormal protein structure by applying analytical techniques or by reverse engineering an experiment to discover the source of a disease is a paradigm of medical diagnostics. The experimentation process can discover more information than sequencing data alone. ML-assisted MMs can analyze the unique expression characteristics of a gene mutation or an abnormal protein that are essential to understanding the unique complexity of an individual's disease. In addition to the structural abnormalities of a patient's disease profile, the experiments also analyze the operational dynamics of patient pathology. The MMs generate and test hypotheses about operational dynamics of abnormal DNA, RNA, protein and cells behaviors. Once a pathology target is identified, for example, molecular or cellular processes that reveal a pathology, then the pathology can be precisely solved by applying a drug.

The in silico experimentation processes in MMs are dynamic since the modeling characterizes the physiology and biopathway mechanics of unique patient diseases. In silico experiments are well suited to identifying pathology protein pathway mechanics. In another example of application of in silico experiments, complex multivariate diseases, or multiple diseases, may require extensive computer analysis to understand the disease(s). Regression and classification analyses applied by ML in MMs and in silico experiments may solve these complex multivariate medical problems.

The IMM system reviews medical research articles and medical libraries in order to obtain insights into reference medical data to inform its models. For example, medical research articles are mined by applying NLP techniques in order to identify drug targets. When a model identifies a specific set of gene mutations that express as a particular disease, the MMs collect a list of drug candidates that may match the specific pathology.

MMs can apply in silico experiments to test for numerous biomedical phenomena, including dysfunctional proteins, dysfunctional cells, protein targets, protein binding sites, protein-ligand binding and protein-protein interactions. In an embodiment of the invention, the MMs can conduct experiments in order to test multiple simultaneous parallel interactions. Proteins can be tested simultaneously with other proteins, ligands, lipids and small molecules.

If a patient has a cancer with 85 genetic mutations, the MMs can apply combinatorial logic, combinatorial algebra and/or partial differential calculus in order to identify the specific genetic variant combinations that are most likely causing the patient pathology in order to accurately identify disease targets.

In silico experiments enable testing of assumptions, hypotheses or variables in complex biomedical diagnostic problems. PHAs are applied to experiments to accelerate the testing process. PHAs apply ML techniques and algorithms to develop sims of experiments in order to accelerate diagnostic analytics.

The MM system activates in silico experiments involving protein-protein interaction, drug-target interaction, drug-disease interaction and drug-drug interaction. The modeling system builds sims of processes as well, including intracellular processes, dysregulation of protein pathways, DNA to RNA anomaly transcription and dysfunctional RNA to protein translation processes. In addition, the modeling system generates molecular protein models and sims, including models of healthy protein to dysfunctional protein interactions, molecular docking, molecular receptors, molecular inhibitors, protein-ligand interactions, protein-lipid interactions, protein to small molecule interactions and RNA translation into proteins or peptides. The modeling system also generates molecular models of non-coding DNA and RNA into peptides that may regulate DNA or RNA. In addition, the modeling system generates models involving cellular interactions, cellular regulation, inter-cellular signal transduction networking and inter-cellular dynamics. Moreover, the modeling system performs computational analysis of protein docking predictions, including the most probable docking candidates. Finally, pathogens can be analyzed in the IMM system, including the possible reaction scenarios of patients with and without vaccination.

Dozens of AI and ML techniques may be applied to in silico experimentation in MMs as listed in FIG. 2.

In an embodiment of the invention, the IMM system can build two sets of models for each patient diagnostic. The first set of models is a reference model of healthy biological processes that mirrors healthy reference multiomics databases. This set of models may refer to the patient's past healthy history. The second set of models refers to the patient's disease analysis. This second set of models analyzes the patient pathology by employing ML and in silico experiments of multiomics data sets in order to discover the patient disease. Possessing the two sets of models is useful for comparison with the patient herself rather than with a reference database and enables the useful tracking of the disease progress by constant reference to the healthy reference models.

Without a precise identification of a pathology, how can one apply a safe and effective therapy? Once we have found a precise disease source (target), such as dysfunctional genes or proteins, then we can begin the process of discovering or designing precisely targeted therapies.

The in silico experiments embedded in MMs for application to diagnostics are organized on Levels 4 and 5 of the present system.

### Applications of ML and GenAI to Diagnostics in IMMs

GenAI, ML and DL are applied to diagnostics in MMs by analyzing data on molecular biomarkers, cellular features, gene expression and protein interactions. MMs model, simulate and analyze molecular biomarker data with GenAI, ML and DL.

First, variational auto encoders (VAEs) are applied to the identification of gene expression stimulated by a chemical compound.

Second, natural language processing (NLP) is applied to an analysis of the translational language of amino acids sequences and relations.

Third, large language models (LLMs) and NLP are applied to the identification of relations between genes and disease targets.

Next, geometric deep learning techniques are applied to a 4D functional analysis of molecular biomarkers.

Further, manifold valued neural networks (MVNs) are applied to the non-Euclidean 3D analysis of molecular features of biomarkers.

Finally, generative convolutional neural networks (GCNNs) are applied to analysis of protein-protein interactions and protein-ligand interactions.

PHAs utilize one or more of these techniques in order to analyze and identify pathologies for diagnostics in MMs.

### IMMs Applied to Analyzing Diagnostics in Critical Diseases

Several classes of diseases are associated with a high percentage of mortality in many countries worldwide. These diseases are cardiovascular pathologies, neurological and psychiatric pathologies and oncology pathologies. Not coincidentally, these three classes of diseases are involved with diseases of aging and are associated with rapid decline in the quality of life for millions of patients worldwide. MMs are applied to analyzing diagnoses in these diseases.

In addition to these disease categories, there are thousands of orphan genetic diseases that will likely benefit from MM analytics and diagnostics.

### Cardiovascular Applications

Cardiovascular diseases have been the number one cause of death in the US for the last century. Arteriosclerosis, hypertension, hypercholesterolemia, arrhythmia, vascular disease and stroke are among the top categories of heart disease. Also refer to the discussion below of preemptive medicine for an analysis of cardiovascular diseases.

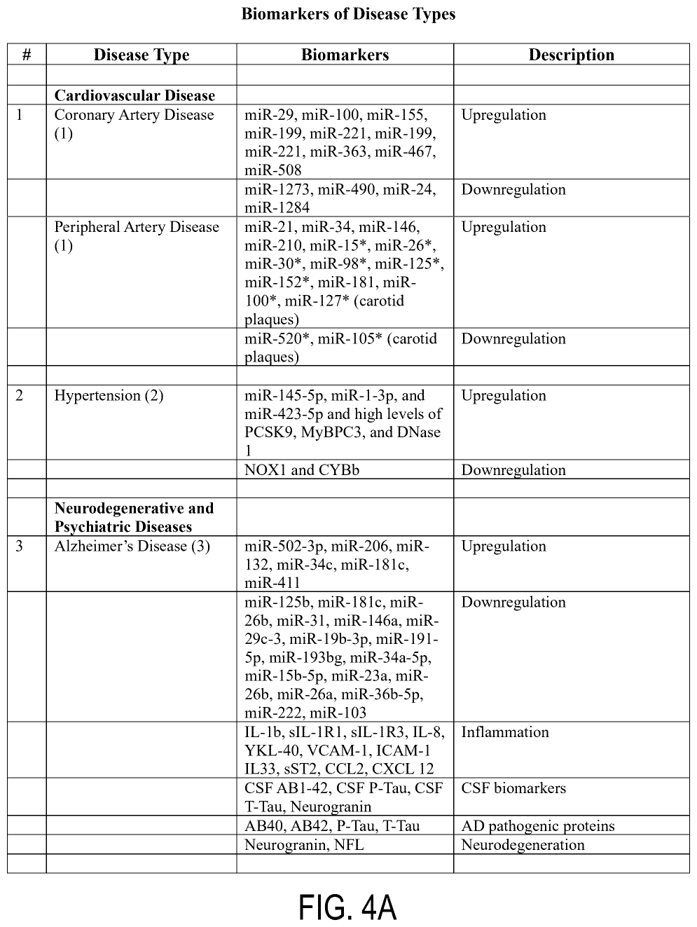

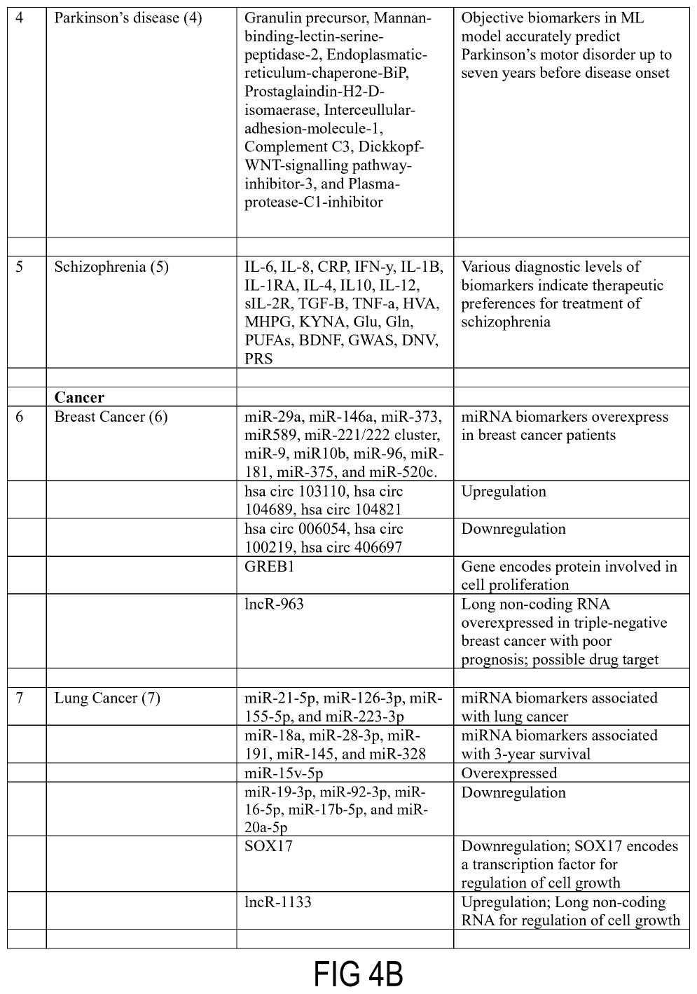

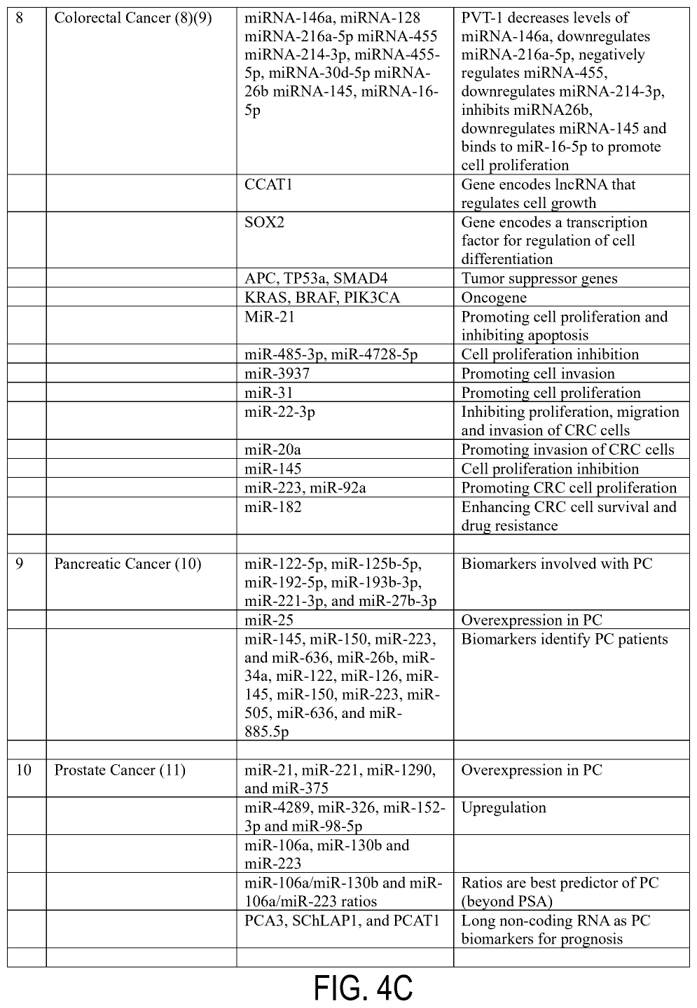

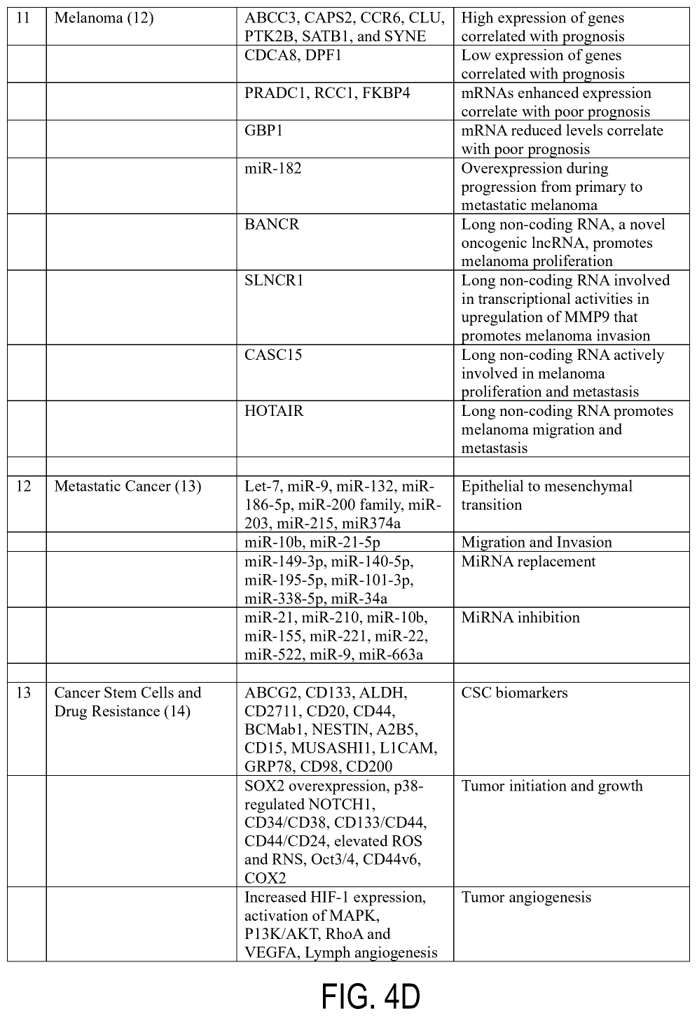

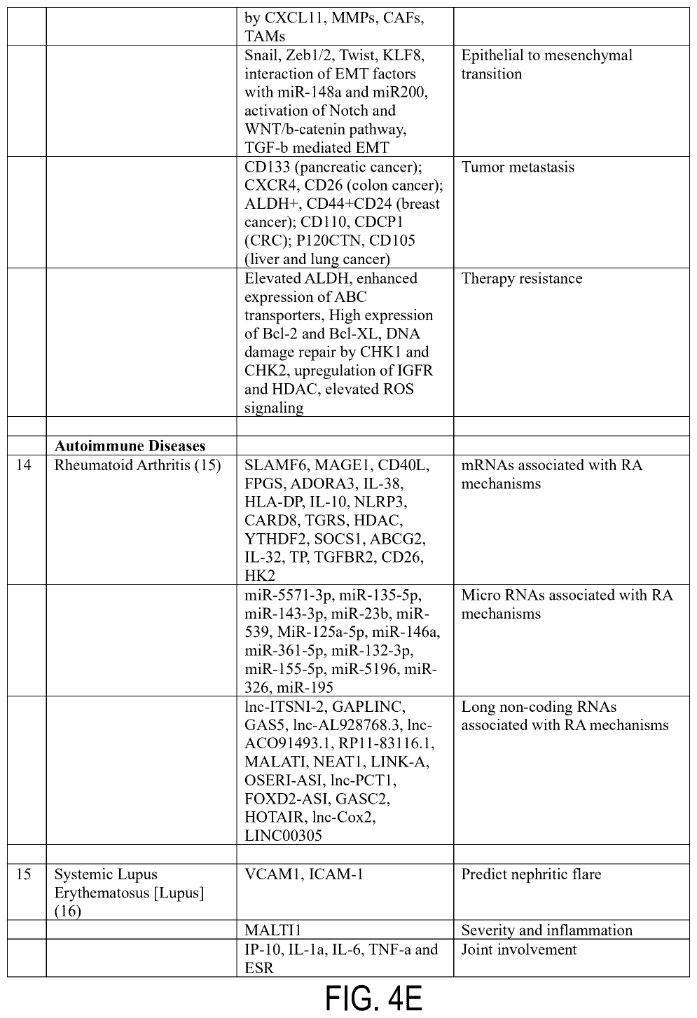

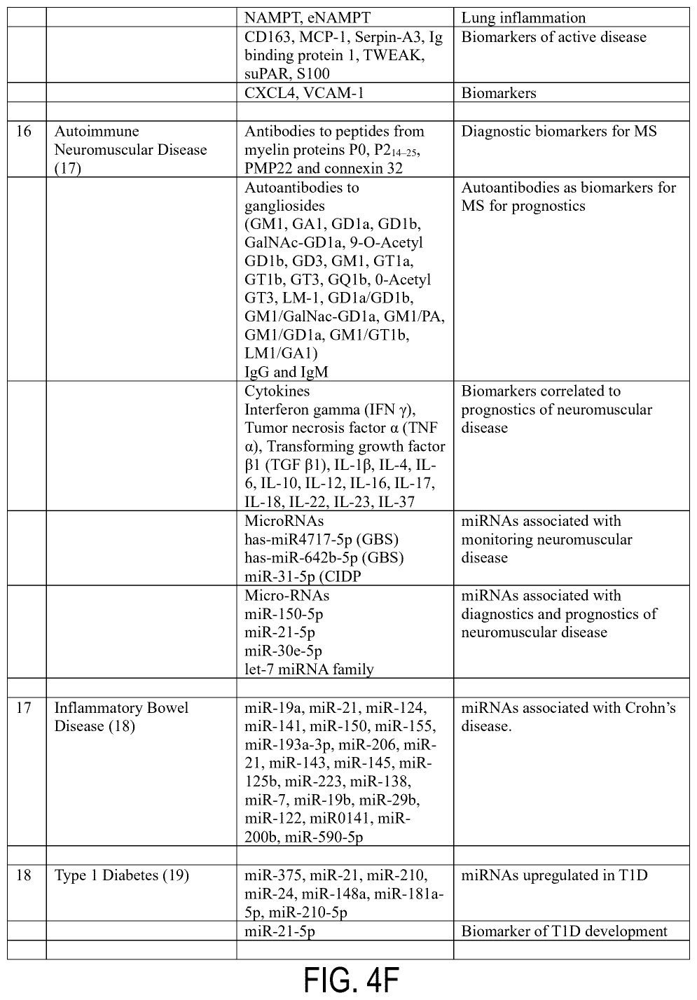

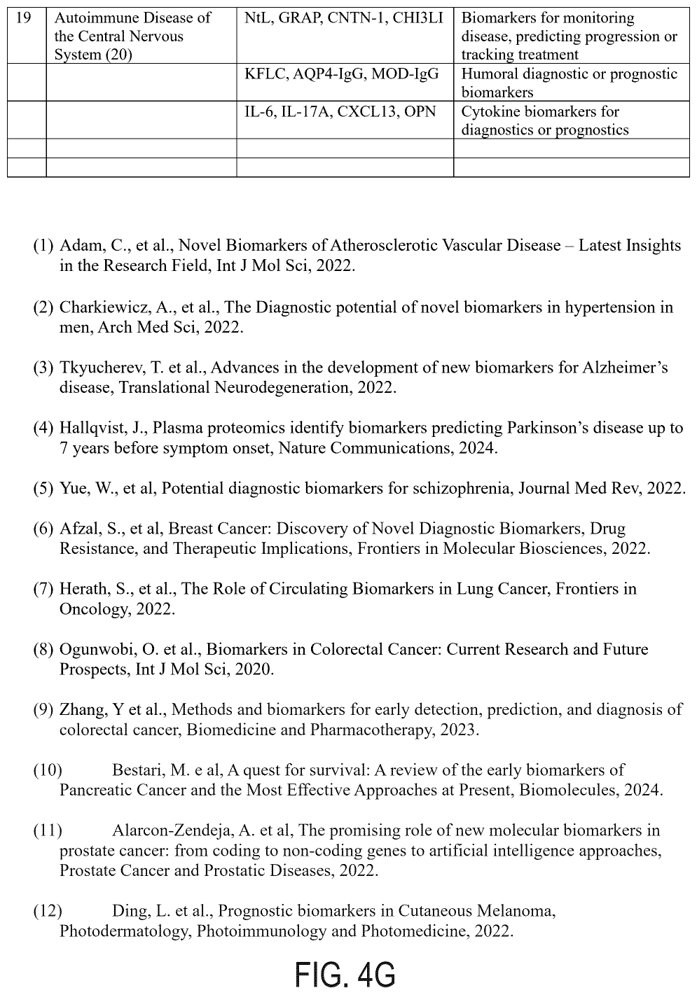

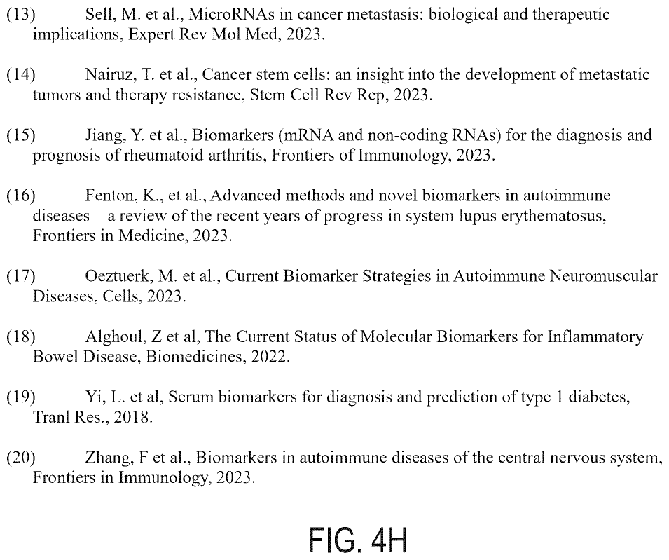

Biomarkers involving coronary artery disease are identified that are upregulated (miR-29, miR-100, miR-155, miR-199, miR-221, miR-199, miR-221, miR-363, miR-467 and miR-508) and downregulated (miR-1273, miR-490, miR-24 and miR-1284). Please refer to FIG. 4. Biomarkers involving peripheral artery disease identify biomarkers that are upregulated (miR-21, miR-34, miR-146, miR-210, miR-15*, miR-26*, miR-30*, miR-98*, miR-125*, miR-152*, miR-181, miR-100* and miR-127* (*=carotid plaques)) and downregulated (miR-520* and miR-105* (*=carotid plaques)). Regarding hypertension, biomarkers are identified that are upregulated (miR-145-5p, miR-1-3p and miR-423-5p and high levels of PCSK9, MyBPC3 and DNase 1) and downregulated (NOX1 and CYBb). MMs are applied to analyze the biomarkers indicating components of cardiovascular disease.

### Neurological and Psychiatric Applications

Neurological and psychiatric disorders are responsible for about ten percent of deaths in the US. Neurodegenerative pathologies include Alzheimer's disease, Parkinson's disease and Huntington's disease. Psychiatric pathologies include schizophrenia, bipolar disorders, depression and addiction.

Biomarkers involving Alzheimer's disease are identified that are upregulated (miR-502-3p, miR-206, miR-132, miR-34c, miR-181c and miR-411) and downregulated (miR-125b, miR-181c, miR-26b, miR-31, miR-146a, miR-29c-3, miR-19b-3p, miR-191-5p, miR-193bg, miR-34a-5p, miR-15b-5p, miR-23a, miR-26b, miR-26a, miR-36b-5p, miR-222 and miR-103). In addition, inflammatory biomarkers include IL-1b, sIL-1R1, sIL-1R3, IL-8, YKL-40, VCAM-1, ICAM-1 IL33, sST2, CCL2 and CXCL 12. Please refer to FIG. 4.

Biomarkers for Parkinson's disease indicate motor disorder up to seven years before disease onset. These biomarkers include Granulin precursor, Mannan-binding-lectin-serine-peptidase-2, Endoplasmatic-reticulum-chaperone-BiP, Prostaglaindin-H2-D-isomaerase, Interceullular-adhesion-molecule-1, Complement C3, Dickkopf-WNT-signaling pathway-inhibitor-3 and Plasma-protease-C1-inhibitor.

Biomarkers for Schizophrenia include IL-6, IL-8, CRP, IFN-y, IL-1B, IL-1RA, IL-4, IL10, IL-12, sIL-2R, TGF-B, TNF-a, HVA, MHPG, KYNA, Glu, Gln, PUFAs, BDNF, GWAS, DNV and PRS.

For the most part, these biomarkers provide diagnostic certitude. However, these biomarkers are also applicable to diagnostic prognostics in estimating and projecting the course of each disease. MMs are useful in analyzing these biomarkers for diagnostics and diagnostic prognostics.

### Oncology Applications

Cancer is the second leading cause of death in the US. The top six solid tumor cancer types, in order of incidence, are breast cancer, lung cancer, colorectal cancer, pancreatic cancer, prostate cancer and melanoma. Please refer to the discussion below regarding the application of IMMs to metastatic cancer, which is responsible for 90% of cancer deaths.

Regarding breast cancer, some biomarkers indicate overexpression in breast cancer patients, including miR-29a, miR-146a, miR-373, miR589, miR-221/222 cluster, miR-9, miR10b, miR-96, miR-181, miR-375, and miR-520c. Other biomarkers identify upregulation (hsa circ 103110, hsa circ 104689 and hsa circ 104821) and downregulation (hsa circ 006054, hsa circ 100219 and hsa circ 406697).

Regarding lung cancer, some biomarkers are associated with the presence of the disease (e.g., miR-21-5p, miR-126-3p, miR-155-5p and miR-223-3p) and with a 3-year survival (miR-18a, miR-28-3p, miR-191, miR-145, and miR-328). While one biomarker (miR-15v-5p) is associated with overexpression, others (miR-19-3p, miR-92-3p, miR-16-5p, miR-17b-5p and miR-20a-5p) are associated with downregulation.

Regarding colorectal cancer, some biomarkers (miRNA-146a, miRNA-128 miRNA-216a-5p miRNA-455 miRNA-214-3p, miRNA-455-5p, miRNA-30d-5p miRNA-26b miRNA-145, miRNA-16-5p) are associated with the presence of the disease, while others (MiR-21, miR-485-3p, miR-4728-5p, miR-31, miR-223 and miR-92a) are associated with cell proliferation or inhibition.

Regarding pancreatic cancer, biomarkers (miR-122-5p, miR-125b-5p, miR-192-5p, miR-193b-3p, miR-221-3p, miR-27b-3p, miR-145, miR-150, miR-223, and miR-636, miR-26b, miR-34a, miR-122, miR-126, miR-145, miR-150, miR-223, miR-505, miR-636 and miR-885.5p) are associated with the presence of the disease. One of the main challenges of pancreatic cancer is the difficulty of timely diagnosis. By the time most people are diagnosed, the cancer has spread and has progressed to a terminal stage IV, leaving a poor prognosis. This situation of late diagnosis illustrates the need to identify methods for early detection, which RNA biomarker analysis may well provide.

Regarding prostate cancer, biomarkers (miR-21, miR-221, miR-1290 and miR-375) are associated with overexpression of the disease while other biomarkers (miR-4289, miR-326, miR-152-3p and miR-98-5p) indicate upregulation. Additional biomarkers, such as long non-coding RNA (PCA3, SChLAP1 and PCAT1) are associated with prognosis.

Regarding melanoma, various RNAs are associated with melanoma proliferation (BANCR and CASC15) while others are involved with (poor) prognosis (PRADC1, RCC1 and FKBP4). MMs are important in analyzing these biomarkers for diagnostics and prognostics.

### Individualized Medical Modeling for Diagnostic Prognostics

The term “prognosis” derives from the Greek term for “foreseeing” or “foreknowing.” A prognosis of a disease refers to the probable progress of a disease, particularly without (therapeutic) intervention. While a diagnosis of a disease indicates where we are and where we have been, a prognosis of a disease indicates where we are going. An accurate diagnosis is critical to understanding the course of a disease; therefore, a disease diagnosis establishes a starting point. In general, the way to understand the progress of a disease is to apply objective methods of evaluation, such as imaging, digital tracking or molecular biomarkers. In a sense, as the disease evolves, the diagnosis itself shifts in such a way that the diagnostics is itself a moving target. Tracking the changes in the evolution of a disease is important to understanding its probable outcomes. In general, there are several different outcomes of a disease: a complete cure (e.g., elimination of a head cold), the death of the patient (e.g., some cancers), the cessation or limited further evolution of a disease (e.g., LDL increase is offset by a change in diet) and disease management (e.g., drugs manage, but do not cure, the disease to reduce further decline).

Most genetic-involved diseases are progressive. In many cases, the affected genes in a disease encounter a process of deterioration over time that increases the production of abnormal proteins. The disease degradation tracks the gene condition decline. The disease tracks dysfunctional protein scenarios that manifest in escalating disease expressions. In a microscopic sense, prognosis involves the tracking process of protein and cellular degradation. According to this view, prognostics is the field that identifies phases in the evolution of gene and RNA mutations or protein dysfunctions and maps these deteriorating molecular and cellular conditions on the evolution of a disease. Since tracking molecular biomarkers are a critical method to understanding a patient's disease, measurement and analysis of the changes of these biomarkers provides a critical qualitative analysis of the evolution of the disease.

One of the main goals of prognostics is to develop accurate predictions of the course of a disease. Yet, in order to develop accurate predictions, we need more information than merely biomarker data from a single patient. Patient disease evolution data must be compared to a database of other patient's which have had a similar disease in order to assess their probable course(s) of a disease's development. The patient's disease is forecast based on a comparative analysis of groups of other patients with similar profiles and past similar diseases. The analogy is to actuarial charts in the insurance industry in which aggregated data are compared to individuals with similar profiles in order to assess the probability of specific outcomes with similar conditions.

Amore pragmatic view of prognostics suggests a multifactorial analysis to predict scenarios from different situations that are not completely analogous. In addition to genetic factors, there are broad differences among patients involving environmental factors that may influence the development of a disease. For instance, a disease may evolve differently for a non-smoking vegetarian and for a meat-eating smoker. There is a need to compare the patient disease evolution data to an aggregated database of patients, but the multifactorial analysis dimension elicits a broader set of variables.

Diagnostic prognostics is related to diagnostics, therapeutics and clinical trials. Prognostics is related to diagnostics as diagnostics identifies a disease while prognoses measure and predict the disease's evolution. Early diagnostics of a disease may anticipate the emergence of a disease even before symptoms appear and tracking the predictions of the early diagnostic prognostics suggests the materialization of a new field of preemptive medicine which anticipates the development of diseases before symptoms actually appear. Examples of these preemptive medicine categories include arthritis or arteriosclerosis. Diagnostic prognostics is applied to clinical trials since the control arm of a drug trial can be represented as a set of virtual patients that embody aggregated patient data. In the control arm of a clinical trial, the patient disease evolves without intervention. Finally, diagnostic prognostics is related to therapeutic prognostics. Whereas diagnostic prognostics identifies the progress of a disease without medical intervention, therapeutic prognostics begins with the diagnostics model and then applies different medical therapies, i.e., drugs, to identify whether the disease can be stopped or managed. In therapeutic prognostics, the prognosis is updated in relation to feedback from drug applications. Consequently, the decline in patient condition associated with no treatment is contrasted with the therapy options the addition of which provide measurable benefits to the trajectory of the patient's disease evolution.

The present system develops a grading approach, a “prognosis score,” in order to predict disease outcomes. Emulating credit scores that predict an individual's financial performance, the prognosis score identifies, and projects, the changes over time of a patient's medical condition. The scoring system is overlaid onto a 70-100 scale, with 100 being the highest, and healthiest, score and 70 being the lowest and unhealthiest score. While, superficially, patient symptoms can be a guide to anticipating patient health, this primitive approach to estimating future pathology development lacks empirical foundations. Rather, the scoring system applies an analysis of biomarkers in order to evaluate trends in the pathology data in order to probabilistically anticipate changes in a patient's condition over time. The pathology can be mapped over time with different scenarios based on the different possible inputs, such as patient behavior or environmental changes. The patient scores can be compared to other patient scores with similar diseases and prognoses. The patient prognosis of disease is a moving picture. The patient scoring changes over time when the pathology data change; as new data are available, the prognosis is updated to reflect the deterioration or improvement of the patient condition. For instance, as the quantity of biomarkers indicate the increased velocity of a disease progression, the patient's prognosis score is updated to reflect this deterioration. The tracking of a patient's disease is analogized to the changes of public opinion in a poll as new data influences and reflects the latest opinions.

IMMs are applied to diagnostic prognostics to model diseases by inputting biomarker and other medical data and analyzing the medical data over time in order to assess and predict the evolution of the patient's disease.

### Biomarker Analysis in IMMs for Prognostics

The prognosis of diseases is presented in MMs in the context of predictive probabilistic scenarios. A MM may provide a short-term outcome prediction and a long-term outcome prediction under different conditions. MMs rely primarily on an analysis of biomarkers in order to develop accurate projections of disease prognostics. The biomarker analysis enables insight into disease mechanisms. On one level, the quantitative biomarker data show the simple evolution of a disease. On another level, the qualitative transformation in the degradation of biomarker (i.e., gene, RNA, protein, lipid or small molecule) data reveals important disease characteristics that inform the prognosis. In effect, MMs track the progress of biomarkers as a proxy for a disease (or diseases). The models build a picture of the progress of the disease and make predictions from the model based mainly on the biomarker analysis.

ML techniques are applied in MMs to analyze and predict biomarker evolution. Identification of changes in biomarker condition suggest the ability of the models to ascertain different stages of a patient's disease progress. In the case of cancer, there are several stages of a disease's evolution, wherein the compression between a second phase and a fourth phase is increased. Therefore, there is an increased need to identify the first stage diagnosis in order to implement therapy so as to stop or slow the disease progress. But the MMs can predict the intensity or aggressiveness of a disease's progress given the biomarker data at different points in time.

In a sense, the assessment of biomarker data is simply a reference to snapshots in time of separate diagnoses. Comparing and analyzing these different snapshots across time suggests our ability to develop a prognosis that projects these trends into the future. Numerous biomarker readings over time yield data sufficient to identify trends that enable accurate predictions. For example, taking multiple readings of PSA for prostate cancer can indicate a trend that may portend probable outcomes; in some ways, the main factor in this analysis is the quantity of the biomarker exuded by the prostate gland that may indicate the phase of evolution of the disease.

MMs combine patient biomarker data with biomedical library data in order to compare the patient to reference data. The MMs can also compare a patient's biomarker data to other patient's pathology biomarker data which are structured with disease outcome information. The MMs apply a micro-prognostic analysis to assess the evolution of molecular biomarkers. In an embodiment, the MMs can predict the presence of a future biomarker that will forecast a stage of the disease progress if later confirmed.

The MMs identify specific predictive biomarkers, the presence of which may forecast a particular phase of the evolution of a disease. The biomarkers are classified, sorted and ranked in order to assess the relative weight and value of particular biomarkers in making predictions about a pathology. In some cases, a super-regulator biomarker is identified that is useful in indicating the progress of a disease. In other cases, targeting this super-regulator biomarker is critical to blocking the evolution of a disease; generating a drug to target this biomarker is the key to stopping the pathology. Biomarker analyses for a pathology enables the matching of a pathology with drug therapy options suggesting that biomarker analyses may be critical for drug discovery.

### In Silico Experiments for Diagnostic Prognostics in IMMs

MMs are useful for conducting in silico experiments for prognostics. MMs track disease evolution by analyzing biomarker conditions and expressions over time. PHAs automate in silico experiments of biomarkers by developing simulations from biomarker analytical data. Specifically, in silico experiments analyze protein-protein interactions, protein-ligand interactions, protein-lipid interactions and protein-small molecule interactions. MMs project 3D protein structure and function predictions by analyzing biomarker data sets. Protein dysfunction data are modelled in 3D and 4D, with 4D simulations estimating scenarios of projected protein functions. In silico experiments enable MMs to analyze biomarker data over time and to plot scenarios of probable pathology behaviors.

Micro-prognostics analyses in MMs reveal protein geometric structure predictions that delineate the protein interactions in cellular pathways that generate and manifest disease. In effect, mapping the degradation of protein structure reveals protein functional decay. From these models of protein dysfunction, the MMs reveal cellular mechanisms of operation, from healthy to pathological. The patient's pathology analysis is contrasted to a healthy protein function model. For instance, the MMs may compare the patient aberrational biomarker data to a theoretical reference model in a protein structure prediction LLM in order to show the degradation of the patient's protein biomarkers. From these biomarker measurement data, the MM maps various prediction scenarios based on multiple factors, including a stable genetic mutation and environmental changes. By analyzing biomarkers, in silico testing in MMs enable the prediction of disease within a range of scenarios and probabilities. Intracellular protein pathway behaviors are predicted from multiomics data analyses in MMs.

MMs and in silico analyses enable not only disease progression analysis, but also drug-target prediction and drug-disease prediction. In the case of drug-target prediction, the biomarker analysis identifies the target sufficiently to specify a drug tailored for that target. Even though a pathology is a moving picture that changes over time, the target, typically an abnormal protein, is specified as a focus or objective of drug development. In the case of drug-disease prediction, the disease evolution is tracked in such a way as to identify a drug that will manage or cure the disease at different stages of the disease's progress. Drug reaction simulations can be modelled in MMs.

### Applications of ML and GenAI to Diagnostic Prognostics in IMMs

3D GDL is applied to prediction and forecasting of protein dysfunctional states.

Variational autoencoders are applied to predict cell states from compound attributes.

GDL, diffusion models and generative convolutional NNs are applied to 3D protein structure prediction.

GDL is also applied to 3D analysis and prediction of molecular structures.

Graph attention networks (GATs) are applied to the analysis and prediction of properties of molecules.

Graph NNs are applied to problems associated with the extraction of features from graphs to predict molecular geometry. In some cases, graph nodes and edges are weighted.

Graph convolutional NNs (GCNs) are applied to predict molecular properties as well.

### Individualized Medical Modeling for Therapeutics

The two main therapeutic modalities include traditional targeted drug discovery and novel synthetic drug design. Both therapeutic modalities involve first identifying a target, typically a protein, which a drug agent candidate aims to resolve. Identifying with precision the molecular pathways and targets of a disease are critical to developing a drug that can act on the target.

For centuries, drugs were discovered by accident. Typically, a chemical from a plant would be extracted to solve a specific pathology. In some cases, a random drug would be discovered first and then a search would be made of its possible uses, effectively reversing the modern approach of searching for solutions to pathology challenges. Eventually, chemical extraction from plants established the foundation for traditional pharmacology. The extraction of pure chemicals from plants enabled the creation of drugs that could treat patient pathologies. For example, morphine was extracted and purified from poppies or cocaine was extracted and purified from coca.

But the deciphering of the human genome established the fields of genomics, proteomics, metabolomics, etc., and focused attention on molecular causes of many diseases. The advent of microarrays and next generation sequencing (NGS) enabled testing for DNA, RNA and proteins that allow insight into gene variants and dysfunctional proteins. Typically, small molecules are identified or developed to match or solve a cellular pathology mechanism. The goal is to search for a drug agent candidate to modify the function of the affected target in order to reverse a disease state or improve symptoms. The idea is to identify small molecules that influence the behavior of proteins (targets), that is, to interfere with the mechanism of dysfunctional proteins that characterize a disease state or to initiate a specific biological process in the body.

The molecular target, generally a dysfunctional protein or a set of proteins, is involved in a dysfunctional cellular pathology mechanism. It is necessary to understand the functional pathways of operation of the protein target as it operates in the dysfunctional cellular pathology. The main ways to understand the pathology pathway mechanisms and their processes of dysfunctional operation are to run NGS or high throughput screening (HTS) tests in order to track dysfunctional biomarkers for a particular disease. The validation of dysfunctional biomarker candidates reveals the operations of pathological cellular mechanisms.

Three examples of small molecules eliciting a therapeutic reaction include enzyme inhibitors, receptor agonist/antagonist and ion channel modulators. In the case of enzyme inhibitors, enzymes (proteins) catalyze organic chemical processes, with small molecules interfering with the enzymatic responses. Statins are a class of enzyme inhibitors, which block cholesterol generation in the liver. In the case of receptor agonist/antagonist, small molecule drugs interact with cell surface proteins such as agonists that activate the receptor or antagonists that inhibit the binding of the signaling process. In the case of ion channel modulators, ion channels are proteins in cell membranes that regulate the flow of ions in cells. Small molecule drugs can modulate the opening and closing of the cellular regulatory channels.

Traditional drug discovery is a laborious process involving the trial-and-error process of discovery coupled with the intensive process of testing many chemical compounds for efficacy. In many ways, modern drug discovery merely automates and accelerates the traditional drug discovery model by applying HTS and chemical assays to identify and test many chemical compounds in a limited time. Drug candidates are screened, tested and eventually winnowed in drug clinical trials.

Modern drug discovery focuses on two main classes of drugs: Small molecules and biologicals. Small molecules, which comprise about 80% of approved medicines, are typically synthetic chemical compounds. Biologicals include recombinant proteins, antibodies, long peptides, genes, RNAs and vaccines.

Once drug candidates are identified in medical research, they are tested in pre-clinical approaches. These basic research or pre-clinical testing phases of drug testing include in vitro (tested in a test tube), ex vivo (tested in tissues or organs), in vivo (tested in a living organism such as mice, rats, dogs or pigs) or in silico (tested in a computer). Once a drug candidate passes these pre-clinical testing phases, it passes from drug discovery to drug development as the drug candidate initiates drug clinical trials. The basic research phase of drug discovery and the pre-clinical phases of drug development could collectively take a decade, with only about twenty percent surviving the gauntlet.

Even when the surviving drug candidates enter clinical trials, which typically consist of three phases of random blind studies, only about ten percent of drug candidates survive to the point of FDA approval. The average period from drug discovery to drug approval is 10-15 years and about $2.5B. A drug discovered today may be approved in over a decade at a cost of over $4B. This process begs for a simpler, more targeted, cost-effective and quicker method of drug discovery and development. MMs, ML and GenAI will likely revolutionize these processes in the next generation.

Personalized Medicine with IMMs Applied to Drug Discovery

Personalized medicine endeavors to identify the precise pathology of a patient on a genetic or molecular level. Typically, genetic mutations or RNA aberrations generate dysfunctional proteins that cause or express a pathology. An assessment of a patient's molecular biomarkers can identify the precise combination of abnormal proteins that generate the pathology. By identifying these dysfunctional proteins, we not only identify the precise patient pathology diagnosis, but also accurately identify the protein targets on which we can focus a medicinal solution.

MMs, in combination with ML and GenAI, are well suited to understand the patient pathology by identifying the unique combination of biomarkers that signify genetic mutations, RNA aberrations and dysfunctional proteins. These aberrant proteins become targets for drug discovery. The MM represents an intermediary analytical phase in a personalized drug therapy development, between the collection and analysis of empirical biomedical data (biomarker data), which data and analysis are crucial in order to identify and understand the nature of the patient disease, on the one hand, and to identify therapeutic drug options, on the other, that will cure or manage the disease.

In a sense, the protein targets represent multi-objective optimization problems, the solutions of which are drug therapies. The MMs generate solution options for the multi-objective optimization problems by analyzing the protein targets.

Once a patient's protein targets are identified, the MMs search drug libraries and protein databases to identify drug candidate solutions. MMs apply ML to analyze the drug candidate options and to identify similar protein targets. The MMs select drug candidates in order to test against the protein targets. The MMs predict drug candidate effects, including potential adverse side effects, as applied to the protein targets.

The MMs search for alternative solutions than small molecules, including biologicals such as antibodies, recombinant proteins, RNA or long peptides. The MMs apply ML to analyze these drug candidate options against protein targets.

Ideally, the MMs identify an existing drug therapy for a unique patient pathology. The ability to match individual patient proteomic pathology to an existing targeted drug therapy is useful.

The selected drug candidates are scored for different probable medicinal solutions for each pathology by the MM. The MM selects the best medicine option based on the probability score and then ranks the drug options to solve the medical problem.

The MM will map the protein pathway and show the effects of a dysfunctional protein. The MM will show how different drug candidates will act in the dysfunctional model with different levels of therapeutic benefits. The details of a patient pathology, including genetic, RNA, proteomic and metabolomic dysfunctional details, are mapped so one can compare the dysfunctional model to a healthy patient model.

MMs enable the focusing of the drug discovery process. The process begins with the discovery of the genetic, RNA and protein source of a pathology, which represents a precise diagnosis. From this understanding of identification of protein targets, drug therapy options are generated, selected and tested. ML and GenAI are applied to predict probable outcomes of different drug candidate options. For instance, different drug types can target different kinds of genetic mutations of similar genes that generate differentiated dysfunctional proteins. MMs also identify optimum drug dosage and drug timing (i.e., drug release characteristics) by simulating, predicting and optimizing drug delivery mechanisms.

In an embodiment of the invention, MMs predict drug toxicity by analyzing drug candidate interaction in protein pathway cellular operations.

IMMs and In Silico Laboratory: Drug Discovery Modeling and Experiments

In silico experimentation enables computational drug testing in IMMs.

Modern drug discovery is accelerated by in silico experimentation and testing in MMs. Drug candidates are identified by matching protein targets with viable drug agents. MMs accelerate drug candidate assessment by confirming the optimum fit of drug agent candidates with protein targets in the context of an analysis of dysfunctional protein interactions in protein pathways and cellular mechanisms. The MMs generate optimal drug agent candidates and narrow the list of prospective candidates by removing those that lack probable protein interaction or protein pathway matches.

MMs analyze a patient pathology, including gene mutations, abnormal RNAs and dysfunctional proteins. Once the pathology is diagnosed on a molecular level, the MMs can tailor a unique drug protocol to the patient disease by searching drug libraries and protein databases in order to find suitable drug candidates to solve the patient's pathology.

The MMs employ an in silico laboratory by utilizing computer-aided drug design (CADD) and modeling drug experiments to search for and test specific drug candidates. MMs predict the outcomes of application of different drug candidates to the patient pathology by analyzing the application of different drug candidates on different instantiations of the dysfunctional proteins at the source of the pathology. In effect, the MMs construct a hypothesis, or set of hypotheses, which are tested by actively testing different candidate drug options on the patient's identified dysfunctional proteins. In a sense, the MMs reverse engineer protein structure solutions to the dysfunctional proteins by starting the process of finding solutions to the core problem of the protein target. One set of hypotheses may be to develop a drug to block the dysfunctional protein target. Another set of hypotheses may be to correct, enhance or improve (i.e., fix) the dysfunctional protein target. Still another set of hypotheses may be to bypass the dysfunctional protein target. Each of these experiments seek out different therapeutic solution modalities to address the patient pathology.

Beyond the purely personalized medicine model that seeks a unique drug therapy to a unique patient pathology that may consist of a limited set of mutated genes or aberrant proteins, the MMs may also be applied to generalized precision medicine in which we address a typology of a pathology that clusters of patients share with similar, though not exact, pathology features. Solutions to a generalized precision medicine model are capable of clinical trials on a wider population than a very refined set of patients with radically unique pathology features. In such a case, the MMs search for drug therapy candidates that multiple patients target with similar, though not exactly the same, protein targets. The MMs apply ML to identify clusters of patients with similar pathologies and molecular aberrations, with an aim to search for common solutions to the shared pathologies.

While drugs are discovered and developed in the MM system, they are tested on another level.

MMs test drug-target interactions and drug-disease reactions. In the case of drug-target interactions, the MMs run in silico experiments to identify, test and select the best drug candidates. In silico experiments can analyze drug binding and molecular docking probabilities of different drug candidates. In many cases, the drug candidates do not satisfy the criteria of omitting toxicity and side effects. In other cases, the drug candidates do not solve the problems associated with the pathology protein pathways in intracellular networks. While drug candidates can emulate protein interactions and target reactions in a computer, it may still be necessary to test drug candidates in vivo.

VAEs, ML and GenAI are applied in MMs to predict drug reactions with diseases. These ML analyses of drug reactions with diseases reveal broader parameters than exist in drug-target interactions alone, much as a macro analysis is broader and more systematic than a micro analysis.

In some respects, the concept of therapy is itself a sort of experiment in search of a solution. The diagnosis of a disease presents a puzzle with incomplete information, the completion of which is considered a therapeutic solution. The application of a drug therapy option requires constant reassessment of a patient's condition as new feedback informs the next stage of therapy options. In the context of this process, MMs are useful tools that process information from obtaining data on a precise diagnosis to searching for and testing drug candidates that may solve the patient pathology. The MMs are uniquely suited to understand and map the protein interactions and protein pathways of unique dysfunctional proteins' effects on intracellular functionality as well as the drug operational pathway and optimal drug-target and drug-disease interactions. MMs are well suited to perfecting each phase of drug development and refinement of each unique therapy.

Since there are different therapeutic modalities, MMs are available to track the different approaches. These therapies include small molecule, ligand, recombinant proteins, antibodies, siRNAs, long peptides, vaccines and gene editing approaches. Each of these therapeutic modalities may be tested in MMs.

Application of ML and GenAI to Therapeutics Drug Discovery with IMMs

VAEs are applied to generate a chemical compound search space to show compound library diversity in MMs.

Machine learning is useful to generate, identify and select drug candidates. ML is also useful for drug candidate validation, that is, to confirm that a drug is applied to a drug target. ML is applied to drug dose, drug timing, drug side effects and drug-drug interaction analyses as well. ML is applied to statistical ranking of probability of the fit of a drug relative to a particular protein target.

Novel Synthetic Drug Design with Individualized Medical Modeling

Macro indicators of disease manifest as symptoms. Yet, as traditional medicine treats the symptoms, we are no closer to solving the problems associated with a disease. Rather, since the sources of many diseases lie on the molecular level of gene variants, abnormal RNA and dysfunctional proteins, it is necessary to address the molecular causes of the diseases. The tools of advanced medicine enable us to view in detail the molecular problems associated with diseases, the sources of which can now be addressed by applying novel therapeutics modalities.

IMMs are a critical component of the toolkit to assist in the identification of a patient's genetic mutations, aberrant RNA and dysfunctional proteins. MMs provide modeling capabilities to view the intracellular protein pathway mechanics of a unique disease. The advent of MMs enables a revolution in personalized medicine not only in the capacity to identify the sources of disease but also in presenting novel solutions that may treat diseases. Two key elements that allow MMs to identify both disease problems and solutions are the availability of molecular data on a disease's abnormal gene, RNA and protein states and the availability of advanced computational technologies in the form of AI and ML.

For the most part, AI and ML applies to MMs in two main ways. First, AI and ML supply descriptive capabilities in order to understand the anatomical and physiological descriptions of the molecular or cellular characteristics of a disease. Second, AI and ML supply prescriptive capabilities in the power to configure, or to generate, novel solutions to a molecular or cellular dysfunction. While the former, descriptive, capability is critical for diagnostics, the latter, prescriptive, capability is critical to enable a new generation of therapeutics.

AI and ML algorithms can be configured to design novel synthetic proteins as a solution to a diagnostic challenge. Specifically, AI and ML techniques are applied to design a unique protein solution for a unique patient pathology. While AI and ML are applied to solve novel therapeutic challenges, MMs are applied to design and test these novel protein solutions in silico.

The first step in a therapeutic challenge is to identify a pathology problem, viz., to precisely diagnose a disease on a molecular level, most notably to identify genetic mutations, abnormal RNA and abnormal or dysfunctional proteins. From the identification of the underlying disease molecular dysfunctions, the MMs identify a protein target or targets that represent the source of the disease. Identification of a patient's biomarkers supplies evidence towards the goal of describing the patient's disease targets, typically a dysfunctional protein. In addition, MMs identify and map out the mechanisms of intracellular pathways of the dysfunctional proteins. These dysfunctional protein pathway maps verify the protein target. Furthermore, MMs identify the precise geometry of dysfunctional proteins. Geometric deep learning (GDL) algorithms, several classes of which are specified in FIG. 2, are applied in order to understand the geometrical configurations of dysfunctional proteins. These analyses are critical to supplying a precise diagnosis of the molecular sources of a disease. One cannot find a drug to treat a disease without first identifying the disease protein target that it is required to address. While biomarkers provide clues to disease targets, it is necessary, in an embodiment, to design tracker proteins that test an individual's protein pathways in order to trace the sources of a disease.

There are times when an existing drug can be applied to the protein target in order to cure or manage the disease. However, many drugs are small molecule chemical compounds that are not precisely targeted to a particular protein target, but, rather, present with numerous unintended side effects and toxicity. In many cases, the cure may be worse than the disease when the applied drug misses the mark by presenting with many unwanted side effects.

Rather than seeking existing drugs that may not precisely target the disease, an optimal therapeutic modality is to custom design a drug for the patient's specific protein targets. This therapeutic modality requires inventing a novel synthetic protein in order to precisely target a specific dysfunctional protein or proteins. Such a model represents the ideal of personalized medicine by optimizing the approach of finding a precise drug treatment to a unique patient disease.

Large language models (LLMs) have been applied to solving complex problems by analyzing and training vast data sets and inferring new data in order to solve a prompt within specific parameters. LLMs are applied to protein structure prediction. For example, AlphaFold 3 (by applying multiple sequence alignment (MSA) algorithms in which a protein sequence is compared to similar proteins to deduce its structure) has solved the protein folding problem of accurately predicting protein structures from amino acid sequences. While this is interesting in predicting protein structures from amino acid sequences, these technologies are primarily descriptive in their ability to mainly elucidate protein structural properties.

Protein language models (PLMs), however, are configured to take this process one step further by actually designing novel protein structures. PLMs, such as Salesforce Research's 1.2B parameter ProGen, train on protein databases. By analyzing hundreds of thousands of actual protein structure configurations, these PLMs are programmed to generate specific novel protein structures on demand. By so configuring novel synthetic protein structures, these PLMs can develop a novel protein to match a dysfunctional protein target. Similarly, when a gene mutation is identified that produces a dysfunctional protein, a PLM can generate a novel synthetic small molecule design in order to apply to treat protein targets in dysfunctional cells. These PLMs apply a class of GenAI algorithms to generate novel drugs to solve the puzzle of matching a target. The custom novel drug design is analogized to finding a unique synthetic key to a unique protein target lock. This new generation of technologies endeavors to solve difficult and unique patient disease challenges with customized molecular solutions by applying AI and ML algorithms.

In some ways, this new approach to novel drug design involves two sets of AI and ML. In the first case, GDL is applied to analyze a protein target in order to carefully describe its geometry. In the second case, GenAI is applied to reverse engineer a custom design drug solution from the protein target geometry. Application of these technologies represents a new paradigm in medicine.

The process of designing a novel synthetic drug takes several steps. First, a disease target, typically a dysfunctional protein, is identified and described. Second, MMs configure a novel drug candidate design to address the disease target. Third, MMs predict the binding of the new drug candidate with the disease target. Fourth, MMs predict drug effects in solving the patient's disease. Fifth, in silico drug testing confirm the efficacy of the drug's effects on the disease. Finally, the novel drug candidates are tested in clinical trials. AI and ML techniques are applied at all stages of this process of searching for custom drug solutions to a unique patient disease.

While GDL is applied to describing the geometry of the dysfunctional protein target, generative GDL (GenGDL) is applied to designing a novel synthetic drug. In some cases, 3D GDL is a more precise approach to protein structural geometrical description, mainly applying 3D graph modeling approaches. Similarly, generative 3D graph techniques are specified to generate accurate solutions for novel synthetic drug designs in order to optimally fit dysfunctional protein targets.

GenAI, including GenGDL, can design different kinds of novel drug therapies. These different drug therapies include novel proteins, novel RNA (e.g., siRNA) sequences, novel ligands, novel antibodies, novel small molecules and novel enzymes. Each type of drug therapy requires a different specialized database of biological or chemical types in order to inform and train the different specialized types of LLMs or PLMs.

Antibody-specific protein LLMs (AbLMs) require antibody databases (such as iReceptor or Observed Antibody Space (OAS)) to train general antibody data, antibody-antigen pair data, paired-chain antibody sequence data and natural antibody data. There are about a dozen AbLMs. Since antibodies are critical configurable proteins that interact with antigens and behave as a lock and key, antibody targets are exemplar for novel synthetic drug design. The MMs apply GenAI and GenGDL to construct a novel antibody to solve a protein or antigen disease target problem.

Since siRNA sequences configure 3D protein structures, after identification of a protein target, MMs can apply GenAI or GenGDL to reverse engineer an siRNA code to apply to a unique protein target or to block the target. Similarly, novel ligands may be precisely designed with GenAI or GenGDL to block a protein component in a target. Novel enzymes are also configured with GAN or GenGDL algorithms by MMs in order to apply to protein targets in order to catalyze or block a natural enzymatic process in an intracellular protein pathway.

One advantage of designing a novel protein drug solution is the ability to configure a protein structure to correspond to specific properties. In other words, it is optimal to design a novel synthetic drug with preferred parameters. Alternatively, the traditional drug discovery model tests many random molecules with different structural attributes. With the opportunity to design a novel synthetic drug to match a specific protein target, the drug structural properties relative to the target structural properties are optimized. In this sense, the drug-target interactive attributes are optimized in the novel design model relative to the traditional drug discovery approach.

MMs enable the prediction of drug properties in the interaction of a drug candidate and protein target. MMs describe and predict the binding of a protein target and an optimized novel synthetic drug design. For instance, when a MM reverse engineers a novel drug from a protein target, the model analyzes the amino acid sequences and peptide configurations in order to develop an optimized novel synthetic drug chemical structure. An advantage of de novo design methodologies of a synthetic drug is the precise configuration of a drug to fit a protein target so as to minimize side effect interactions with non-target proteins.

In an embodiment of the invention, MMs apply GenAI and GenGDL for novel design of customized synthetic biologics. These biologic therapeutic modalities include recombinant DNA, recombinant therapeutic proteins, monoclonal antibodies, vaccines, TNF inhibitors, JAK inhibitors, IL inhibitors, SiP modulators and anti-adhesion molecules. Biologics have application to genetic diseases, cancer and autoimmune disorders.

MMs are uniquely suited to manage all stages of the disease discovery process, the therapeutics drug discovery and design process and the drug testing process. MMs provide simulations of functional transcription processes, translation processes, structural protein development processes, functional protein behavior and interaction processes, molecular (ligand) binding and docking processes, protein-protein interactions, protein-lipid interactions, intra-cellular behaviors and intercellular behaviors. These models and sims are driven by AI and ML algorithms in order to characterize these object relations as 3D- and 4D-spatio-temporal representations. In many cases, the MMs compare patient dysfunctional molecular and cellular processes to healthy reference databases.

The MMs conduct in silico experiments that include descriptive simulations and hypothesis testing in order to precisely identify and describe diagnostic challenges, to predict scenarios of diagnostic prognostics, to identify or design precise therapeutics drugs and to predict the probable scenarios of therapeutic prognostics.

In an embodiment, PHAs are applied to design, collect data for and conduct in silico experiments. PHAs are endowed with AI and ML algorithms in order to analyze diagnostic, therapeutic and prognostic features. PHAs can conduct autonomous experiments in MMs, including parallel simultaneous diagnostic, therapeutic and prognostic analyses. PHAs are, consequently, the workhorses of MM laboratories that solve problems and build models. PHAs draw on biological and chemical databases, specialized LLMs, patient biomarker, empirical biological data and AI and ML techniques in order to build personalized medicine models, precisely describe patient diseases on a molecular level, predict disease evolution scenarios and accurately solve therapeutic challenges for each patient by designing novel synthetic drugs.

Applications of ML and GenAI to Novel Synthetic Drug Design with IMMs

GenAI is applied to novel synthetic drug design. Generative adversarial networks (GANs) are applied to the design of novel molecules and the configuration of novel protein and peptide designs. Natural language processing (NLP) is applied to de novo target-specific drug compound design. Generative pre-trained transformers (GPTs) design proteins with targeted properties. Generative graph neural networks (GGNNs) generate novel molecules to accelerate drug design, predict drug-target interactions and forecast drug-drug interaction events. Generative convolutional NNs (GCNNs) design novel proteins and predict protein-ligand interactions and protein-protein interactions. GGNNs and GCNNs are examples of GenGDL.

Generative 3D graph neural networks (3D-GGNNs) generate novel synthetic 3D proteins with particular attributes, process 3D graph structured data to predict drug-target interactions in 4D model simulations and identify and forecast drug-drug interaction events in 4D model simulations. 3D-GGNNs also identify and predict drug binding to protein-ligand sites in 3D and 4D models and sims and predict protein-molecule interactions in 4D model simulations.

Generative 3D convolutional neural networks (3D-GCNNs) design novel synthetic 3D proteins with well-defined properties and predict functional protein-protein interactions and functional protein-ligand interaction prediction in 4D model simulations.

Generative 3D graph attention networks (3D-GGATs) generate novel synthetic 3D proteins with identifiable characteristics and predict 3D protein attributes from weighted values in 3D graphs. 3D-GGATs also predict 3D protein geometry in 4D model simulations from extracted features.

Generative 3D manifold valued neural networks (3D-GMVNs) generate novel synthetic 3D proteins with unique features. 3D-GMVNs also model protein structures and attributes and provide 4D model simulations of non-Euclidean protein-protein and protein-ligand interactions.

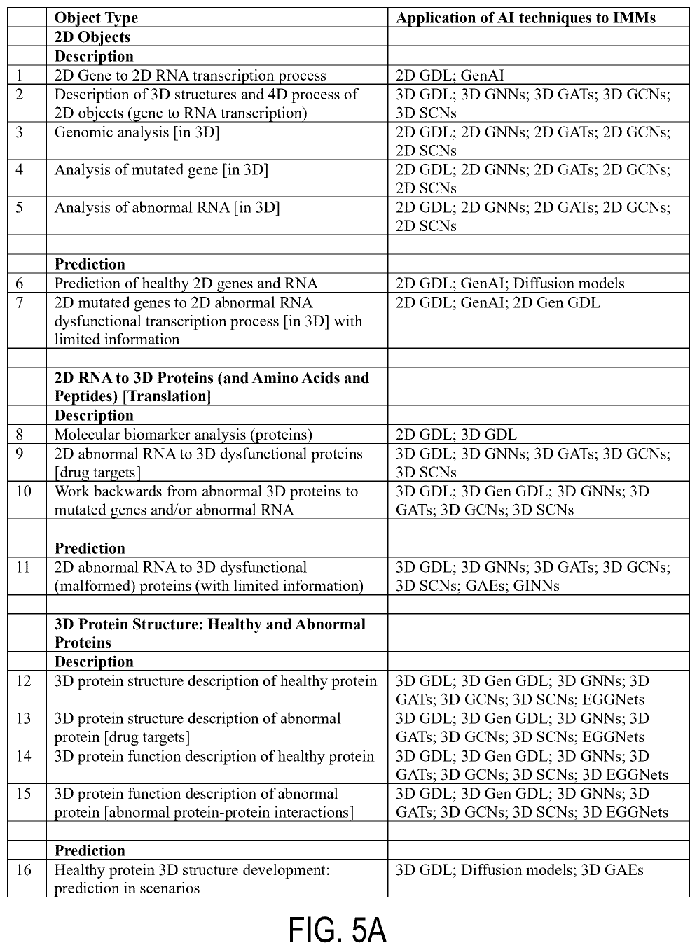

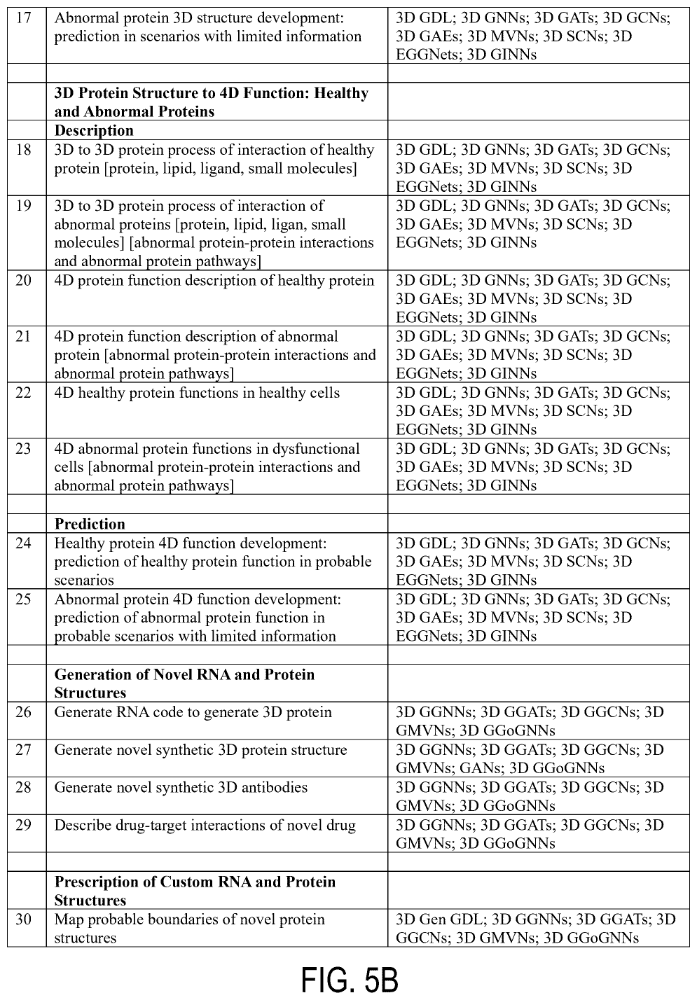

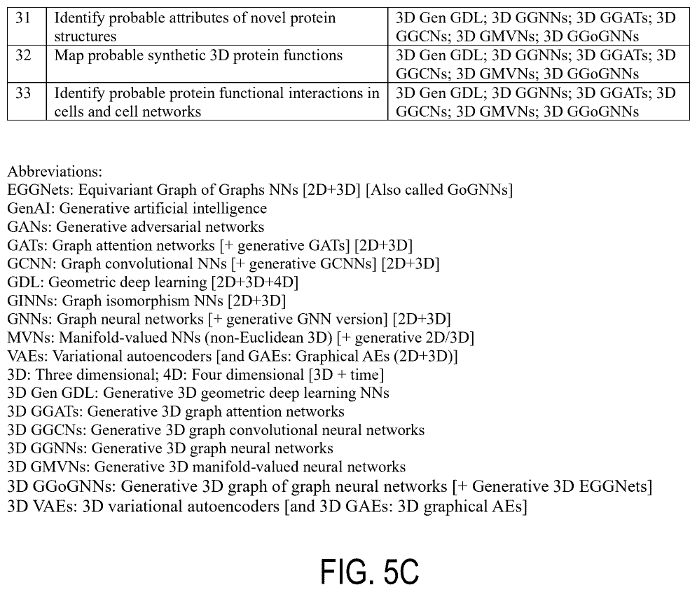

Please refer to FIGS. 2 and 5 for a list of GenAI, GDL, GenGDL, 3D GDL and 3D GenGDL techniques and applications to biomedicine.

### Individualized Medical Models for Therapeutic Prognostics

Diagnostic prognostics is the art and science of projecting or predicting the progress of the course of a disease without therapeutic intervention. A prognosis elucidates the actual patient condition and the expected progress of a patient medical state in the future from an understanding of the patient condition. Therapeutic prognostics, on the other hand, seeks to project or predict the progress of a disease with therapeutic, typically a drug, intervention. The therapy prognosis estimates the progress of a disease state in light of therapeutic inputs on a disease. The main idea is to predict the effectiveness of a drug on the improvement of the course of a disease. A corollary of the main idea states that instead of a drug input, an environmental change or feedback can alter the course of a disease. For example, if a patient quits smoking, a disease may be minimized without drug intervention.

There are two main approaches to therapeutic prognostics, viz., the descriptive and the predictive. In the descriptive approach, a disease progress is tracked in light of different drug options, identifying changes in disease progress with different drug option scenarios. In the predictive approach, a drug effect on a disease progress is predicted. In the case of prediction, we need information on the aggregated history of similar patient diseases and evidence of drug effects on diseases. With more precision, we can more readily predict a drug's effect on a disease when we have identified a close match of a genetic mutation to a tailored drug so as to identify the drug's fit to the molecular configuration of a disease.

Therapeutic prognostics can thus be seen as understanding a disease evolution in light of different drug treatment options. Therapeutic prognostics maps possible drug inputs to treat a disease over time.

The stratification of prognostics reveals therapies that closely match genetic mutations or abnormal protein targets generated from these genetic mutations. For example, a drug may have different reactions in patients with different genetic profiles.

Drug reaction probabilities can be predicted in various scenarios to track the evolution of a disease with various therapy inputs.

MMs are applied to analyze patient prospective disease states of evolution without drug therapy intervention and with drug reactions. The MMs analyze a patient's genetic profile and estimates the prospective reaction of a patient's disease to application of particular drugs on the disease. In one analysis, the MMs analyze the difference between the prospective progress of a patient's disease without intervention and the expected progress of the patient's disease with application of a particular drug or drugs. In the case of a targeted therapy, the prognosis accounts for a delineation of the patient's genetic and proteomic profile to understand the underlying characterization of a disease and the specific targeted drug therapy expected to alter the particular disease.

Overall, the MMs are important in identifying the patient's disease diagnosis on a molecular level, particularly genetic mutations or an abnormal protein target. In addition, the MMs analyze the patient's disease to identify a target and develop a small molecular chemical to fit the protein target or to design a novel synthetic protein or biologic to fit the protein target. Once the patient's genetic, protein and molecular profile is analyzed, the MMs estimate the progress of the patient's disease without intervention as well as with application of a particular proposed drug candidate solution. In order to further the therapy towards an optimal solution, the MMs track the disease progress by analytically comparing at least two drug interventions and developing disease scenarios with prospective reactions to the different drug options.

Biomarkers in Therapeutics Prediction with Feedback

Both genetic and imaging biomarkers are useful in order to assess the progress of a disease. In cancer prognosis, for example, imaging biomarkers are important for an assessment of the progress of solid tumors. When a drug protocol shrinks tumors imaging biomarkers detect the changes to these tumors and indicate the positive impact of the therapy. While imaging biomarkers are useful indicators of disease progress, our main focus is on molecular biomarkers as tangible evidence of changes in a patient's disease evolution. Cancer biomarkers from tumor biopsies and liquid biopsies also provide tumor markers to enable precise drug treatments tailored to genetic mutations.

Genetic biomarkers, which are gene variations showing a pathology risk, pathology development and drug impact on a disease, are useful in therapeutic prognostics. Genetic biomarkers, and by inference, RNA and protein biomarkers, can be useful in enabling the prediction of a drug response outcome on a disease. For example, genetic biomarkers can be useful in showing minimal drug adverse reactions, drug interactions or drug side effects. In general, genetic biomarkers represent an optimal tool for illustrating pharmacodynamics of the feedback of drug therapies on the progress of a disease.

Molecular biomarkers are essential in providing information about a patient's disease diagnosis. The existence of specific biomarkers provides clues to protein aberrations which cause disease states. These abnormal proteins, often a product of a genetic variance, are disease targets to which therapies are developed and matched. Beyond the diagnosis, tracking the evolution of the condition of these biomarkers are useful for an understanding of diagnostic prognostics, viz., the actual or expected evolution of disease states as the pathology progresses over time. These molecular biomarkers then become critical beacons of evidence of the disease condition over time as well as beacons of evidence of the improvement of the disease states with the intervention of specific therapeutic drug applications to the protein target. If the disease's adverse evolution can be blocked by application of a targeted drug, the biomarker analysis can assess the value of the targeted therapeutic drug intervention. In addition to being useful for the assessment of disease states over time, biomarkers are also useful to signal a need to modify or adjust a drug intervention, particularly if the expected benefits of a targeted drug application are suboptimal. But the assessment of biomarkers is crucial to providing an empirical tool in order to understand the evolution of a disease and an assessment of the value of a drug therapy.

MMs are useful for analysis of these biomarkers across the pathology spectrum from diagnosis to therapy option applications to therapeutic prognostics.

Not all therapies work well. Biomarker data and biomarker analyses in MMs enable the constant updating of therapy drug options with the latest biomarker data. This process of continual biomarker assessment across the life cycle of a pathology shows the ability of MMs to track, update and adapt therapeutic options as the demands for changes to the therapy are required over time. For instance, early treatment progress may reveal a good fit of a small molecule drug to stop the progress of a disease, but a later analysis of biomarkers in a later phase of the disease may reveal a need to apply a novel synthetic designed drug to optimize the solution to an evolved pathology state. This iterative adaptation of the therapeutic protocol requires a MM analysis of the evolving character of biomarkers over multiple phases of pathology development.

Updated biomarker information about a patient's pathology over time enables feedback evidence about a drug application to a particular protein target. The most recent biomarker data informs the MM analysis which selects an alternative drug therapy to fit the pathology vector change. Not only do biomarkers provide a diagnostic assessment of a patient's condition at a particular time, but the biomarkers inform MMs with an assessment of a drug's effect. Biomarker assessments reveal a therapy prognosis over time as a series of multiple snapshots that comprise a moving picture of the disease.

MMs identify the genetic mutations at the source of a disease, the abnormal RNA and proteins that manifest from the genetic variances, the protein targets to which drug therapies are directed and the continuing state of a patient's pathology over time. MMs are useful for predicting drug candidate performance for a protein target fit. In addition, the MMs can map the mechanics of cellular protein pathways in both healthy and dysfunctional pathways. The molecular biomarkers represent critical empirical evidence to understand these underlying molecular elements that characterize a patient's disease.

MMs also predict the likelihood of the effectiveness of a targeted drug from an assessment of biomarkers data. MMs identify the quality of the fit of a drug candidate to a target and the fit of a drug candidate to a disease. MMs map the potentialities of a drug's effects on a disease. For example, a model estimates the percentage probabilities of the effectiveness of a drug candidate on different disease and genetic profiles.

In addition, MMs can estimate the potentialities of drug interactions and drug side effects under different conditions. Side effects are typically (unintended) off-target effects of a drug therapy, some of which are predictable. Some adverse drug interactions are also predictable.

MMs are applied to tracking complex multivariate disease scenarios. MMs can solve the problem of multivariate diseases which require the tracking of two or more drug inputs in complex diseases.

MMs can compare different prognostics situations. MMs can compare diagnostic prognostics without intervention to therapeutic prognostics with a specific drug therapy targeting a specific drug target. We realize that the diagnostic prognostics without intervention depicts a raw disease that will show progression without any prospective therapy. The gap between a therapeutical option and no applied therapy can be substantial, which the model's main scenarios of pathology evolution reveals. These differences in predicted trajectories with and without therapeutic intervention can be plotted on a graph. Similarly, the MMs may use the diagnostic prognosis as a control while proposing two different therapeutic options, which are then mapped to show the predicted differentiated evolutionary developments of the patient's pathology over time in order to propose an optimum drug therapy. The multiple therapy options can be assessed in the MM by developing a table of drug therapy options that are ranked according to a match of the patient's profile. In an embodiment, these different therapy options can be viewed as comparative predictive trajectories plotted on a graph. The MMs can then predict the various therapy options based on their analysis of the patient's pathology condition, evolving biomarkers and therapy candidate options. In another embodiment of the invention, a MM can design a novel synthetic drug therapy for a specific patient genetic profile in order to match a patient protein target with the model comparing various drug therapy options, the optimized and preferred novel synthetic drug therapy option and the control case of lack of any therapeutic intervention. These predictions and options are then presented to the patient's physician as therapeutic preferences.

Therapeutic prognostics can predict patient responses to different treatments by applying IMMs to analyze specific patient biomarkers that indicate specific protein targets and then by evaluating different treatment options relative to the probability of success in addressing these targets. In this way, MMs can predict treatment responses. Similarly, MMs can identify biomarkers that match a particular therapy protocol that is targeted to these biomarkers in order to predict a successful therapeutic application.

As the patient disease progresses along a time series with application of various drug therapies, the updated disease status is revealed in biomarker collection and analysis in the MMs in order to identify the patient's most recent disease condition in the context of the specific treatment. The updated biomarker data indicating the patient pathology condition is then evaluated in order to consider updating the patient medication. The physician will review the MM and assess alternative drug therapy options in light of the most recent observations of the status of the patient's disease.

The prognostics scoring system is useful in the therapeutic prognostics context. Whereas in the case of a diagnostic prognostics in which the disease progresses without intervention, the pathology trajectory is generally downward precisely because of the lack of therapeutic options, while in the case of therapeutic prognostics the goal of applying therapy options is to manage or reverse the pathology. Consequently, in the context of prognostics scoring, the likelihood of improvement increases when measuring the application of therapy options vis-à-vis no therapeutic intervention optionality. These improvements in patient conditions are reflected in the prognostics score.

### Applications of ML and GenAI to Therapeutic Prognostics in IMMs

RBMs are applied to predict drug-target interactions. RBMs are also applied to forecast drug-disease relations and to identify drug repositioning tasks in drug-disease relation networks.

Generative GNNs are applied to predict drug-target interactions with graph structured data. In addition, GNNs are applied to forecast and identify drug-drug interaction events.

NLP is applied to forecast and classify drug-target interactions.

### Applications of Individualized Medical Modeling System

There are numerous biomedical applications of the IMM system. Critical medical challenges include cardiovascular disease, neurodegenerative disease, cancer, metabolic diseases and orphan, rare or genetic diseases. Each of these medical categories is well-suited to applications of IMMs. Many diseases, perhaps as many as 90% of human diseases, have a genetic component or a complex genetic-epigenetic dynamic interaction, that require personalized analysis and treatment of each individual's disease. The emergence of precision medicine has grown with the increase in knowledge of the human genome, proteomics and our understanding of the molecular sources of disease. Converging with our theoretical understanding of disease, which includes the ability to gather empirical evidence from genomic, RNA and proteomic testing, is the development of a new generation of advanced sequencing tools that analyze biomolecular data. These biomarker data have revolutionized personalized medicine by presenting real empirical data on human diseases that can be analyzed in order to identify diagnostics, prognostics and therapeutics solutions for each patient.

The combination of MMs with AI and ML can also revolutionize medicine in future years. The ability to analyze massive data sets in human medical studies has enabled the illumination of our understanding of numerous complex diseases. Nevertheless, the ability to make these technologies useful to individual patients in order to identify and treat real diseases has been elusive.

IMMs enable the next generation of clinical medicine. Physicians and researchers now have powerful tools that precisely analyze individual patient diseases. From these analyses, doctors are now able to solve individual patient's previously insoluble medical challenges, which save patients time and money and improve their quality of life.

Though IMMs are applicable to a broad range of (human and animal) diseases, there are several areas for which they are particularly well suited. The most optimized applications for IMMs include medical categories for which the complexity and personalization require intense analytical focus. Only by applying AI and ML to IMMs can these complex individualized diseases be understood and treated.

One area of application of IMMs are to drug clinical trials. While the traditional model of clinical trials, which has embodied conventional medical solutions, has presented an approach to testing drugs in human populations, the application of IMMs presents multiple layers of precision and efficiency. Ultimately, the application of IMMs to drug clinical trials reduces the timeline for approval of drugs and, correspondingly, reduces the costs of drug trials. Since these time and cost issues have been a key reason for the extremely expensive R&D costs behind drug development, application of IMMs to drug clinical trials can be revolutionary.

Next, IMMs are applied to preemptive medicine. Preemptive medicine is an emerging medical field that endeavors to identify patient diseases before the actual physical development of the disease. Applied mainly to chronic diseases, and particularly to chronic cardiovascular, neurodegenerative, autoimmune and metabolic diseases, IMMs are useful for preemptive medicine by modeling and predicting potential individual pathologies from patient biomarker data. An enormous amount of time and money can be saved by correctly anticipating and treating debilitating chronic diseases such as cardiovascular, neurodegenerative, autoimmune or metabolic diseases before they manifest.

One of the most complex and difficult disease categories involves autoimmune diseases, in which an individual's immune system mistakenly attacks an organ or biosystem. These diseases are difficult to analyze, often misdiagnosed and rarely present with significant therapeutic options. IMMs are optimized for identifying and solving these complex and varied autoimmune diseases.

Finally, one of the most complex and insidious diseases in the world involves metastatic cancer. Cancer is the second leading cause of death worldwide, with 90% of mortality caused by metastases. Most metastases have been shown to be drug resistant, suggesting very poor prognosis. However, cancer metastasis may be an ideal category for application of IMMs. Particularly in the case of individualized treatment of metastatic cancer in which each patient has a unique set of genetic features, IMMs present analytical solutions for accurate diagnoses and presentation of therapeutic solution options.

Drug Clinical Trials with IMMs

Once a drug is discovered or designed, it must be tested in a random blind study of drug clinical trials in order to confirm its safety and efficacy. Drug testing includes pre-clinical testing on animals (mice, rats, dogs or pigs) before clinical testing. Generally, clinical trials occur in three phases with increasing numbers of patients. Most drug trial designs have a control arm which administers a placebo and an active arm which administers a novel drug candidate.

Developing a new drug typically takes at least a decade and costs an average of at least two billion dollars. Only about 10% of drug candidates survive the third phase of clinical trials, mainly because of the discovery of adverse side effects, indicating the high risks and costs of drug development. Clearly, one of the major costs associated with drug development involve drug clinical trials, which provide a fundamental bottleneck to final drug approval. The question becomes: How can the application of IMMs accelerate the research and development components of drug discovery as well as the clinical trials components of drug testing?

In the main, IMMs are critical in the identification of molecular (protein) targets, in the analysis of cellular and protein pathway dynamics of drug assimilation, in the identification of patient biomarkers that detect the presence and trajectory of disease evolution, in the identification of drugs that may treat the disease target, in the novel synthetic design of a drug to optimally treat the drug target, in the identification of drug-drug interactions, in the identification of drug toxicity and side effects of off-target drug interactions and in the identification of therapeutic prognostics of ongoing drug treatment of a disease. The application of IMMs to these important biomedical elements are projected to transform the nature of the drug development and testing process with an aim to radically increase the precision of drug testing and vastly reduce the time and costs associated with drug development and trials.

Preclinical testing reviews data to determine the efficacy of clinical trials in humans. MMs perform in silico experiments on patient pathologies to diagnose a disease and identify disease targets. In addition, MMs simulate drug development options in order to optimize drug candidates to match particular patient protein targets so as to address a patient's disease.

Basic research has historically laboriously performed some of these valuable functions, which can now be performed analytically in MMs. Traditionally, basic research identified possible targets to treat diseases and then screen molecules and compounds to assess their utility in application to the disease targets.

The ability to combine MMs and AI with the identification of molecular biomarkers has enabled an understanding of a patient's disease by elucidating genetic, RNA and protein abnormalities that allow us to detect disease targets and then develop novel targeted drug therapies to complex diseases.

In the case of mice or rats, which have brief gestation cycles to enable rapid breeding, a critical advantage of animal studies is the ability to genetically engineer rodents that may fit a particular targeted drug test. In effect, these genetically engineered rodents provide proxies for real human diseases that represent disease targets for drug development. Therefore, once genetically engineered, these rodents possess the specific genetic characteristics that are optimized for precision targeted drug testing.

In a sense, MMs now make animal studies obsolete. While data on mouse or rat experiments are useful, particularly in genetically altering and testing specific targeted drugs on altered mice or rats, the in vivo testing on animals can now be minimized. In some cases, a hybrid of MM analytics in combination with targeted animal experiments can yield promising prognostics.

But MMs alone can provide data on a control group of a disease study because there is no therapeutic intervention required and thus no drug to test. These diagnostic prognostics merely identify a disease diagnosis and project or predict the disease progress based on an analysis of a group of similarly situated patients. The control group can be represented in a MM by virtual patients from analysis of synthetic data on similar patients. In other words, a preclinical testing can include a group of virtual animals that are emulated from synthetic data about a disease diagnosis and non-interventionist prognosis that is identical to a control group. Such an analysis can be done in a computer without harming a single animal.

Traditional clinical trials consisted of symptom-based general disease patient testing. A general drug study is applied to a general pathology, such as high blood pressure or high cholesterol. With generalized random drug discovery organized to identify a drug candidate, typically a small chemical compound, drug testing of the drug candidate would occur in a randomized double-blind study of patients with a general disease and patients without the disease. Patients with a condition like high blood pressure or high cholesterol would test a drug candidate in order to identify a positive effect on the patient's condition. Patient candidates with overlapping conditions would be removed from the active arm of the clinical trials so as to focus on patients with testable pathology conditions. Patients with observable symptoms would apply to and qualify for the clinical trial.

Modern clinical trials go beyond the traditional model by selecting patients on the basis of the presence of biomarkers indicating the characterization of a disease state. This indication of a genetic mutation, RNA abnormality or dysfunctional protein provides evidence of the presence of a particular disease that can be precisely targeted and treated. Clinical trial patients are selected on the basis of the presence of an underlying disease state, with empirical evidence attributing to the presence of the disease state. When the active arm of a clinical trial applies a drug candidate to qualified patients, patient biomarkers are tested to objectively identify patient improvement through active drug testing. Increasingly, the U.S. Food and Drug Administration (FDA) and the European Medicines Agency (EMA) require data showing not only biomarkers data on eligibility and progress, but data on protein pathway interactions revealing the biochemical operations of drugs.

In order to obtain a sufficient quantity of (mutated gene or protein target) qualified patients, it is necessary to transcend space beyond a single central physical location. Hybrid clinical trials may include hundreds of different locations in order to attract sufficient qualified patients.

In addition to identifying patients according to unique genetic mutations, patients are stratified according to particular combinations of biomarkers that indicate a form of a disease and the stage of progress of the disease.

In the case of the control arm, to which a placebo is administered in the clinical trials, the patient candidates have confirmation of the presence of a disease, including a specific genetic variance and particular biomarkers. The control arm patients' disease continues to develop unaided by an active drug. Therefore, diagnostic prognostics techniques of tracking the disease progress apply to patients in the control arm. We know that they have the targeted disease and that the disease progressed without intervention. Biomarkers that track the disease development indicate the disease progression as though they are not in the (active) drug trial at all.

In modern clinical trials, a protein target is identified and the drug candidate is selected to apply to the drug target. Patients are selected with an identifiable disease state that enables the precise targeting of the protein target by the drug candidate.

IMMs can track the control arm of the drug clinical trials. MMs identify qualified patients with confirmation of the presence of genetic variance(s) and dysfunctional RNA and protein biomarkers. The MMs apply ML to compare these qualified patients with other similar patients in medical databases. These databases track the progress of patients with the presence of abnormal genetic, RNA and protein data. From these comparisons of patients and analyses of the disease progress, the MMs are able to project patient disease progress without drug treatment intervention. Consequently, MMs can plot projected progress of the patients with the particular disease that is untreated relative to the progress of the patients that are treated with a drug candidate in the active arm of the drug clinical trials. Some of the diagnostic prognostics of the untreated control arm patients can be done by MMs in silico with virtual analyses by simply tracking or projecting the trajectory of the development of the disease. MM models apply ML to aggregate general patient data on the disease from electronic health records independently of the clinical trials since these data of disease projection without intervention should be identical to the progress of the disease in patients in the control arm. The MMs build models that enable the hybridization of the control arm of clinical trials into virtual patients and actual patients. For example, the biomarker data that are tracked in patients with the precisely identified disease in the control arm of the clinical trials should be the same biomarker data that are tracked from patients with the precisely identified disease outside of the clinical trials since no treatment is applied in either case and the diagnostic prognosis should be identical in both cases.

In an embodiment, GANs generate synthetic data of patient medical characteristics from aggregated electronic health records that show statistically reliable enlarged data for models. The medical synthetic data enhance and enlarge the data sets for MM analysis of patient clinical trial data.

Some advanced clinical trials now apply AI for analysis of results from both the control arm and the active arm of the drug trials. For instance, Unlearn.AI applies conditional restricted Boltzmann machines (CRBMs), a form of ML, to clinical trials. CRBMs are shallow, single layer, NNs that consist of only one hidden layer. Single layer NNs have a limited “feature learning” capacity relative to deep learning NNs that have more hidden layers. Nevertheless, CRBMs generate synthetic patient features in a control arm that enable the extrapolation of disease progress in some conditions. For instance, these models can extrapolate the presence of virtual biomarkers in order to aggregate virtual diseased patient features to estimate untreated pathology evolution. Sadly, however, virtual patients in clinical trials are not themselves models of patients since they emulate the existence of an artificial patient rather than track the progress of real patients. Therefore, virtual patients in the control arm are not tracked per se because the models generate instead virtual patients that are tracked through extrapolation of an analysis of real patient disease progress. The analysis of the virtual patients can reveal data that predicts behavior of real patient progress in the control group, thereby reducing the need for many patients in the control group. The suggestion is that possessing knowledge of the prediction of patient disease progress without treatment intervention should enable the reduction of the control group via generation of a virtual patient group. By reducing the size of the control group, with the application of modeling and AI techniques, time and money are conserved with this approach. In other words, if about half the control arm patients can be replaced with virtual patients, indicating a hybrid control arm consisting of both real and virtual patients, then there would be efficiencies that can accelerate clinical trials. These models do not apply to real patients but rather to emulated virtual patients in order to maximize the efficiency of the control arm of clinical trials. This approach also promotes patient anonymity on the control arm side of the clinical trials.

IMMs are applied to drug clinical trials in order to optimize personalized medicine. Precision clinical trials need MMs to analyze and track actual active arm patients. MMs are useful for precision diagnosis of actual patients and for precision prognostics tracking of real patients' disease progress. Actual patients are tracked with MMs in clinical trials. MMs can be used to generate virtual patients by emulating actual patients; these virtual and actual patients are then analyzed and compared in order to assess precise disease diagnoses and prognostics.

A new class of clinical trials applies MMs to narrow the focus of testing on unique genetic pathologies. MMs are applied to the active arm of drug trials.

In one embodiment, patient data are aggregated in the active arm of clinical trials. The MMs analyze a set of patient data in trials overtime. The MMs assess the patients' drug candidate reactions. MMs identify and analyze biomarkers to track patients to enable drug reaction predictions. MMs compare data from active arm patients over time. MMs predict reactions to drugs in patients with similar characteristics. MMs identify and assess patient drug reaction trajectories.

MMs are applied to track active arm patient progress of an application of a new drug candidate. The actual drug response is tracked by tracking periodic updated biomarker data. The MMs then compare the actual drug candidate application in the active arm of the clinical trials to a placebo application in the control arm of the clinal trials. In other words, the MMs construct a diagnostic prognostics analysis to project the progress of the disease without intervention in the control arm of the clinical trials in comparison to a therapeutic prognostics analysis to project the progress of the disease with drug candidate application in the active arm of the clinical trials in order to compare the two prospective trajectories of the disease. The MMs analyze the updated biomarker data over a time series in order to track the drug candidate application in the active arm of the clinical trials.

By assigning an MM to each patient in the control arm and in the active arm of the drug clinical trials, drug trial administrators have access to data sets on each patient's unique genetic mutations, abnormal RNA and protein aberrations in addition to each patient's ongoing (gene, RNA and protein) biomarkers. These data can be aggregated into a general model for analyses of the control arm and the active arm of the clinical trials. In addition, MMs track and construct models for disease prognoses under different conditions in order to compare patient prognostic data over time. For instance, MMs can compare the placebo in the control arm to the actual drug effects in the active arm.

These data enable the MMs to develop a picture of the mechanics of the disease development on a molecular and cellular level. Since the patients are selected on the basis of genetic mutations and dysfunctional proteins that operate in protein interaction networks, it is possible for the MMs to track the molecular interaction process underlying the generation or interference of the disease. For example, precisely because the clinical trials select specific patients based on their genetic profile for the presence of mutations or protein targets, the drug candidates are similarly selected for the clinical trial for their prospective ability to address the disease target(s). These interactions are mapped and tracked by the MMs as the drug is applied in the active arm and the patients' biomarkers are tracked. The biomarker data are initially applied to diagnostically identify the presence of genetic mutations, RNA abnormalities and dysfunctional proteins that may act as disease targets, while continuing biomarker data are assessed to track the development of a disease without drug intervention in a control arm or assessed to track the treatment of a disease with drug intervention in the active arm of the clinical trials. This fine-grained application of precision medicine in clinical trials is optimized by application of MMs.

In an embodiment of the invention, MMs are applied parallel to clinical trials. While MMs are applied to drug discovery and drug design because of their use of in silico drug experimentation after prospective disease targets are identified, MMs are applied to diagnostics by identification of a patient's molecular biomarkers for assessment of genetic, RNA and protein dysfunctions to which the drug candidates are targeted. In addition, MMs are applied to test in silico many prospective drug candidates on multiple differentiated disease targets. In effect, many prospective drug candidates can be tested on virtual patients in order to accelerate drug development and winnow down the most probable effective drug candidates. These disease target identification and drug development and testing processes provide preliminary pre-clinical analysis preparatory to clinical trials.

With increased precision of disease targeting from increased exactitude in identification of specific genetic mutations, clinical trials are substantially narrowed. On the other hand, if the clinical trials target a somewhat broader range of similar genetic mutations for disease targets with a broader drug regimen, the clinical trials can be broadened to include more generally similarly situated patients.

The advantage of precision focus of personalized medicine in clinical trials is the ability to not only precisely target a disease with a uniquely targeted drug, but also the ability to continuously track the drug candidate efficacy over time. MMs analyze the drug candidate tracking, via biomarker time-series analyses, in order to assess the continuing effects of the drug on the underlying disease. Drug trials should detect a positive effect on a disease protein target quickly after application of the drug candidate. The therapeutic prognostics biomarker data are tracked in order to empirically demonstrate the positive effects of the drug on the patient's disease. On the other hand, if the application of a drug candidate on a patient, as revealed in biomarker tracking data, is as ineffective as application of a placebo, then these data also reveal important information about the lack of efficacy of the drug candidate. In effect, the MMs analyze the spectrum of options between a placebo and a major positive effect on a disease, such as complete remission. The effective differentiated results for each patient of the application of drug candidates, reflected in biomarker data analyses in MMs, reveals the relative success or failure of each drug.

Given enough information about a disease's precise diagnostics and prospective drug candidates, MMs are able to predict how a patient responds to different treatment options. From these predictions, the actual drug candidate performance is measured. When the actual drug performance is more effective than predicted, this feedback information is useful for updating the model and prospective predictions. On the other hand, when the actual drug performance is less effective than predicted, the active arm can be stopped or modified pending review.

When the MMs reveal that a drug candidate works remarkably well on a large share of the active arm of the trial, it is unethical to continue the trial by disallowing the control arm patients to have access to the drug. Contrarily, when the MMs reveal that a drug candidate is simply not working well on any patients in the active arm, relative to the control arm, the clinical trial should be terminated for lack of efficacy. The MMs can reveal a large space between these poles on the spectrum of relative efficacy of the prospective drug candidate. These data obtained during clinical trials at mid-stream, effectively after phase II, enable the ability to change the design of the clinical trials by narrowing the patient genetic profiles or by modifying application of the drug candidate, such as changing the drug dose and/or drug timing. The data obtained at the mid-stream point in the clinical trials are useful for MMs to analyze drug side effects and drug toxicity. MMs identify problems with the drug candidates mid-stream and redirect the clinical trial. It may be necessary, for instance, to modify a drug candidate in order to optimize the fit with actual patient disease targets.

Not every patient's genetic anomaly may represent an exact match for a drug candidate. There may be a 50% match or a 75% match, but not a 100% match for a genetic variance. It is therefore necessary to develop drug therapies that are “generally” precise. For example, in the case of cancer, there may be several hundred genetic mutations at the source of a neoplasm, yet only about 20% of these are active mutations that may represent the genesis of a disease, with only 4 of these representing dysfunctions of regulatory genes at the source of the solid tumors. In this sense, MMs apply combinatorial optimization algorithms to identify disease targets. Different sets of genetic mutations or resulting dysfunctional proteins in different patients may be compared by the MMs in order to target overall active genetic mutations common to different sets of patients. In other words, patients are stratified into different groups based on sets of genetic mutations, protein dysfunctions or abnormal biomarkers. MMs treat different sets of patients as a separate “class” with different sets of precise dysfunctional genes, RNA, proteins or biomarkers, each different class of which may be treated separately with a different single drug or combinations of drugs. These subgroups of different patients with similar but slightly differentiated genetic or protein profiles are categorized into different clusters for application of drugs that match their specific profiles. The testing of patients for biomarkers provides a useful tool for establishing this patient profiling and stratification. Once categorized into clusters, these patient groups are then treated with differentiated drugs targeted to their specific diseases. Such stratification of patients into disease subtypes enables application of different clinical trial drug protocols. MMs are applied to track the therapeutic prognostics of the predicted drug response in the active arm of clinical trials that target each disease subtype.

There are over 8,000 rare orphan genetic diseases. As we are able to identify with greater specificity the genetic dysfunctional sources of these diseases, it is necessary to structure clinical trials to assess drug candidates for these diseases. Next-generation clinical trials will precisely target these diseases with new generations of targeted drugs. In some cases, new drugs will be custom designed with MMs in order to apply to a specific small set of patients. Consequently, clinical trials will be required with smaller patient samples because of a dearth of similar genetic anomalies. MMs will be essential to enable effective clinical trials by zeroing in precisely on the disease's origins as well as specific therapeutic solutions. Ultimately, MMs and ML techniques will rapidly identify the source of the disease and rapidly identify drug solutions. The precision of the diagnoses and the therapeutics solutions will include analyses of the detailed mechanisms of disease operation, including maps of protein pathways and protein-protein interactions, in order to accelerate drug trials. MMs may not only effectuate personalized medicine but also effectively accelerate the drug discovery and testing processes as well, saving time and money.

MMs enable the automation of clinical trials. Personal health assistants (PHAs) are software agents that perform functions to automate MM processes such as collecting patient medical data, analyzing patient medical data and applying AI algorithms to analyze patient medical data. PHAs cooperate with multiple MMs to aggregate multiple patients' medical data.

Physicians and drug clinical trials administrators work together to identify prospective patients for clinical trials. Specialist physicians may have patients that have identified specific genetic criteria for a specific drug clinical trial. Clinical trial administrators screen prospective patients and invite the qualified patients into the clinical trials. Drug companies network with specialist physicians (particularly at university hospitals) in order to establish specialized drug clinical trials.

Patient relationship management (PRM) software coordinates the eligibility and selection of patients into drug clinical trials at the invitation of pharmaceutical or biotech companies. The PRM software utilize PHAs to track patients and coordinate biomarker testing. PRM, PHAs and MMs work together in order to collect and analyze patient medical data.

In the case of approved drugs, MMs and PHAs work together to identify an appropriate drug for a particular patient pathology.

In the case of drug discovery or novel synthetic drug designs, MMs and PHAs work together to identify appropriate drug clinical trials for patients based on the patients' unique genetic or dysfunctional protein profiles. In some ways, this precision medicine model of discovery and clinical trials is the exact reverse of traditional drug development in which a drug was randomly discovered and then efforts were made to identify the new drug's possible medicinal uses. Personalized medicine, on the other hand, optimizes the therapeutics process by first identifying the patient's disease on a molecular level, mainly by analyzing biomarkers for an understanding of the genetic, RNA or protein source of the disease, including disease targets, and then seeks to identify drug therapies specifically tailored for these molecular targets. In a sense, then, after specialist physicians have identified the molecular sources of a patient's disease, the specialists outsource the drug development to drug companies. Once they receive the request for a new drug to treat the patient disease, the drug companies wait to receive a number of orders for similar drug therapies from other specialist physicians with similar patient profiles. The drug companies then initiate the coordination of a cluster of patients by generating a doctor network to focus on clinical trials to treat a specific malady for these patients. The drug company and the specialist physicians coordinate the narrow drug clinical trials.

Because the numbers of highly specialized genetic diseases are relatively small, it may be necessary in some cases, for the drug clinical trials to require a virtual control group that consists of MMs that analyze the diagnostic prognostics set of qualified patients. These can be represented by patients in other geographic regions or at other historic times that also qualify for a drug study because of similar genetic, protein or biomarker profiles. The virtual control group is computationally similar to a real control arm. However, since there are so relatively few quantities of patients with a very narrow genetic profile that may qualify for a drug study, the virtual control group either stands alone as a proxy for a control group in the clinical trial or as a component of a hybrid control group.

In an embodiment of the invention, MMs are applied to in silico pre-clinical trial design. GANs generate synthetic patient data from protein language models (PLMs). In these virtual pre-clinical trials, MMs apply ML algorithms to analyze disease features, genetic mutation categories, dysfunctional proteins, protein-protein interactions, intra-cellular protein pathways and drug-target relations. MMs generate drug candidate options to target particular disease targets, which are virtually tested in hybrid-virtual animal testing in which the control arm can be substantially virtual. Once the drug candidates pass this phase, the clinical trials apply MMs to analyze synthetic patient data. In some cases, the control arm of the clinical trials is comprised of a synthesis that includes partial virtual patients. In other cases, the control arm may be comprised of entirely virtual patients that consist of synthetic data compiled from diagnostic and diagnostic prognostics data on similar patients. The MMs can also predict the effects of the drug candidates on patient's diseases by applying therapeutic prognostics. These predictions are continually updated in the MMs as more biomarker data are provided along the process of updating the active arm of the clinical trials.

Examples of diseases and drug candidates that are applied to clinical trials with IMMs include cardiovascular diseases, neurodegenerative diseases, cancer, metabolic disorders, autoimmune diseases and orphan genetic diseases. Many of these diseases require targeted drug therapies and application of personalized medicine approaches that involve IMMs and AI.

Preemptive Medicine with IMMs

The idea of preemptive medicine is relatively new. The main principle is that it is possible to predict the development of a disease before the disease has presented symptoms and to initiate treatment options in order to minimize the onset of the disease. The notion of preemptive medicine is attributed to Imura's examination of the development and progression of non-communicable diseases. Particularly in the West, chronic diseases such as cardiovascular disease, type II diabetes and neurodegenerative diseases are prevalent because of a combination of genetic components and epigenetic lifestyle or environmental components. If it were possible to anticipate the development of chronic diseases before they occur, would it not be useful in order to save patients years of wellness and high costs associated with patient care? Interestingly, preemptive medicine is tailored to each individual in order to decipher a particular disease condition. Such individuation of medical analysis is well suited to individualized medical models to gather information about potential diseases, to analyze and predict the nature of the diseases for each individual and to propose a tailored treatment plan for each patient.

Information about an individual's genetic risk factors provide important clues for preemptive medicine. IMMs are able to assess genetic variances and hereditary information in order to build models that analyze a patient's propensity for specific diseases. Whereas MMs are useful in order to analyze an actual patient disease by collecting information about genetic, RNA and protein dysfunctions, the models are able to analyze molecular data in order to predict the patient's risks for developing chronic diseases in the future and to recommend a targeted treatment plan.

Preemptive medicine relies in part on the collection and analysis of risk biomarkers and predictive biomarkers. Risk biomarkers evaluate the prospect of a future onset of a disease before symptoms actually appear. For example, recent research has identified biomarkers that assess the risk of onset of Parkinson's disease several years before symptoms appear. In effect, risk biomarkers provide a proxy for a diagnosis of the presence of a disease but without symptoms. Predictive biomarkers anticipate the progress of a disease at some time in the future wherein symptoms do not yet appear but are expected to appear. MMs apply AI and ML in order to analyze biomarkers.

If symptoms do not yet present, the MMs provide analysis of disease states that are expected to present symptoms in the future. Practically speaking, the MMs make a pre-diagnosis of the prospects of a disease. The probable development of a disease is then analyzed in the MMs in order to develop a predictive diagnostic prognosis of a disease. Ultimately, the MMs develop a treatment plan and therapeutic prognosis in order to anticipate the future development and management of the disease.

Examples of preemptive medicine include hypertension and hyperlipidemia, both of which are critical silent killers that behave as a source of heart disease, which has been the leading cause of death in the U.S. for 100 years. If we can take biomarker samples from patients that anticipate with a significant probability that they will develop hypertension and/or hyperlipidemia, then it is optimal to prepare the patient years in advance for the likelihood of the onset of these diseases. One way to prepare for the development of these diseases is to change one's lifestyle. This may include reducing smoking, alcohol consumption and overeating as well as increasing cardio activity. However, even after engaging in these healthy activities, it may be necessary to initiate a modest drug therapy program that includes a statin for hyperlipidemia or an ACE inhibitor or a beta blocker for hypertension.

MMs gather biomarker data from patients and perform analysis of the data in order to build individualized patient models to ascertain future probable disease risks. The MMs compare the patient biomarker data to a database of genetic, RNA and proteomic data of similar patients' pathologies. The MMs analyze the patient data and develop predictions of probable scenarios of chronic disease development for each patient. In a sense, this analysis does not diagnose a particular disease because there is a lack of symptoms. Rather, the analysis develops a pre-diagnosis in which a propensity of a disease is probabilistically identified. The MMs predict the prospects of a disease in the future but also predict the probable progression of the disease. For instance, the rate of the development of the disease is predicted based in part on patient environmental choices that include food, alcohol, smoking and sedentary lifestyle. The diagnostic prognosis of a disease in the context of preemptive medicine, then, becomes a sort of pre-diagnostic prediction of the progress of a probable disease development over time. The hypertension will likely develop more slowly, according to an IMM, if the patient is neither overweight nor a smoker than if she is both, the evidence of which is established by comparison of the patient MM to a database that aggregates patients with hypertension to various outcomes.

Whereas hypertension and hyperlipidemia may have lifestyle and epigenetic components, other diseases, such as neurogenerative diseases, may have more genetic components. These genetic components are tracked by assessing and analyzing biomarkers in the patient MMs. While onset of Alzheimer's disease may not present at age 60, there may be a substantial probability of presentation of the disease by age 75 or 80. Although genetic biomarkers are useful in predicting future disease development, imaging biomarkers are also advantageous for MMs to building models to anticipate the probability of the disease. The trajectory of the progression of the disease's development can be predicted by an analysis of biomarkers over time, which are analyzed in the MMs. In addition, a genetic analysis in the MMs can reveal the significant probability of pre-disease of Alzheimer's development in the future. While an initial biomarker assessment at age 60 will seek to develop an assessment of an actual pathology condition, the continued development and assessment of biomarkers over a period of years can build a picture in the MMs of the probability of the development of Alzheimer's disease in the near future. The assessment of multiple biomarkers over time provides the model with a clear pattern that enables a prediction of the development and progression of the disease. The combination of biomarker assessments over differentiated time frames enables the development of models that are able to predict a future disease state in a sort of pre-diagnosis and pre-prognosis.

AI and ML are useful tools for MMs in order to develop their analysis of preemptive medicine. While general medical databases are useful as reference tools to enable the aggregation of groups of patients in order to understand the multivariate sources of diseases, MMs are well suited to analyzing individual patient data and, by utilizing AI and ML algorithms, assessing individual patient potentialities for future disease onset. The biomarker data in individual patient MMs are compared to the large medical databases in order to identify probable future disease progression scenarios. The AI and ML algorithms are applied to the MM analysis to identify patterns of potential future disease presentation as well as trends of prospective probable disease scenario development. Such analysis of future prospective diseases enables physicians to monitor patients for clues about the likelihood of the onset of symptoms.

Once the disease has been probabilistically predicted, the MMs will develop therapeutic options. These future drug therapies will seek to tailor drugs to the patient's prospective disease. The MM analysis of the patient's biomarkers will identify the best drug therapeutic protocol as well as predict specific drug therapy reactions. This drug treatment approach provides a proactive personalized intervention modality for a prospective disease as it progresses. In effect, the therapeutic interventions are tailored to anticipate the disease progression. AI and ML are applied in the MMs to assess the therapy option scenarios across the early stage of disease progression. In a later step, therapeutic prediction of probable pathology in preemptive medicine identifies the progress of the therapy by assessing therapeutic feedback. Biomarkers can be analyzed by MMs in order to assess therapeutic feedback. For instance, if a drug is applied that does not address the evolving underlying disease, the drug therapy can be modified. The MMs identify scenarios of disease trajectories with different therapeutic options. The MMs map different therapy scenario options and recommend different options in different circumstances. In some cases, the onset of a disease is delayed with the application of an interventionist drug therapy. In other cases, the disease progresses without an effect from the drug therapy, suggesting the need for a different drug therapy. In either case, the MMs tailor the intervention therapy to a unique patient disease prognosis. If the feedback from data on therapy options in altering the disease progression course suggests a new therapeutic approach, the MM may recommend a personalized therapy that consists of a novel synthetic drug tailored to the patient's unique disease complexion. In an embodiment, preemptive pharmacogenomics (PGX) can be applied by MMs to assess an individual's genomic data and identify the optimal tailored patient therapy.

The ultimate goal of preemptive medicine is to treat pre-disease and buy time before the onset of a probabilistic forecast of a disease. If a patient can increase their wellness term five or ten years before the onset of rheumatoid arthritis, Alzheimer's or cardiovascular disease, this could provide a great deal of benefit in the quality of life as well as substantially reduce the costs associated with dealing with a debilitating disease. In the case of cancer, however, an early diagnosis can make a major difference in the outcome; preemptive medicine provides a set of tools to enable earlier diagnosis; in some cases, such as in pancreatic cancer, where many cases are diagnosed far too late for therapeutic interventions, this early detection is transformational. Therefore, preemptive medicine can become integrated into personalized medicine as an important diagnostic and therapeutic modality that will dramatically improve the quality of life for many patients.

Although it is not considered preventive medicine, preemptive medicine shares some elements with its preventive med cousin. Preventive medicine has three aspects, viz., primary prevention, which consists of addressing risky behaviors such as smoking, drinking and overeating, secondary prevention, which consists of early disease detection in order to limit the progress of a disease, and tertiary prevention, which seeks to constrain the impact of a disease on symptomatic patients. Clinicians deal with tertiary prevention while public health officials deal with primary prevention. A popular example of primary prevention consists of public vaccinations, which seeks to prevent infectious diseases. Most of clinical medicine is suited to only deal with patients with clear symptoms of diseases, almost limited to a reactive position. This leaves secondary prevention as the odd man out, without a clear home in medicine for anticipating pre-disease. As medicine moves inexorably towards a personalized and precision model of the practice, with applications of modeling and AI as routine and with the regular utility of gene, RNA and proteomic biomarker analyses, it is clear that the idea of the practice of medicine may be expanded beyond a merely reactive model and may include an expansive understanding that includes preemption and anticipation of future prospective diseases.

Increasingly, medicine is seen as more complex than merely the fixed genetic structure we are born with. Such a broader view of medicine includes epigenetic elements as well as environmental components. As an example, some preemptive diseases may have a prenatal component, suggesting a developmental origin of diseases that can influence the onset of future diseases. How environmental influences in the womb affect later disease developments may explain in part the explosion of type II diabetes, making some people more susceptible to dietary factors later in life. Similarly, the deprivation of patients in early life may burden them later with health problems. In addition, while some genetic diseases have a high likelihood of presenting with a health challenge, other genetic diseases may require an epigenetic element in order to manifest. These examples suggest that preemptive medicine and MMs provide a new category of thinking about medicine and provide a new, broader, spectrum for understanding diseases over a life cycle.

Autoimmune and Inflammatory Diseases Analyses with IMMs

The human immune system consists of two subsystems, the humoral (innate) immune system and the adaptive immune system. As a general response to a pathogen, the humoral immune system generates a rapid inflammatory response by activating enzymes that mark germs as targets and by activating cytokines and natural killer cells to attack the invading germs.

The adaptive immune system generates T lymphocytes (T cells), from the thymus, which employ surface receptors to identify and attach to pathogens, and B lymphocytes (B cells), from bone marrow, which generate antibodies that attach to a closely matched antigen like a key in a lock. Killer T cells become active after their cell surface receptors bind to a specific antigen. Helper T cells are active in initiating B cells and antibodies. Antibodies (proteins) are configured in a Y position with the Y components organized to identify and geometrically fit into antigens. Once a pathogen is identified, the T cells, B cells and antibodies identify the antigen, remember the precise configuration of the pathogen and pass this information on to the humoral immune system.

The general architecture of the human immune system describes the healthy mechanics of a functioning network of cells and antibodies in a “balanced” position. A dysregulated immune system presents autoimmune disorders. Little is known about the pathogenesis of autoimmune diseases, which present as a diverse set of pathologies with only common attributes of abnormal functioning of T cells, B cells and/or antibodies. In general, most autoimmune diseases present with autoantibodies that attack self-antigens comprising a patient's own organs, tissue or cells. Autoimmune diseases emerge if B lymphocytes or T lymphocytes create functional damage to organs or tissue that present as the target autoantigen, indicating that the auto-reactive lymphocytes are the source of the diseases.

Autoimmune diseases include pathologies as diverse as rheumatoid arthritis (joints), Type 1 diabetes (pancreas), multiple sclerosis (central nervous system), inflammatory bowel disease, Graves disease (thyroid), systemic lupus erythematosus (general) and about eighty other conditions. There is a plausible theory that Alzheimer's disease may involve autoimmune components. Additionally, some diseases may be misdiagnosed but may include an autoimmunity factor, including some kidney diseases. Each of these diseases present with immune dysregulation. For example, rheumatoid arthritis presents with an abnormal surplus of T cells that attack the synovium (tissue surrounding joints), while in lupus, there appears to be both a surplus of T cells and an insufficient function of a T cell receptor, viz., a protein (aryl hydrocarbon), which, in turn, changes the function of T cells to stimulate abnormal B cells.

While the sources of this broad range of diseases vary, one theory suggests that inflammation caused by an infection can be a catalyst of an autoimmune disease. For example, long Covid may manifest a new class of general autoimmune disease stimulated by inflammation associated with the virus. The long-term manifestation of long Covid may be the result of a new equilibrium of the immune system that presents as a novel autoimmune condition stimulated by an inflammatory (e.g., an extreme cytokine) response to the virus. Similarly, autoantibodies may be stimulated by inflammation in a specific organ, with the autoantibodies not programmed to understand their clear error of confusing the autoantigens with the target organ.

Although autoimmune diseases likely have a genetic component, such as a gene, RNA or protein aberration, additional elements may be in play as well, including epigenetic and environmental components that combine to express each patient's experience with a disease's differentiation and stratification.

A paradox lies at the root of autoimmune disorders. While the immune system targets different proteins (antigens) with different disorders, suppressing the whole immune system opens a window to the body's defense system. Therefore, if the immune system is tuned down, the subsequent suppression allows pathogens to harm the host. On the other hand, if the immune system is tuned up, the overactivation has a tendency to attack the host as well as the pathogens. Hence, it is essential to realize that the immune system is in an equilibrium state of delicate balance that requires targeted solutions to autoimmune disorders. There is thus a need for selectively targeted solutions to focus or dial back only on an autoimmune disorder, without suppressing the whole immune system.

One theory of autoimmune disorders suggests that regulatory T cells (Tregs) are key to maintaining a balance of the immune system. Tregs are important to suppressing auto-immune over-reaction, while proteins can be instrumental to activating Tregs. Johns Hopkins engineers have identified a novel protein that combines interleukin-2 cytokine and anti-cytokine antibody FS111 in order to stimulate Treg activity. The novel protein can be administered to T cells in the form of an encoded mRNA.

One of the great challenges of autoimmune disorders lies in the difficulty of correctly diagnosing each disease. Many of these diseases remain undiagnosed because of their complexity and the lack of clinical tools available. As a consequence, the patients remain unsure of the parameters of their disease and lack specificity on a prognosis. Without a correct diagnosis, therapeutic modalities become a distant hope without practical realization. Yet because they are so varied and complex, autoimmune disorders require a personalized approach to analyzing each patient's condition. IMMs are well suited to provide individualized models in order to collect biomarker data, apply AI and ML algorithms and precisely diagnose each disease.

Biomarker data are critical for correct identification of each type of autoimmune disease. In the case of rheumatoid arthritis, SLAMF6, MAGE1, CD40L, FPGS, ADORA3, IL-38, HLA-DP, IL-10, NLRP3, CARD8, TGRS, HDAC, YTHDF2, SOCS1, ABCG2, IL-32, TP, TGFBR2, CD26 and HK2 refer to mRNAs associated with RNA mechanisms, while miR-5571-3p, miR-135-5p, miR-143-3p, miR-23b, miR-539, MiR-125a-5p, miR-146a, miR-361-5p, miR-132-3p, miR-155-5p, miR-5196, miR-326, miR-195 are miRNAs indicating RA and lnc-ITSNI-2, GAPLINC, GAS5, Inc-AL928768.3, Inc-AC091493.1, RP11-83116.1, MALATI, NEAT1, LINK-A, OSERI-ASI, Inc-PCT1, FOXD2-ASI, GASC2, HOTAIR, Inc-Cox2 and LINC00305 refer to lncRNAs associated with RA. Please refer to FIG. 4 for a detailed analysis of biomarkers involved in autoimmune diseases.

Regarding systemic lupus erythematosus (lupus), VCAM1 and ICAM-1 are biomarkers that predict nephritic flair, MALTI1 refers to inflammation, NAMPT and eNAMPT refer to lung inflammation and CD163, MCP-1, Serpin-A3, Ig binding protein 1, TWEAK, suPAR and S100 refer to biomarkers of the active disease.

Regarding autoimmune neuromuscular (CNS) disease, particularly multiple sclerosis (MS), GM1, GA1, GD1a, GD1b, GalNAc-GD1a, 9-O-Acetyl GD1b, GD3, GM1, GT1a, GT1b, GT3, GQ1b, 0-Acetyl GT3, LM-1, GD1a/GD1b, GM1/GalNac-GD1a, GM1/PA, GM1/GD1a, GM1/GT1b, LM1/GA1 IgG and IgM represent autoantibody biomarkers for MS, particularly for prognosis, Cytokines Interferon gamma (IFN 7), Tumor necrosis factor a (TNF a), Transforming growth factor β1 (TGF β1), IL-1β, IL-4, IL-6, IL-10, IL-12, IL-16, IL-17, IL-18, IL-22, IL-23, IL-37 refer to biomarkers correlated to MS, miR-150-5p, miR-21-5p, miR-30e-5p and let-7 miRNA family are associated with diagnostic and prognostics.

Regarding inflammatory bowel disease (Crohn's), iR-19a, miR-21, miR-124, miR-141, miR-150, miR-155, miR-193a-3p, miR-206, miR-21, miR-143, miR-145, miR-125b, miR-223, miR-138, miR-7, miR-19b, miR-29b, miR-122, miR0141, miR-200b and miR-590-5p are biomarkers associated with diagnostics or prognostics of the disease.

Regarding Type 1 diabetes, miR-375, miR-21, miR-210, miR-24, miR-148a, miR-181a-5p and miR-210-5p represent biomarkers that show upregulation of T1D.

Given the availability of information about biomarkers in each of these autoimmune diseases, it is necessary to analyze the biomarkers by applying AI and ML in the IMMs. Different patients can be stratified into autoimmune disease subgroups by analyzing biomarkers. Not only do IMMs analyze abnormal gene, RNA and protein biomarkers that present as a disease, but they analyze new classes of biomarkers, including lipid biomarkers, cytokine biomarkers and small molecule metabolite biomarkers. Because autoimmune conditions may include both genetic and epigenetic components, abnormal gene, RNA and protein biomarkers are supplemented with epigenetic small molecule abnormalities. The IMMs analyze the two types of the genetic and epigenetic components that comprise complex interacting abnormal proteins and small molecules. In addition, Tregs may represent a new class of important biomarkers that require evaluation in IMMs.

IMMs are not only well positioned to model and understand the diagnosis of autoimmune disorders, but they are well suited to model prognostics as well. Because of the complexity of these diseases, and the substantial variability between the broad spectrum of each class of disorder, it is essential to understand each patient's disease on an individualized basis. In general, clinical medicine struggles to merely find a correct precise diagnosis of complex autoimmune conditions. However, with the advent of IMMs, analytical models, AI and ML algorithms and a new generation of biomarker identification, these detailed diagnoses can be performed. From these diagnoses and analyses of models in the IMMs, the prognoses are also performed. Particularly because these classes of autoimmune diseases are complex, and diagnoses as well as precision therapeutics are elusive, a great deal of emphasis is placed on diagnostic prognostics to predict the evolution and various trajectories of the disease in light of both genetic and epigenetic data.

So far, only general therapies have been available to treat superficial symptoms of autoimmune disorders. However, there are several therapeutic modalities that may be successful in managing or addressing these diseases. Ideally, we want to tailor a therapy to a unique target. Since autoantibodies and autoantigens are at the root of these diseases, it is critical to correctly identify the source of these autoimmune diseases.

Monoclonal antibodies (mAb) are therapies for inflammatory diseases. mAb's and antibody conjugates may be effective to treat some autoimmune diseases, including RA, Crohn's disease and others. Some mAbs bind to and inhibit TNF-a, while others inhibit IL-2 receptors on T cells. Targets of mAb's include TNF-a, and interleukin IL-12 and IL-23, which are blocked by the antibodies. Interestingly, these therapeutic modalities are also applied to immunotherapies for cancer and infectious diseases.

IMMs can be configured to build models that design novel synthetic proteins, including novel synthetic antibodies. When a T cell receptor or an antibody (i.e., an autoantibody) is targeted, a novel protein is designed to fit like a key in a unique abnormal protein. The development of synthetic antibodies is linked to complex antibody libraries that generate antibody populations tuned for a particular disease's cellular or molecular receptors. Fragments of antibodies are isolated and produced with unique variants in order to match particular antigens or autoantigens. IMMs are applied to identify an optimal match of synthetic antibodies with prospective targets.

In another therapeutic modality, IMMs can be involved in designing novel Treg cells in order to reprogram immune system circuitry. In an embodiment, the IMMs design novel synthetic proteins that stimulate Treg cells in order to re-equilibrate the patient's immune system. Once applied, the IMMs track and adapt the therapy to optimize the patient's immunity while solving the abnormal autoimmune behaviors.

IMMs are suited to developing mRNA and gene editing therapies to target specific abnormal proteins at the source of the autoantigens and/or autoantibodies.

An additional therapeutic modality for autoimmune disorders includes CAR T lymphocytes that act as an inhibitor of B cells that may overreact to create an abundance of autoantibodies. CAR T therapy provides T cells with artificial receptors (chimeric antigen receptors (CARs)), which are designed to identify and attack cells the receptors are configured to bind to, such as misdirected B cells. But this model is generally not selective and is designed to attack all B cells, which is overkill that harms the underlying immune system capabilities. A more precise and selective approach targets specific antigens, or autoantigens. In this approach, the receptor can be reprogrammed to target a specific protein. As they are reprogrammed to target a specific protein, the B cell receptor will bind to a specific antibody target, thereby leaving other B cells free to behave normally in the immune system. This new CAAR T (chimeric autoantibody receptor) is a precise update of the CAR T therapy that enables careful targeting of an autoimmune disease. Effectively, the engineered T cells are programmed to attack specific B cells that overproduce antibodies that become autoantibodies to attack autoantigens. Both modalities were developed by researchers at the University of Pennsylvania. However, IMMs are well suited to identify the precise antigens and autoantibodies that optimize the specialized targeting of B cells, T cells and antibodies in each patient's immune system to target specific autoimmune diseases.

A third major therapeutic modality for autoimmune diseases includes mesenchymal stromal cells (MSC). This approach applies stem cells to replace T cells or B cells in order to effectively reprogram the immune system to redirect energy away from generating autoantibodies to address autoantigens. IMMs are well suited to modeling and analysis for application of MSCs to specific autoimmune conditions.

Metastatic Cancer Analysis with IMMs

Cancer is the second leading cause of death worldwide after heart disease. About 90% of cancer patients die from the metastases of the primary cancer, suggesting that metastatic cancer is one of the most complex and difficult challenges in medicine. The traditional view of cancer suggested a homogeneous development of cancer cells in various tissues in which cancer cells proliferate uncontrollably. However, the current view suggests numerous factors explaining a heterogeneous development of cancer in which tumor cells interact with, and adapt to, their microenvironment, thereby providing a high level of complexity for each patient's unique presentation of the disease. Cancer is a prime example of the application of precision medicine, which addresses unique features of a disease for each individual. Since each individual has a different presentation and development of cancer, including the metastatic process of cancer development, cancer analysis presents the ideal application of IMMs.

All cancers generate from a combination of gene mutations. In some cases, a patient may present with hundreds of genetic mutations. While many of these mutations are benign, a few mutations will stimulate abnormal cell growth, suggesting that each cancer tumor is unique. Except for leukemia, the majority of common cancers involve solid tumors, which remain the focus of cancer research. Solid tumors may be classified by subtypes based on their unique combinations of genetic mutations. For example, the BRAF gene is often mutated in melanoma, the BRCA1 and BRCA2 genes are often mutated in breast cancer, the EGFR gene is often mutated in lung cancer, the HER2 gene may be mutated in breast cancer, the IDH1 and IDH2 genes are often mutated in leukemia, the KIT gene is often mutated in gastrointestinal stromal tumors, the KRAS gene is often mutated in colorectal, lung and pancreatic cancers and the PIK3CA gene is often mutated in some breast and bladder cancers. The most common solid tumor cancer types, from the most prevalent to the least prevalent, are breast, lung, colorectal, prostate, pancreatic, melanoma, leukemia, endometrial, bladder, kidney, thyroid, lymphoma and liver.

In many cases, tumors have a genetic mutation source, but also a protein pathway genesis process. P53, for instance, is a tumor suppressor gene, sometimes called the “guardian of the genome.” Mutations in P53 increases the risk of developing cancer. A healthy P53 activates P21 that interacts with cell division stimulating protein (cdk2). A P21 and cdk2 combination stops cell division. A mutation in P53, however, stops production of P21, which results in indefinite cell division. This single example illustrates that there are many genes and protein pathways that must be understood in order to track the development and progress of cancer.

Single-tumor tumorigenesis of primary solid tumors have a specific orientation of prognostic prediction and therapeutic endeavor. The traditional diagnostic tool for discovery of solid tumors is imaging technologies, including CT, PET and MRI. The traditional therapeutic approaches for solid tumors include chemotherapy, radiation and surgery. Radiation is targeted to specific solid tumors while chemotherapy is typically applied systemically.

In recent years, immunotherapies have harnessed monoclonal antibodies to apply to cancer tumors. When the immune system is dysregulated or tricked by a tumor not to attack the tumor, immunotherapies proceed to activate the identification of the tumor (as an antigen) in order for the immune system to recognize and address the tumor. Immunotherapy provides training or reprogramming of the immune system to identify or target the neoplasms. Monoclonal antibodies are applied to correctly identify the tumors to enable the immune system to attack the tumors.

In some cases, it is necessary to biopsy a solid cancer tumor in order to analyze its composition. However, in recent years, it is possible to obtain a liquid biopsy via a blood test in order to detect cancer cells and dysfunctional DNA, RNA and proteins. Consequently, liquid biopsies are a major tool for detecting and monitoring cancer. While miRNA are non-coding RNAs that regulate mRNA, it is becoming evident that miRNAs are useful biomarkers that demonstrate involvement in the spread, or blocking of the spread, of cancer from a primary site to other distant sites. miRNAs that predict metastatic cancer are metastamiRs.

Interestingly, specific cancer types tend to spread to specific main sites. Breast cancer spreads to bone, brain, liver and lung sites. Lung cancer spreads to bone, brain and liver sites. Colon cancer spreads to liver, lung and peritoneum. Prostate cancer spreads to adrenal gland, bone, liver and lung. Pancreatic cancer spreads to liver, lung and peritoneum. Melanoma cancer spreads to bone, brain, liver, lung, skin and muscle. Endometrial (uterine) cancer spreads to bone, liver and lung. Bladder cancer spreads to bone, liver and lung. Kidney cancer spreads to adrenal gland, bone, brain, liver and lung. Thyroid cancer spreads to bone, liver and lung.

Once a primary tumor is established, the process of metastasis occurs over a number of stages. Each stage represents a major event hurdle that must be surpassed before engagement with the next stage. The metastatic cascade consists of the following stages:
1. Cancer cells separate from a primary tumor site
2. Invasion of cancer cells into adjacent tissue (Micro-environment angiogenesis and epithelial-mesenchymal transition (EMT))
3. Migration of circulating tumor cells (CTCs) to blood or lymphatic vessels
4. Exit of CTCs in blood or lymphatic vessels at another organ (MET and EMT at another site)
5. Tumor genesis oligometastasis (formation of several micrometastatic nodules)
6. Systemic metastasis (adaptation and reprogramming the surrounding stroma)
7. Drug resistance of tumor cells (generation of cancer stem cells (CSCs) and reprogramming of tumor cells to resist drugs)

Each of these steps represents an identifiable stage among the state of evolution of metastasis. There are many theories of the causes of metastasis, including a compromised immune system, limited cell oxygen (hypoxia), mitochondria DNA (mtDNA) mutations, sodium leak channel non-selective protein (NALCN) as regulator, cell plasticity and reprogramming, epigenetic modifications and cell death (autophagy). In many cases, once the cancer has spread to other organs, the patient's prognosis is poor. However, there may be unique differentiated biomarkers available at each stage of the process of metastasis that enable application of identification of specific diagnostic cognizance and targeted therapeutic interventions.

The functioning of the mechanics of tumor metastasis is likely very difficult and inefficient. For instance, only a few cancer cells with unique functional capabilities are likely to survive a long trip to a distant organ. A circulating cancer cell is required to survive in the vasculature or lymphatic system and to subsequently survive and grow in a distant organ or tissue. In many cases, the circuiting tumor cells that survive to inhabit a distant organ may have a different genetic profile than the original tumor's cellular genetic profiles. Interestingly, given the evidence of the transformation of genetic profiles between the primary tumor cancer cells and the metastasized cancer cells, the focus of therapeutic interventions on the original tumor genetic profile often explains the poor prognosis after metastasis has occurred. In other words, after metastasis has occurred, the rules have changed and there is a need to update therapeutic modalities to track the updated disease status. These observations explain the positive prognosis of therapies with a strictly localized primary cancer versus the same therapeutic model applied to metastasized tumors resulting in poor prognosis.

The separation of cancer cells from a primary tumor site may mimic a normal process of tissue shedding cells. While healthy circulating cells, with no mutations, do not form a secondary tumor, those cells with mutations may piggy-back on a normal repair mechanism of the cell shedding process. The cell shedding process is a sort of repair system that renews cells by destroying dying cells while generating or replacing cells.

The invasion of adjacent tissue includes the processes of microenvironment angiogenesis and epithelial-mesenchymal transition (EMT). Microenvironment angiogenesis is the process of generation of blood vessels by a tumor to provide oxygen and nutrients to cancer cells and to allow the expelling of waste. Angiogenesis feeds the tumor. As the tumor grows, it invades neighboring tissue.

EMT enables the process of invasion by which carcinoma cells in the primary tumor break through the membrane of adjacent tissue. Once cancer cells of the primary tumor break through and invade other tissue cells, some of these tumor cells become circulating tumor cells (CTCs) and enter the bloodstream. In other cases, depending on the location of the primary tumor, the CTCs enter a lymph node and gain access to the lymph system.

The migration of CTCs in the bloodstream or lymphatic system enables the surviving cancer cells to travel to distant organs.

Once the CTCs exit the blood or lymphatic vessels at a distant organ site, the cancer cells form microtumor cell clusters at the new site. Once immigrated to the new site, the cancer cells engage in a process of mesenchymal-epithelial transition (MET), the reverse process of EMT, which anchors cancer cells to epithelial surface locations of the new organ. When the colony establishes a foothold at the new locations, the cancer cells engage in the EMT process to invade the new organ tissue cellular layers.

The EMT process may lead to the propagation of evasive CTCs which escape immune surveillance. These CTCs may have adaptive mechanisms that effectively modify their gene mutations in the new organ locations to promote immune evasion.

CTCs embody circulating tumor DNA (ctDNA), which represent detectible biomarkers that can be applied to monitoring a cancer state or to predict a treatment response. The differentiated ctDNA biomarkers across the metastatic process delineate distinct diverse phases of metastasis. These discriminated ctDNA's at each stage of metastasis can identify the precise phase of the disease for accurate diagnosis and monitoring. Once a specific stage of metastasis is identified, therapeutic approaches can be precisely modeled and applied to each phase.

In addition, ctDNA can be used to identify a location of a primary tumor since the CTCs contain mutations of an original tumor. Interestingly, some CTCs may be dormant and will not form a secondary tumor, while others will be dormant for years and eventually form multiple tumors at different sites.

The tumor genesis oligometastasis process generates new neoplasms by forming micrometastatic cell cluster nodules at the new organ(s)' locations. This early stage of the metastatic process only recognizes the spread of a few CTCs to new organs. In many cases, the spread of cancer occurs in organs nearby the original primary tumor site. Cancer cell clones are selected for fitness in order to colonize different organs.

The later stages of metastasis recognize the spread of CTCs from the primary tumor site to multiple differentiated organ sites, illustrating the completion of a process of systemic metastasis. The newly colonized cancer cells adapt to and reprogram their surrounding environment. In many cases, the genetic composition of the metastasized cancer cells at the distant secondary sites varies from the genetic architecture of cancer cells in the original primary neoplasm.

One of the main challenges of metastatic cancer is that the cancers at secondary locations adapt in such a way as to be resistant to drugs. Drugs that may have been effective for the primary tumor may likely not work on tumors at the secondary sites. Both metastasis and drug resistance are critical events that adversely affect cancer prognosis and therapeutic modality potentialities.

A major theory that explains the phenomena of drug resistance in metastatic cancer involves the presence of cancer stem cells (CSCs). CSCs show the potential for self-renewal and may reprogram both the tumor and the microenvironment of the secondary tumor sites. CSCs have features of propagation that differentiate them from other cancer cells. These cells have different features of adult stem cells that can configure into differentiated cell types, since they can generate cells that have only limited proliferation capabilities and since they lack the original cellular mutation architecture.

CSCs play a role in drug resistance. Drug resistance in the primary tumor may be the result of the self-renewal capabilities of CSCs. CSCs may modulate their protein pathway functions in order to protect themselves from drugs designed to attack them. The plasticity and reprogrammability features of CSCs also explain their ability to evade native immune system mechanisms designed to search out and destroy cancer cells. Understanding these mechanisms of plasticity is important in order to design effective therapies to address metastatic cancer. In addition to tumorigenesis and cancer proliferation, CSCs may play a key role in cancer recurrence after application of traditional cancer therapies. Furthermore, CSCs may be a major feature of metastasis, which explains the evasion of drug treatment options as the stem cells renew and regenerate differentiated molecular protein pathways. Observations of the therapy evasion capabilities of CSCs in metastatic cancer reveals the need to search for therapeutic solutions beyond the traditional chemotherapy, radiation and immunotherapy functional modalities.

Different classes of biomarkers are critical in diagnosing, monitoring and predicting the progress of metastatic cancer. ctDNA, CSC's and RNA represent major categories of biomarkers that have fine-grained application to understanding metastatic cancer.

Micro-RNAs play a major role in the reprogramming and dysregulation of the cancer cells as they change their locations in different organs. Let-7, miR-9, miR-132, miR-186-5p, miR-200 family, miR-203, miR-215 and miR374a represent biomarkers that describe EMT. Snail, Zeb1/2, Twist, KLF8, interaction of EMT factors with miR-148a and miR200, activation of Notch and WNT/b-catenin pathway and TGF-b mediated EMT are biomarkers that reveal EMT processes. Please refer to FIG. 4.

SOX2 overexpression, p38-regulated NOTCH1, CD34/CD38, CD133/CD44, CD44/CD24, elevated ROS and RNS, Oct3/4, CD44v6 and COX2 are biomarkers that reveal tumor initiation and growth. Increased HIF-1 expression, activation of MAPK, P13K/AKT, RhoA and VEGFA, Lymph angiogenesis by CXCL11, MMPs, CAFs and TAMs are biomarkers that reveal tumor angiogenesis.

miR-10b and miR-21-5p are biomarkers that reveal migration and invasion by CTCs. miR-149-3p, miR-140-5p, miR-195-5p, miR-101-3p, miR-338-5p and miR-34a are biomarkers that show miRNA replacement and miR-21, miR-210, miR-10b, miR-155, miR-221, miR-22, miR-522, miR-9 and miR-663a are biomarkers that show miRNA inhibition.

CD133 (pancreatic cancer); CXCR4 and CD26 (colon cancer); ALDH+ and CD44+CD24 (breast cancer); CD110, CDCP1 (CRC); P120CTN and CD105 (liver and lung cancer) are biomarkers that reveal tumor metastatis. ABCG2, CD133, ALDH, CD2711, CD20, CD44, BCMab1, NESTIN, A2B5, CD15, MUSASHI1, L1CAM, GRP78, CD98 and CD200 are biomarkers that show cancer stem cells.

Elevated ALDH, enhanced expression of ABC transporters, high expression of Bcl-2 and Bcl-XL, DNA damage repair by CHK1 and CHK2, upregulation of IGFR and HDAC and elevated ROS signaling are biomarkers that reveal therapy (drug) resistance.

miRNAs may be active players in the presentation of metastatic cancer. For example, two tumor suppressor miRNAs, miR-15 and miR-16 show that their deletion can affect proliferation of cancer cells. Similarly, miR-34 and miR-200 may affect p53, a cancer regulator gene that is deactivated in many cancers. In addition, miR-17-5p and miR-20a may affect the activation of MYC, an oncogene that is overexpressed in some cancers.

Given these observations and challenges, it would be logical to target these elements of the metastatic cancer and drug resistance phenomena along the different stages of cancer development. Biomarkers are critical to identifying each of these definable stages of cancer development from primary neoplasm genesis to systemic metastasis and drug resistance.

Fortunately, individualized medical models (IMMs) provide an essential tool in the oncologist's toolkit. IMMs are useful in order to identify and monitor a patient's precise cancer, predict an accurate prognosis and to tailor, track and adapt a therapy protocol for each patient. First, IMMs map the composition and progress of a patient's cancer. If the cancer metastasizes, the IMM tracks this process over several stages. Regarding the primary tumor, the IMM identifies a therapeutic treatment plan. If the cancer metastasizes, the IMM updates the model and the therapeutic treatment plan accordingly. Because of the complexity of each metastasized cancer, in which each cancer profile is unique, the IMM develops a diagnostic prognostics analysis at each stage of the process with and without a progression to each next stage of cancer development, by generating different scenarios of potential progression with the most current empirical data about the disease.

IMMs are applied to molecular models, cellular models, organ and tissue models, DNA, RNA and protein models, drug-target models and protein-protein interaction models. Each of these model categories provides important data about patient condition, progress scenarios and therapeutic optimization and adaptation.

In order to optimize the IMMs, the models apply GDL and GenAI for different analyses. For diagnostics, the IMMs apply GDL in order to analyze biomarker data. For prognostics, the IMMs apply GDL and GenAI to model prediction scenarios. For therapeutics, the IMMs apply GenAI to generate models that provide novel therapeutics solutions, including development of unique combination therapies, drug development and novel synthetic drug design tailored for each patient. Therapeutic prognostics applies AI and ML algorithms in order to develop models to identify predictive scenarios at each stage of the disease progression.

The recognition of the differentiation of stages in the development process of metastatic cancer enables identification of novel biomarkers across the developmental cycle of the disease. Recognition of some of these biomarkers assist in the development of novel therapies to treat patients across each phase of this developmental process. Different proteins are identified as targets at each stage of the metastatic process. IMMs are optimized to track a patient's biomarkers that mirror the developmental phases of metastasis. Biomarkers are essential tools used to identify therapeutic targets; such biomarkers are analyzed by utilizing IMMs. In many cases, the targeted protein that is identified by the biomarker analysis reveals the need for tailored treatments for only a unique subset of patients. For example, anti-miRNAs, or antagomirs, may present as excellent targets for which to develop drugs to target a specific phase of metastatic cancer. In another therapeutic modality, RNAs that control the immune system may be ideal targets for therapeutic development in order to constrain or reverse metastatic processes. Specifically, IMMs are applied to analyze specific oncogenic miRNA inhibition or tumor-suppression miRNA restoration. The emergence of anew class of drugs for metastatic cancer is closely linked with AI-enabled IMMs.

### DETAILED DESCRIPTION OF THE DRAWINGS

The individualized medical modeling architecture is comprised of several main components. These IMM system components include (a) scores of medical modeling categories configured in a periodic table of levels and layers representing diagnostic, prognostic and therapeutic modeling types, (b) artificial intelligence, GenAI and machine learning techniques and algorithms for medical modeling and simulations, for description, prediction and generation of biomedical phenomena, for artificial intelligence-enabled software agents and for software applied to integrated health records, patient relationship management and patient data security, (c) IMMs applied to diagnostics, diagnostic prognostics, therapeutics and therapeutic prognostics for personalized medicine solutions and (d) utilization of IMMs in personalized medicine applications of cardiovascular disease, cancer, neurodegenerative disease, immune system disease and genetic diseases as well as to drug clinical trials and pre-emptive medicine.

One technology that enables deep analytics of genomic and medical data involves medical modeling. Medical models apply tools to duplicate each individual's medical data in a digital model. Increasingly, AI is applied to medical models in order to supply insight on the molecular, cellular and system level.

Individualized medical models take individual genetic, biomarker and imaging data in order to build a model of each individual medical condition. AI tools enhance medical model insights by building diagnostic models of specific diseases.

Medical models are well suited to assess disease prognostics as well. Medical models, guided by diagnostic inputs and by AI, are able to evaluate disease progression scenarios. Since diseases evolve in different directions and at different paces based on the behavioral or biochemical inputs, medical models are able to simulate the disease progression changes in models.

Medical modeling of disease prognostics is able to develop complex time-elapsed prognosis representations based on various inputs and assess vectors of disease evolution within a range of probabilities. For instance, a medical model on an individual disease can track biomarker assessment over time to evaluate or predict probable disease progression.

Medical models are computer models. Medical models are a computational duplicate representation of objects that enable the visualization of biomedical structures from different angles and the testing of medical processes. In an active mode, as an example, medical models enable surgeons to map out and test surgical procedures before they operate on a patient.

AI models are computer programs that analyze data patterns by examining large data sets. An AI model is trained on data sets in order to analyze data patterns, detect anomalies, solve problems or make predictions from limited data. AI models use algorithms—symbolic code or mathematical language—to apply to a data set to make a decision. While an AI model can be used to make predictions or solve problems, algorithms apply the logic which the model uses to come to a conclusion. Consequently, AI models are used to automate learning and decision making, particularly when machine learning and deep learning techniques and algorithms are applied.

There are different classes of AI models, including generative models, discriminative models, classification models, regression models and foundation models. ML models are trained on data sets; by applying probabilistic analyses, the models learn. Foundation models (also referenced as base models) are pre-trained deep learning models trained on large data sets. These large language models, and the generative AI that rely on them, are a form of artificial neural networks and can have trillions of parameters. Large language models facilitate natural language processing (NLP), which analyzes and predicts LLM text patterns. These LLMs can be fine-tuned to specific AI applications. For instance, generative AI chatbots are derived from foundational LLMs; the chatbots are intelligent (autonomous) agents that apply generative adversarial networks (GANs). Intelligent agents are fine-tuned from generative AI (GenAI) to perform certain functions, such as problem solving, pattern matching or prediction. These AI operations are useful for application to medical models. As an example, GPT is an acronym for generative pre-trained transform. These GPTs, or transforms, enable the tracking of connections between proteins and genes. From the analyses of structural proteomic patterns, the transforms can make predictions of protein folding.

Machine learning is applied to medical models by applying algorithms, or instructions that provide a recipe for machines to analyze data, execute tasks and make decisions. AI algorithms are classified as supervised, unsupervised and reinforcement learning.

AI algorithms can be tailored for medical models to supply personal medicine solutions. Algorithms are applied to medical models for pattern matching, problem analysis, progression analysis, personalization and prediction. In the case of pattern matching, medical model algorithms are structured for problem finding, that is, to identify an anomaly in genetic or protein structures. The medical model algorithms are then configured for problem solving in order to analyze the problem, typically by applying classification and sorting techniques. The medical model algorithms personalize the medical model to a particular patient by fine-tuning the model in order to provide a level of customization; for example, the algorithms endeavor to match a cure to a specific patient disease. The medical model algorithms are organized to make predictions; in the context of prognoses of diagnoses and therapeutics, the algorithms analyze prediction scenarios within a range of probabilities. To make predictions, the algorithms apply progression analysis within the constraints of limited information. The medical model algorithms are programmed to automate processes with AI, ML, DL and GenAI techniques.

Guided by AI, medical models are even better able to identify risk scenarios—including the statistical chances of each scenario—very well. If a patient engages in unhealthy behaviors, a disease can be tracked in a negative scenario, whereas if a patient engages in healthy behaviors, a disease can be tracked in a positive scenario.

By applying inference algorithms, the medical models also supply risk-based predictions of patients and are able to supply healthy behavior recommendations. Disease progression scenarios can sometimes be contingent on specific inputs. Medical models can then be applied to anticipate or predict specific disease scenarios based on different inputs. Medical models can then update its prognostics scenarios with new data inputs.

While medical models and AI algorithms are useful for diagnostics and prognostics, they are also applicable to therapeutics. Medical models can also anticipate optimal health scenarios with application of precision therapeutics.

Before the discovery of DNA, it was impossible to trace the source of a disease to its cause. But with the deciphering of the human genome, we now have the tools to observe the genesis of disease with great specificity. Each of thousands of genes can be damaged in different ways and thus embody mutations that generate uniquely dysfunctional proteins. These dysfunctional proteins manifest in each individual's distinctive disease manifestation. Without understanding precisely which gene is mutated and exactly how this mutation is manifest, it is not possible to find a solution to this disease.

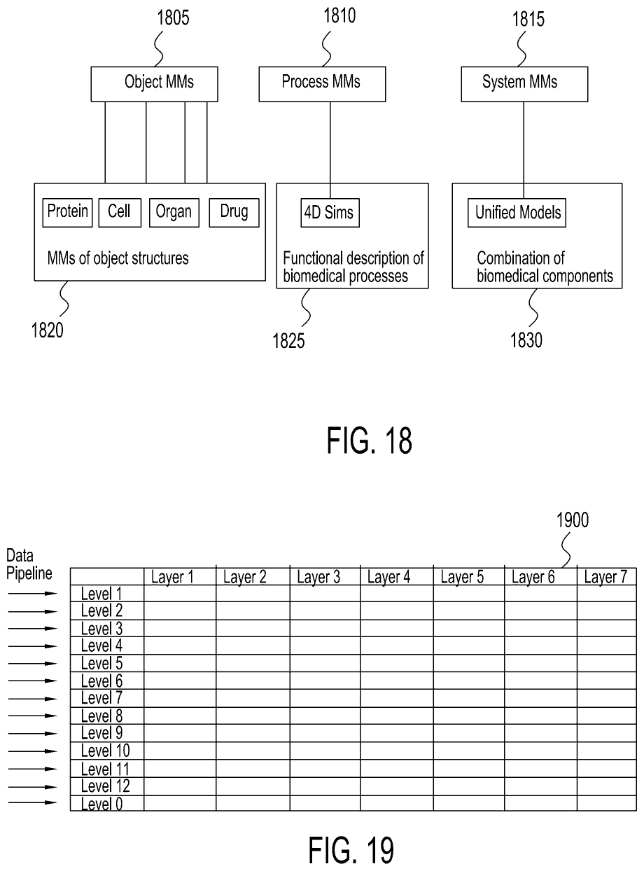

The medical modeling architecture is comprised of a typology of modeling categories. The medical modeling typology is generally organized into several main groupings. These modeling categories are shown on thirteen levels, with each level pertaining to a class of biomedical phenomena. Level 1 includes general patient models. Level 2 includes diagnostics, bioinformatics, organ and body system analyses. Level 3 includes molecular and cellular description and analysis. Level 4 includes structural genetic variant combination pathology identification. Level 5 includes functional molecular and cellular pathology diagnosis. Level 6 includes diagnostic prognostics simulations. Level 7 includes general therapy solutions. Level 8 includes unique therapy solution genesis. Level 9 includes therapy option testing and simulations. Level 10 includes therapy prediction scenarios for therapeutic prognostics. Level 11 includes unified patient modeling that integrates other levels into an integrated model. Level 12 includes human population modeling that provides a platform for public medicine. Level 13 (0) includes master individualized medical modeling that shows a broad system view of the modeling system beyond a single individual pathology. The main medical modeling map is described in FIG. 1. There are extensive dynamics between the MM categories. See FIGS. 18-30 for a description of these MM dynamics.

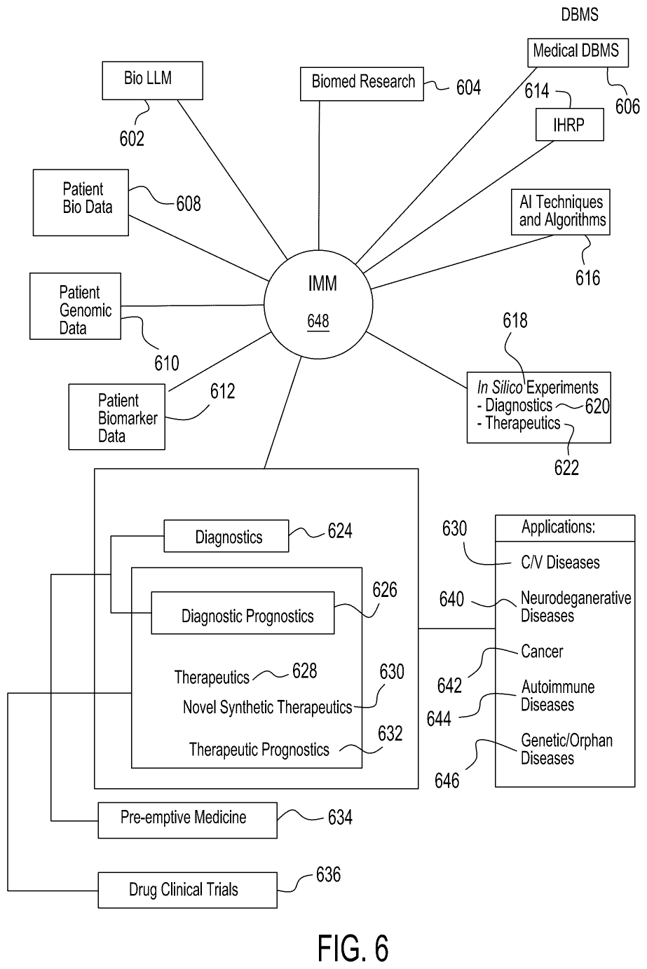

Microbiological analysis is a main feature of the IMM system. FIG. 3 shows an RNA typology and FIG. 4 shows a list of biomarkers organized by disease types. FIGS. 6-17 show a general review of IMM biological system analysis, particularly in the context of analyzing abnormal proteins.

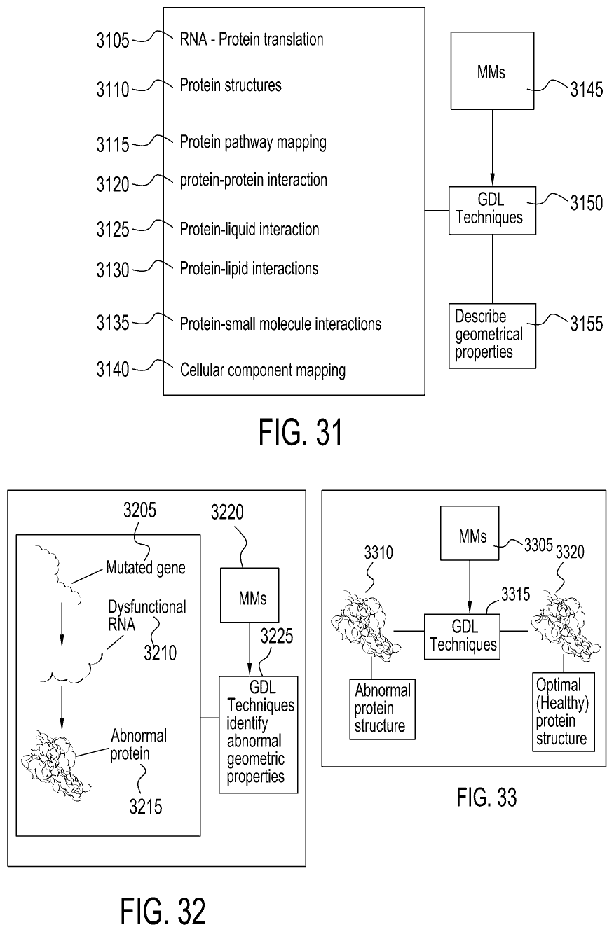

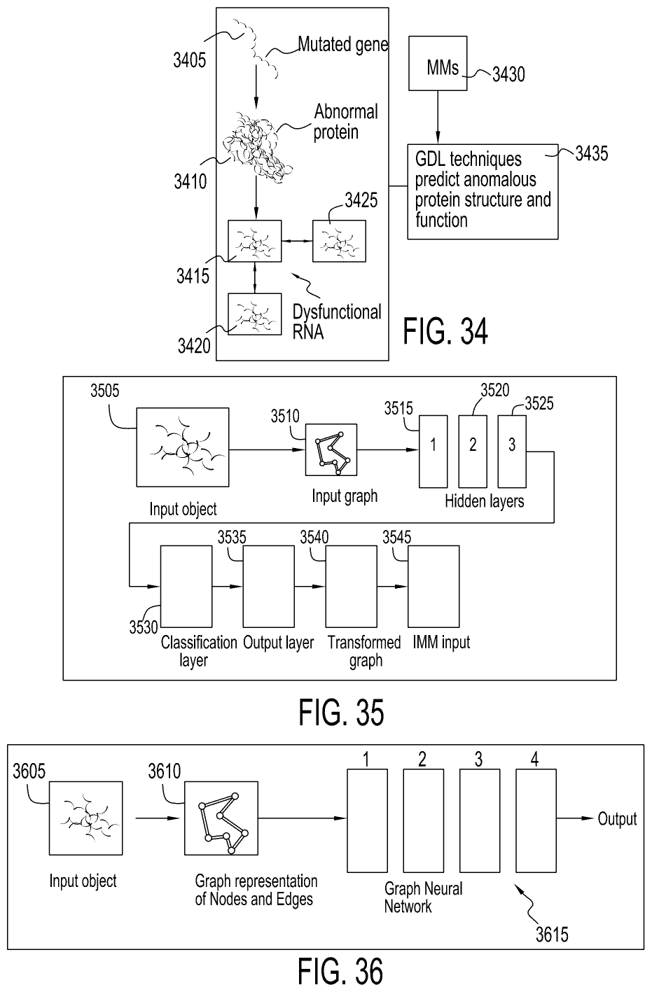

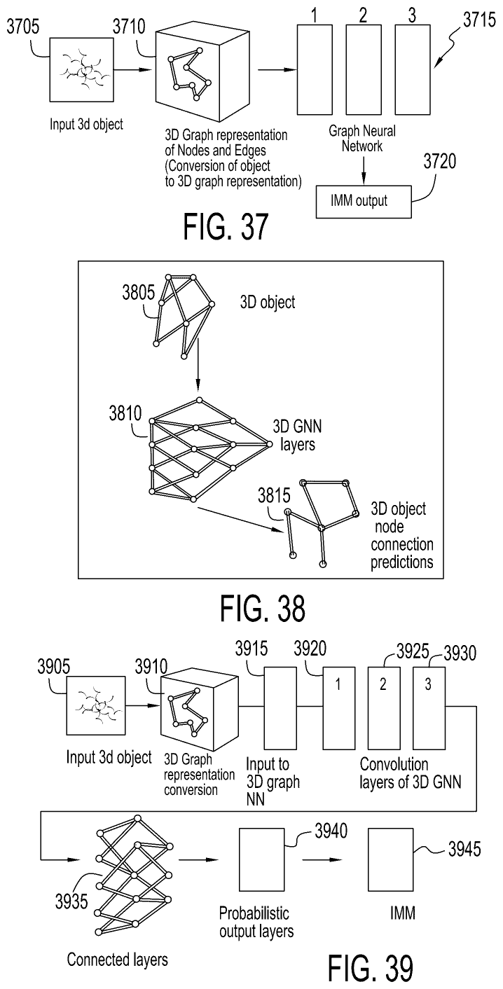

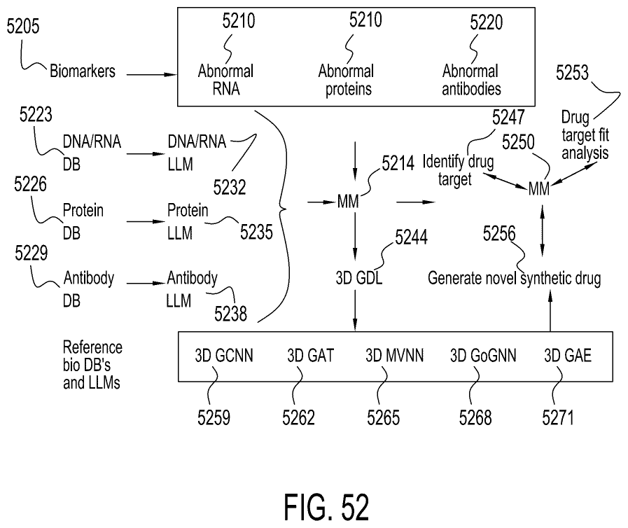

AI and ML are applied to medical modeling. ML, GenAI, geometric deep learning (including graph machine learning), generative GDL, novel 3D GDL and novel generative 3D GDL techniques and algorithms are applied to IMMs for application to diagnostics, diagnostic prognostics, therapeutics and therapeutic prognostics. FIG. 2 reviews a typology of AI categories applied to biomedical modeling technologies. FIG. 5 shows a protein object structure classification system and neural network type matching. General GDL analysis is reviewed in FIGS. 31-36. 3D GNN analysis is shown is FIGS. 37-51. LLMs are shown combined with 3D GDL in FIGS. 52-60.

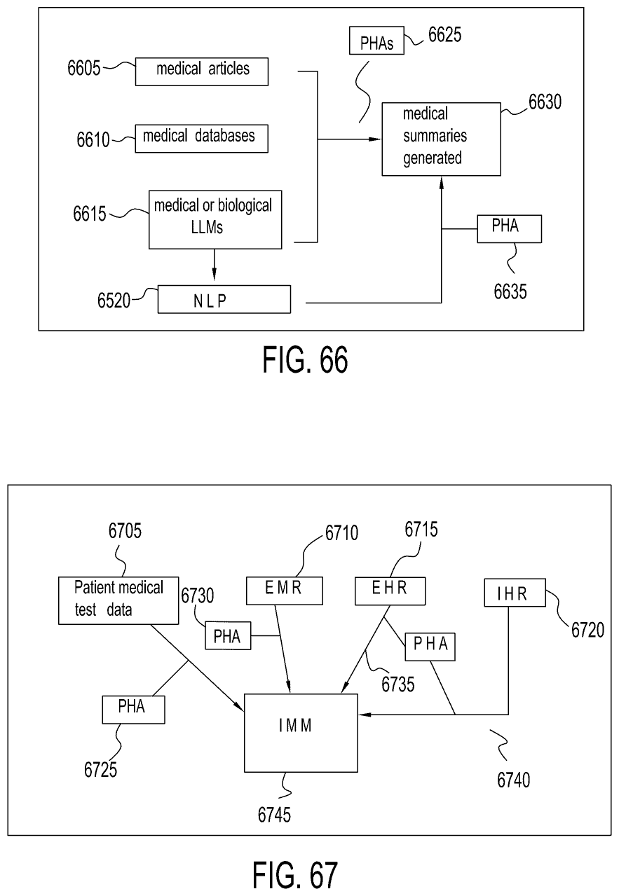

Personal health assistants (PHAs) are multifunctional intelligent software agents applied to IMMs. PHAs are shown to build and analyze IMMs in order to solve medical problems. PHAs are shown in FIGS. 66-76.

Several categories of software are described, including an integrated health record platform that integrates IMMs, health data management, medical patient data security and patient relationship management. These software categories are shown in FIGS. 61-65 and 77-81.

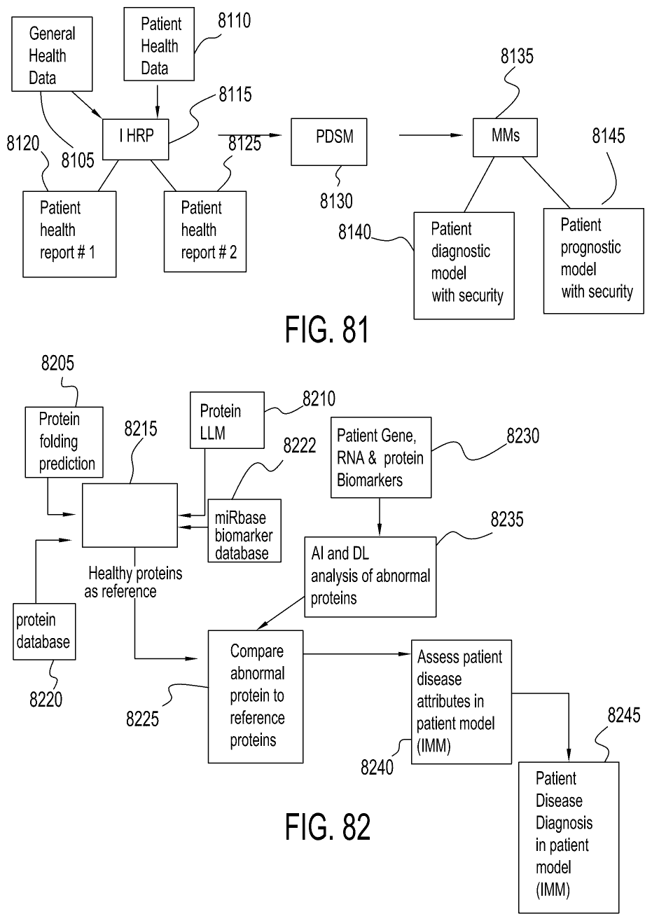

IMMs are applied to medical diagnostics. IMMs are shown for personalized medicine diagnostics, biomarker analysis, identification of novel biomarkers and development and analysis of in silico experiments for diagnostics. IMMs are applied to analyzing diagnostics in critical diseases, including cardiovascular applications, neurological applications and oncology applications. IMMs applied to diagnostics are shown in FIGS. 82-98.

IMMs are applied to diagnostic prognostics, including biomarker analysis and in silico experiments for diagnostic prognostics. IMMs applied to diagnostic prognostics are shown in FIGS. 99-110.

IMMs are applied to medical therapeutics. IMMs are applied to drug discovery and in silico drug experimentation. IMMs applied to therapeutics are shown in FIGS. 111-115. In addition, IMMs are applied to development of novel synthetic drug design. IMMs applied to novel synthetic drug design are shown in FIGS. 116-124.

IMMs are applied to therapeutic prognostics, particularly by analyzing therapeutic prediction with feedback. IMMs for therapeutic prognostics are shown in FIGS. 125-139.

AI, GenAI and ML techniques are applied to each category of medical diagnostics, diagnostic prognostics, therapeutics (drug discovery and drug generation) and therapeutic prognostics.

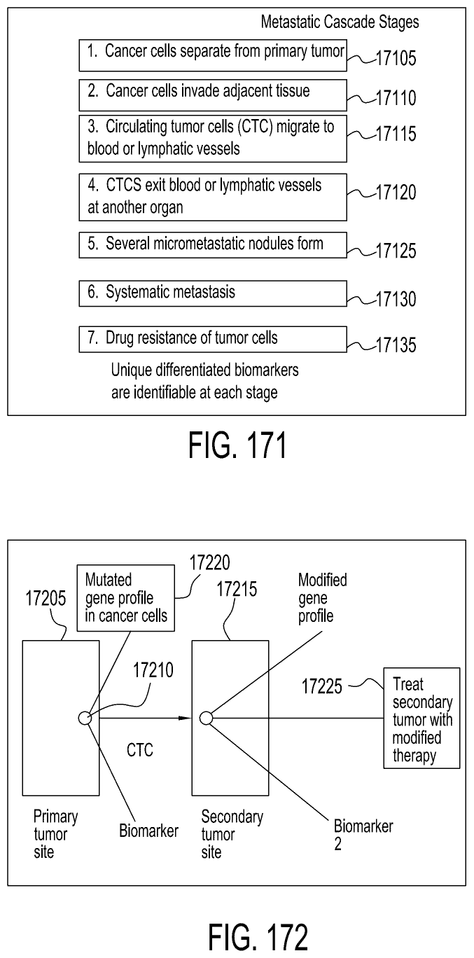

Applications of the IMM system are made to drug clinical trials, pre-emptive medicine, autoimmune disease analysis and metastatic cancer analysis. IMMs applied to drug clinal trials are shown in FIGS. 140-157. IMMs applied to pre-emptive medicine are shown in FIGS. 158-163. IMMs applied to auto-immune disorders are shown in FIGS. 164-170. IMMs applied to metastatic cancer analysis are shown in FIGS. 171-180.

FIG. 1 is a table describing medical modeling architecture and modeling typology categories. The table shows 13 levels and 6-7 layers for each level. Inside these 80 main categories are represented by different modeling types. Medical models and individualized medical models are terms typically used interchangeably. Please refer to the description of the invention for a detailed description of FIG. 1. While the 80 categories are representative, they are not intended to be a complete list of major categories. Moreover, the main biomedical categories also include a number of minor biomedical categories. In addition to a typology of MMs, the table also refers to computational AI and ML solution categories for each level. For example, for level 1, LLM and NLP techniques or algorithms are applied. Similarly, for level 2, GenAI, LLMs, NLP, GANs and 3D and 4D GDL techniques or algorithms are applied. These AI technique and algorithm types, and hybrid AI algorithms, are applied to the various layers of each respective level.

FIG. 2 is a table showing artificial intelligence categories applied to biomedical modeling technologies. The main AI categories of (I) Generative AI, (II) Geometric Deep Learning, (III) Generative GDL, (IV) 3D GDL and (V) Generative 3D GDL are shown. Each of these AI categories are applied to biomedical modeling technologies, particularly in the context of the mechanics of AI, GenAI and graph NN functional operation in the context of describing, predicting or generating biological molecular or cellular phenomena. Note the discussions in the description of the invention and in the detailed description of the drawings at FIGS. 31-60.

FIG. 3 is a table showing an RNA typology. The RNA categories include post-transcriptional modification RNAs, protein synthesis RNAs and regulatory RNAs. In addition to specifying the RNA categories, the table also refers to the RNA biomechanical functionality for each category and further describes the specifications for particular RNA types.

FIG. 4 is a table showing biomarkers of disease types. The table is organized according to disease types, including cardiovascular diseases, neurodegenerative and psychiatric diseases, cancer types and autoimmune diseases. The biomarker types include a range of microRNA, lncRNA, gene and micro-molecule types. In some cases, a description of the biomarker effects and operating mechanisms are provided. Note that these biomarkers are referenced in the context of specific C/V, neurodegenerative, cancer and autoimmune diseases analyzed in IMMs in the present invention.

FIG. 5 is a table showing a protein object structure classification system and neural network type matching. Several classes of molecular objects are shown, including 2D objects (genes and RNA) for description and prediction, 2D to 3D proteins (and amino acids and peptides) for description and prediction, 3D protein structure (healthy and abnormal proteins) for description and prediction, 3D protein structure to 4D function (healthy and abnormal proteins and cells) for description and prediction, generation of novel RNA and protein structures and prescription of custom RNA and protein structures. Each of these biomolecular and cellular object categories references a corresponding set of AI techniques applied to IMMs. The IMMs apply the GenAI, 2D GDL, 2D Gen GDL, 3D GDL and 3D Gen GDL categories to respective protein object types.

FIGS. 6-17 show a general review of IMM biological system analysis, particularly in the context of analyzing abnormal proteins. FIG. 6 is a block diagram of the general medical modeling system architecture. The IMM (648) is shown receiving data from a bio LLM (602), biomedical research (articles) (604), medical databases (606), patient biological data (608), patient genomic data (610), patient biomarker data (612) and integrated health record platform (IHRP) (616). In addition, the IMM receives AI techniques and algorithms (616). The IMM conducts in silico experiments (618), including diagnostics (620) and therapeutics (622) analyses. The IMM develops models for diagnostics, (624), diagnostic prognostics (626), therapeutics (628), novel synthetic therapeutics (630) and therapeutic prognostics (632). The diagnostics and diagnostic prognostics are applied to pre-emptive medicine (634). The diagnostic prognostics, therapeutics, novel synthetic therapeutics and therapeutic prognostics are applied to drug clinical trials (636). The diagnostics, diagnostic prognostics, therapeutics, novel synthetic therapeutics and therapeutic prognostics modeling types are applied to C/V diseases (638), neurodegenerative diseases (640), cancer (642), autoimmune diseases (644) and genetic and orphan diseases (646).

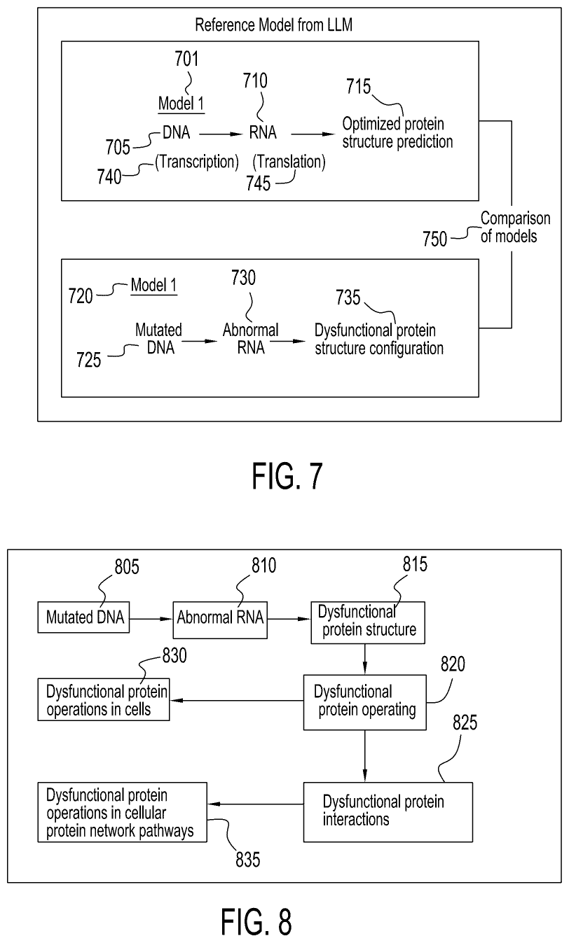

FIG. 7 is a diagram illustrating a comparison of healthy protein structure and unhealthy protein structure models. Model 1 (701) refers to the reference model from an LLM. In this model, healthy DNA (705) is shown to be transcribed (740) to RNA (710) and the RNA is shown to be translated (745) to an optimized protein structure (715). In the context of the LLM, the LLM predicts the healthy protein structure from the DNA or RNA sequence data. In model 2 (720), mutated DNA (725) is shown transcribed into abnormal RNA (730) and the abnormal RNA is shown to be translated into a dysfunctional protein structure configuration (735). The two models are then compared (750) in order to show the relationship between the healthy protein structure and the abnormal protein structure.

FIG. 8 is a diagram showing dysfunctional protein structural functionality. A mutated DNA (805) is transformed into abnormal RNA (810) and into dysfunctional protein structure (815). The dysfunctional protein structure configuration is shown with dysfunctional protein operations (820). The dysfunctional protein operations are then shown in the context of dysfunctional protein interactions (825) and dysfunctional protein operations in cells (830). From the dysfunctional protein operations in cells, the process shows the dysfunctional protein operations in cellular protein network pathways (835). These processes show the transformation from a mutated gene to an abnormal protein structure and then from an abnormal protein configuration to dysfunctional protein operational dynamics in cells.

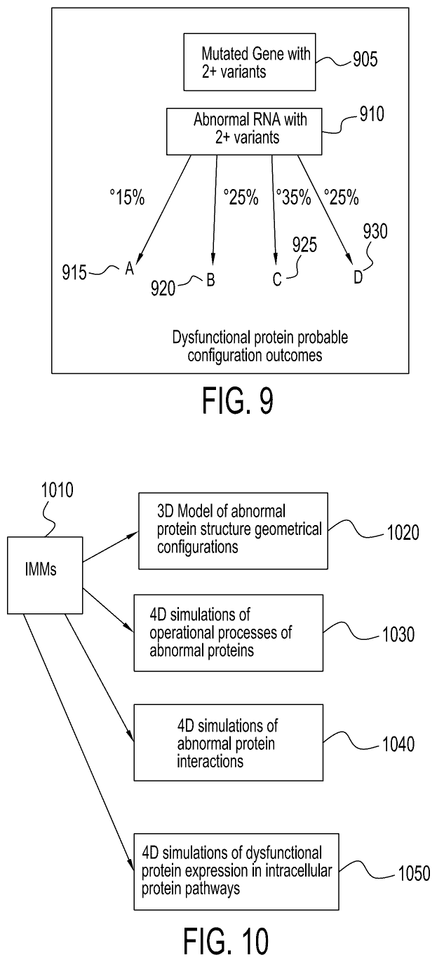

FIG. 9 is a diagram showing dysfunctional protein outcome probabilities. A mutated gene is shown with two or more variants (905). The mutated gene then produces abnormal RNA with two or more variants (910). There are four different possible scenarios for production of dysfunctional protein configurations, particularly A (915), B (920), C (925) and D (930). Scenario A has a 15% probability of developing from the gene and RNA variants. Scenario B has a 25% probability of developing from the gene and RNA variants. Scenario C has a 35% probability of developing from the gene and RNA variants. Scenario D has a 25% probability of developing from the gene and RNA variants.

FIG. 10 is a diagram showing 3D and 4D models of abnormal protein structure and function. IMMs (1010) produce at least one 3D model of abnormal protein structure geometrical configurations (1020), 4D simulations of operational processes of abnormal proteins (1030), 4D simulations of abnormal protein interactions (1040) and 4D simulations of dysfunctional protein expression in intracellular protein pathways (1050).

FIG. 11 is a diagram showing IMMs analyzing abnormal protein structure and configuring solutions. Data for abnormal protein structure analysis (1105) and abnormal protein function analysis (1110) are input into an IMM (1120). The IMM generates a model to reverse engineer a protein solution to an abnormal protein problem (1125). A solution is applied in the form of a synthetic protein to solve an abnormal protein problem (1130). A solution is applied in the form of RNA instructions to encode for a novel protein to correct for a protein abnormality (1135).

FIG. 12 is a diagram showing IMM analysis of biomarkers to identify patient pathology. The patient condition evolution is shown at 1210, 1215, 1220, 1225 and 1230. At stages 1215, 1220 and 1225, 3D RNA and protein structure biomarkers are assessed (1235). The IMM assesses the snapshot data (1240) at each stage and develops 4D simulations of dysfunctional protein interactions and abnormal cell dynamics (1245). From these analyses, the IMM describes the biomolecular anatomy and physiology of the source of a patient pathology (1250).

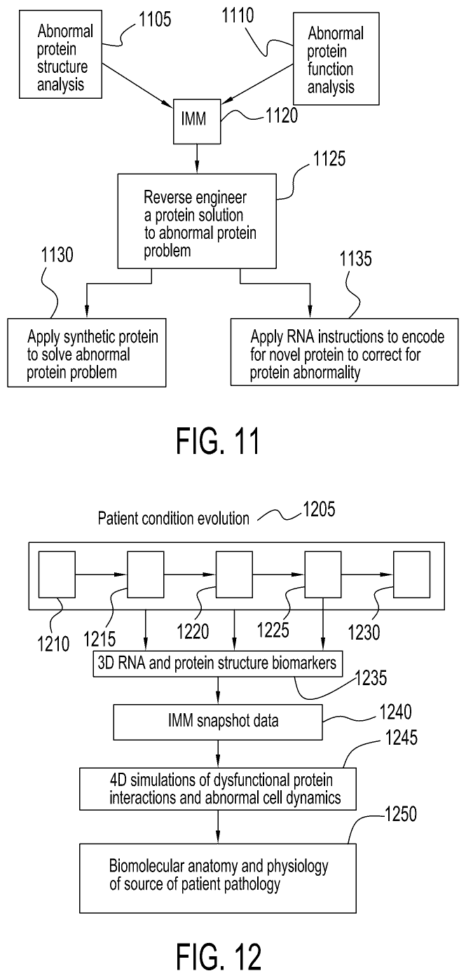

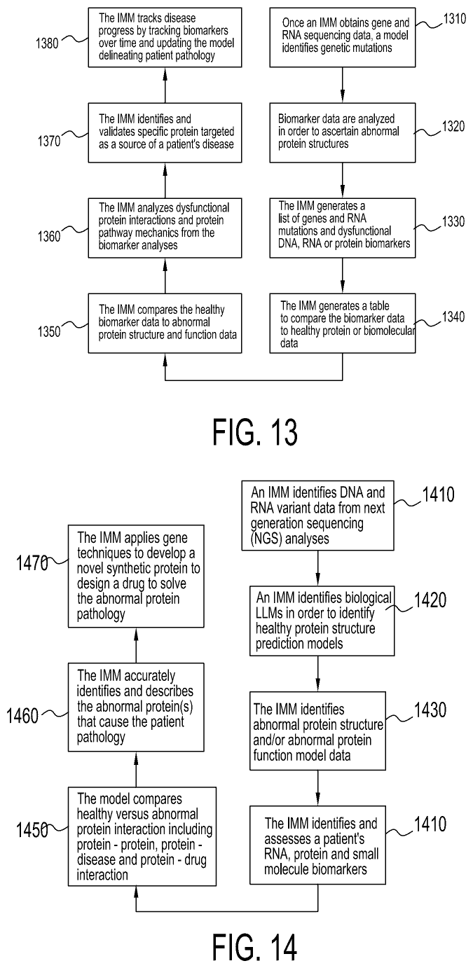

FIG. 13 is a flow chart showing the process of disease discovery utilizing IMMs. Once an IMM obtains gene and RNA sequencing data, a model identifies genetic mutations (1310) and biomarker data are analyzed in order to ascertain abnormal protein structures (1320). The IMM generates a list of gene and RNA mutations and dysfunctional DNA, RNA or protein biomarkers (1330). The IMM then generates a table to compare the biomarker data to healthy protein or biomolecular data (1340) and the IMM compares the healthy biomarker data to abnormal protein structure and function data (1350). The IMM then analyzes dysfunctional protein interactions and protein pathway mechanics from the biomarker analyses (1360). The IMM identifies and validates specific protein target(s) as a source of a patient's disease (1370) and the IMM tracks a disease progress by tracking biomarkers over time and updating the model delineating the patient pathology (1380).

FIG. 14 is a flow chart showing the process of moving from diagnostics to therapeutics by utilizing IMMs. An IMM identifies DNA and RNA variant data from next-generation sequencing (NGS) analyses (1410) and the IMM identifies biological LLMs in order to identify healthy protein structure prediction models (1420). The IMM identifies abnormal protein structure and/or abnormal protein function model data (1430) and the IMM further identifies and assesses a patient's RNA, protein and small molecule biomarkers (1440). The model compares healthy versus abnormal protein interactions, including protein-protein, protein-small molecule, protein-disease and protein-drug interactions (1450). The IMM accurately identifies and describes the abnormal protein(s) that cause the patient pathology (1460) and the IMM applies GenAI techniques or algorithms to develop a novel synthetic protein to design a drug to solve the abnormal protein pathology (1470).

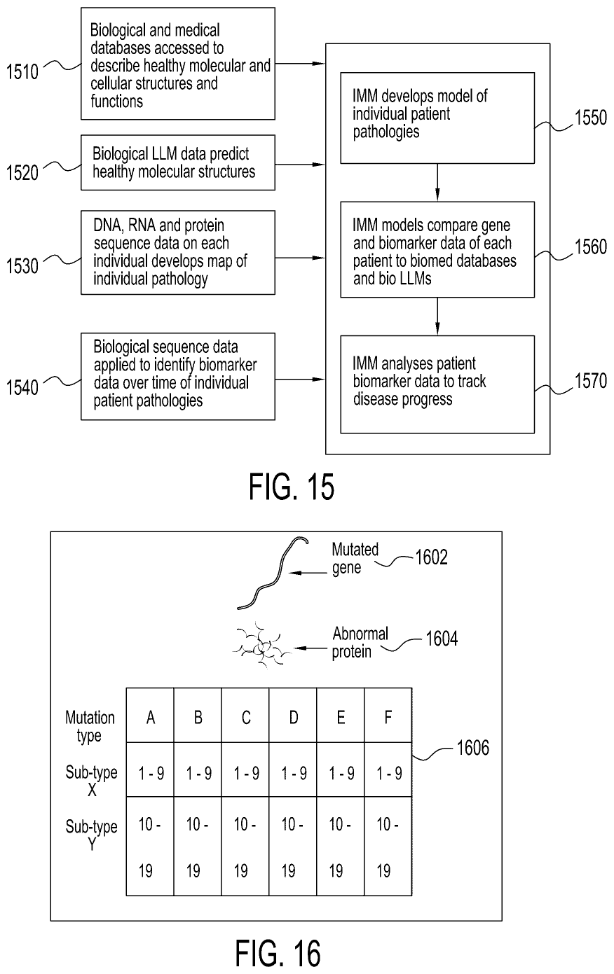

FIG. 15 is a diagram showing IMMs applied to personalized medicine to assess a patient's disease diagnosis and prognosis. Several classes of data are imported into IMMs. These data include data from biological and medical databases accessed to describe healthy molecular and cellular structure and functions (1510), biological LLM data to predict healthy molecular structures (1520), DNA, RNA and protein sequence data on each individual that develops a map of individual pathology (1530) and biological sequence data applied to identify biomarker data over time of individual patient pathologies (1540). The combination of these data sets is imported into an IMM that develops models of individual patient pathologies (1550). The IMM models compare gene and biomarker data of each patient to biomedical databases and biological LLMs (1560). The IMM analyzes patient biomarker data to track disease progress (1570).

FIG. 16 is a diagram showing a database table describing abnormal protein expression on a spectrum. A mutated gene (1602) is shown developing into an abnormal protein (1604). The table (1606) shows six different mutation types (A-F) and subtypes X (1-9) and Y (1-9) of each mutation type.

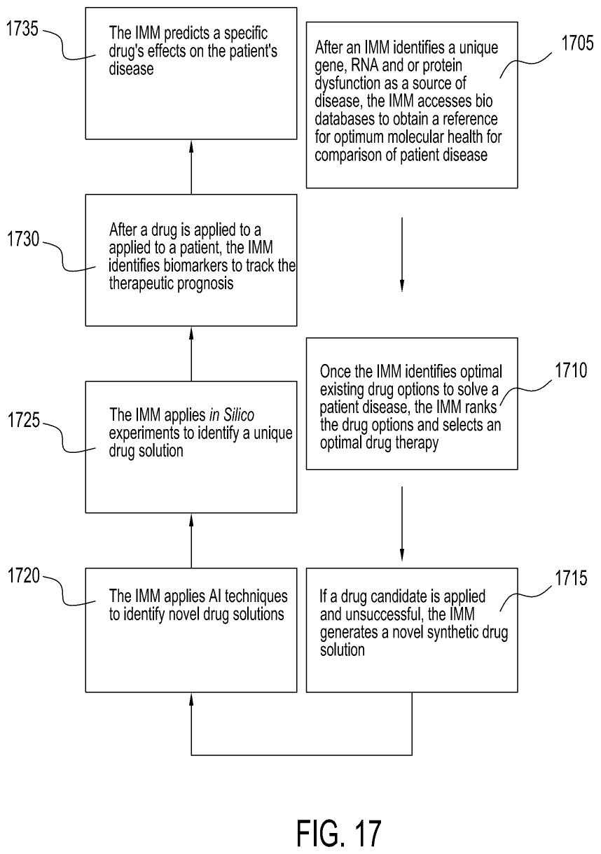

FIG. 17 is a flow chart showing the process of applying IMMs to identify therapeutic solutions to unique pathologies. After an IMM identifies a unique gene, RNA and/or protein dysfunction as a source of disease, the IMM accesses bio databases to obtain a reference for optimum molecular health for comparison of patient disease (1705). Once the IMM identifies the optimal existing drug options to solve a patient disease, the IMM ranks the drug options and selects an optimal drug therapy (1710). If a drug candidate is applied and unsuccessful, the IMM generates a novel synthetic drug solution (1715). The IMM applies AI techniques or algorithms to identify novel drug solutions (1720) and the IMM applies in silico experiments to identify a unique drug solution (1725). After a drug is applied to a patient, the IMM identifies biomarkers to track therapeutic prognosis (1730) and the IMM predicts a specific drug's effects on the patient disease (1735).

FIGS. 18-30 show IMM category dynamics. FIG. 18 is a diagram showing IMM categories. In FIG. 18, object MMs (1805) are configured to identify and describe object structures (1820), including protein, cell, organ and drug structures. Process MMs (1810) are configured to describe 4D functional biomedical processes (1825). System MMs (1815) are configured to describe unified models that may include combinations of biomedical components (1830). In additional MM categories, MMs may predict biochemical phenomena or behaviors and MMs may generate novel synthetic biochemical phenomena.

FIG. 19 is a diagram showing the medical modeling architecture outline with data pipelines. FIG. 19 shows the IMM architecture structure from FIG. 1, with data pipelines received at each corresponding level (1-13).

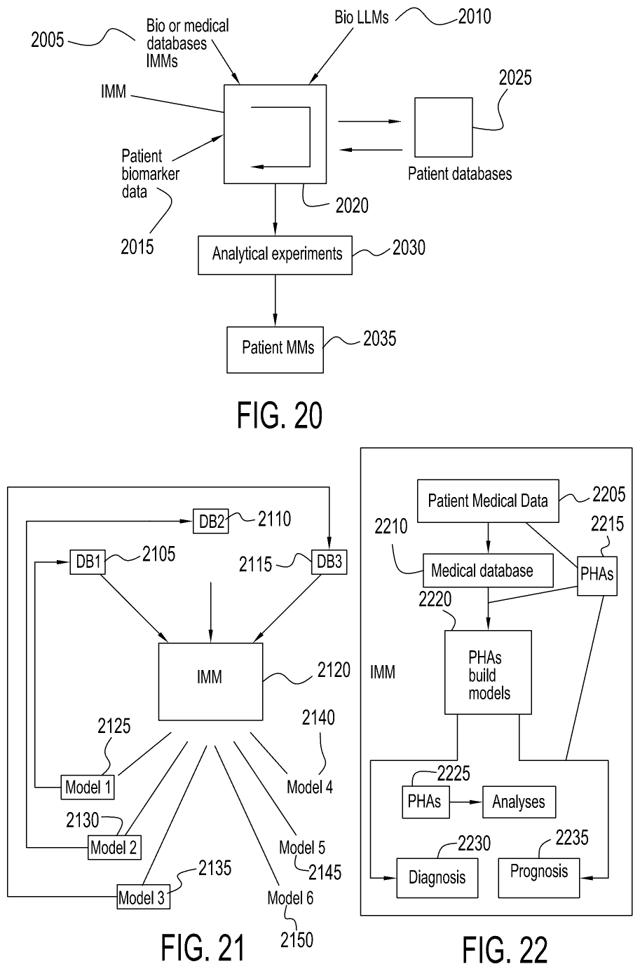

FIG. 20 is a diagram showing IMMs as active models. The IMM (2020) is shown receiving data from biological or medical databases (2005), at least one biological LLM (2010) and patient biomarker data (2015). The IMM interacts with patient databases (2025). The IMM generates analytical experiments (2030). From the analytical experiments, the LLM generates patient models (2035).

FIG. 21 is a diagram illustrating databases inputting data into an IMM that generates models. Three databases—DB 1 (2105), DB 2 (2110) and DB 3 (2115) input data into the IMM (2120). The IMM generates models 1-6 (2125, 2130, 2135, 2140, 2145 and 2150). Model 1 is shown providing data to DB 1. Model 2 is shown providing data to DB 2. Model 3 is shown providing data to DB 3.

FIG. 22 is a diagram showing PHAs performing functions in IMMs. Patient medical data (2205) are imported via PHAs (2215) into a medical database (2210) and then into an IMM (2220). The PHAs (2225) build models for diagnostic (2230) and prognostic (2235) analyses. PHAs are applied to building and analyzing IMMs. Please refer to FIGS. 27 to 30 and 66 to 76 for further descriptions of PHAs and their operational dynamics.

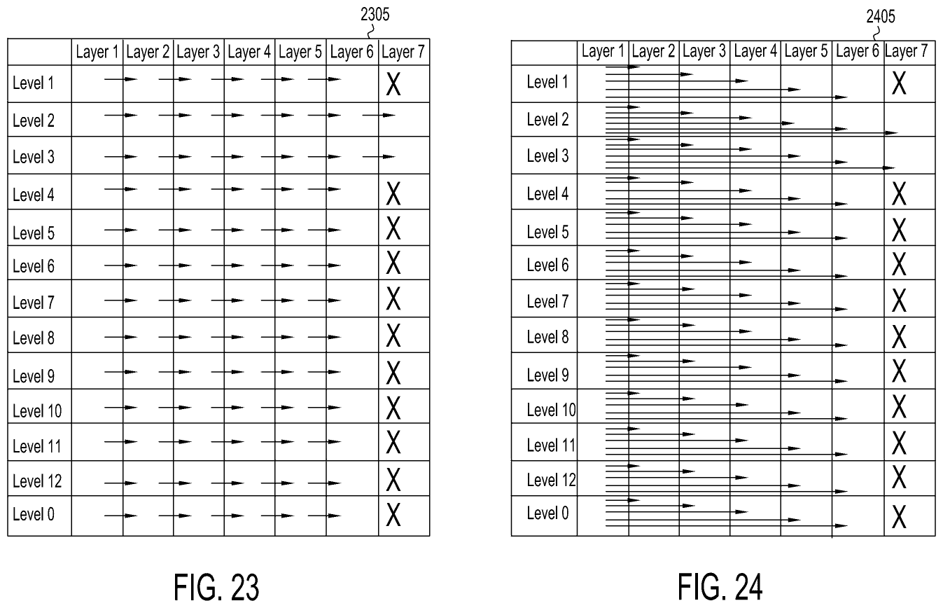

FIG. 23 is a diagram showing data flows between layers of the IMM system. The table (2305) shows the general structure of the IMM architecture. In this drawing, the layers within each level process data from the initial layer to successive layers.

FIG. 24 is a diagram showing IMM inter-layer dynamics of layers within each level. The table (2405) shows the layers within each level sharing data with multiple layers within each respective level. In an embodiment, a second layer of each level shares data with multiple layers; a third layer of each level share data with multiple layers, etc.

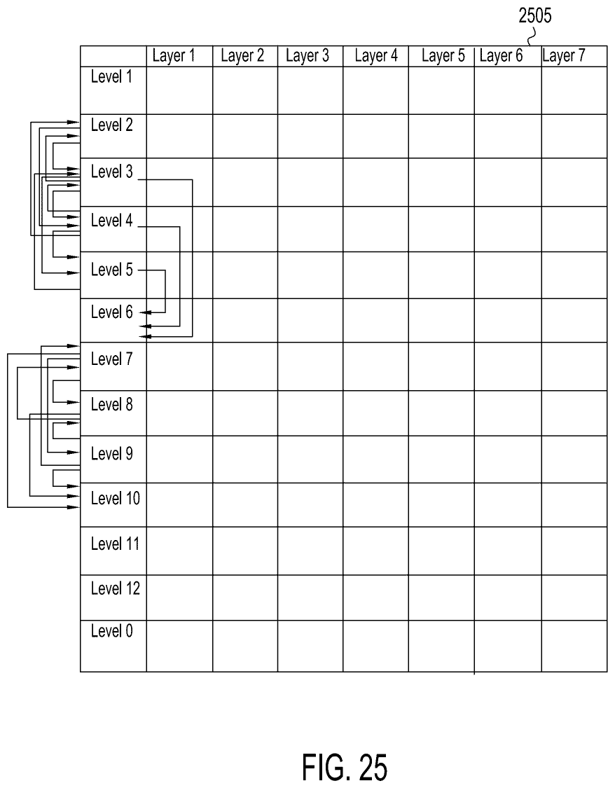

FIG. 25 is a diagram showing intra-level data sharing within the IMM system. The table (2505) shows relations between levels. The diagnostic categories of Levels 2-5 share data between themselves and the diagnostic categories share data with the diagnostic prognostic categories of Level 6. Similarly, the therapeutic categories of Levels 7-9 share data between themselves and the therapeutic categories share data with the therapeutic prognostic categories of Level 10.

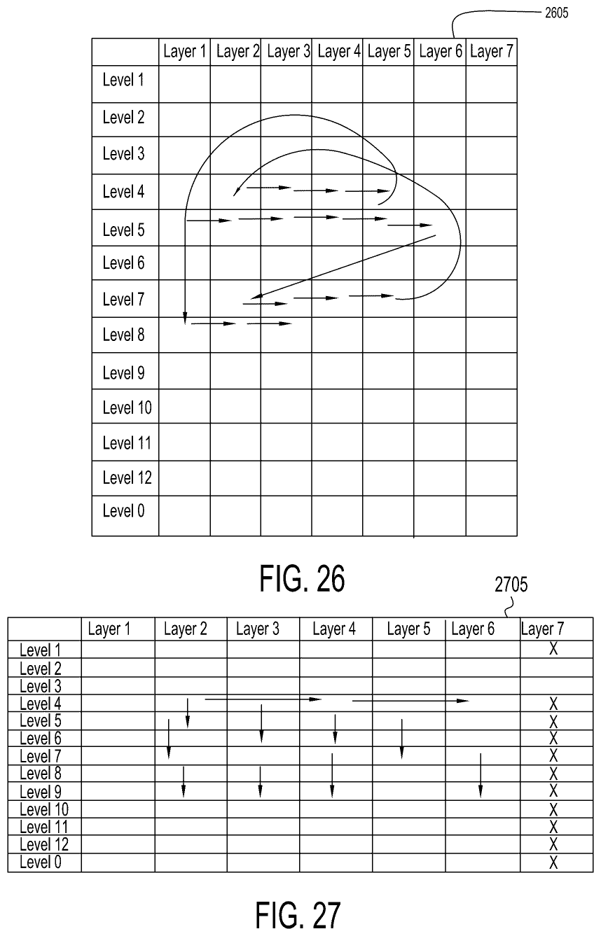

FIG. 26 is a diagram showing dynamics of relations between layers of different levels. The table (2605) shows data shared between diagnostic Levels 4 and 5 and therapeutic Levels 7 and 8.

FIG. 27 is a diagram showing PHAs facilitating two or more simultaneous data exchanges between layers. Multiple modeling categories can be activated simultaneously. In FIG. 27, the table (2705) shows several modeling categories activated on diagnostic levels 4 and 5, on the diagnostic prognostic level 6 and on the therapeutic levels 8 and 9. The therapeutic levels receive diagnostic data in order to analyze the data to generate therapies.

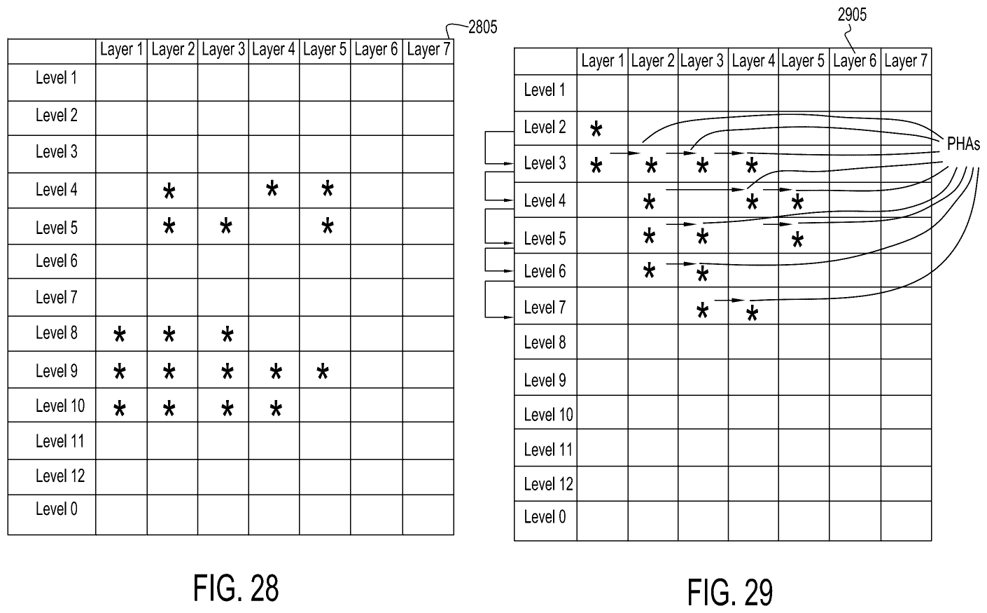

FIG. 28 is a diagram showing the simultaneous processing of two or more MMs or simulations in the IMM system. FIG. 28 shows the table (2805) with levels 4 and 5 and 8, 9 and 10 analyzing data simultaneously. These simultaneity features optimize system efficiency.

FIG. 29 is a diagram showing the application of APIs between levels and PHAs between layers of some levels connecting MM types in the IMM system. In the table (2905) in FIG. 29, APIs 1-5 are shown connecting levels 2-7. While they are shown here connecting levels 2-7, APIs connect all levels. In addition, PHAs are shown connecting models in different layers within each level. While they are shown here connecting data in layers of levels 2-7, PHAs may be applied to data in all categories in the MM system. The APIs and PHAs may work together. These software program elements are organized to work together in order to manage the reception and analysis of data in the model categories in the modeling system.

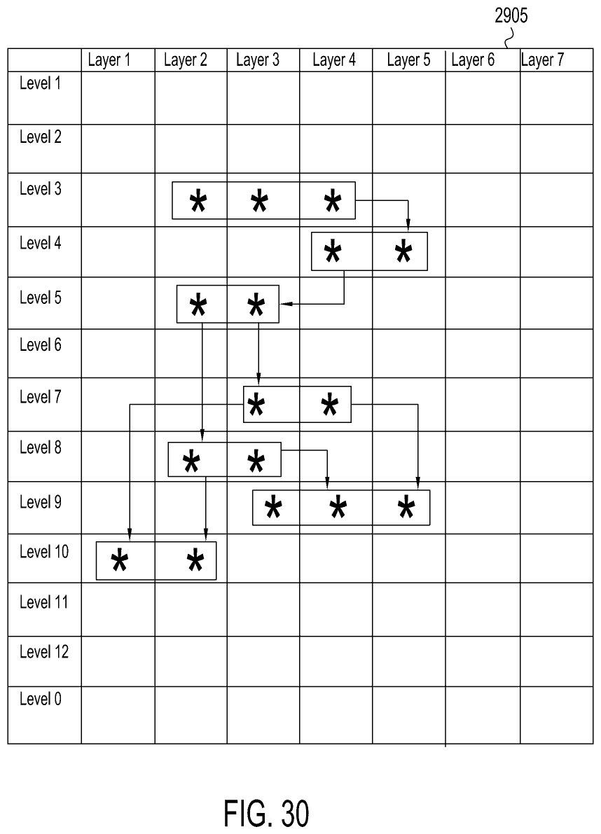

FIG. 30 is a diagram showing two or more models on two or more layers communicating data to other models on different layers in the IMM system. The table (3005) in FIG. 30 shows sets of layers combined into specific aggregated groups of model categories. These specified aggregated groups communicate data to other aggregated groups of model categories. In this example, modeling categories in Layers 2-4 on Level 3 communicate data to modeling categories on layers 4-5 on Level 4, which communicate data to modeling categories on layers 2-3 on Level 5. The aggregated group of layers 2-3 on Level 5 then send data to modeling categories on layers 3 and 4 on Level 7 and to modeling categories on layers 2 and 4 on Level 8. The modeling categories on Level 7 send data to modeling categories on layers 3-5 on Level 9 and layers 1-2 on Level 10. Similarly, modeling categories on layers 2-3 on level 8 send data to modeling categories on layers 3-5 on Level 9 and to modeling categories 1-2 on Level 10.

GenAI has the advantage of enabling graphics visualization. GenAI can present simulations or animations of healthy gene, protein and cell pathways. An animation is a functional representation of object relations over time. These animations can present different levels of simulations, from cellular interaction simulations and gene-to-protein simulations to protein-protein pathway simulations.

While it is useful to model and simulate healthy biochemical and cellular processes, it is optimal to model and simulate dysfunctional biochemical and cellular processes because this is the source of many diseases. These GenAI powered simulations are well suited to convert data from the medical model into precise diagnostic simulations to show the effects of specific genetic mutations on protein structure development and function as well as the effects of dysfunctional protein development on cellular operations.

In addition to these useful simulations, the medical modeling system is beneficial in applying GenAI to develop simulations of treatment options and prognostic probabilistic scenarios under different conditions. By generating simulations with GenAI powered agents, the medical modeling system investigates and conducts experiments to demonstrate proposed precision therapies.

AI is applied at each level of the MM spectrum. AI is applied, for instance, to the process of gathering healthcare data for a patient. AI is also applied to analytics in the context of problem finding in order to develop a precision diagnosis. AI is applied to the problem-solving context of seeking medical therapies. Finally, AI is applied to the prediction context of diagnostic and therapeutic prognoses in order to track a disease.

Not only is AI, including GenAI, neural networks, deep learning and machine learning applied to medical models, but different mathematical calculations are applied to medical models as well. These mathematical equations include algebraic calculations, differential equations and calculus. Calculus in particular is useful in order to identify temporal phenomena of molecular and cellular behaviors. Differential equations are useful for solving problems with incomplete information.

While the application of mathematics is critical to study medical models, the application of computers is critical as well. Specifically, particular semiconductors are useful to perform sophisticated modeling. These chips include GPUs, ASICs, FPGAs, CPLDs, TPUs, CPUs, SoCs and neuromorphic circuits. In addition to these logic circuits, advanced memory circuits, particularly DRAM, SRAM and high bandwidth memory (HBM) circuits are useful computer hardware components. The advent of supercomputer level GPUs in particular in the last year have advanced the field of medical models. The Nvidia H100 (and H200) series [and GH100 (and GH200) [combine GPUs with CPUs]) series], the B100 series, the R100 series and X100 series (and beyond) are powerful circuits, arranged in ASIC arrays, with from eighty billion transistors (H100) and two hundred billion transistors (B100) to a projected trillion (or trillions) transistors in coming years. Also, GPUs from AMD (MI300 [150B transistors] and MI400 families, etc.) and Intel (Gaudi 3, Falcon Shores, etc.) are useful in developing modeling hardware. When combined with multiple advanced (version 3, 3E, 4, 4E, etc.) HBM memory circuits (from Micron, Samsung and SK Hynix), these advanced logic circuits are beneficial in developing inference and training of large data sets. The trillion-transistor logic circuit is enabled in part by multi-layer semiconductor packaging. The advent of sub-3 nm CPUs, microprocessors and SoCs have also advanced computer modeling technologies.

A new era in computing began in 2023. Whereas before 2023, terabyte and petabyte scale computing were possible in order to generate individualized medical models in discrete computing apparatuses, in 2023, the AI revolution changed the traditional computing paradigm. The advent of powerful GPUs enabled rapid analysis of data sets for GenAI modeling. After 2022, the computing paradigm focused on large data centers consisting of large GPU clusters capable of exabyte and zettabyte computing functionality. Millions of logic circuits, each possessing hundreds of billions and trillions of transistors, are now aggregated in vast networks of data centers. Instead of a single supercomputer calculating medical models for each individual's personalized medical model at great expense, we see data centers renting computer time to third party vendors that enable physicians and researchers to access computability in order to create sophisticated models and simulations. It is projected that within a decade about ten percent of electricity in industrial countries could be allocated to data centers. In light of these developments, the yottabyte (a million times exabytes, which are a million times terabytes) era is inevitable. In order to manage these massive data sets, it will be necessary to apply compression and decompression algorithms (as well as encryption and security algorithms) at computing and communications junctions in order to optimize real-time medical modeling.

While computer modeling, including medical modeling, goes beyond large language model (LLM) analyses, these LLM training and inference tasks represent a new paradigm in computer modeling functionality. Of course, medical models represent a graphic modeling approach, which is well suited to GPUs, FPGAs and ASICs. But the massive computing power only available in these recent advanced semiconductors enable the work that previously was performed by large supercomputers in weeks to be performed in a cluster of a dozen or fewer GPU chips in hours. As an analogy, while the human genome was decoded in 2000 at a cost of millions of dollars, currently a human genome can be decoded for less than $1000. The advent of a new generation of GPUs enables the possible construction of human medical models for less than $1000 in the next decade. Slices of a human MM (that is, small parts of a medical model accessed for a particular purpose) can be obtained and analyzed for less than $100.

Medical models are accessed by physicians, researchers, administrators and patients via desktop computers, laptop computers, tablet computers and smartphones. The modeling system is accessed via the internet in most cases, while modeling data and databases can be stored in user computer systems and data center computer systems. The data center computers are accessible by using software as a service (SaaS) in many cases. The medical modeling system is accessible using operating systems and software graphic user interfaces from Apple, Microsoft, open source vendors and various third party vendors. The GUIs utilize a programmable dashboard for managing MM operations. The medical modeling system can be accessed with wireless (3G, 4G, 5G, 6G, etc.) and hard-wire (copper or fiber optic) communications networks.

Artificial intelligence, machine learning and deep learning are key features of the present invention. General geometric deep learning (GDL) analysis is reviewed in FIGS. 31-36. 3D graph neural networks (GNN) analysis and 3D GDL analysis are shown in FIGS. 37-51. Generative 3D GDL, which combine LLMs with 3D GDL are described in FIGS. 52-60. Also note the discussion above on general AI and ML analysis in the description of the invention.

FIG. 31 is a diagram showing GDL techniques applied to analyze protein and cellular geometric properties. RNA-protein translation (3105), protein structures (3110), protein pathway mapping (3115), protein-protein interactions (3120), protein-ligand interactions (3125), protein-lipid interactions (3130), protein-small molecule interactions (3135) and cellular component mapping (3140) are examples of microbiological phenomena and processes to which GDL techniques (3150) are applied to describe geometrical properties (3155) in MMs (3145).

FIG. 32 is a diagram showing GDL techniques applied to identify abnormal gene, RNA and protein geometric properties. A mutated gene (3205) is transcribed into a dysfunctional RNA (3210) and into an abnormal protein (3215). GDL techniques or algorithms are applied in MMs (3220) to analyze the mutated gene, the dysfunctional RNA sequence and the abnormal protein structure in order to identify abnormal geometric properties (3225).

FIG. 33 is a diagram showing GDL techniques applied to compare abnormal proteins to optimal proteins. MMs (3305) apply GDL techniques (3315) or algorithms to analyze abnormal protein structure (3310) and an optimal (healthy) protein structure (3320).

FIG. 34 is a diagram showing GDL techniques applied to predict anomalous protein structure and function. MMs (3430) apply GDL techniques (3435) or algorithms to predict anomalous protein structure and function of a mutated gene (3405), an abnormal protein structure (3410) and dysfunctional protein function of a protein (3425) with other proteins (3415 and 3420).

FIG. 35 is a diagram showing graph neural network general architecture. Data describing an input object (3505), such as a protein structure, is input into the input graph (3510). The data are next input into the hidden layers of the GNN (3515, 3520 and 3525). The data are then input into the classification layer (3530) and into the output layer (3535) of the GNN. The output layer data describing or analyzing the input object is then input into a transformed graph (3540) and then input into an IMM (3545) for analysis.

FIG. 36 is a diagram showing graph representation of an input object in a GNN. An input object (3605), such as a protein structure, is input into a graph representation of nodes and edges (3610) and into the Graph Neural Network (3615) which consists of at least four layers.

GDL and GNN algorithms are applied to description of biomedical entity structures and functions, such as molecular bio sequences including DNA and RNA structures, protein structures, functions and interactions and cellular anatomy and physiology. In particular, these algorithms describe mutated DNA, abnormal RNA and dysfunctional protein structure and function. The GDL and GNN algorithms are optimized for biomedical diagnostics. In addition, GDL and GNN algorithms are applied to prediction of molecular and cellular behaviors.

3D GDL and 3D GNN algorithms are similarly applied to description of biomedical phenomena structures and functions, but add the third dimension of the Z axis in order to optimize the precision of the descriptive and predictive analyses.

General graph neural networks, including 3D GNN, describe and predict objects in graphs to analyze node and edge relations. While GNNs (and GDL) are a general class of neural networks, there are several sub-categories of neural networks within the general class, each with a different focus. Graph convolutional NNs (GCNNs) apply filters to analyze node and edge relations. Graph attention networks (GATs) apply weighted nodes to analyze nodes and edge relations; GATs apply attention to particular clusters of nodes and/or edges to focus the analyses. Manifold valued neural networks (MVNNs) analyze spherical objects and curved surfaces. Graph of graph neural networks (GoGNNs), or equivariant graph of graph neural networks (EGGNets), analyze nodes and edges as micro graphs in order to test hypotheses and analyze protein structures and relations. Graph auto encoders (GAEs) encode and decode search space to predict object functions. Each of these categories of GNNs have a 3D graph neural network application. Each of these 3D GNN techniques and algorithms are applied to different aspects of protein structure, function and interaction analysis. These techniques may be combined into hybrid synthetic GDL applications.

3D GNNs are applied to analyze abnormal protein structure, identification of an RNA solution to an abnormal protein, drug-target binding prediction and protein interaction prediction. 3D GCNNs are applied to analysis of abnormal protein interaction description and prediction, peptide binding solutions for abnormal proteins, identification of projection options for abnormal protein solutions and identification of RNA solutions to abnormal protein configurations. 3D GATs are applied to analysis of abnormal proteins in a graph of weighted set of nodes and edges in X, Y and Z axes, active 3D experimentation of abnormal protein structure, function and interactions, analysis of peptide binding solutions for abnormal proteins, prediction of abnormal protein functions and projection of abnormal protein solution options. 3D MVNs are applied to analysis of protein folding geometries, curved protein bonds and interactions, curved protein surfaces, abnormal protein binding with proteins, lipids and ligands, abnormal protein interactions, and prediction of peptide binding solutions. 3D GoGNNs are applied to analysis of abnormal protein structures to test hypotheses of node relations, to peptide binding to repair abnormal proteins, to predict drug-target interactions and to project abnormal protein solution options. 3D GAEs are applied to analyze, describe or predict abnormal protein relations to analyze abnormal protein binding and to predict abnormal protein interactions. See the tables in FIGS. 2 and 5 for a reference to 3D GDL and 3D GNN types and their biomedical applications.

FIG. 37 is a diagram showing a 3D graph representation of a 3D graph neural network input. Data on an input 3D object (3705), such as a protein structure, is input into a 3D graph representation (3710) of nodes and edges as the 3D graph converts the object to 3D graph representation. The data are then input into the 3D GNN (3715), which includes at least three layers. After the data are analyzed in the 3D GNN, they are output into the IMM (3720).

FIG. 38 is a diagram showing a 3D GNN analysis of a 3D object and prediction of node connections. Data on a 3D object (3805) are input into multiple layers of a 3D GNN (3810). The 3D GNN makes 3D object node connection predictions (3815).

FIG. 39 is a diagram showing 3D GNN with convolutional layers to output probabilistic options, with convolution layers applying different filters. Data on a 3D object (3905), such as a 3D protein structure, is input into a 3D graph as a 3D graph representation conversion (3910). The 3D graph is input into a 3D graph NN (3915) and into several convolution layers of the 3D GNN (3920, 2925 and 3930). The convolution layers input data into connected layers (3935) and into a probabilistic output layer (3940). From this output layer, the data are input into the IMM (3945). In an embodiment, note that convolution layers apply different digital filters.

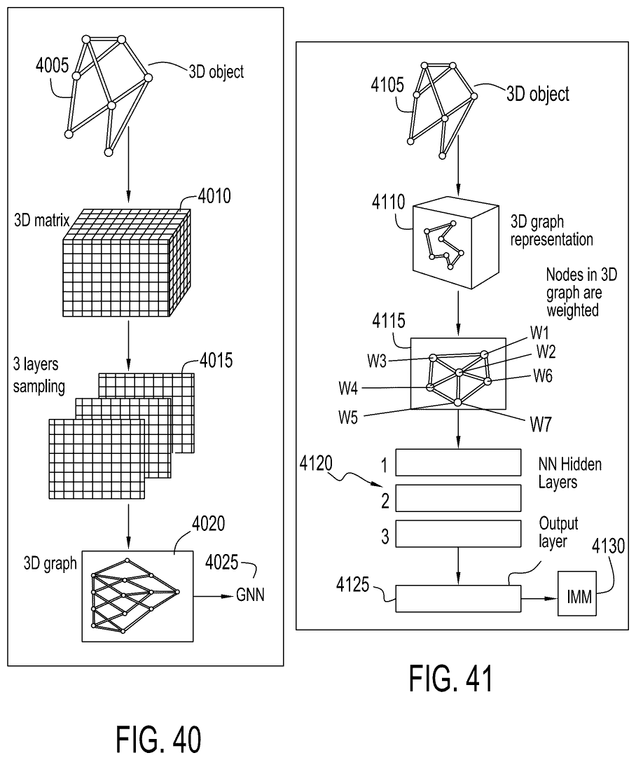

FIG. 40 is a diagram showing a 3D object converted to a 3D matrix and layer sampling for conversion to a 3D graph. Data on a 3D object (4005), such as a 3D protein structure, are input into a 3D matrix (4010). The data are input into three layer sampling (4015), into a 3D graph (4020) and into a GNN (4025). The GNN may be 2D or 3D. This process of converting data on a 3D object into a 3D graph for input into a 3D GNN prepares the digital object for analysis in the GNN.

FIG. 41 is a diagram showing a 3D object converted to a 3D graph and nodes weighted in preparation for 3D GAT NN. Data on a 3D object (4105), such as a 3D protein, is input into a 3D graph representation (4110). The nodes (W1 to W7) in the graph (4115) are weighted. These weighted object node representation data are input into NN hidden layers (4120) 1-3 and then into an output layer (4125). The output layer inputs the 3D object data analysis into the IMM (4130).

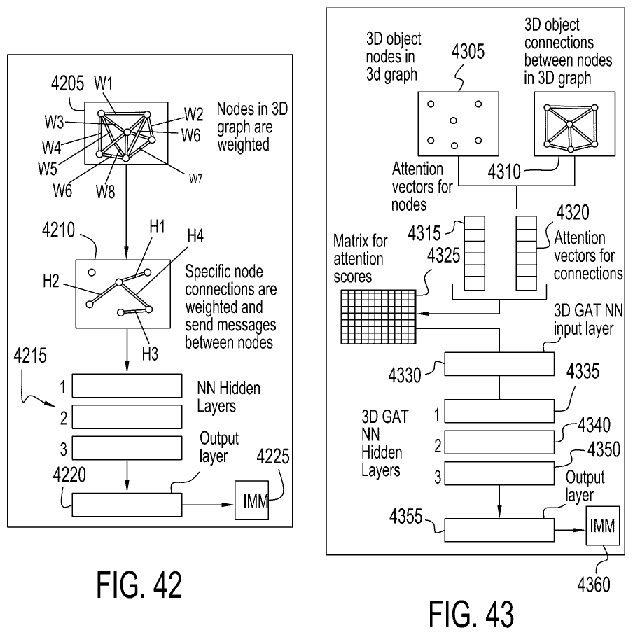

FIG. 42 is a diagram showing connections between 3D object nodes that are weighted and messages sent between nodes in a 3D GAT NN. Data on the connections between 3D object nodes (W1-W8) (4205) are weighted. The weighted node connections then send messages between nodes (H1-H4) in the 3D object analysis (4210). The 3D object data are then sent to GNN hidden layers (4215) 1-3 for analysis and to the output layer (4220) and to the IMM (4225).

FIG. 43 is a diagram showing attention scores aggregated for nodes and connections for presentation to 3D GAT NN. Data on the 3D object nodes in a 3D graph (4305) and the 3D object connections between nodes in a 3D graph (4310) are converted to attention vectors for nodes (4315) and attention vectors for connections (4320). The node and connection data are then input into a matrix for attention scores (4325) and into a 3D GAT NN input layer (4330). The data are input into 3D GAT NN hidden layers (4335) 1-4 and into an output layer (4355). These data and analyses are then input into the IMM (4360).

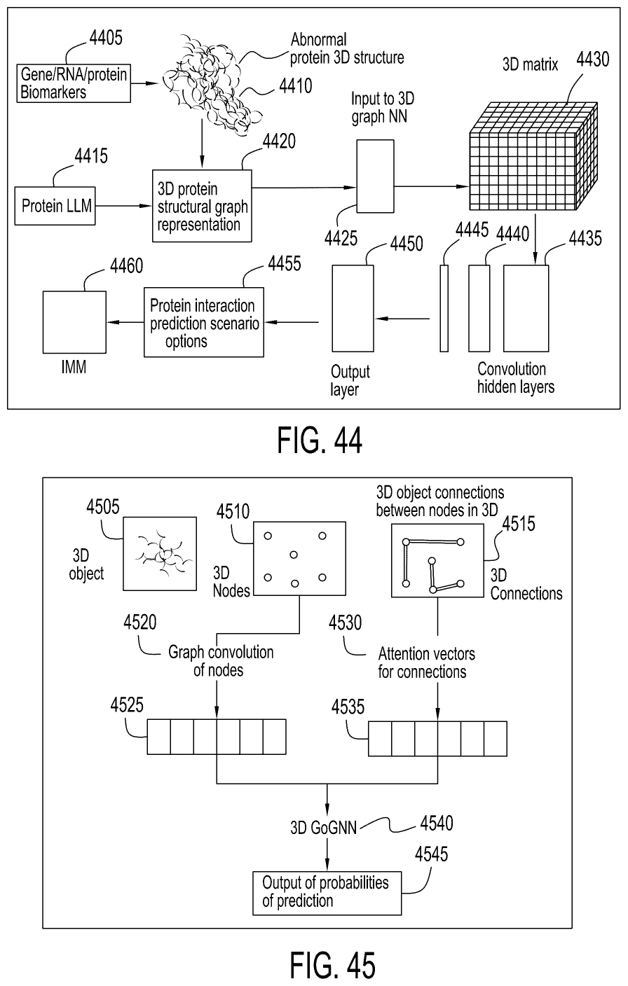

FIG. 44 is a diagram showing a 3D GNN with convolutional and GAT hybrid configuration to predict protein interaction. Data on gene, RNA and/or protein biomarkers (4405) are converted into an abnormal protein 3D structure (4410). These data on abnormal protein 3D structure and data from a protein LLM (4415) are input into a 3D protein structure graph representation (4420). These data on a 3D protein are input into a 3D graph NN (4425) and into a 3D matrix (4430). These data are input into three convolution hidden layers of the GNN (4435, 4440 and 4445) and then into an output layer (4450). The GNN generates protein interaction prediction scenario options (4455) and inputs these data into the IMM (4460).

FIG. 45 is a diagram showing a 3D graph of graph NN inputting two types of node and connection analyses. Data on a 3D object (4505), such as a 3D protein structure, are represented as 3D nodes (4510) and 3D connections (4515). The 3D nodes are input into a graph convolution of nodes (4520) and into hidden layers (4525). The 3D connections are input into attention vectors for connections (4530) and into hidden layers (4535). The two sets of hidden layers are input into a 3D GoGNN (4540), which outputs an analysis of probabilities of prediction (4545).

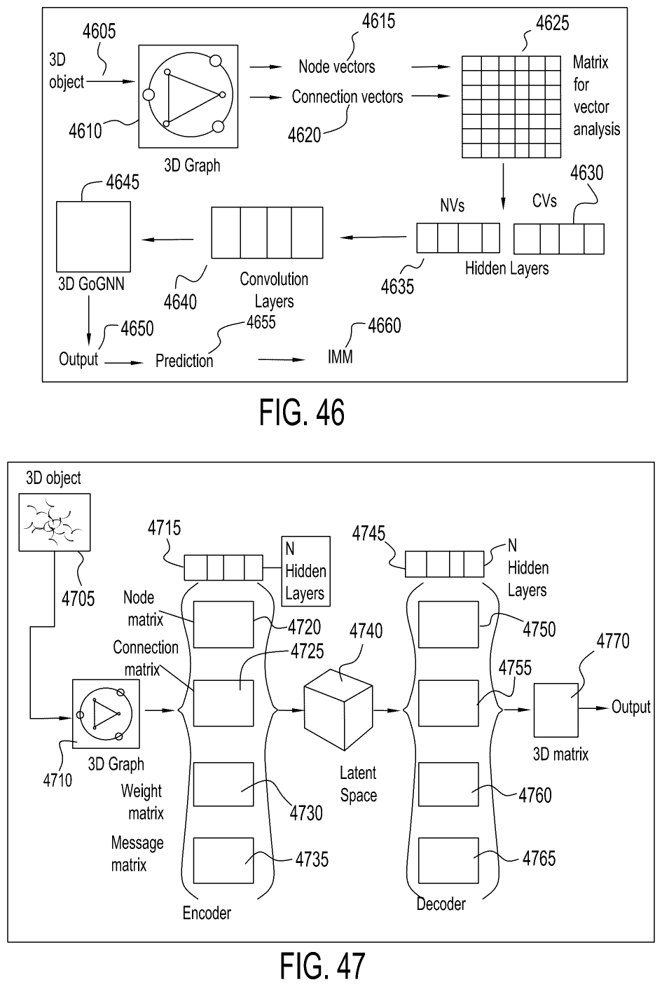

FIG. 46 is a diagram showing two types of vectors analyzed in a 3D GoGNN. Data on a 3D object (4605) are input into a 3D graph (4610). The node vectors (4615) and connection vectors (4620) are input into the matrix for vector analysis (4625) and are then input into hidden layers—node vectors into hidden layers (4635) and connection vectors into hidden layers (4630). These data are input into convolution layers (4640) and into the 3D GoGNN (4645) and output (4650). The output data enables the NN to make predictions (4655) about objects and object relations, which are entered into the IMM (4660).

FIG. 47 is a diagram showing a 3D autoencoder GNN model. Data on a 3D object (4705), such as a 3D protein structure, are input into a 3D graph (4710). These data are input into a 3D GNN with N hidden layers (4715) for encoding. The encoder layers include a node matrix (4720), connection matrix (4725), weight matrix (4730) and message matrix (4735). These data and analyses are input into a latent space (4740) and into a 3D GNN with N hidden layers for decoding. These decoding layers may include corresponding node matrix (4750), connection matrix (4755), weight matrix (4760) and message matrix (4765) in the decoder. These data are input into a 3D matrix (4770) and output. The output data of the 3D autoencoder GNN may be input into an IMM.

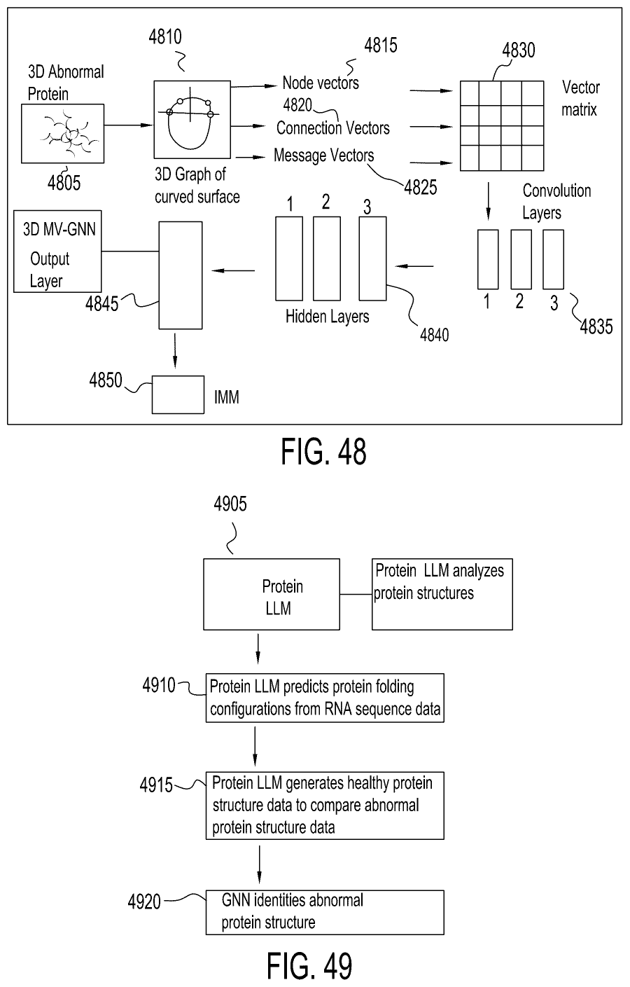

FIG. 48 is a diagram showing a 3D MV-GNN of a 3D abnormal protein with curved surfaces. Data on a 3D abnormal protein structure (4805) are input into a 3D graph of a curved surface (4810) which outputs data analyzing node vectors (4815), connection vectors (4820) and message vectors (4825). The vectors and vector analyses are input into a vector matrix (4830) and into convolution layers (4835) and hidden layers (4840) 1-3. These data and analyses are input into a 3D MV-GNN output layer (4845) and then into an IMM (4850). The MV-GNNs are well suited to analysis of curved surfaces of complex protein 3D structures which contain novel non-Euclidean description challenges.

FIG. 49 is a diagram showing a protein LLM comparing healthy protein structure data to abnormal protein structure data. A protein LLM (4905) analyzes protein structures, with the protein LLM predicting protein folding configurations from RNA sequence data (4910). The protein LLM generates healthy protein structure data to compare abnormal protein structure data (4915) and a GNN identifies an abnormal protein structure (4920).

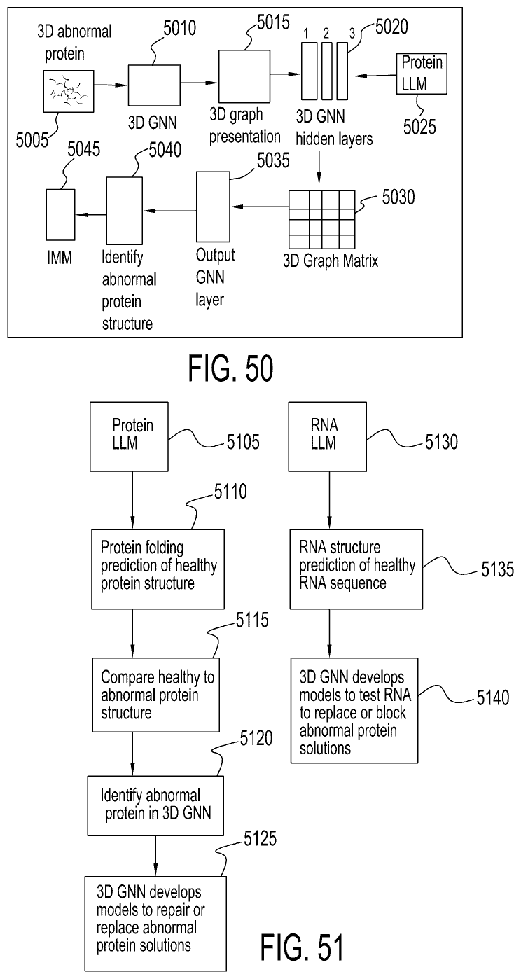

FIG. 50 is a diagram showing a 3D GNN analyzing an abnormal protein structure. Data from a 3D abnormal protein (5005) are input into a 3D GNN (5010) and converted to a 3D graph representation (5015). The data are input into 3D GNN hidden layers (5020) 1-3. Data from a protein LLM (5025) on health proteins are input into the 3D GNN for comparison to the abnormal protein. The abnormal protein data are input into a 3D graph matrix (5030) and to an output GNN layer (5035). The data on the abnormal protein structure (5040) and analysis are input into an IMM (5045).

FIG. 51 is a diagram showing a 3D GNN analyzing abnormal protein structure to generate solution options. A protein LLM (5105) outputs data on protein folding prediction of a healthy protein structure (5110), which data are compared to abnormal protein structure (5115). These data identify an abnormal protein structure in a 3D GNN (5120). A 3D GNN develops models to repair or replace abnormal protein solutions (5125). An RNA LLM (5130) outputs data to an RNA structure prediction of a healthy RNA sequence (5135), from which data a 3D GNN develops models to test RNA to replace or block abnormal protein solutions (5140). Once the abnormal protein structure configuration is identified, a 3D GNN may apply RNA solutions.

In an embodiment, graph isomorphism NNs are applied to compare similar graph representations or to highlight distinctive differences of similar graphs. In another embodiment, 3D graph isomorphism NNs are applied as a new class of 3D GNN.

While 3D GDL and 3D GNN algorithms are optimized for analysis of mutated DNA, dysfunctional RNA and abnormal protein structures in their descriptive mode, these algorithms are also applied to prediction of protein behaviors. For example, 3D GDL and 3D GNN algorithms are applicable to predict functional interaction behaviors of abnormal proteins. As such, these algorithms are applied to project abnormal behaviors of abnormal proteins generated from abnormal genes and RNA. 3D GDL and 3D GNN algorithms are applied to predict an abnormal protein configuration from an abnormal RNA, which is quite different from the exercise of predicting a normal healthy protein structure from a normal healthy gene or RNA sequence. Protein language models are configured to predict healthy protein structures from healthy RNA sequences but are mute on the real challenge of predicting abnormal protein structure from abnormal RNA sequences which represent the source of many diseases.

In addition to predicting the structure of abnormal proteins, 3D GDL and 3D GNN algorithms are also applied to prediction of abnormal protein functions and abnormal protein interactions in protein networks. It is precisely this abnormal protein functionality in protein networks that interfere with normal protein network operation mainly due to lack of binding to healthy proteins by abnormal proteins that cause diseases. The nature of the dysfunctional protein structure, function and interaction enables doctors and researchers to diagnose the precise patient disease and to develop a diagnostic prognosis of the disease. An analysis and description of a unique abnormal DNA, RNA or protein structure and function indicates optimal therapy solution options. In many cases, the abnormal protein structure results in failure to properly bind with other proteins or molecules in protein networks, thereby disrupting the protein networks and manifesting as a pathology. Identifying the precise nature of the structural dysfunction of the protein is critical to understanding the underlying nature of a disease and predicting the disease's probable outcomes. Accurate descriptions of these abnormal proteins then represent the targets for therapeutic solutions.

While LLMs and PLMs can predict healthy protein structures, 3D GDL and 3D GNN algorithms are applied to predict abnormal protein structures and functions from a mutated gene or abnormal RNA sequence. These algorithms are applied to backwards engineer a description of the dysfunctional RNA sequence from an abnormal protein structure description.

3D GDL and 3D GNN algorithms can be combined with LLMs and PLMs in order to generate novel protein structure in order to solve a particular problem involving a mutated gene or abnormal protein structure. In this sense, the combination of these technologies enables the generation of novel synthetic proteins. Once an abnormal protein structure is identified by a 3D GDL or 3D GNN algorithm, the novel synthetic protein can be configured by applying the PLM, which generates a healthy RNA sequence or a healthy protein structure. Generative 3D GDL and generative 3D GNN applies PLM techniques to generate novel synthetic proteins, with the 3D GNN giving descriptive form to the PLM analyses for protein creation. The functional application of generative synthetic proteins is to block (i.e., inhibit) abnormal RNA from producing abnormal proteins or peptides, to repair abnormal proteins with supplemental peptides or to replace a protein with a novel protein structure. In effect, the generation of a novel synthetic protein is related to the prediction of a healthy protein's behavior because the prediction of a healthy protein enables the construction of a normal protein that becomes the target objective of the novel protein synthesis.

FIG. 52 is a diagram showing an MM analyzing abnormal biomarkers and comparing the abnormal biomarkers to healthy DNA, RNA, proteins and antibodies, with an MM applying 3D GDL types to construct a novel synthetic drug to match to the drug target. Biomarkers (5205) identify abnormal RNA (5210), abnormal proteins (5215) and abnormal antibodies (5220), which data are input into an MM (5241). DNA and RNA database (5223) data are input into a DNA and RNA LLM (5232), protein database (5226) data are input into a protein LLM (5235) and antibody database (5229) data are input into an antibody LLM (5238). These DNA, RNA, protein and antibody databases and LLMs represent reference data, which are input into the MM (5241). The MM applies a 3D GDL (5244) that apply various ML algorithms involving 3D GCNN (5259), 3D GAT (5262), 3D MVNN (5265), 3D GoGNN (5268) and 3D GAE (5271). The MM identifies a drug target (5447) and the ML algorithms generate a novel synthetic drug (5256), which is applied by an MM (5250) to perform a drug-target fit analysis (5253).

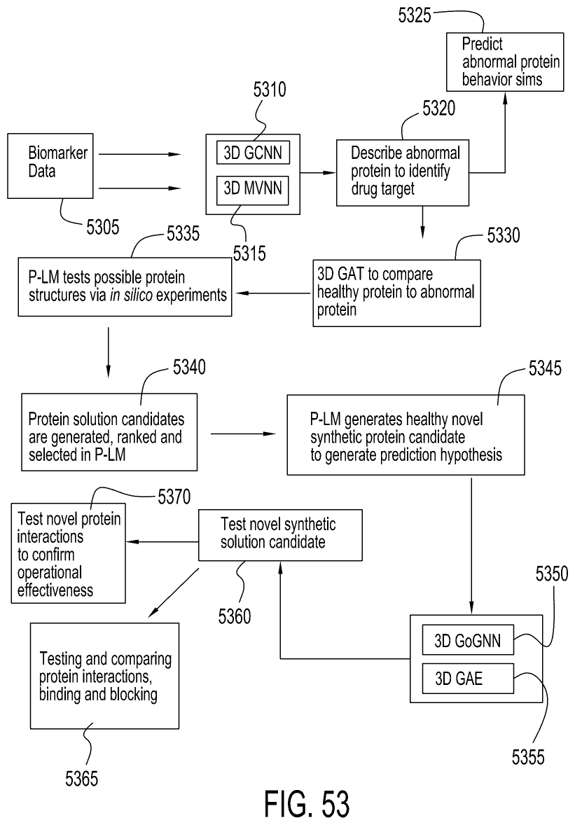

FIG. 53 is a diagram showing synthesis of an LLM and 3D GDL to identify, generate and test a novel synthetic protein. Biomarker data (5305) are forwarded to a 3D GCNN (5310) and 3D MVNN (5315) to describe an abnormal protein to identify as a drug target (5320). The GNNs generate abnormal protein behavior prediction simulations (5325). After the GNNs describe an abnormal protein to identify a drug target, a 3D GAT compares a healthy protein to an abnormal protein (5330). A protein language model tests possible protein structures via in silico experiments (5335) and protein solution candidates are generated, ranked and selected in the protein language model (5340). The P-LM generates a healthy novel synthetic protein candidate to generate a prediction hypothesis (5345). These data are input into a 3D GoGNN (5350) and/or 3D GAE (5355). The 3D GNNs test novel synthetic solution candidates (5360) by testing and comparing protein interactions, binding and blocking scenarios (5365). The 3D GNNs test novel protein interactions to confirm operational effectiveness (5370). The IMMs apply these tools to identify, generate and test a novel synthetic protein.

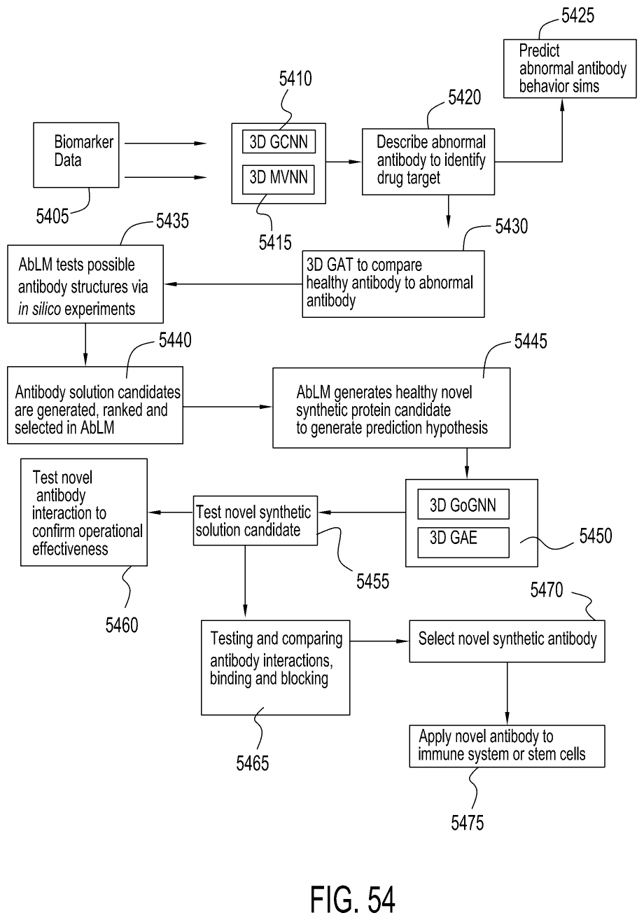

FIG. 54 is a diagram showing the synthesis of an LLM and 3D GDL to identify, generate and test a novel synthetic antibody. Biomarker data (5405) are input into a 3D GCNN (5410) and a 3D MVNN (5415) to describe an abnormal antibody to identify a drug target (5420). The GNNs general abnormal antibody prediction simulations (5425). After the GNNs describe an abnormal antibody to identify a drug target, a 3D GAT compares a healthy antibody to an abnormal antibody (5430). An antibody language model tests possible antibody structures via in silico experiments (5435) and antibody solution candidates are generated, ranked and selected in the AbLM (5440). The AbLM generates a healthy novel synthetic antibody candidate to generate prediction hypothesis (5445). These data are input into a 3D GoGNN (5447) and/or 3D GAE (5450). The 3D GNNs test novel synthetic solution candidates (5455) by testing and comparing antibody interactions, binding and blocking scenarios (5465). The 3D GNNs test novel antibody interactions to confirm operational effectiveness (5460). After testing, the 3D GNNs select a novel synthetic antibody (5470). The IMMs apply these tools to identify, generate and test a novel synthetic antibody. The novel antibody is then applied to a patient's immune system or to stem cells (5475).

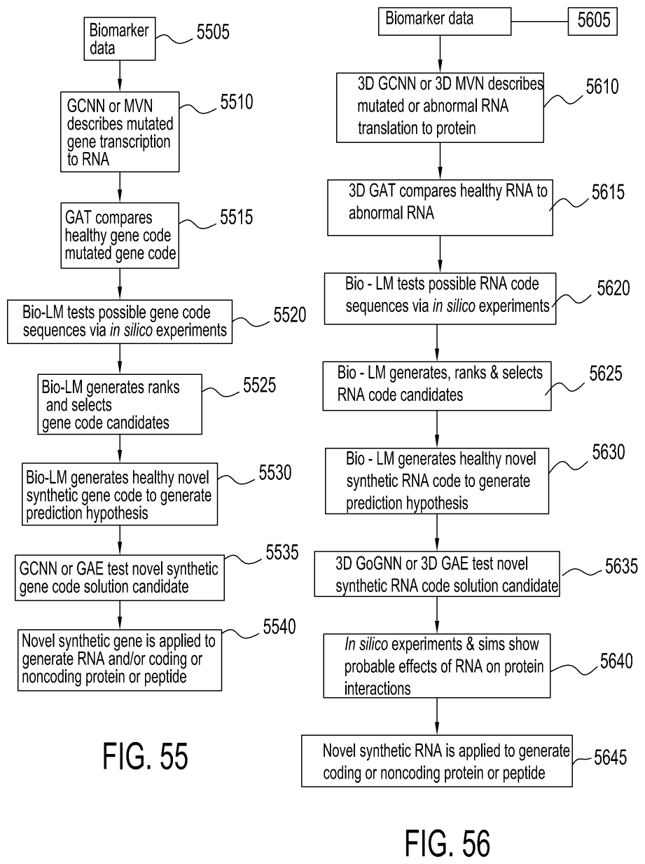

FIG. 55 is a flow chart showing the synthesis of an LLM and GDL to identify, generate and test a novel synthetic gene and transcription process. Biomarker data (5505) are input into the modeling system. A GCNN or MVN describes a mutated gene transcription to an RNA (5510). A GAT compares a healthy gene code to a mutated gene code (5515). Abio-LM tests possible gene code sequences via in silico experiments (5520) and generates, ranks and selects gene code candidates (5525). The bio-LM generates healthy novel synthetic gene code to generate a prediction hypothesis (5530). A GoGNN or GAE test a novel synthetic gene code solution candidate (5535) and the novel synthetic gene is applied to generate RNA and/or coding or noncoding protein or peptide (5540).

FIG. 56 is a flow chart showing the synthesis of an LLM and 3D GDL to identify, generate and test novel synthetic RNA and translation process. Biomarker data (5605) are input into a modeling system. A 3D GCNN or 3D MVN describe mutated or abnormal RNA translation to a protein (5610). A 3D GAT compares a healthy RNA sequence to an abnormal RNA (5615). A bio-LM tests possible RNA code sequences via in silico experiments (5620). The bio-LM generates, ranks and selects RNA code candidates (5625) and generates healthy novel synthetic RNA code to generate a prediction hypothesis (5630). A 3D GoGNN or 3D GAE test a novel synthetic RNA code solution candidate (5635). In silico experiments and simulations show probable effects of RNA on protein interactions (5640) and the novel synthetic RNA is applied to generate a coding or non-coding protein or peptide (5645).

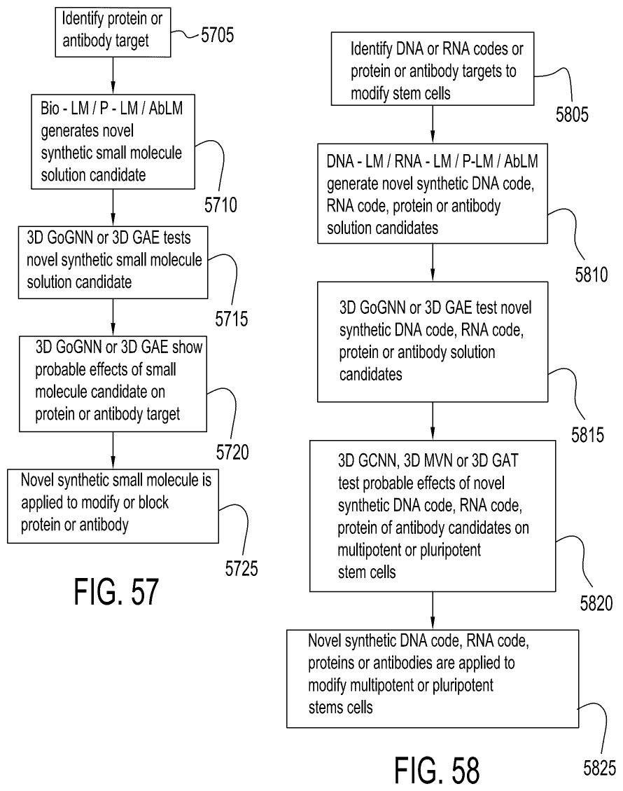

FIG. 57 is a flow chart showing the synthesis of an LLM and 3D GDL to identify, generate and test a novel synthetic small molecule. After a protein or antibody target is identified (5705), a bio-LM, PLM or AbLM generates novel synthetic small molecule solution candidates (5710). A 3D GoGNN or 3D GAE tests a novel synthetic small molecule solution candidate (5715) and a 3D GoGNN or 3D GAE show probable effects of a small molecule candidate on a protein or antibody target (5720). The novel synthetic small molecule is applied to modify or block a protein or antibody (5725).

FIG. 58 is a flow chart showing the synthesis of an LLM and 3D GDL to identify, generate and test a novel synthetic DNA, RNA, protein or antibody to modify stem cells. After DNA or RNA codes or protein or antibody targets are identified to modify stem cells (5805), DNA-LM, RNA-LM, PLM and/or AbLM generates novel synthetic DNA code, RNA code, protein or antibody solution candidates (5810). 3D GoGNN or 3D GAE test novel synthetic DNA code, RNA code, protein or antibody solution candidates (5815). 3D GCNN, 3D MVN or 3D GAT test probable effects of novel synthetic DNA code, RNA code, protein or antibody candidates on multipotent or pluripotent stem cells (5820). The novel synthetic DNA code, RNA code, proteins or antibodies are applied to modify multipotent or pluripotent stem cells (5825).

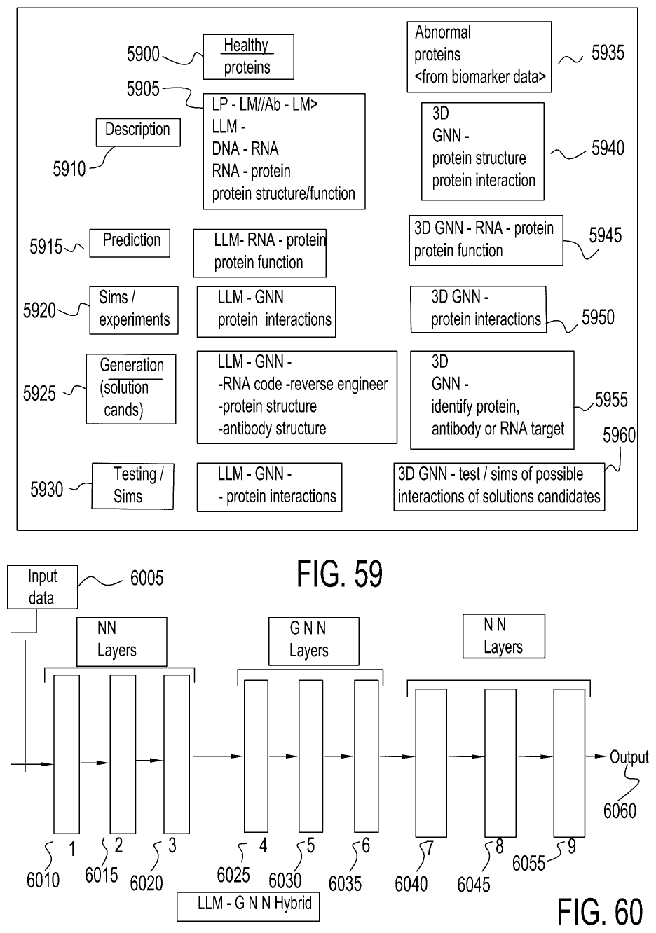

FIG. 59 is a diagram showing a 3D GNN as descriptive of an abnormal protein and predictive of abnormal protein interactions. Healthy proteins (5900) are analyzed in a PLM and a AbLM (5905). LLMs describe healthy DNA to RNA transcription, RNA-protein translation and protein structures (5910). LLMs predict RNA-protein translation and protein functions (5915). Bio LLMs and GNNs are applied in simulations and in silico experiments to analyze healthy protein interactions (5920). Bio LLMs and GNNs are applied in simulations (via reverse engineering) to generate RNA code, protein structures and antibody structures (5925). The bio LLMs and GNNs test simulations of protein interactions (5930). Abnormal proteins are analyzed from biomarker data (5935). 3D GNNs are applied to describe abnormal protein structure and abnormal protein interactions (5740). 3D GNNs are applied to RNA-protein translation prediction of abnormal protein structures and functions (5945). 3D GNNs are applied to describe and predict abnormal protein interactions (5950) in simulations and in silico experiments. 3D GNNs are applied to identify abnormal protein, antibody or RNA targets (5955) for which novel synthetic protein, antibody or RNA can be generated. 3D GNNs test and simulate possible interactions of solution candidates (5960).

FIG. 60 is a diagram showing an LLM-GNN hybrid model. A multi-layer hybrid LLM-GNN system is shown with three input NN layers (6010, 6015 and 6020) 1, 2 and 3 receiving input data (6005) at layer 1. The GNN layers are represented by layers 4, 5 and 6 (6025, 6030 and 6035). The NN layers (6040, 6045 and 6055) of layers 7, 8 and 9 of the system output data (6060). The GNN layers are integrated into the LLM. While this diagram shows three GNN layers and six NN layers of the LLM, in an embodiment, there may be many thousands of layers, with some layers focused on specialized or parallel analyses in order to describe, predict or generate biomolecular objects.

Different classes of LLMs generate varied solutions. Protein language models (PLMs) generate novel synthetic proteins while antibody language models (AbLMs) generate novel synthetic antibodies. Similarly, DNA LLMs generate novel synthetic healthy DNA sequence code solution candidates while RNA LLMs generate novel synthetic healthy RNA sequence code solution candidates. These four different LLM types also generate novel synthetic antibody, protein, DNA code and RNA code solution candidates applied to programming stem cells. Biological LLMs generate novel synthetic small molecule solution candidates as well.

GNNs, both 2D and 3D GNN varieties, describe DNA, RNA, protein and antibody entity structures and predict entity functions and interactions. In addition, GNNs test novel synthetic DNA, RNA, protein and antibody solution candidates. For example, the 3D GoGNNs may test and compare protein or antibody binding or blocking behavior characteristics to test novel synthetic protein or antibody candidates.

The 3D GNNs describe abnormal protein or antibody targets, and compare a healthy protein or antibody to an abnormal protein or antibody. A PLM will generate a novel synthetic protein and an AbLM will generate a novel synthetic antibody to block or bind to the protein or antibody target. The 3D GNNs will then test candidate solutions generated from the language models. The 3D GNNs describe the abnormal protein, predict abnormal protein behaviors (functions and interactions), compare the abnormal protein to a healthy protein and identify a drug target. The LLM (e.g., a PLM or an AbLM) will test possible protein structures via in silico experiments and generate protein solution candidates that are ranked and selected. The LLM then generates a healthy novel synthetic protein or antibody solution candidate and generates a prediction hypothesis for the novel protein or antibody behavior. The 3D GNNs test novel synthetic protein or antibody solution candidates by testing and comparing protein or antibody binding, blocking and possible interactions. The specialized biological LLMs and 3D GDL NN's cooperate to identify and solve problems

While 3D GNNs are applied to describing the structure of abnormal protein and antibody structures that manifest as disease, predicting abnormal protein and antibody functions by performing in silico experiments enables the GNNs to identify a protein or antibody target. LLMs are not configured to deal with abnormal protein or antibody structures because such dysfunctional structures are unpredictable and random. Abnormal protein structures embody random structural dysfunctions that biological LLMs are not trained on and that are too varied. On the other hand, biological LLMs are well suited to working with, and generating, healthy normal protein and antibody structures. These LLMs are designed to predict healthy structures, from DNA to RNA transcription and from RNA to protein translation. From these prediction analyses, these biological LLMs can be configured to generate healthy proteins.

When the 3D GNNs identify and describe abnormal protein or antibody structures, these discoveries enable the identification of a protein or antibody target. Once the protein or antibody target is identified, the biological LLMs focus on building a healthy protein or antibody. Once a healthy novel synthetic protein or antibody is generated, the 3D GNNs test the novel synthetic protein or antibody solution candidates for probabilistic effectiveness. For example, the 3D GNNs can compare abnormal protein or antibody structure, function and interaction potentialities with proposed healthy protein or antibody candidates. In addition to protein or antibodies, the system is applied to small molecule and stem cells as well. In the case of 3D GNN analysis of (abnormal or healthy) protein or antibody structures and functions or in the case of biological LLM prediction and generation of novel synthetic candidates, the system applies simulations to conduct experiments. The three dimensional analysis of protein structures, for example, can be extrapolated to four dimensional simulations of functional operations and interactions. The 3D GNNs are optimized for these complex analyses. The 3D GNNs cooperate with the LLMs to perform these analyses. In an embodiment, since both the GNNs and the biological LLMs are comprised of neural networks and deep learning algorithms, different specialized LLMs may be integrated with different varieties of GNNs for specific analytical tasks. The aforementioned description of the combination of 3D GDL and biological LLMs provide a unique approach to identifying the sources of disease, predicting the behavior of these abnormal components, generating novel solutions to solve the pathologies and test and predict the solution options.

In an embodiment, some of these tasks may be performed simultaneously by dividing the analytical components of the operations in order to accelerate the process.

Several categories of software that are related to IMMs, including personal health assistants (PHAs) and the integrated health record platform, are described in FIGS. 61-81.

FIG. 61 is a diagram showing 3D GNNs connected to a 3D database management system. Bio data (6105) are input into a 3D database management system (6110). The 3D DBMS stores data for the 3D GNN techniques. The 3D DBMS is accessed by various 3D GNN techniques or algorithms, including 3D GCNN (6115), 3D GAT (6120), 3D MVNN (6125), 3D GoGNN (6130) and 3D GAE (6135). These 3D GNN techniques and algorithms store and access 3D data in the 3D DBMS. In an embodiment, the database may be multidimensional. 3D databases include 3D tables to store 3D graph data sets. In an embodiment, multi-dimensional databases may be applied to 3D GNNs, 3D GDL, 3D generative GNNs and 3D generative GDL. In another embodiment, in-3D graph analysis may be performed in 3D databases and in 3D GNNs, which test 3D node weights and analyze 3D node relations.

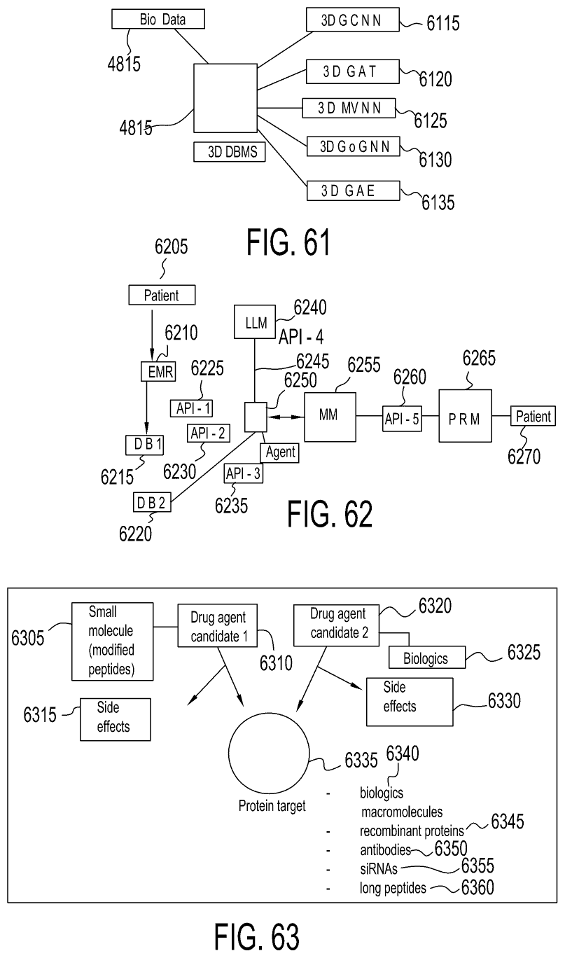

FIG. 62 is a diagram showing APIs in the MM system. The patient (6205) supplies medical data that are stored in an EMR (6210), which is stored in a database (DB 1) (6215). The EMR, DB 1, DB 2 (6220), and an LLM (6240) interface with a software agent (6250) via API's (API-1 (6225), API-2 (6230), API-3 (6235) and API-4 (6245)). The software agent interfaces with the MM (6255). The MM interfaces with a PRM (6265) via API-5 (6260) and the patient relationship management (PRM) software interfaces with the patient (6270).

FIG. 63 is a diagram showing the process of novel synthetic drug design. Two drug agent candidates, including drug agent candidate 1 (6310) and drug agent candidate 2 (6320) are directed to a protein target (6335). Drug agent candidate 1 is a small molecule (modified peptides) (6305) and drug agent candidate 2 is a biologic (6325). The first drug agent candidate reveals side effects (6315) and the second drug agent candidate reveals other side effects (6330). Biologic macromolecules (6340) represent recombinant proteins (6345), antibodies (6350), siRNAs (6355) and long peptides (6360).

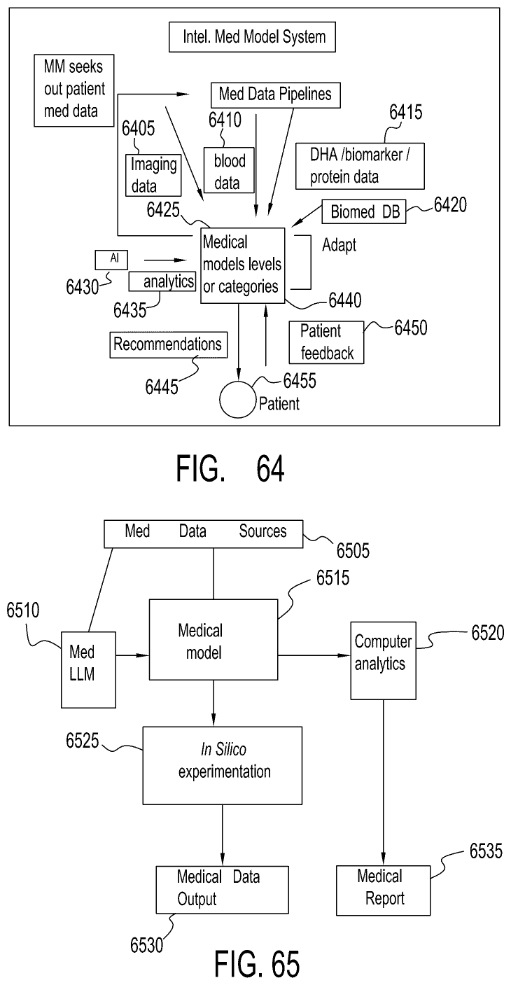

FIG. 64 is a diagram showing an intelligent medical modeling system. The IMM system shows medical data pipelines that include imaging data (6405), blood data (6410), DNA, biomarker and protein data (6415) and biomedical database data (6420). The medical data are input into the MMs at various levels and categories. AI (6430) and analytics (6435) are input into the MMs. The MMs perform analyses and process the data to solve medical problems. The MMs produce recommendations (6445) for the patient (6455), with the patient providing feedback (6450). In addition, the MMs seek out patient medical data from the medical data pipelines in order to collect data to solve medical problems.

FIG. 65 is a diagram showing MM data interaction. Medical data sources (6505) supply data to a medical LLM (6510) and the MM (6515). The MM performs computer analytics (6520) and in silico experimentation (6525). From the computer analytics, the system produces a medical report (6525). From the in silico experimentation, the system produces medical data output (6530).

FIG. 66 is a diagram showing PHAs generating medical summaries from medical articles, databases or LLMs. PHAs (6625) facilitate the generation of medical summaries (6630) from medical articles (6605), medical databases (6610) and medical or biological LLMs (6615). Medical or biological LLMs generate medical data by accessing NLP (6620). A PHA (6635) then facilitates the generation of medical summaries.

FIG. 67 is a diagram showing PHAs accessing patient medical test data and EMR, EHR and IHR data to build an IMM. A PHA (6725) facilitates the input of patient medical test data (6705) into an IMM (6745). Another PHA (6730) facilitates the input of EMR (6710) data into the IMM. An additional PHA (6735) facilitates the input of EHR (6715) data into the IMM.

Finally, another PHA (6740) facilitates the input of IHR (6720) data into the IMM.

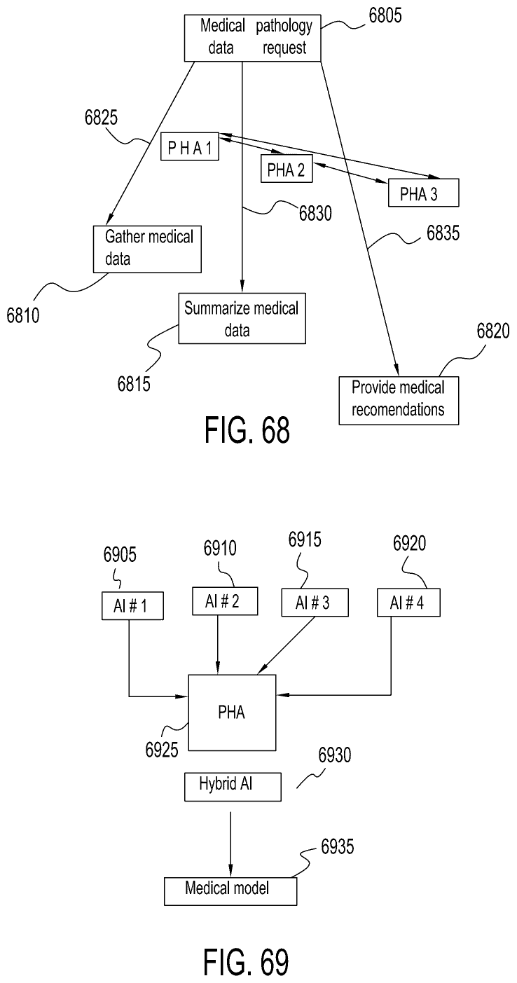

FIG. 68 is a diagram showing specialized PHAs in a multi-agent system applying skills to perform functions and communicate with each other. Medical pathology data are requested (6805) and three PHAs perform functions to process the data. PHA 1 (6825) facilitates the gathering of medical data (6810), PHA 2 (6830) facilitates the summarization medical data (6815) and PHA 3 (6835) facilitates the provision of medical recommendations (6820). The three PHAs interact with each other.

FIG. 69 is a diagram showing a PHA combining two or more AI techniques or algorithms into a hybrid AI technique or algorithm and applied to an MM. AI techniques or algorithms (1 (6905), 2 (6910), 3 (6915) and 4 (6920)) are input into, or accessed by, the PHA (6925). The PHA combines the two or more AI techniques or algorithms into a hybrid AI technique or algorithm (6930). The hybrid AI technique or algorithm is then input into or accessed by the MM (6935). In an embodiment, PHAs can also actively synthesize AI techniques (into hybrid AI algorithms) to solve an MM problem.

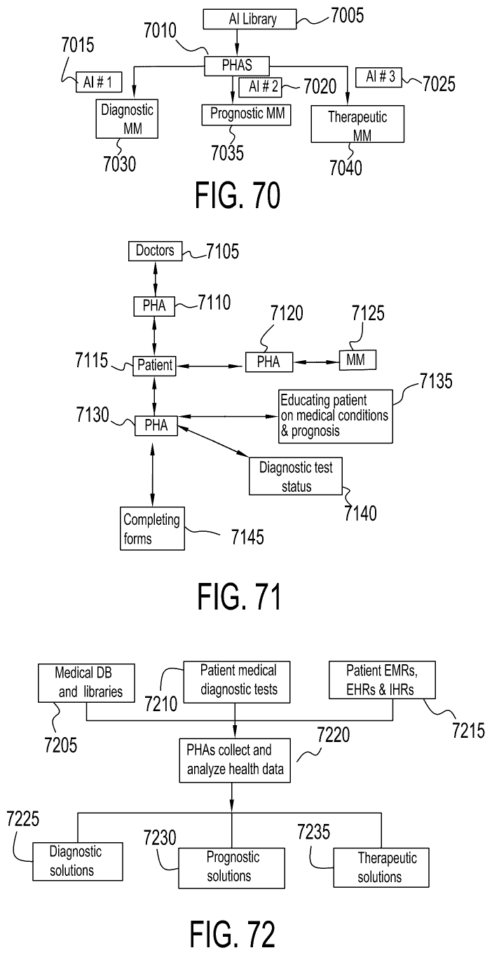

FIG. 70 is a diagram showing PHAs supplying different AI techniques or algorithms to different types of MMs. An AI library (7005) is shown supplying PHAs (7010) with AI #1 (7015) for a diagnostic MM (7030), AI #2 (7020) for a prognostic MM (7035) and AI #3 (7025) for a therapeutic MM (7040).

FIG. 71 is a diagram showing PHAs acting as interfaces with doctors, a patient MM and patient tasks. Doctor(s) (7105) interface with a patient (7115) via a PHA (7110). The patient interfaces with an MM (7125) via a PHA (7120). The patient uses a PHA (7130) to complete forms (7145), identify diagnostic test status (7140) and educate the patient on medical conditions and prognosis (7135).

FIG. 72 is a diagram showing PHAs collecting and analyzing health data to develop diagnostic, prognostic or therapeutic solutions. A medical database and libraries (7205), patient medical diagnostic test data (7210) and patient EMRs, EHRs and IHRs (7215) are collected and analyzed by PHAs (7220). The PHAs identify diagnostic solutions (7225), prognostic solutions (7230) and therapeutic solutions (7235).

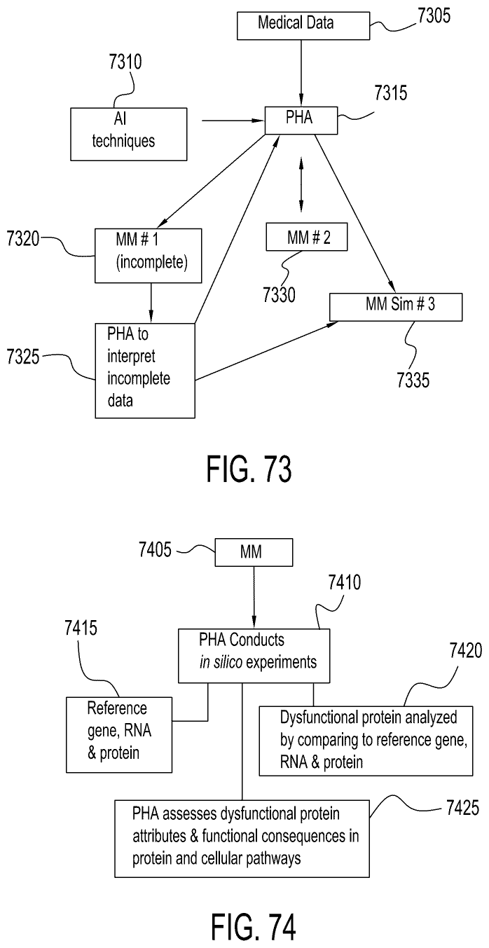

FIG. 73 is a diagram showing PHAs generating MMs, analyzing incomplete data and solving MM problems over time. Medical data (7305) and AI techniques or algorithms (7310) are forwarded to a PHA (7315), which supplies the data and AI techniques to MM #1 (7320), MM #2 (7330) and MM simulation #3 (7335). APHA (7325) interprets incomplete data in the three MMs.

FIG. 74 is a diagram showing PHAs conducting in silico experiments to compare dysfunctional proteins to reference genes, RNA and proteins. A PHA (7410) conducts in silico experiments in an MM (7405). The experiments analyze reference gene, RNA and protein data (7415), analyze a dysfunctional protein by comparing it to a reference gene, RNA and protein (7420) and assesses dysfunctional protein attributes and functional consequences in protein and cellular pathways (7425).

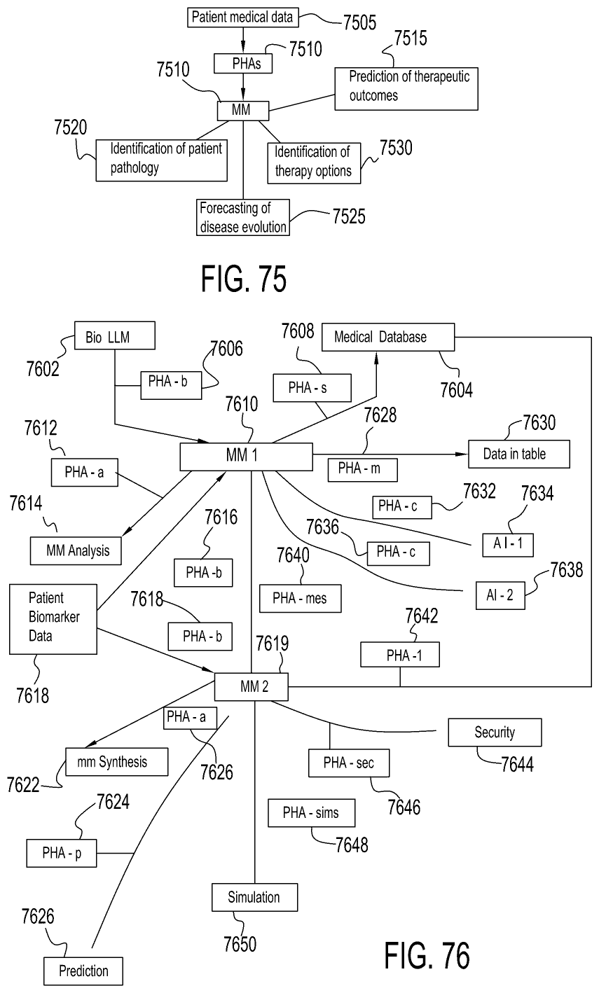

FIG. 75 is a diagram showing PHAs enabling an MM to supply diagnostic, prognostic and therapeutic solutions. Patient medical data (7505) is forwarded to an MM (7510) by PHAs (7510). The MM identifies a patient pathology (7520), forecasts a disease evolution (7525), identifies therapy options (7530) and predicts therapy options (7515).

In an embodiment, PHAs receive medical data from an MM analysis, triggers a physician intervention and automatically communicates with a physician.

FIG. 76 is a block diagram showing PHA system dynamics. Two MMs (MM 1 (7610) and MM 2 (7619) interact with various inputs and operations by working with various types of PHAs. MM 1 receives data from a Bio LLM (7602) via PHA-b (7606) and stores data in a Medical Database (7604) via PHA-s (7608). MM 1 places data in a table (7630) via PHA-m (7628) and M/I conducts MM analysis (7614) with PHA-a (7612). Two AI algorithms, AI-1 (7634) and AI-2 (7638) interface with M/I via PHA-c's (7632 and 7636). Patient biomarker data (7618) are forwarded to M/I via PHA-b (7616). The two MM's (MM 1 and MM 2) interface via PHA-mes (7640). Patient biomarker data are also forwarded to MM 2 via a PHA-b (7618). MM 2 is connected to MM synthesis (7622) via PHA-a (7620), prediction (7626) via PHA-p (7624), simulation (7650) via PHA-sims (7648) and security (7644) via PHA-sec (7646). PHA-m refers to model builders, PHA-a refers to analyzers, PHA-s refers to searchers, PHA-c refers to combiners, PHA-i refers to interrogators, PHA-mes refers to messengers, PHA-b refers to brokers, PHA-sec refers to security, PHA-p refers to predictors and PHA-sims refers to simulators.

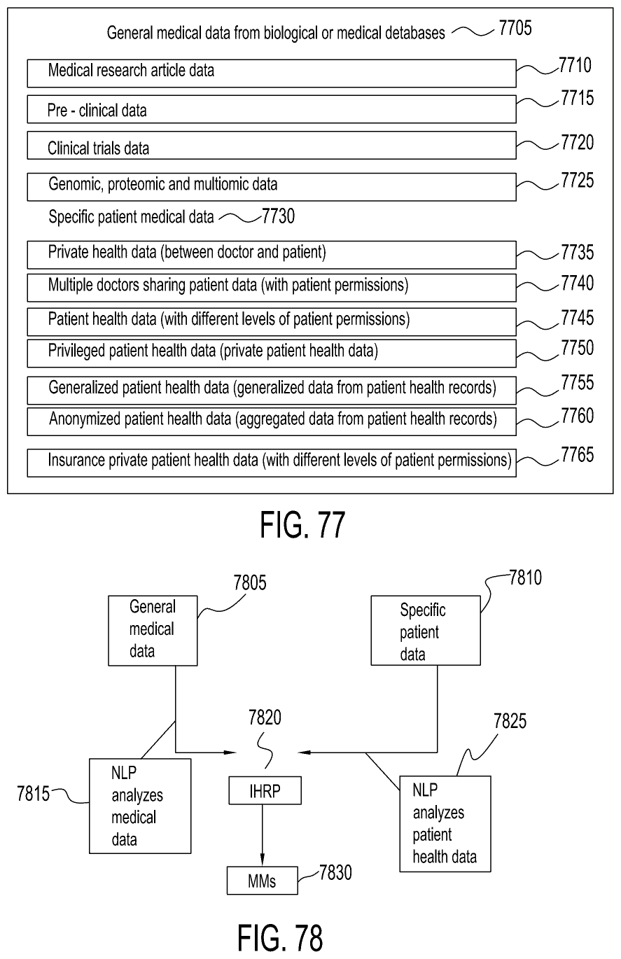

FIG. 77 is a list of IHRP levels. General medical data from biological or medical databases (7705) includes medical research article data (7710), pre-clinical data (7715), clinical trials data (7720) and genomic, proteomic and multiomic data (7725). Specific patient medical data (7730) includes private health data (between doctor and patient) (7735), multiple doctors sharing patient data (with patient permissions) (7740), patient health data (with different levels of patient permissions) (7745), privileged patient health data (private patient health data (7750), generalized patient health data (generalized data from patient health records) (7755), anonymized patient health data (aggregated data from patient health records) (7760) and insurance private patient health data (with different levels of patient permissions) (7765).

FIG. 78 is a diagram showing a natural language processing program analyzing health data that are input to the IHRP and MMs. General medical data (7805) and specific patient data (7810) are input into an IHRP (7820). The IHRP applies NLP to analyze medical data (7815) and patient health data (7825). The medical data and patient data are input into MMs (7830).

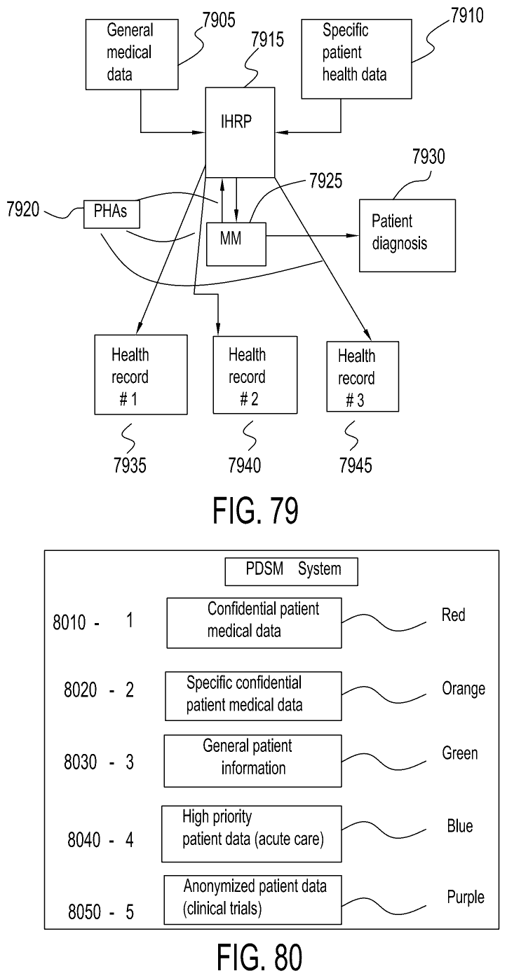

FIG. 79 is a diagram showing an IHRP interacting with MMs and generating patient health records. General medical data (7905) and specific patient health data (7910) are input into an IHRP (7915). The medical and patient health data are input into an MM (7925), which produces a patient diagnosis (7930). The IHRP also generates three health records, namely, health record #1 (7935), health record #2 (7940) and health record #3 (7945).

In an embodiment, the IHRP receives, interprets, analyzes and integrates medical codes. A patient chart can be automatically converted to an IHR either directly or through an EMR or EHR translation. Medical codes are input into the IHR.

FIG. 80 is a diagram showing the PDSM system layers. The PDSM system includes confidential patient medical data (8010), assigned a red color; specific confidential patient medical data (8020), assigned an orange color; general patient information (8030), assigned a green color; high priority patient data (acute care) (8040), assigned a blue color; and anonymized patient data (e.g., for clinical trials) (8050), assigned a purple color.

FIG. 81 is a diagram showing the PDSM filtering patient security for MMs. General health data (8105) and patient health data (8110) are input into an IHRP (8115), which produces patient health report #1 (8120) and patient health report #2 (8125). The IHRP supplies data to the patient data security management (PDSM) system (8130), which forwards the data to the MMs (8135), which produce a patient diagnostic model with security (8140) and patient (diagnostic) prognostic model with security (8145).

FIGS. 82 to 98 describe IMMs applied to medical diagnostics.

FIG. 82 is a diagram showing patient abnormal proteins analyzed and compared to healthy proteins to assess a patient disease in an IMM. Data from a protein database (8220), a protein folding prediction (8205), a protein LLM (8210) and a miRbase biomarker database (8222) are input into a model (8215) describing healthy proteins as a reference. The data on patient gene, RNA and protein biomarkers (8230) and AI and DL algorithms are applied to an analysis of abnormal proteins (8235). An MM (8225) compares abnormal protein data to reference protein data. An IMM assesses the patient disease attributes (8240) and the patient disease is diagnosed in a patient model (8245).

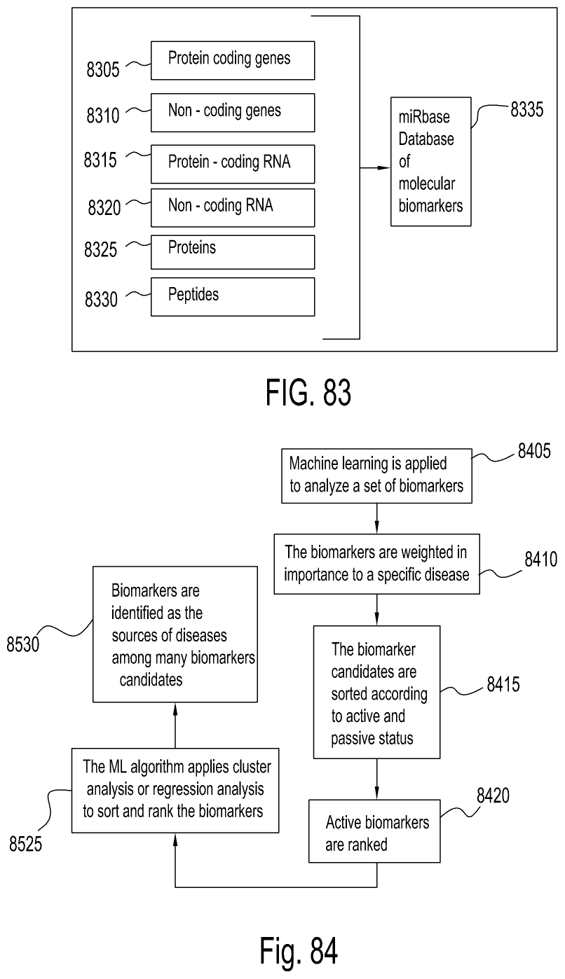

FIG. 83 is a diagram showing a MiR database of biomarker types that indicate the presence of a disease. Data for protein coding genes (8305), non-coding genes (8310), protein coding RNA (8315), non-coding RNA (9320), proteins (8325) and peptides (8330) are input into the miRbase database of molecular biomarkers (8335).

FIG. 84 is a flow chart showing how multiple biomarkers are analyzed to assess the sources of diseases. After machine learning algorithms are applied to analyze a set of biomarkers (8405), the biomarkers are weighted in importance to a specific disease (8410). The biomarker candidates are sorted according to active and passive status (8415) and active biomarkers are ranked (8520). The ML algorithm applies a cluster analysis or regression analysis to sort and rank the biomarkers (8525) and the biomarkers are identified as the sources of diseases among many biomarker candidates (8530).

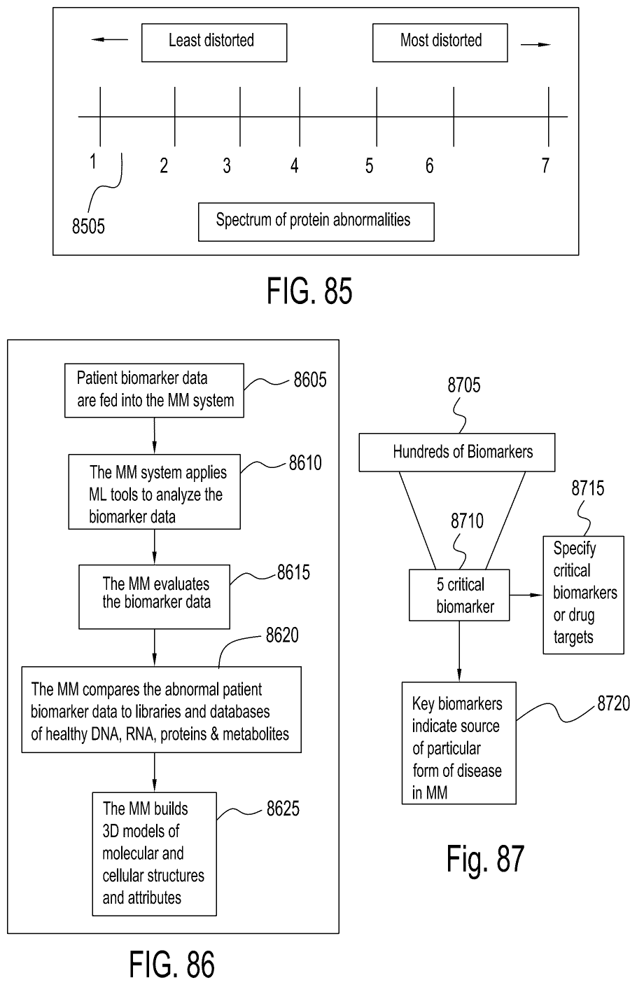

FIG. 85 is a diagram showing protein abnormalities ranked on a scale based on geometrical configuration distortion degree. A spectrum of protein abnormalities (8505) from 1, the least distorted, to 7, the most distorted, is shown.

FIG. 86 is a flow chart showing MMs performing biomarker analyses. After patient biomarker data are fed into the MM system (8605), the MM system applies ML tools to analyze the biomarker data (8610). The MM evaluates the biomarker data (8615) and compares the abnormal patient biomarker data to libraries and databases of healthy DNA, RNA, proteins and metabolites (8620). The MM then builds 3D models of molecular and cellular structures and attributes (8625).

FIG. 87 is a diagram showing an MM analyzing many biomarkers to identify several critical biomarkers as a source of disease and as drug targets. From hundreds of biomarkers (8705), five critical biomarkers (8710) are identified as key biomarkers that indicate the source of a particular form of disease in an MM (8720). The specific critical biomarkers are analyzed and identified as drug targets (8715).

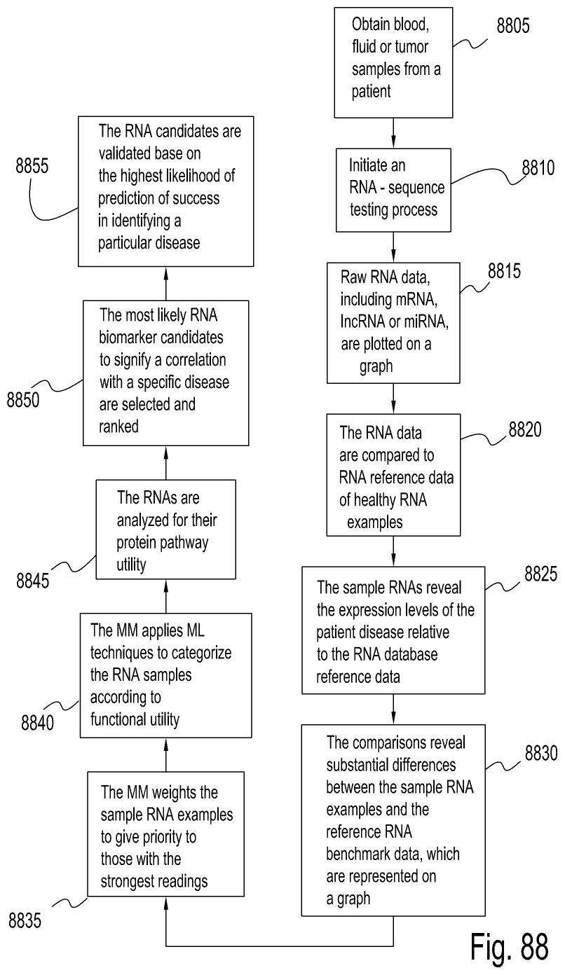

FIG. 88 is a flow chart showing the reverse engineering process for identifying novel biomarkers. Once blood, fluid or tumor samples are obtained from a patient (8805), an RNA-seq testing process is initiated (8810). Raw RNA data, including mRNA, lncRNA or miRNA, are plotted on a graph (8815) and the RNA data are compared to reference data of healthy RNA examples (8820). The sample RNAs reveal the expression levels of the patient disease relative to the RNA database reference data (8825). The comparisons between sample RNA and reference RNA reveal substantial differences which are represented on a graph (8830). The MM weights the sample RNA examples to give priority to those with the strongest readings (8835) and the MM applies ML techniques or algorithms to categorize the RNA samples according to functional utility (8840). The RNAs are analyzed for their protein pathway utility (8845). The most likely RNA biomarker candidates to signify a correlation with a specific disease are selected and ranked (8850) and the RNA candidates are validated based on the highest likelihood of prediction of success in identifying a particular disease (8855).

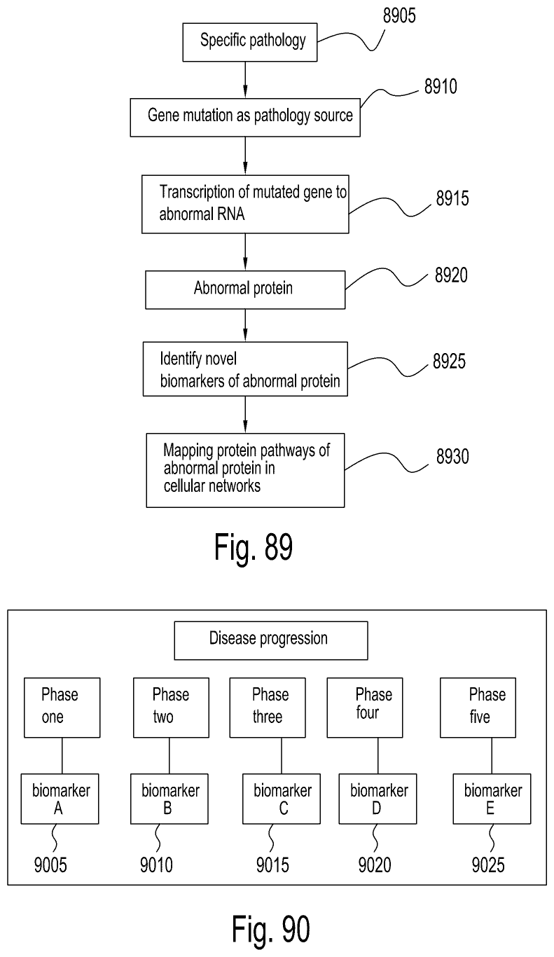

FIG. 89 is a flow chart showing the process of pathology analysis from a gene mutation to tracking abnormal protein pathways. From a specific pathology (8905), a gene mutation is identified as the source of the pathology (8910). The transcription of the mutated gene into an abnormal RNA is described (8915) and the translation from the abnormal RNA to an abnormal protein (8920) is shown. Novel biomarkers of the abnormal protein are identified (8925) and protein pathways of the abnormal protein in cellular networks are mapped (8930).

FIG. 90 is a diagram of different biomarkers associated with different phases of disease progress. Disease progression is shown over five phases. Different biomarkers (A-E) (9005-9025) are shown corresponding to the different phases of the disease progression.

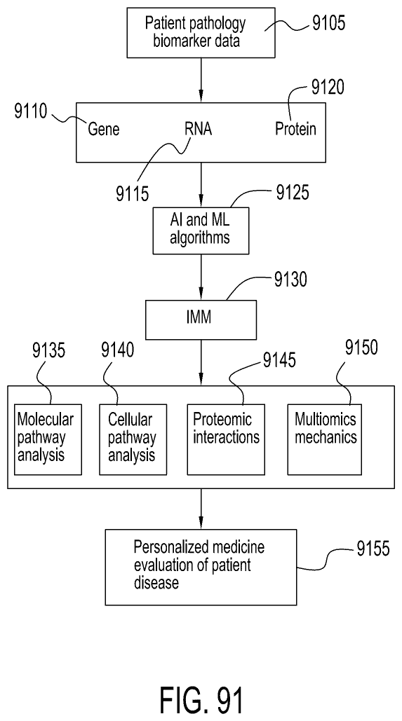

FIG. 91 is a diagram of AI and ML algorithms applied in an IMM to patient pathology biomarker data to evaluate protein and cellular dynamics. Patient pathology biomarker data (9105), including data on genes (9110), RNA (9115) and proteins (9120) are analyzed by AI and ML algorithms (9125) and input into an IMM (9130). The IMM analyzes molecular pathway analysis (9135), cellular pathway analysis (9140), proteomic interactions (9145) and multiomics mechanics (9150). The IMM develops a personalized medicine evaluation of the patient's disease (9155).

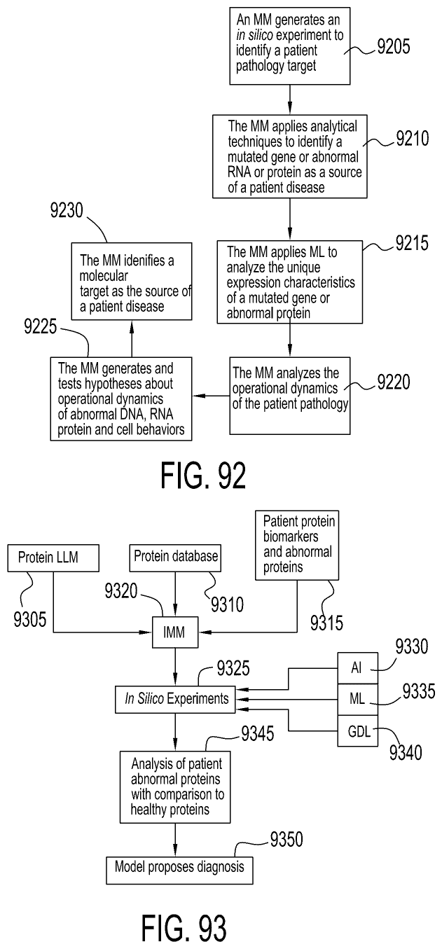

FIG. 92 is a flow chart showing an MM generating in silico experiments to test and analyze patient biomarkers to identify the source of disease. Once an MM generates an in silico experiment to identify a patient pathology target (9205), the MM applies analytical techniques to identify a mutated gene or abnormal RNA or protein as a source of a patient disease (9210). The MM applies ML, including deep learning and neural network, algorithms to analyze the unique expression characteristics of a mutated gene or abnormal protein (9215). The MM analyzes the operational dynamics of the patient pathology (9220) and generates and tests hypotheses about operational dynamics of abnormal DNA, RNA, protein and cell behaviors (9225). The MM then identifies a molecular target as the source of a patient disease (9230).

FIG. 93 is a diagram showing an IMM performing in silico experiments to assess patient abnormal proteins and propose a diagnosis. A protein LLM (9305), a protein database (9310) and data on patient protein biomarkers and abnormal proteins (9315) are input into an IMM ((320). The IMM conducts in silico experiments (9315) by applying AI (9330), ML (9335) and GDL (9340) techniques and algorithms. The IMM performs an analysis of patient abnormal proteins with comparison to healthy proteins (9345) and the model proposes a diagnosis (9350).

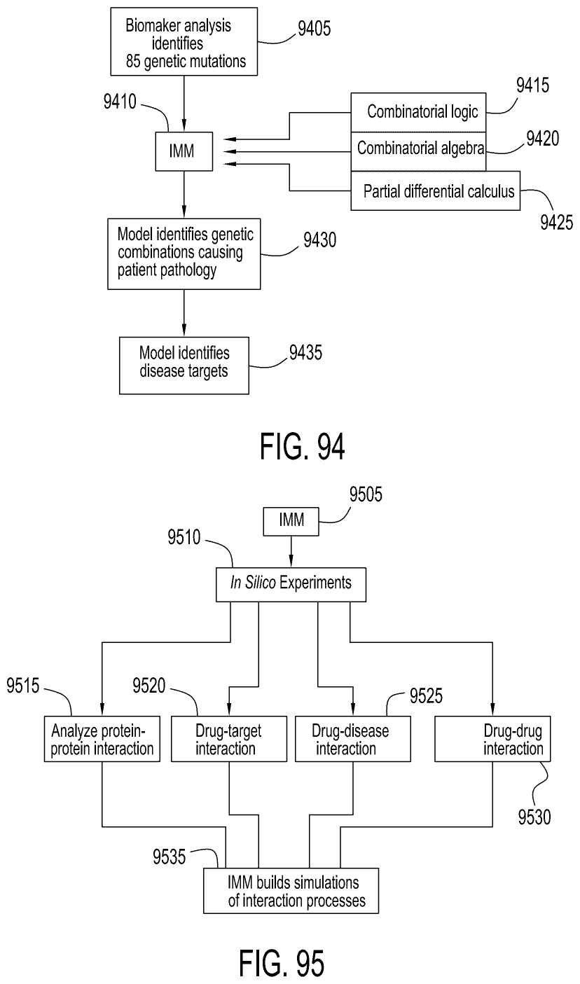

FIG. 94 is a diagram showing an IMM analyzing biomarkers to identify genetic variant combinations that reveal disease targets. A biomarker analysis identifies 85 genetic mutations (9405) in a patient, the data for which are imported into an IMM (9410). The IMM applies combinatorial logic (9415), combinatorial algebra (9420) and partial differential calculus (9425) and the model identifies genetic variant combinations causing a patient pathology (9430) by analyzing the genetic mutations. The model then identifies disease targets (9435).

FIG. 95 is a diagram showing an IMM performing in silico experiments of protein and drug interaction processes and building simulations. The IMM (9505) performs in silico experiments (9510) to perform analysis of protein-protein interaction(s) (9515), drug-target interaction(s) (9520), drug-disease interaction(s) (9525) and drug-drug interaction(s) (9530). The IMM then builds simulations of interaction processes (9535).

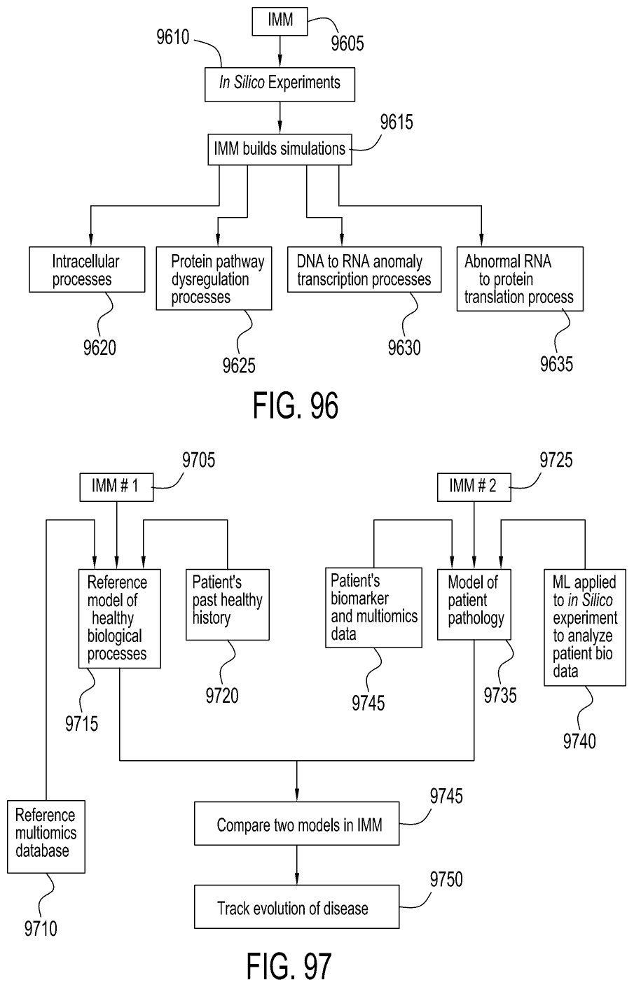

FIG. 96 is a diagram showing an IMM performing in silico simulations of DNA, RNA, protein and cellular processes. The IMM (9605) applies in silico experiments (9610) to build simulations (9615) involving analyses of intracellular processes (9620), protein pathway dysregulation processes (9625), DNA to RNA anomaly transcription processes (9630) and abnormal RNA to protein translation processes (9635).

FIG. 97 is a diagram showing a healthy reference model compared to a patient pathology model in order to assess the evolution of a disease. IMM #1 (9705) generates and analyzes a reference model of healthy biological processes (9715), drawing on a reference multiomics database (9710) and a patient's past healthy history (9720). IMM #2 (9725) generates and analyzes a model of a patient pathology (9735), which draws on patient biomarker and multiomics data (9730) and ML algorithms applied to in silico experiments to analyze patient biological data (9740). The two models in the IMM are compared (9745), which enables the patient and doctor to track the evolution of a disease (9750).

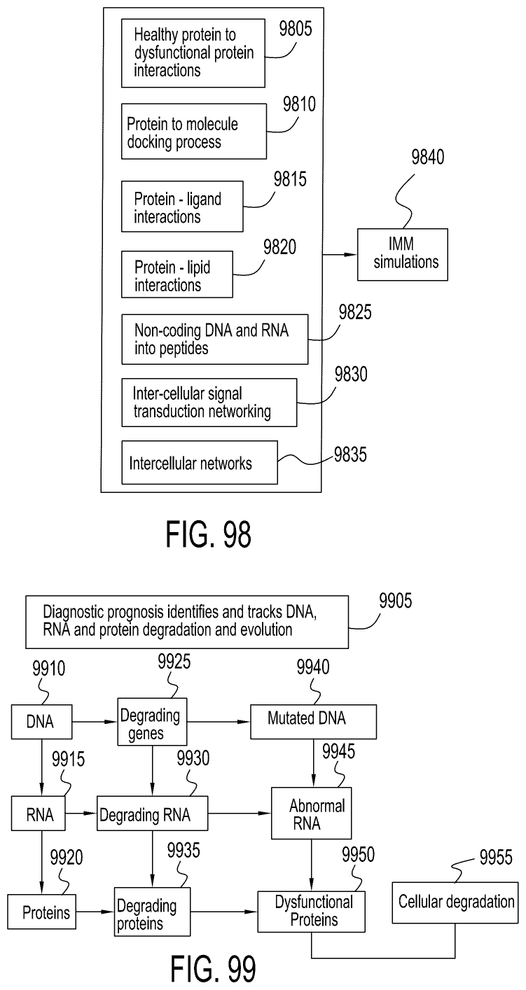

FIG. 98 is a diagram showing protein and cellular interaction processes simulated in IMMs. IMM simulations (9840) are applied to model healthy protein to dysfunctional protein interactions (9805), protein to molecule docking processes (9810), protein-ligand interactions (9815), protein-lipid interactions (9820), non-coding DNA and RNA into peptides (9825), inter-cellular signal transduction networking (9830) and intracellular networks (9835).

FIGS. 99 to 110 describe IMMs for diagnostic prognostics.

FIG. 99 is a diagram showing diagnostic prognosis identifying and tracking DNA, RNA and protein degradation and evolution (9905). DNA (9910) is shown degrading genes (925) and mutated DNA (9940). RNA (9915) is shown degrading RNA (9930) and abnormal RNA (9945). Proteins (9920) are shown degrading proteins (9935) and dysfunctional proteins (9950) and cellular degradation (9955).

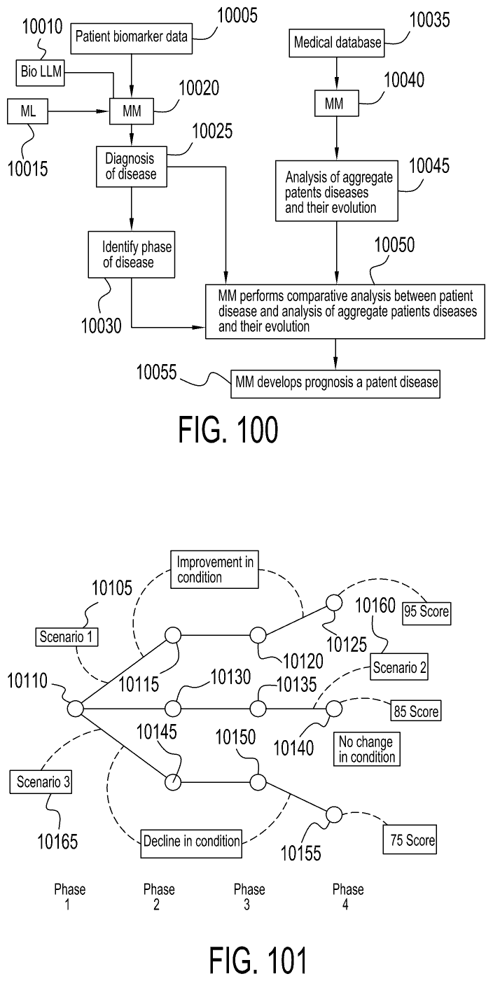

FIG. 100 is a diagram showing an MM comparing patient disease analysis and aggregate patients' diseases and their evolution to develop a prognosis of patient disease. Patient biomarker data (10005), bio LLM data (10010) and ML algorithms (10015) are input into an MM (10020). The MM diagnoses a disease (10025) and identifies the phase of the disease (10030). An MM (10040) analyzes medical database (10035) data and performs analyses of aggregate patients' diseases and their evolution (10045). The MM performs comparative analysis between the patient disease and analysis of aggregate patients' diseases and their evolution (10050) and develops prognosis of patient disease (10055).

FIG. 101 is a diagram showing different patient disease progress scenarios mapped and rated. Three scenarios of disease progress are shown. In scenario 1 (10105), a patient's disease shows improvement over four phases (10110, 10115, 10120 and 10125). At phases 2 and 3, the patient shows an improvement in condition. At phase 4 of scenario 1, the patient shows a 95 score. In scenario 2 (10160), there is no change in condition across the four phases (10110, 10130, 10135 and 10140). At phase 4 of scenario 2, the patient shows an 85 score. In scenario 3 (10165), there is a decline in the patient's condition over the four phases (10110, 10145, 10150 and 10155). At phase 4 of scenario 3, the patient shows a 75 score.

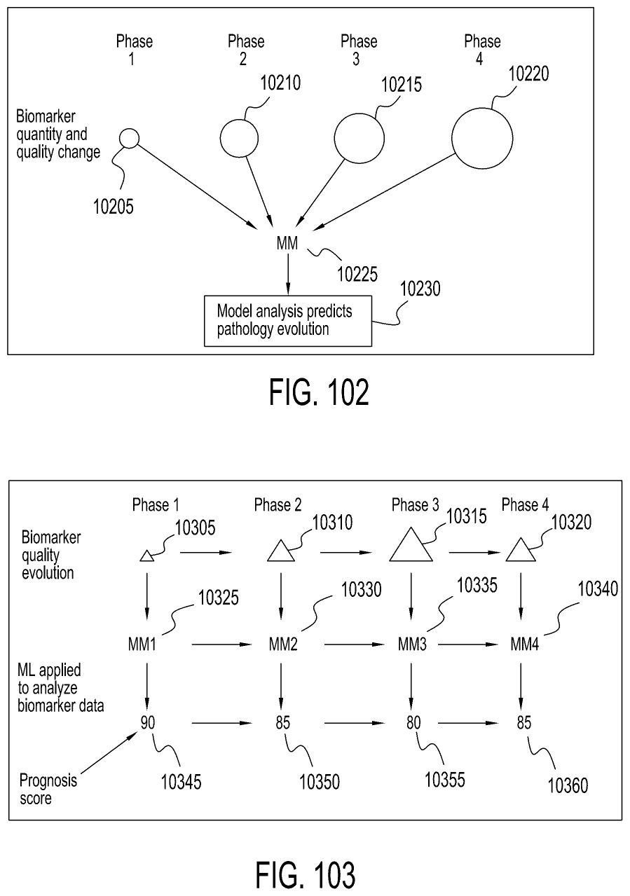

FIG. 102 is a diagram showing MMs receiving and analyzing quality and quantity biomarker data in order to predict a pathology evolution. Over four phases, the biomarker quality and quantity change, with increasing quantity of biomarkers shown from 10205 to 10210 to 10215 to 10220. The MM (10225) analyzes these biomarker changes and a model analysis predicts pathology evolution (10230) from the biomarker data.

FIG. 103 is a diagram showing biomarker data analyzed in MMs to predict disease prognosis and assign a prognosis score. Biomarker quality evolution data are shown in 10305, 10310, 10315 and 10320 over four phases, with an increase in the first three phases and a decrease in the fourth phase. ML is applied to analyze biomarker data in MMs, with M/I (10325) analyzing the biomarker data in phase 1, MM 2 (10330) analyzing the biomarker data in phase 2, MM 3 (10335) analyzing the biomarker data in phase 3 and MM 4 (10340) analyzing the biomarker data in phase 4. A prognosis score of 90 (10245) is allocated by MM 1 in phase 1, a prognosis score of 85 (10350) is allocated by MM 2 in phase 2, a prognosis score of 80 (10355) is allocated by MM 3 in phase 3 and a prognosis score of 85 (10360) is allocated by MM 4 in phase 4.

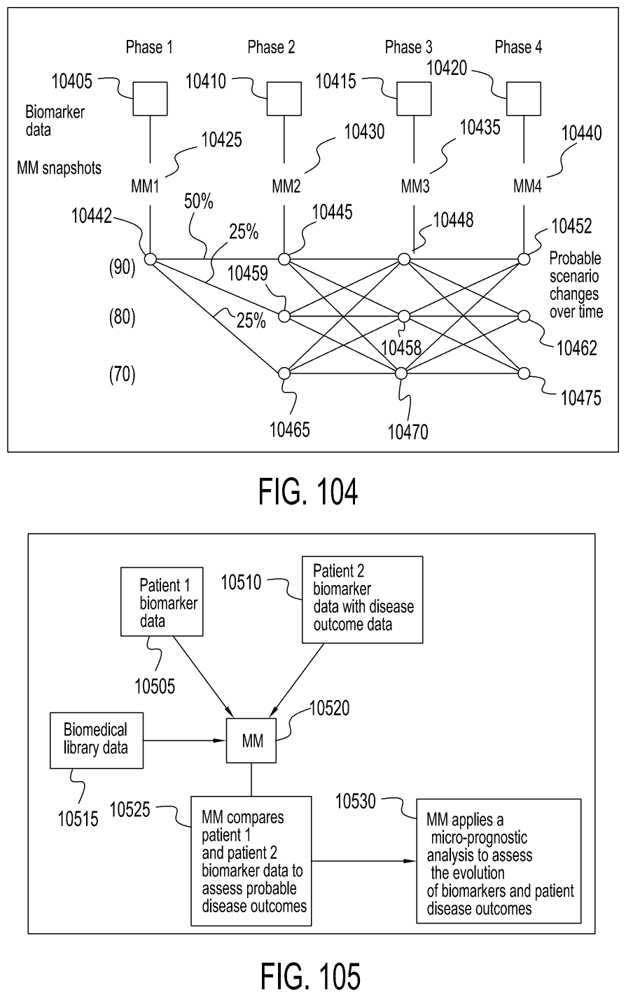

FIG. 104 is a diagram showing biomarker data analyzed in MMs in snapshots over four phases with different probable scenario outcomes over time. Biomarker data are input into MMs over four phases, with biomarker data (10405) in phase 1 input into MM 1 (10425), biomarker data (10410) in phase 2 input into MM 2 (10430), biomarker data (10415) input into MM 3 (10435) and biomarker data (10420) input into MM 4 (10440) in phase 4. These phases represent snapshots of data collection that are built and analyzed in the models. The models generate probable scenario changes of the disease progression over time on a prognosis scoring range. The models present three main scenarios. In the first scenario, which has a 50% chance of occurring, the MMs map out a positive scenario from 10442 to 10445 to 10448 to 10452, with an average prognostic score of 90. In the second scenario, which has a 25% chance of occurring, the MMs map out a moderate scenario from 10442 to 10455 to 10458 to 10462, with an average prognostic score of 80. In the third scenario, which has a 25% chance of occurring, the MMs map out a pessimistic scenario from 10441 to 10465 to 10470 to 10475, with an average prognostic score of 75.

FIG. 105 is a diagram showing an MM analyzing biomarker data to assess the evolution of patient disease outcomes. Biomedical library data (10515), patient 1 biomarker data (10505) and patient 2 biomarker data with disease outcome data (10510) are input into an MM (10520). The MM compares patient 1 and patient 2 biomarker data to assess probable disease outcomes (10525) and the MM applies a micro-prognostic analysis to assess the evolution of biomarkers and patient disease outcomes (10530).

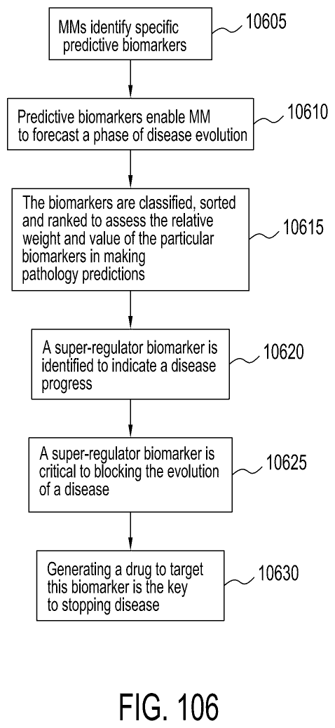

FIG. 106 is a flow chart showing biomarker analysis in MMs to identify a pathology evolution and drug targets. After MMs identify specific predictive biomarkers (10605), predictive biomarkers enable MMs to forecast a phase of disease evolution (10610). The biomarkers are classified, sorted and ranked to assess the relative weight and value of particular biomarkers in making pathology predictions (10615). A super-regulator biomarker is identified to indicate a disease progress (10620) and a super-regulator biomarker is shown to be critical to blocking the evolution of a disease (10625). The MMs generate a drug to target this biomarker as the key to stopping the disease (10630).

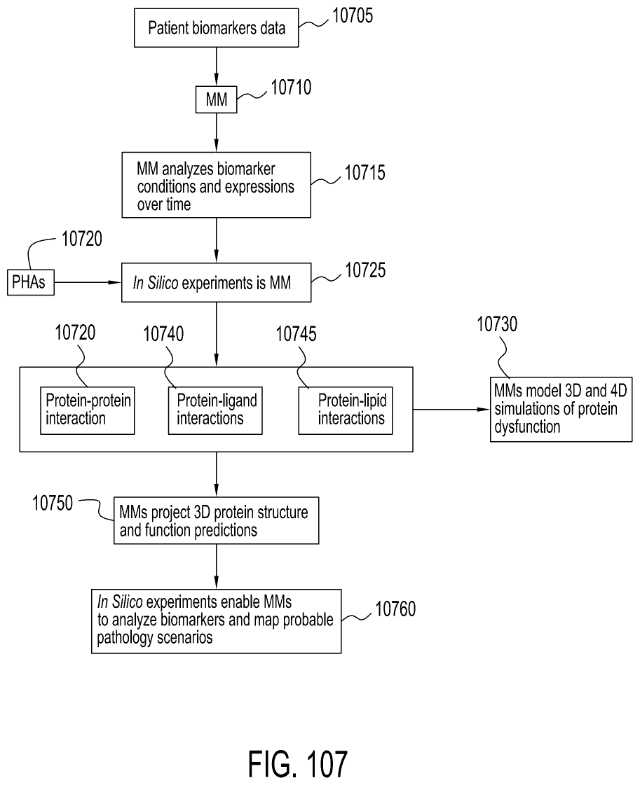

FIG. 107 is a diagram showing MMs applying in silico experiments to analyze biomarker data, develop 3D and 4D simulations and map probable pathology scenarios. Patient biomarker data (10705) are input into an MM (10710). The MM analyzes biomarker conditions and expressions over time (10715) and conducts in silico experiments (10725). PHAs (10720) may assist in processing the in silico experiments. The in silico experiments in the MM include analyses of protein-protein interactions (10735), protein-ligand interactions (10740) and protein-lipid interactions (10745). The MMs project 3D protein structure and function predictions (10750) and model 3D and 4D simulations of protein dysfunction (10730). The in silico experiments enable MMs to analyze biomarkers and map probable pathology scenarios (10760).

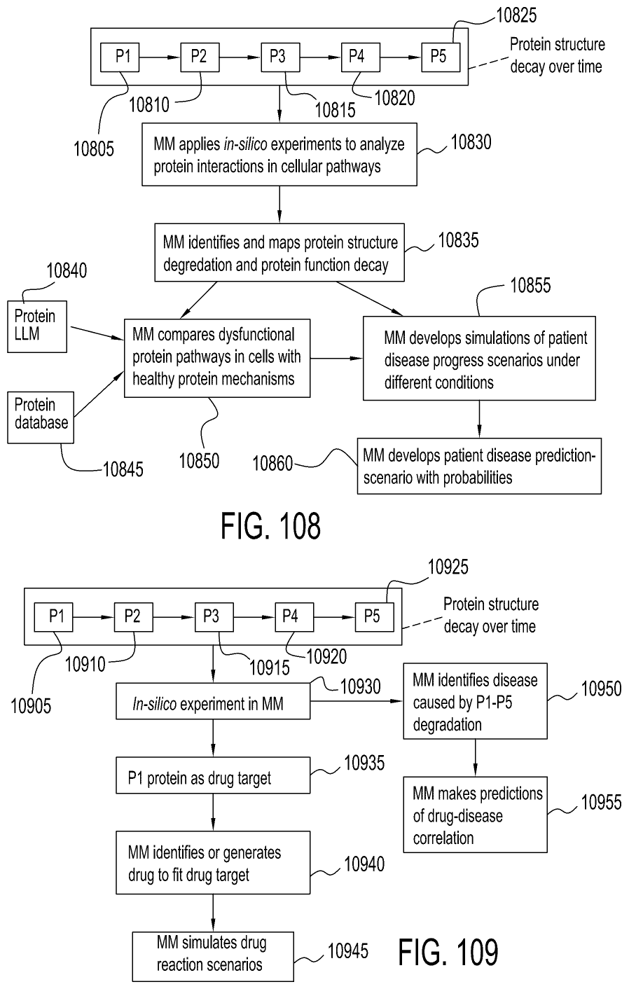

FIG. 108 is a diagram showing a micro-prognostics analysis applied by in silico experiments in MMs to compare healthy and dysfunctional proteins and predict disease progress. A protein structure decay over time is shown from P1 to P5 (10805, 10810, 10815, 10820 and 10825). The MM applies in silico experiments to analyze protein interactions in cellular pathways (10830). The MM then identifies and maps protein structure degradation and protein function decay (10835). The MM compares dysfunctional protein pathways in cells with healthy protein mechanisms (10850) by inputting data from a protein LLM (10840) and a protein database (10845). The MM develops simulations of patient disease progress scenarios under different conditions (10855) and the MM develops patient disease prediction scenarios with probabilities (10860).

FIG. 109 is a diagram showing an MM applying in silico experiments to identify a drug target and drug-target fit and making drug-disease predictions. Protein decay is shown over time from P1 to P5 (10905, 10910, 10915, 10920 and 10925). An in silico experiment is conducted in an MM (10930) to analyze the protein decay process. A P1 protein is identified as a drug target (10935) and the MM identifies or generates a drug to fit the drug target (10940) by either blocking or binding to the protein target. The MM simulates drug reaction scenarios (10945). The MM also identifies disease caused by the P1-P5 protein degradation process (10950) and the MM makes predictions of drug-disease correlation options (10955).

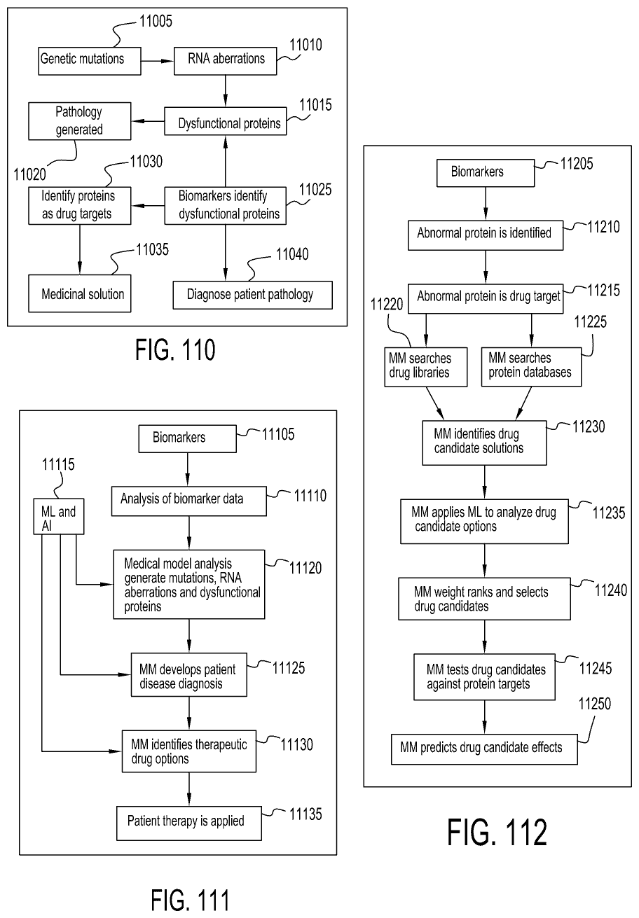

FIG. 110 is a diagram showing a process to identify patient pathology on a molecular level. Genetic mutations (11005) are transformed into RNA aberrations (11010) and into dysfunctional proteins (11015). The dysfunctional proteins are shown to generate a pathology (11020). Biomarkers identify the dysfunctional proteins (11025), which are applied to diagnose the patient pathology (11040) and serve as the source of the dysfunctional protein analysis that shows the pathology generation. The pathology identification of dysfunctional proteins detect proteins as drug targets (11030) and the system identifies a medicinal solution (11035). These functions may be performed in an MM.

IMMs are applied to therapeutics in FIGS. 111 through 124.

FIG. 111 is a diagram showing MMs applying ML and AI to analyze biomarker data to diagnose a patient disease and to develop therapeutic drug options. Biomarkers (11105) are input into an MM and analyzed (11110). AI and ML are input into an MM (11115) and the MM analyzes genetic mutations, RNA aberrations and dysfunctional proteins (11120). The MM applies AI and ML to develop a patient disease diagnosis (11125) and identify therapeutic drug options (11130). The patient therapy is then applied (11135).

FIG. 112 is a flow chart showing MMs identifying and testing drug solutions for a drug target. Biomarkers (11205) are analyzed in an MM to identify an abnormal protein (11215). The MM searches drug libraries (11220) and protein databases (12225). The MM identifies drug candidate solutions (11230) and the MM applies ML to analyze drug candidate options (11235). The MM weights, ranks and selects drug candidates (11240). The MM tests drug candidates against protein targets (11245) and the MM predicts drug candidate effects (11250).

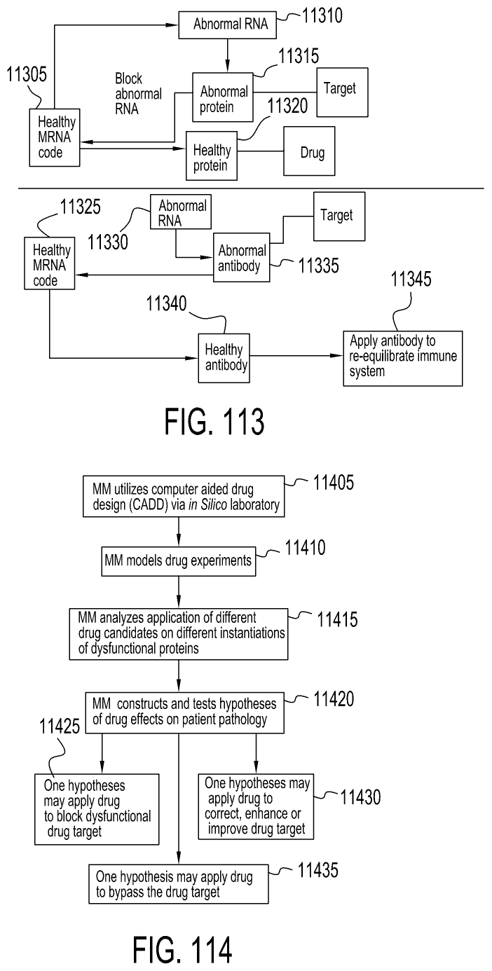

FIG. 113 is a diagram showing abnormal protein and antibody targets and application of mRNA solutions. An abnormal RNA (11310) is translated into an abnormal protein (11315), which represents a drug target. The abnormal protein is configured to identify a healthy mRNA code (11305) which is applied to block the abnormal RNA. A healthy mRNA code is applied to generate a healthy protein (11320), which is configured as a drug. An abnormal RNA (11330) is translated into an abnormal antibody (11335), which represents a drug target. The abnormal RNA is configured to identify a healthy mRNA code (11325) which is applied to block the abnormal antibody. A healthy antibody (11340) is applied to generate a healthy antibody, which antibody is applied to re-equilibrate the immune system (11345).

FIG. 114 is a flow chart showing an MM applying CADD to construct and test different hypothesis to solve a drug target. A MM utilizes computer-aided drug design (CADD) via an in silico laboratory (11405). MM models drug experiments (11410) and analyzes application of different drug candidates in different instantiations of dysfunctional proteins (11415). The MM constructs and tests hypotheses of drug effects on patient pathology (11420). One hypothesis may apply a drug to block a dysfunctional drug target (11425), one hypothesis may apply a drug to correct, enhance or improve a drug target (11430) and one hypothesis may apply a drug to bypass the drug target (11435).

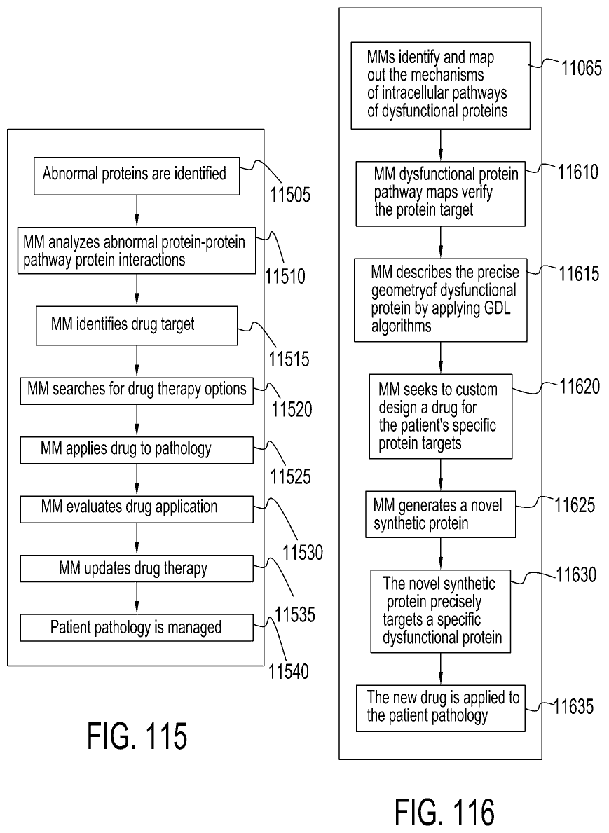

FIG. 115 is a flow chart showing MMs identifying, evaluating and updating drug therapy options to solve patient pathology. After abnormal proteins are identified (11505), an MM analyzes abnormal protein protein-pathway protein interactions (11510). The MM identifies a drug target (11515), searches for drug therapy options (11520) and applies the drug to a pathology (11525). The MM evaluates the drug application (11530) and updates the drug therapy (11535). The patient pathology is then managed (11540).

FIG. 116 is a flow chart showing MMs applied to describe the precise molecular geometry of a dysfunctional protein and to custom design a novel synthetic drug therapy. Once the MM's identify and map out the mechanisms of intracellular pathways of dysfunctional proteins (11605), the MM dysfunctional protein pathway maps verify the protein target (11610). The MM describes the precise geometry of dysfunctional proteins by applying GDL algorithms (11615) and the MM seeks to custom design a drug for the patients specific protein targets (11620). The MM generates a novel synthetic protein (11625) and the novel synthetic protein precisely targets a specific dysfunctional protein (11630). The new drug is applied to the patient pathology (11635).

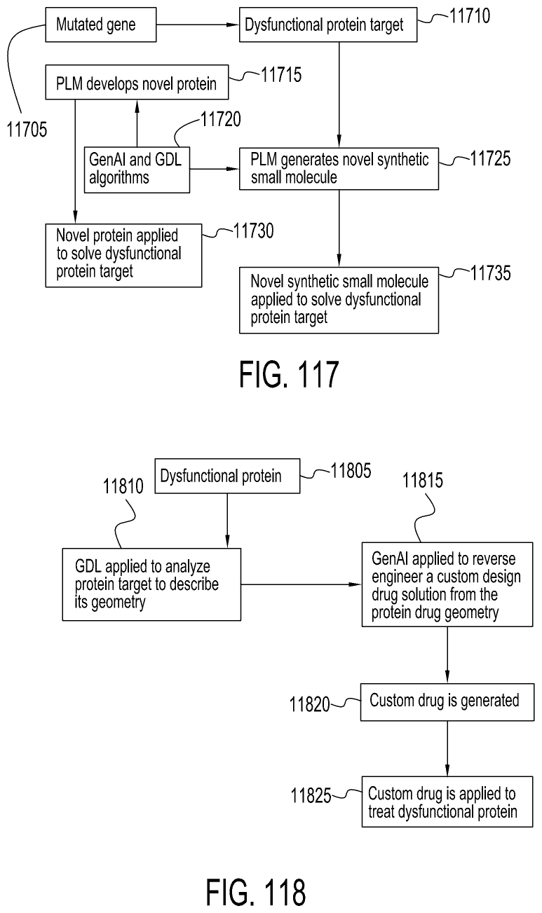

FIG. 117 is a diagram showing GenAI and GDL algorithms applied to a protein language model to develop a novel protein or small molecule to solve a dysfunctional drug target. A mutated gene (11705) develops into a dysfunctional protein target (11710). By applying GenAI and GDL algorithms (11720), a protein LM develops a novel protein (11715) and generates a novel synthetic small molecule (11725). The PLM originated novel protein is applied to solve a dysfunctional protein target (11730). The PLM generated novel synthetic small molecule is applied to solve a dysfunctional protein target (11735).

FIG. 118 is a flow chart showing a GDL applied to describe dysfunctional protein and GenAI applied to custom design a drug solution. A dysfunctional protein (11805) is configured as a drug target and GDL is applied to analyze the protein target to describe its geometry (11810). The GenAI is applied to reverse engineer a custom design of a drug solution from the protein drug geometry (11815) and a custom drug is generated (11820). The custom drug is applied to treat the dysfunctional protein (11825).

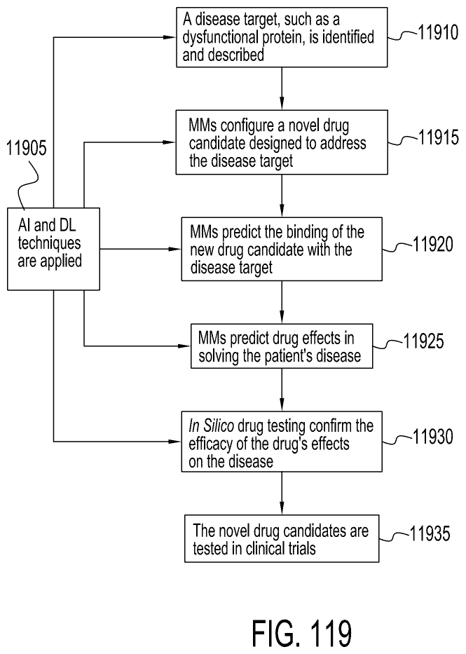

FIG. 119 is a flow chart showing MMs designing and testing novel drugs to match a dysfunctional protein target by applying AI and ML techniques. After a disease target, such as a dysfunctional protein, is identified and described in MMs (11910), the MMs configure a novel drug candidate design to address the disease target (11915). AI and ML techniques and algorithms are applied to the MMs to generate analyses (11905). The MMs predict the binding of the new drug candidate with the disease target (11920) and predict drug effects in solving the patient's disease (11925). In silico drug testing in MMs confirm the efficacy of the drug's effects on the disease (11930) and the novel drug candidates are tested in clinical trials (11935).

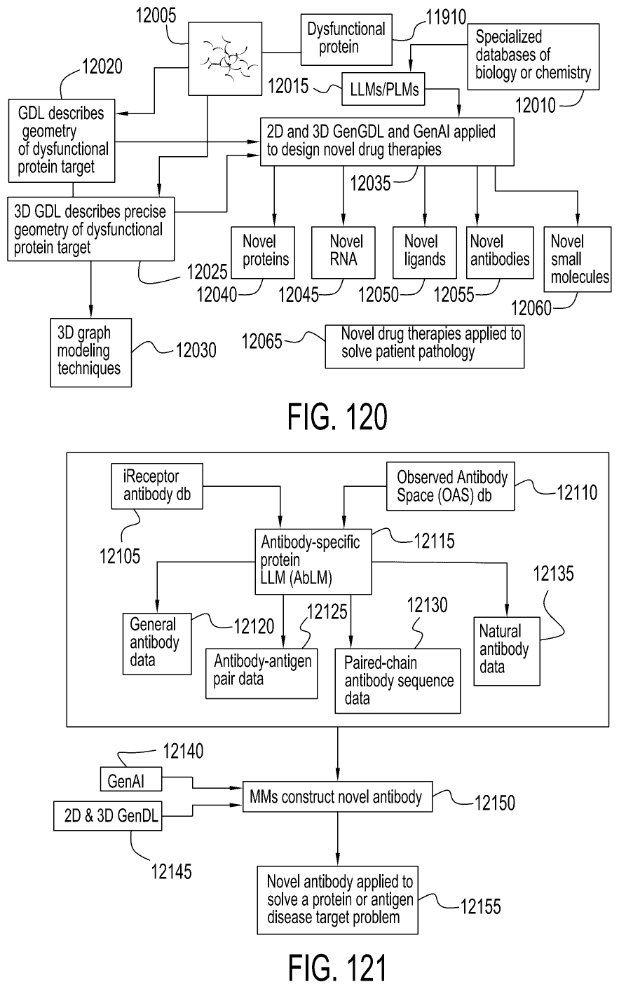

FIG. 120 is a diagram showing 3D GDL applied to describe dysfunctional protein and 2D GenGDL and 3D GenGDL applied to design novel drug therapies. A GDL algorithm describes the geometry (12020) of a dysfunctional protein target (12005) and 3D GDL describes the precise geometry of the dysfunctional protein target (12025). The 3D GDL algorithm applies 3D graph modeling techniques or algorithms (12030), with 2D and 3D GenGDL and GenAI algorithms applied to design novel drug therapies (12035). The Gen GDL and Gen AI draw on LLMs and PLMs (12015) that are fed data from specialized biology and/or chemistry databases (12010). The 2D and 3D GenGDL and GenAI algorithms are applied to design novel proteins (12040, novel RNA (12045), novel ligands (12050), novel antibodies (12055) and novel small molecules (12060). The novel drug therapies are applied to solve patient pathologies (12065).

FIG. 121 is a diagram showing antibody specific protein LLMs, GenAI and GenGDL applied to MMs to construct a novel antibody. The iReceptor antibody database (12105) and the observed antibody space (OAS) database (12110) are accessed by the antibody-specific protein LLM (AbLM) (12115). The AbLM generates data on general antibodies (12120), antibody-antigen pairs, (12125), paired-chain antibody sequences (12130) and natural antibodies (12135). By applying GenAI (12140) and 2D and 3D GenGDL algorithms (12145), MMs construct at least one novel antibody (12150). The novel antibody is applied to solve a protein or antigen disease target problem (12155).

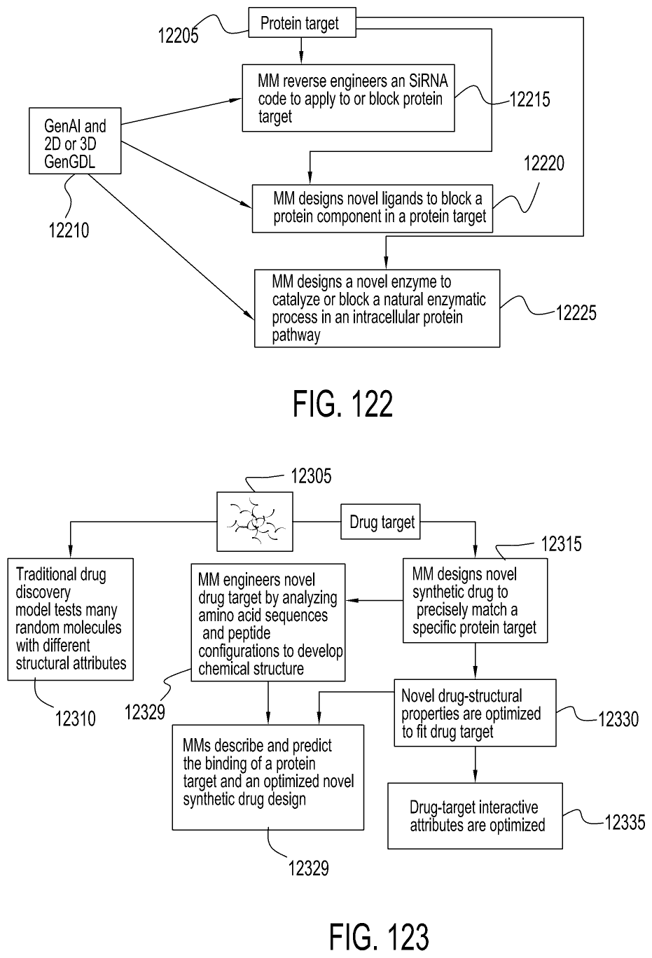

FIG. 122 is a flow chart showing GenAI, 2D GenGDL or 3D GenGDL applied to MMs to design novel siRNA code, novel ligands and novel enzymes. After analyzing a protein target (12205), an MM reverse engineers a siRNA code to apply to or block (inhibit) the protein target (12215). The MM applies GenAI and 2D or 3D GenGDL algorithms (12210). Additionally, the MM designs novel ligands to block a protein component in a protein target (12220). Moreover, the MM also designs a novel enzyme to catalyze or block a natural enzymatic process in an intracellular protein pathway (12225).

FIG. 123 is a diagram showing an MM designing a novel synthetic drug to optimize structural properties to fit a drug target. In one mode, a traditional drug discovery model tests many random molecules with different structural attributes (12310) by analyzing a drug target (12305). In another mode, after analyzing the (protein) drug target, an MM designs a novel synthetic drug to precisely match a specific protein target (12315). The MM engineers a novel drug from the drug target by analyzing amino acid sequences and peptide configurations to develop a chemical structure (12320) and the MM describes and predicts the binding of a protein target and an optimized novel synthetic drug design (12325). Additionally, an MM designs a novel synthetic drug to precisely match a specific protein target (12315). The novel drug structural properties are optimized to fit the drug target (12330) and the drug-target interactive attributes are optimized (12335).

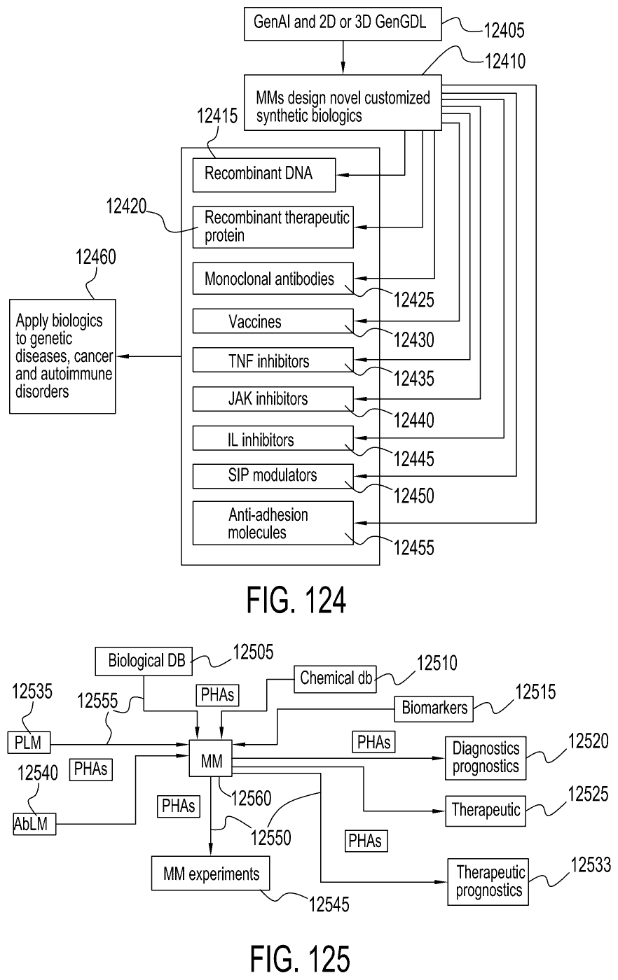

FIG. 124 is a diagram showing MMs designing several classes of novel customized synthetic biologics. By applying GenAI and 2D or 3D GenGDL algorithms (12405), MMs design novel customized synthetic biologics (12410). The synthetic biologics include recombinant DNA (12415), recombinant therapeutic proteins (12420), monoclonal antibodies (12425), vaccines (12430), TNF inhibitors (12435), JAK inhibitors (12440), IL inhibitors (124450), SIP modulators (12450) and anti-adhesion molecules (12455). These custom designed biologics are then applied to genetic diseases, cancer and autoimmune disorders (12460).

FIGS. 125 to 139 show IMMs applied to therapeutic prognostics.

FIG. 125 is a diagram showing AI-endowed PHAs collecting biological data for MM analysis of diagnostics, prognostics and therapeutics. A biological database (12505) and a chemical database (12510), a PLM (12535), an AbLM (12540) and biomarkers (12515) supply data to an MM (12560) via PHAs (12550 and 12555). The MM conducts experiments (12545) to identify solutions to problems. The MM then supplies analyses for diagnostics (12520), diagnostic prognostics (12525), therapeutics (12530) and therapeutic prognostics (12533).

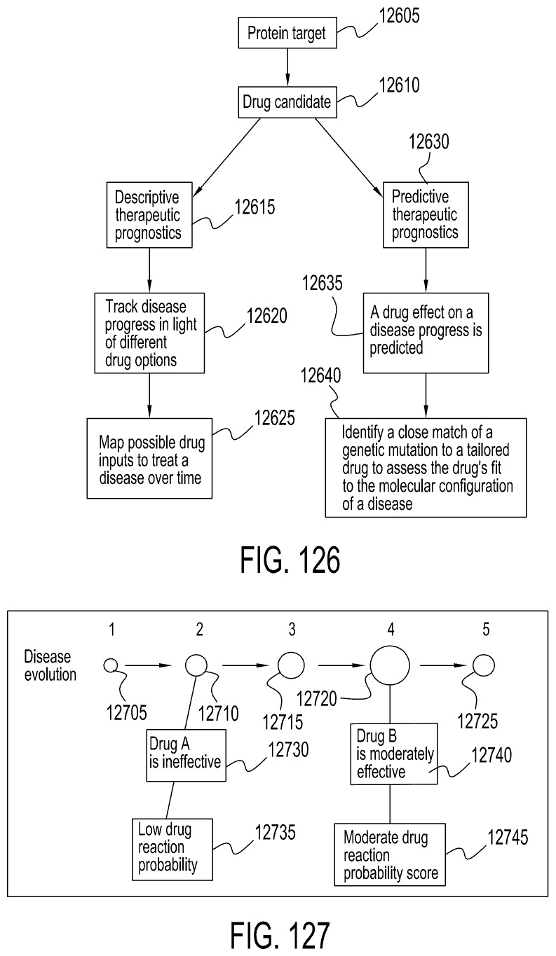

FIG. 126 is a diagram showing therapeutic prognostics describing drug options on disease progress and predicting a drug's effect on a disease. A drug candidate (12610) is matched to a protein target (12605). Descriptive therapeutic prognostics (12615) is applied to track a disease progress in light of different drug options (12620) and map possible drug inputs to treat a disease over time (12625). Prescriptive therapeutic prognostics (12630) is applied to predict a drug's effect on a disease progress (12635) and identify a close match of a genetic mutation to a tailored drug in order to assess the drug's fit to the molecular configuration of a disease (12640).

FIG. 127 is a diagram showing different drugs providing effects on disease evolution and assigning drug reaction probability scores. A disease is shown evolving over five stages (12705, 12710, 12715, 12720 and 12725). At stage two (12710), a drug A (12730) is shown to be ineffective and it is given a low drug reaction probability score (12735). At stage four (12720), a drug B is show to be moderately effective (12740) and it is given a moderate drug reaction probability score (12745).

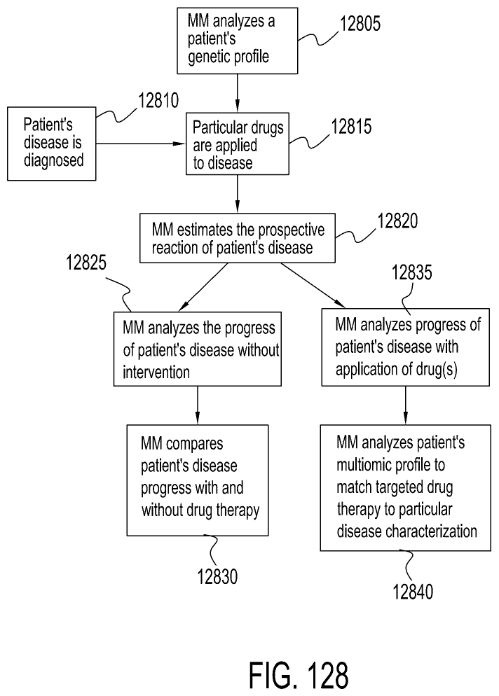

FIG. 128 is a flow chart showing an MM analyzing and comparing a patient's disease progress with and without drug therapy. An MM analyzes a patient's genetic profile (12805). After the patient's disease is diagnosed (12810), particular drug(s) are applied to the disease (12815). The MM estimates the prospective reaction of the drugs of the patient's disease (12820). The MM analyzes the progress of the patient's disease without intervention (12825) and the MM compares the patient's disease progress with and without drug therapy (12830). The MM analyzes progress of patient's disease with application of drug(s) (12835) and the MM analyzes the patient's multiomic profile to match the targeted drug therapy to the particular disease characterization (12840). The MM again compares the patient's disease progress with and without drug therapy (12830).

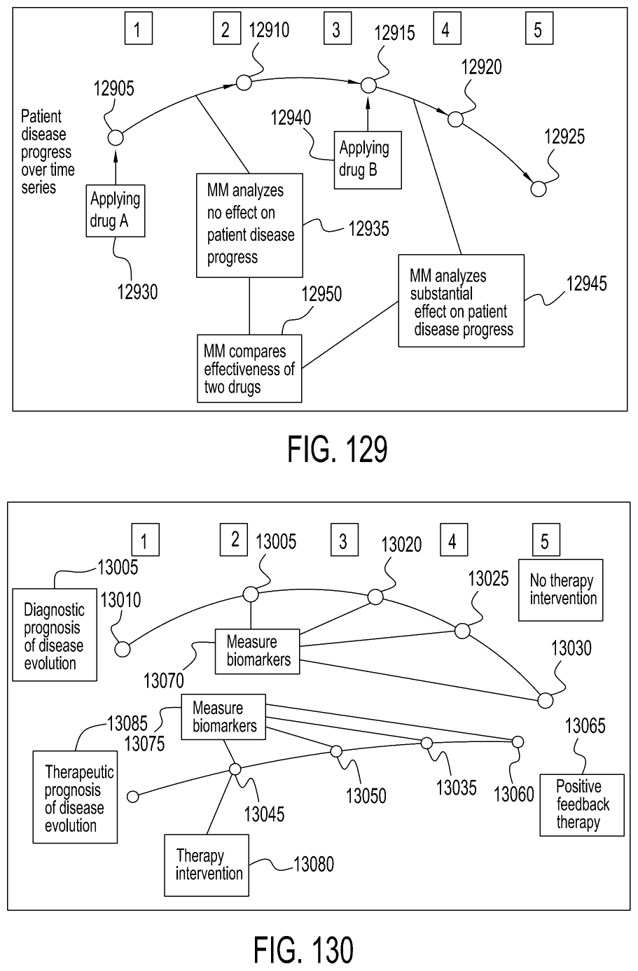

FIG. 129 is a diagram showing an MM analyzing and comparing effectiveness of two drugs on a patient disease progress. A patient disease progress is shown over a time series of five phases (1-5) (12905, 12910, 12915, 12920 and 12925). Drug A is applied to treat the patient disease at phase 1 (12930). The MM analyzes the lack of effect on the patient disease (12935) after the first phase. Drug B is applied to treat the patient disease at phase 3 (12940). The MM analyzes a substantial positive effect on the patient disease progress (12945) at phase 4. The MM compares the effectiveness of the two drugs (12950).

FIG. 130 is a diagram showing biomarker measurements applied to compare disease progress with and without intervention. A diagnostic prognosis of a disease evolution (13005) without therapeutic intervention is shown compared to a therapeutic prognosis of a disease evolution (13085) with therapeutic intervention. In the case of the diagnostic prognosis, a disease evolution is shown over five phases (13010, 13015, 13020, 13025 and 13030). Biomarkers (13070) are measured at phases 2-5 along the course of the disease progression in order to map the evolution of the disease without therapeutic intervention (13035). In the case of the therapeutic prognosis, a disease evolution is shown over the five phases (13040, 13045, 13450, 13455 and 13460) in which biomarkers are measured (13075) in order to detect therapy effectiveness at each stage. A therapy is applied at phase 2 (13080) and the positive feedback of the therapy (13065) is demonstrated by assessing the biomarkers.

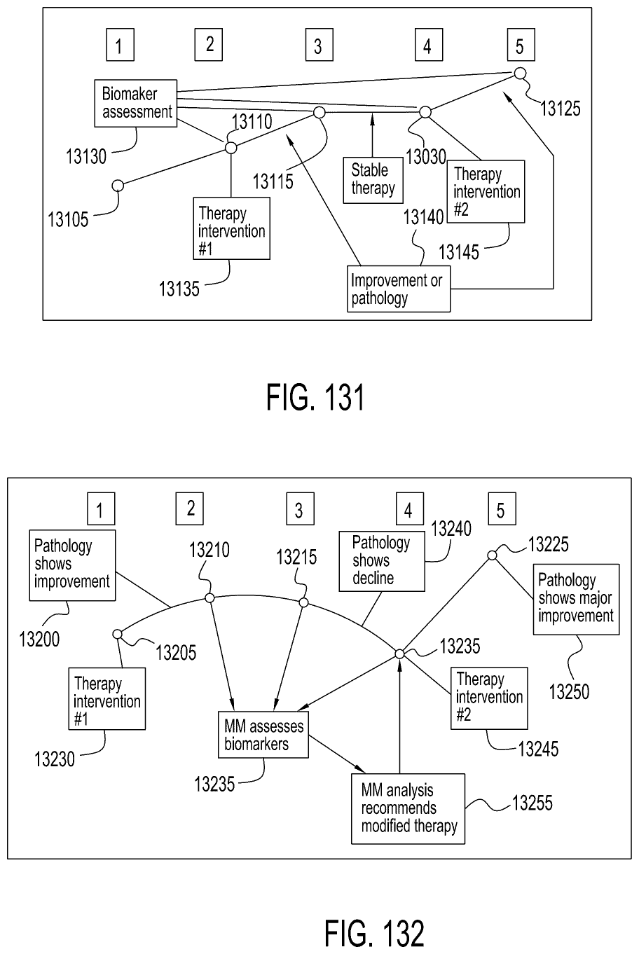

FIG. 131 is a diagram showing drug therapy intervention applied, assessed (via biomarkers) and modified to show pathology improvement over five phases (13105, 13110, 13115, 13120 and 13125). A disease is identified at phase 1 (13105). In phase 2, a therapy intervention #1 (13175) is applied. Biomarkers are applied at phases 2-5 (13130) to assess the effectiveness of the therapy intervention. Biomarker assessment at phases 2 and 3 show an improvement of the pathology condition (13140) and the stability of the therapy between phases 3 and 4. A therapy intervention #2 (13145) is applied at phase 4 and an improvement of the pathology condition after phase 4.

FIG. 132 is a diagram showing an MM analyzing patient biomarkers to assess pathology progress and recommending a modified therapy that shows major improvement. A therapy intervention #1 (13230) is applied at phase 1 (13205) and the pathology shows improvement (13200). An MM assesses biomarkers at phase 2 (13210), phase 3 (13215) and phase 4 (13220). After phase 3, the pathology shows a decline in condition (13240). As a consequence of the deteriorating condition based on biomarker feedback, an MM analysis recommends a modified therapy (13255), therapy intervention #2 (13245). At phase 5, the pathology shows a major improvement (13225).

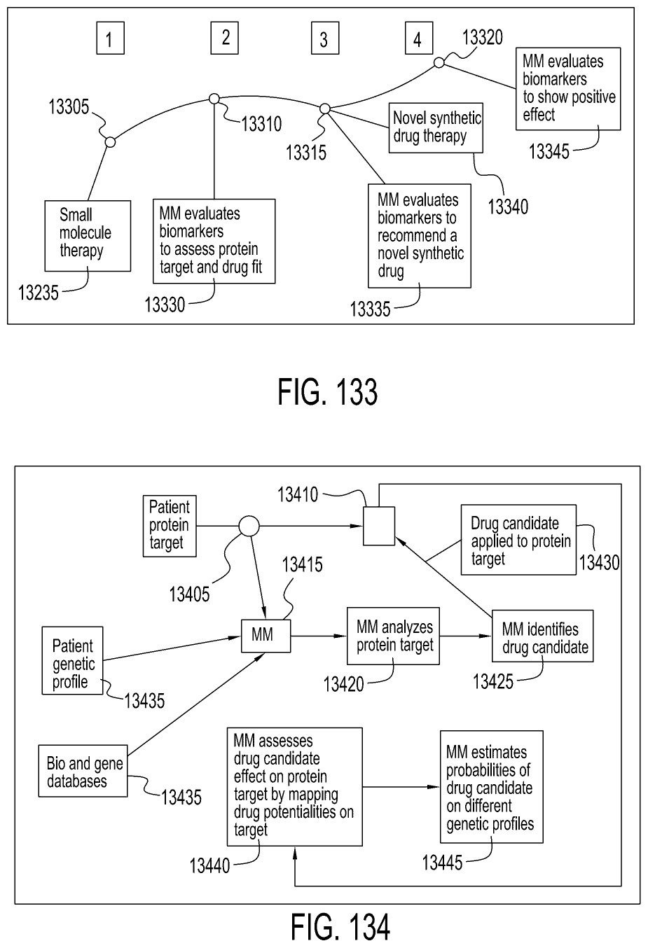

FIG. 133 is a diagram showing an MM evaluating small molecule therapy biomarker feedback and recommending a novel synthetic drug which shows positive effect. A small molecule therapy (13325) is applied at phase 1 (13305) and an MM evaluates biomarkers to assess protein target and drug fit (13330) at phase 2 (13310). The MM evaluates biomarkers and recommends a novel synthetic drug (13335) and the novel synthetic drug therapy is applied (13340) at phase 3 (13315). The MM evaluates biomarkers at phase 4 (13320) to show a positive effect (13345).

FIG. 134 is a diagram showing an MM analyzing a protein target, identifying a drug candidate and assessing the drug candidate effects on the protein target. An MM (13415) analyzes (13420) a patient protein target (13405). The MM inputs patient genetic profile (13435) data and data from bio and gene databases (13435) in order to analyze the patient protein target. The MM identifies a drug candidate (13425) and the drug candidate is applied to the patient protein target (13430). After applying the drug candidate, the MM assesses the drug candidate effect on the protein target by mapping drug potentialities on the target (13440) and the MM estimates probabilities of drug candidate on different genetic profiles (13445).

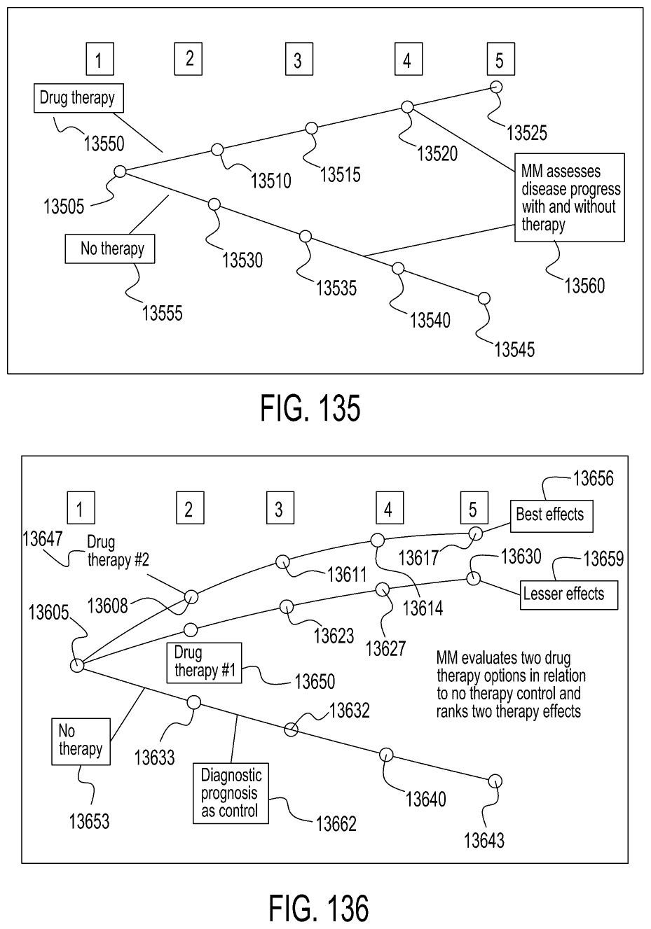

FIG. 135 is a diagram showing an MM assessing disease progress with and without therapy intervention. In one mode, a drug therapy (13550) is applied to a patient at an early stage shown here over five phases (13505, 13510, 13515, 13520 and 13525). In another mode, no therapy (13555) is shown over five phases (13505, 13530, 13535, 13540 and 13545). An MM assesses the disease progress with and without therapy (13560). As shown in this figure, the mode with the drug therapy applied improves the patient's pathology condition, while the mode without drug intervention recognizes a degradation in the performance of the patient's pathology condition.

FIG. 136 is a diagram showing an MM evaluating two drug therapy options in relation to no therapy control and ranking two therapy effects. Over five phases, several options are shown, including no therapy (13653) applied to show a diagnostic prognosis as the control (13662) over the five phases (13605, 13633, 13637, 13640 and 13643). Drug therapy 1 (13650) is applied early in the five phases (13605, 13620, 13623, 13627 and 13630), producing lesser relative therapeutic effects (13659). Drug therapy 2 (13647) is applied early in the five phases (13605, 13608, 13611, 13614 and 13617), producing the best relative therapeutic effects (13656). The MM evaluates the two drug therapy options in relation to the no therapy control option and ranks the two therapy effects.

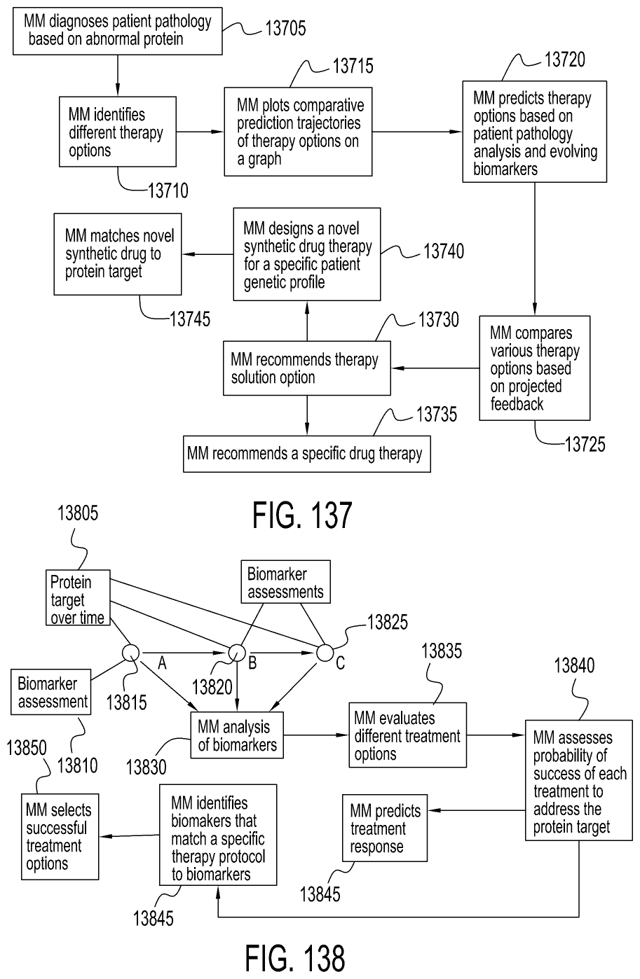

FIG. 137 is a diagram showing an MM identifying, predicting and recommending various drug therapy options to solve a patient pathology. After an MM diagnoses a patient pathology based on an abnormal protein (13705), the MM identifies different therapy options (13710). The MM plots comparative predictive trajectories of therapy options on a graph (13715) and the MM predicts therapy options based on patient pathology analysis and evolving biomarkers (13720). The MM compares various therapy options based on projected feedback (13725) and the MM recommends therapy solution options (13730). The MM designs a novel synthetic drug therapy for a specific patient genetic profile (13740) and the MM matches the novel synthetic drug to the protein target (13745). The MM recommends a specific drug therapy (13735) based on the analysis of options.

FIG. 138 is a diagram showing an MM analyzing biomarkers to predict or select treatment options. A protein target (13805) is assessed over time (13815, 13820 and 13825) and biomarkers are assessed (13810) at each stage (A, B and C). The MM performs an analysis of the biomarkers (13830) and evaluates different treatment options (13835). The MM assesses probability of success of each treatment to address the protein target (13840) and the MM predicts a treatment response (13845). The MM identifies biomarkers that match a specific therapy protocol to biomarkers (13845) and the MM selects a successful treatment option (13850).

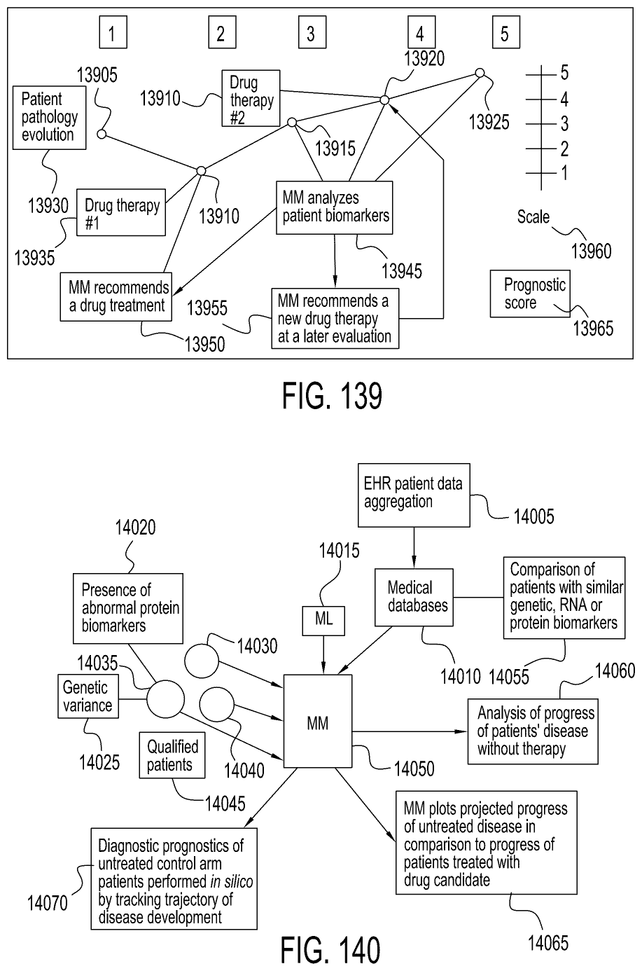

FIG. 139 is a diagram showing an MM analyzing patient biomarkers on a scale and recommending different drug treatments at different times in evaluation of disease progress. A patient pathology evolution (13940) is shown over five phases (13905, 13910, 13915, 13920 and 13925). A drug therapy #1 (13935) is applied at phase 2, a drug therapy #2 is applied at phase 3 and an MM analyzes patient biomarkers (13945) at phases 3, 4 and 5. The MM recommends a new drug therapy at a later evaluation (13955) and the MM recommends a drug treatment (13950). The drug therapy options are mapped to a prognostics score (13965) scale (13960) consisting of a range of five measures.

FIGS. 140-157 describe MMs applied to drug clinal trials.

FIG. 140 is a diagram showing an MM tracing a control arm of drug clinical trials. Electronic health record patient data are aggregated (14005) and analyzed in medical databases (14010). The medical database data, EHR data and ML algorithms (14015) are input into an MM (14050). The MM analyzes patient data on the presence of abnormal protein biomarkers (14020) and genetic mutations (variances) (14025) in a group (14030, 14035 and 14040) of qualified patients (14045). The MM generates an analysis of the progress of the patients' disease without therapy (14060), a projected progress of the untreated disease in comparison to the progress of patients treated with a drug candidate (14065) and a diagnostic prognostics of an untreated control arm of patients performed in silico by tracking the trajectory of the disease development (14070).

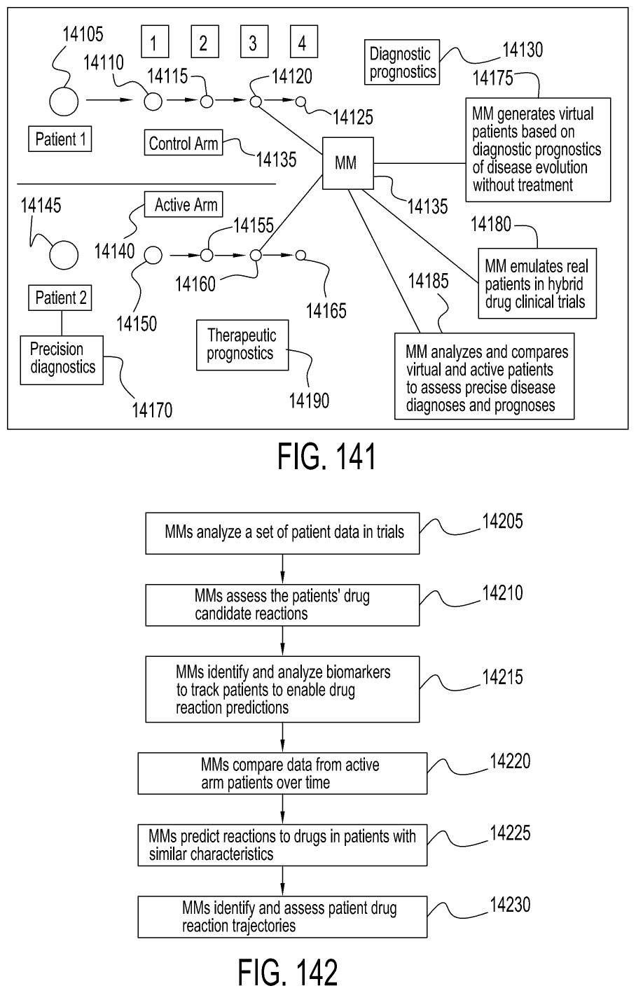

FIG. 141 is a diagram showing an MM applied to drug clinical trials for precision diagnosis and emulation of virtual patients. In a control arm (14135), a patient 1 (14105) is shown progressing with a disease over four phases (14110, 14115, 14120 and 14125) illustrating a diagnostic prognostics (14130) description of the evolution of the disease. In an active arm (14140), a patient 2 (14145) is shown progressing with a disease over four phases (14150), 14155, 14160 and 14165) illustrating a therapeutic prognostics (14190) evolution of the disease after patient 2 was provided with a precision diagnosis (14170) and a therapy candidate in the active arm of the drug clinical trials. The data from the control arm and the active arm are forwarded to an MM (14135). The MM generates virtual patients based on the diagnostic prognostics of disease evolution without treatment (14175). The MM emulates real patients in hybrid drug clinical trials (14180). Finally, the MM analyzes and compares virtual and active patients to assess precise disease diagnoses and prognoses (14185) and to evaluate the effectiveness of the drug candidate.

FIG. 142 is a flow chart showing MMs analyzing and aggregating patient data in the active arm of clinical trials. After the MMs analyze a set of patient data in trials (14205), the MMs assess the patients' drug candidate reactions (14210). The MMs identify and analyze biomarkers to track patients to enable drug reaction predictions (14215) and the MMs compare data from the active arm patients overtime (14220). The MMs predict reactions to drugs in patients with similar characteristics (14225) and the MMs identify and assess patient drug reaction trajectories (14230).

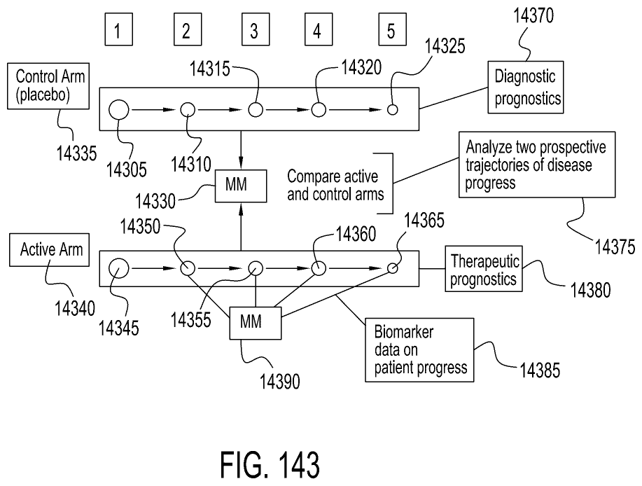

FIG. 143 is a diagram showing MMs applied to track active arm patient progress and compare to control arm patient progress. The control arm (placebo) (14335) is tracked over five phases (14305, 14310, 14315, 14320 and 14325) in a diagnostic prognostics (14370) analysis. The active arm (14340) is tracked over five phases (14345, 14350, 14355, 14360 and 14365) in a therapeutic prognostics (14380) analysis. An MM (14390) analyzes the biomarker data on patient progress (14385) in the active arm. An MM (14330) analyzes the control arm and active arm data in order to compare the effectiveness of the drug candidate relative to the placebo by analyzing the prospective trajectories of disease progress (14375).

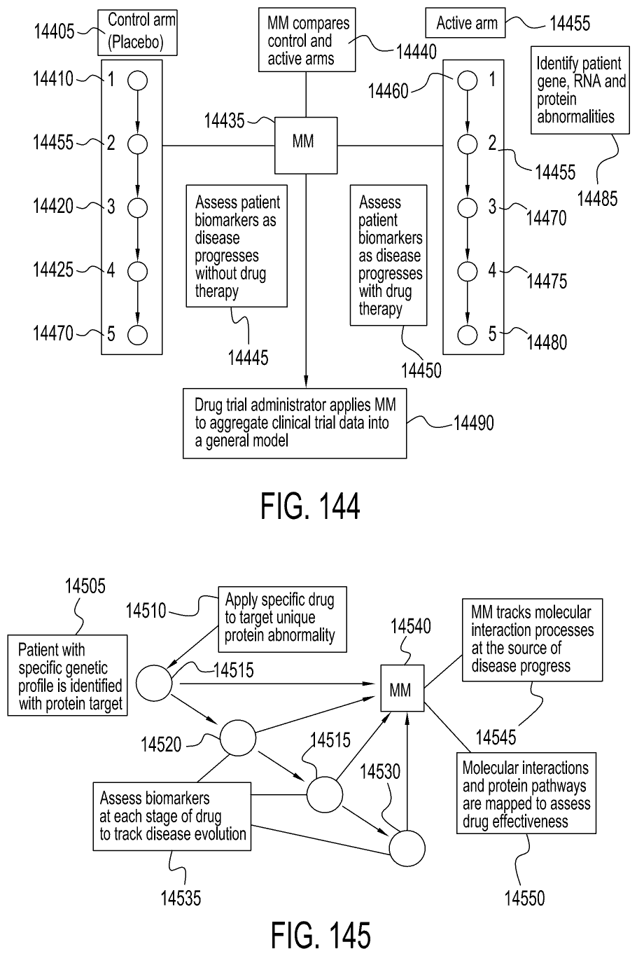

FIG. 144 is a diagram showing an MM comparing and aggregating control and active arms data. The control arm (placebo) (14405) is shown over five phases (14410, 14415, 14420, 14425 and 14430). The control arm patient biomarkers are assessed as the disease progressed without drug therapy (14445). An active arm (14455) is shown over five phases (14460, 14465, 14470, 14475 and 14480), with patient gene, RNA and protein abnormalities identified (14485) in biomarkers. The active arm patient biomarkers are assessed as the disease progresses with drug therapy (14450). An MM 914435) compares the data in the control arm and the active arm of the drug clinical trials. The MM drug trial administrator applies the MM to aggregate clinical trial data into a general model (14490).

FIG. 145 is a diagram showing an MM applied to analyze biomarker data feedback of a drug to target a specific protein and analyze molecular interactions to show drug effectiveness. A patient with a specific genetic profile is identified with a protein target (14505). A specific drug is applied to target a unique protein abnormality (14510). The patient progress is assessed over four phases (14515, 14520, 14525 and 14530). Biomarkers are assessed or interpreted at each stage of the drug treatment in order to track the disease evolution (14535). The patient disease progress and drug reaction data are input into an MM (14540). The MM tracks molecular interaction processes at the source of the disease progress (14545) and molecular interactions and protein pathways are mapped in the MM to assess drug effectiveness (14550).

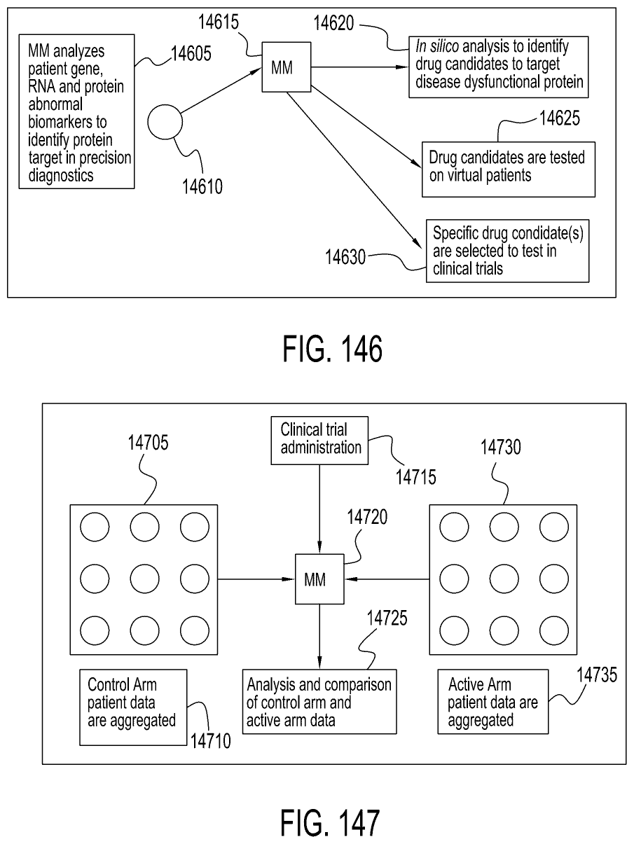

FIG. 146 is a diagram showing an MM diagnosing a precise disease (and identifying abnormal protein targets) and identifying drug candidate options to match to the protein targets. An MM (14615) analyzes patient (14610) gene, RNA and protein abnormal biomarkers in order to identify protein target in precision diagnostics (14605). The MM performs an in silico analysis to identify drug candidates to target disease dysfunction protein(s) (14620). The MM tests the drug candidates on virtual patients (14625) and specific drug candidate(s) are selected to test in clinical trials (14630).

FIG. 147 is a diagram showing an MM analyzing aggregated control arm and active arm data. A group of patients (14705) are shown in a control arm, with the control arm patient data aggregated (14710). A group of patients (14730) are shown in an active arm, with the active arm patient data aggregated (14735). These data are input into an MM (14720), which is managed by a clinical trial administrator (14715). The MM performs an analysis and comparison of the control arm and the active arm data (14725).

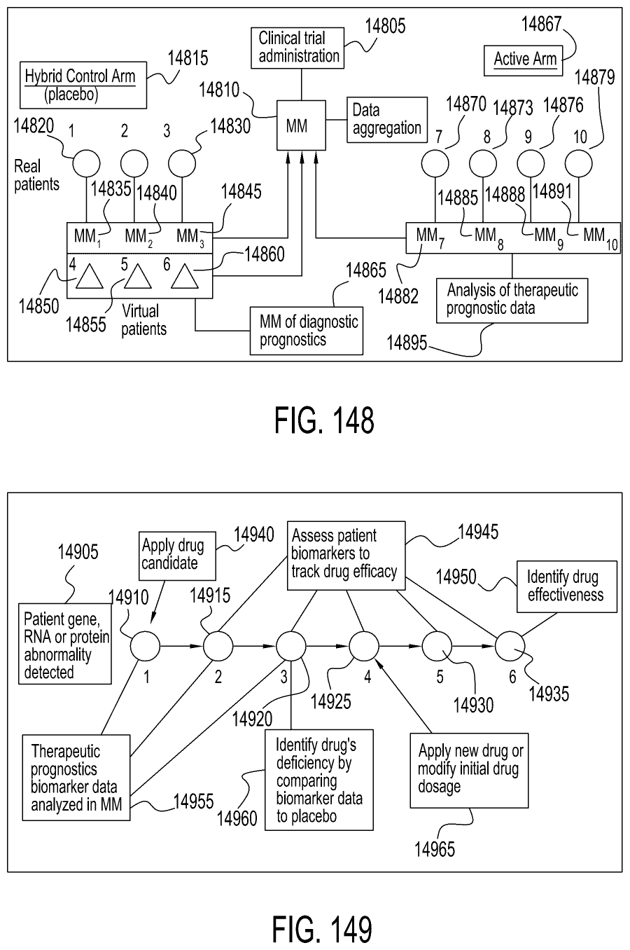

FIG. 148 is diagram showing an MM analyzing hybrid control arm (including virtual patients) diagnostic prognostics data and active arm therapeutic prognostics data. A hybrid control arm (14815) is shown with real patients (1, 2 and 3) (14820, 14825 and 14830). Each patient has an MM. Patient 1 has an MM 1 (14835), patient 2 has an MM 2 (14840) and patient 3 has an MM 3 (14845). Data from the placebo are analyzed in the MMs in order to generate data on virtual patients (4, 5 and 6) (14850, 14855 and 14860); the virtual patient data are constructed from diagnostic prognostic data in the MMs. An active arm (14867) is shown with four patients (7, 8, 9 and 10) with MM7 (14882), MM 8 (14885), MM 9 (14888) and MM 10 (14891). Data from these therapeutic MMs analyze the therapeutic prognostics data (14895). An MM (14810), which is managed by a clinical trial administrator (14805), analyzes the hybrid control arm patient data, including data on virtual patients, and the active arm patient data.

FIG. 149 is a diagram showing an MM analyzing therapeutic prognostics biomarker data from application of a drug candidate and modifying the drug to optimize effectiveness. A drug's effectiveness is assessed in an MM over six phases (1-6) (14910, 14915, 14920, 14925, 14930 and 14935). In phase 1, patient gene, RNA or protein abnormality data are detected (14905) and a drug candidate is applied (14940). The patient biomarkers are assessed to track the drug efficacy (14945) over phases 2-6. At phase 2, therapeutic biomarker data are analyzed in an MM (14955). At phase 3, the drug's deficiency is identified by comparing biomarker data to a placebo (14960). At phase 4, a new drug is applied or the initial drug dosage is modified (14965). At phase 6, the drug effectiveness is identified (14950).

FIG. 150 is a diagram showing an MM predicting drug performance and modifying the drug when actual performance lags. Drugs are assessed over eleven phases (15005-15055). In the first phase, a drug candidate #1 (15060) is applied and in the first five phases, an MM receives biomarker feedback data (15065). In phase 5, the MM indicates a need to change to drug #2 (14070). The MM makes predictions of the drug's performance (15075) in phases 6-9, but the actual performance of drug #2 is mediocre. At phase 9, the MM indicates a need to change to drug #3 (14080). In phases 10 and 11, the effectiveness of the most recent drug shows marked improvement.

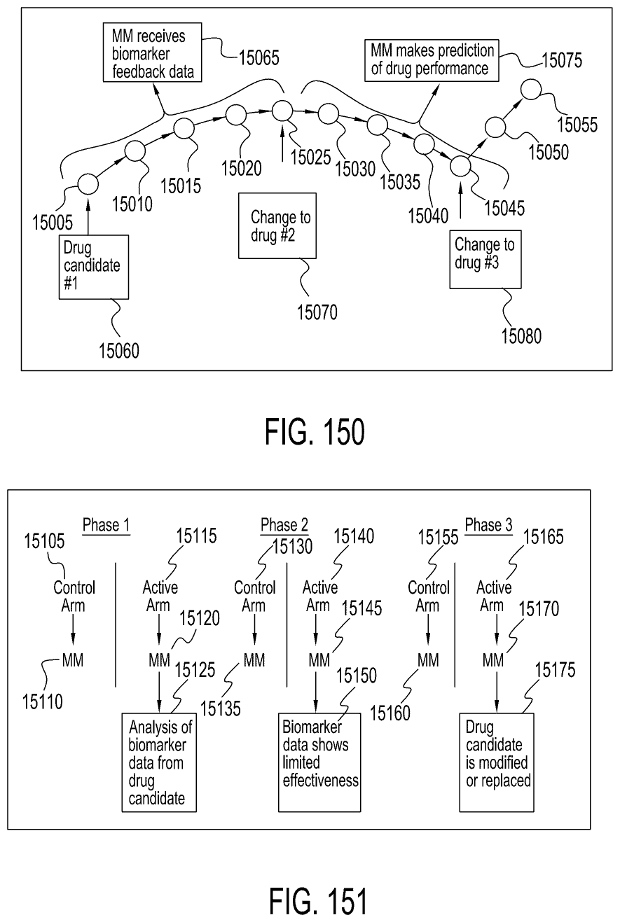

FIG. 151 is a diagram showing MMs analyzing biomarker data from clinical trial phases I and II to assess a drug candidate and modifying or replacing the drug in phase III. In phase I, MMs (15110 and 15120) analyze data from the control arm (15105) and the active arm (15115). The active arm of phase II analyzes the biomarker data from the drug candidate (15125) application. In phase II, an MM (15135) analyzes data in a control arm (15130) and an MM (15145) analyzes data in the active arm (15140). The active arm MM recognizes that the biomarker data reveals limited effectiveness (15150) of the drug application. In phase III, the MM (15160) analyzes control arm data (15155), which shows the progress of the disease without therapeutic intervention. In the phase III active arm (15165), the MM (15170) shows a drug candidate is modified or replaced (15175).

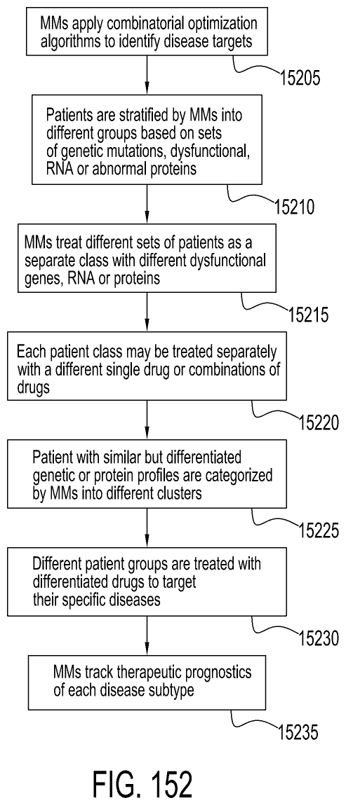

FIG. 152 is a flow chart showing MMs analyzing different patient genetic, RNA or protein abnormalities in stratified sub-types to apply drug candidates to different patient clusters. Once MMs apply combinatorial optimization algorithms to identify disease targets (15205), patients are stratified by MMs into different groups based on sets of genetic mutations, dysfunctional RNA or abnormal proteins (15210). The MMs treat different sets of patients as a separate class with different dysfunctional genes, RNA or proteins (15215) and each patient class may be treated separately with a different single drug or combinations of drugs (15220). Patients with similar but differentiated genetic or protein profiles are categorized by MMs into different clusters (15225) and different patient groups are treated with differentiated drugs to target their specific diseases (15230). The MMs track therapeutic prognostics of each disease subtype (15235).

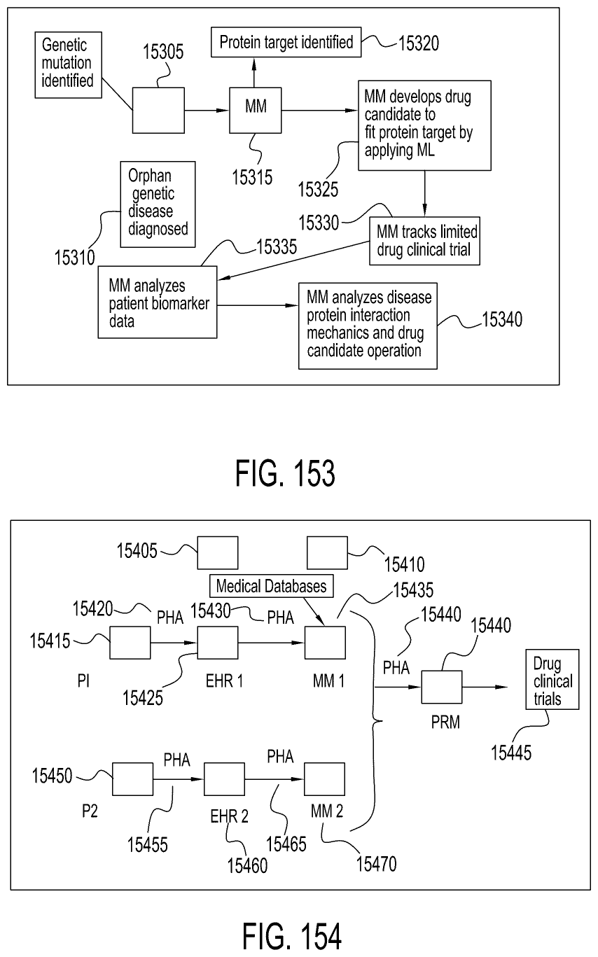

FIG. 153 is a diagram showing an MM applied to analyze the source of a genetic disease and to identification of a drug to treat the disease. After a genetic mutation is identified (15305) and an orphan genetic disease is diagnosed (15310), an MM (15315) identifies a protein target (15320) and develops a drug candidate to fit the protein target by apply ML, DL and/or AI algorithms (15325). The MM tracks limited clinical trial data (15330) and analyzes patient biomarker data (15335) over time. The MM analyzes disease protein interaction mechanics and drug candidate operation (15340).

FIG. 154 is a diagram showing a patient relationship management program coordinating clinical trials with MMs and PHAs. Patient 1 (15415), assisted by a PHA (15420) inputs data into an EHR 1 (15425), data from which is imported into MM 1 (15435) by a PHA (15430). Similarly, patient 2 (15450), assisted by a PHA (15455) inputs data into an EHR 2 (15460), data from which is imported into MM 2 (15470) by a PHA (15465). Medical databases (15405 and 15410) input data into the MMs. A PHA (15440) facilitates the transfer of data from MM 1 and MM 2 to a PRM (15440). These data are then applied to drug clinical trials (15445).

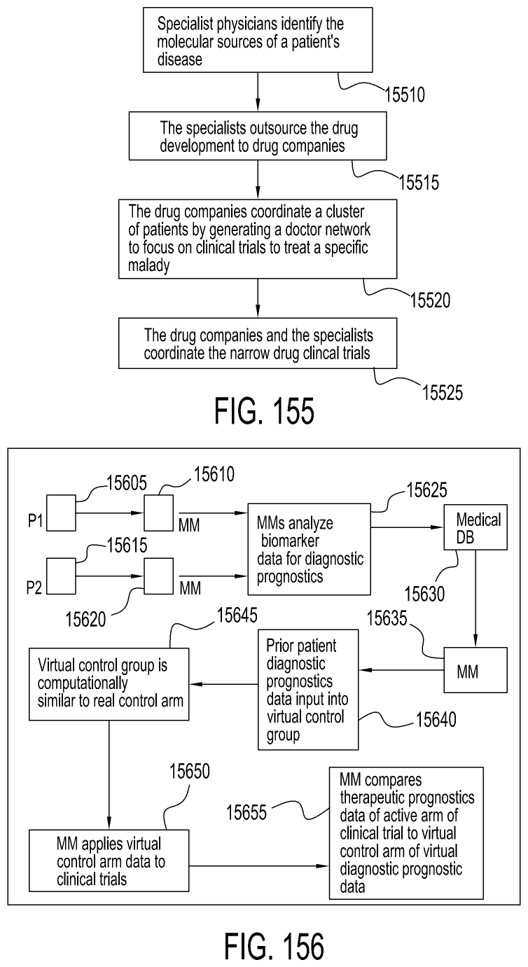

FIG. 155 is a flow chart showing drug companies generating a doctor network to coordinate clinical trials on targeted patients. After specialist physicians identify the molecular sources of a patient's disease (15505), the specialists outsource the drug development to drug companies (15510). The drug companies coordinate a cluster of patients by generating a doctor network to focus on clinical trials to treat a specific malady (15515) and the drug companies and the specialists coordinate the narrow drug clinical trials (15520).

FIG. 156 is a diagram showing an MM generating virtual control arm data from diagnostic prognostics data to compare to therapeutic prognostics data of the active arm. Patient 1 (15605) data are input into an MM (15610) and patient 2 (15615) data are input into an MM (15620). The MMs analyze biomarker data for diagnostic prognostics (15625). The patient data are input into a medical database (15630). An MM (15635) accesses the medical database and the prior patient diagnostic prognostics data are input into a virtual control group (15640). The virtual control group is computationally similar to a real control arm (15645). An MM applies virtual control arm data to clinical trials (15650) and the MM compares therapeutic prognostics data of an active arm of clinical trial to the virtual control arm virtual diagnostic prognostics data (15655).

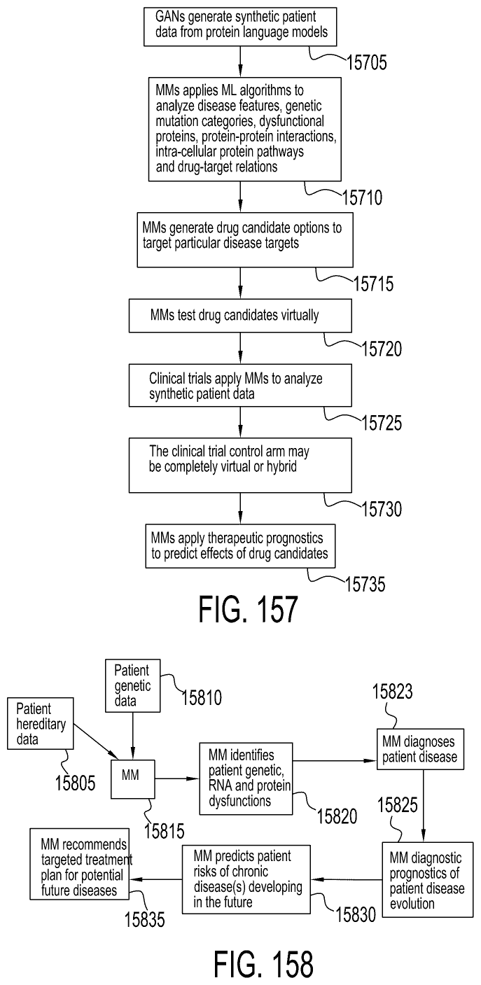

FIG. 157 is a flow chart showing MMs applied to generate synthetic patient data to use as virtual patient data of a hybrid control arm of drug clinical trials. GANs are applied to generate synthetic patient data from protein language models (15705). MMs apply ML algorithms to analyze disease features, genetic mutation categories, dysfunctional proteins, protein-protein interactions, intra-cellular protein pathways and drug-target relations (15710). MMs generate drug candidate options to target particular disease targets (15715) and the MMs test drug candidates virtually (15720). Clinical trials apply MMs to analyze synthetic patient data (15725) and the clinical trial control arm may be completely virtual or hybrid (15730). The MMs apply therapeutic prognostics to predict effects of drug candidates (15735).

FIGS. 158 to 163 refer to applications of MMs to pre-emptive medicine.

FIG. 158 is a diagram showing an MM assessing patient genetic and hereditary data to diagnose, predict and treat patient diseases that may develop in the future. Patient hereditary data (15805) and patient genetic data (15810) are input into an MM (15815). The MM identifies patient genetic, RNA and protein dysfunctions (15820) and the MM diagnoses a patient disease (15823). The MM develops a diagnostic prognostics assessment of the patient disease evolution (15825). The MM predicts patient risks of chronic disease(s) developing in the future (15830). The MM recommends targeted a treatment plan for potential future disease(s) (15835).

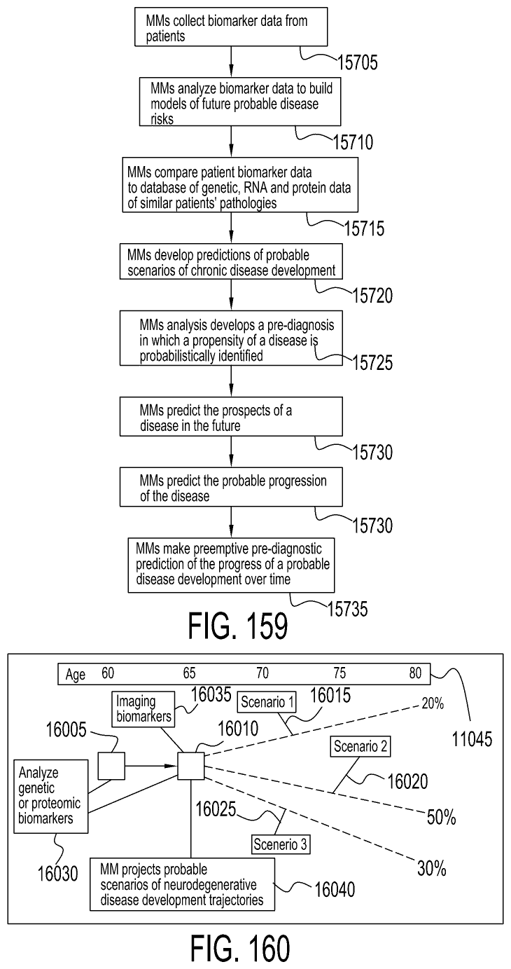

FIG. 159 is a flow chart showing MMs analyzing patient biomarker data to develop pre-emptive pre-diagnostic prediction of a probable future patient disease. After MMs collect biomarker data from patients (15905), MMs analyze biomarker data to build models of future probable disease risks (15910). MMs compare patient biomarker data to a database of genetic, RNA and protein data of similar patients' pathologies (15915) and the MMs develop predictions of probable scenarios of chronic disease development (15920). The MMs analysis develops a pre-diagnosis in which a propensity of a disease is probabilistically identified (15925) and the MMs predict the prospects of a disease in the future (15930). The MMs predict the probable progression of the disease (15935) and the MMs make preemptive pre-diagnostic prediction(s) of the progress of a probable disease development over time (15940).

FIG. 160 is a diagram showing an MM analyzing biomarker data to assess probable scenarios of neuro-degenerative disease development trajectories over time. At age 60, a patient's genetic or proteomic biomarkers (16030) are analyzed and at age 65 a patient's imaging biomarkers (16035) are analyzed in an MM. The MM projects probable scenarios of neurodegenerative disease development trajectories (16040). In scenario 1 (16015), the patient is projected to have a 20% chance of development of a significant neurodegenerative disease by age 80 (16045). In scenario 2 (16020), the patient is projected to have a 50% chance of development of a mild form of neurodegenerative disease by age 80. In scenario 3 (16025), the patient is projected to have a 30% chance of a benign form of neurodegenerative disease by age 80.

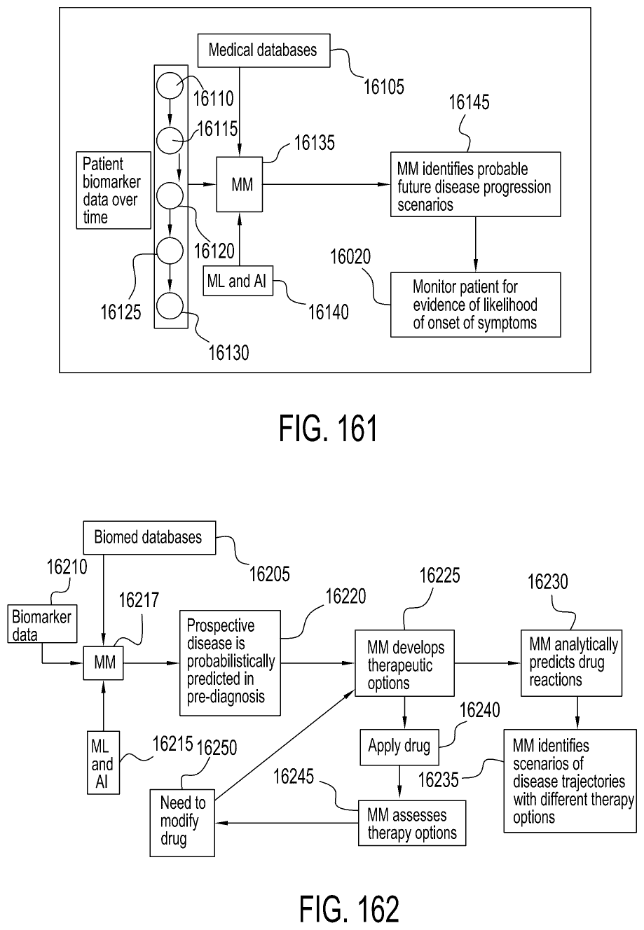

FIG. 161 is a diagram showing an MM applying AI and ML to analyze biomarker and biomedical database data to identify future disease progression scenarios. Patient biomarker data are assessed over five phases (16110, 16115, 16120, 16125 and 16130). These patient biomarker data, medical database (16105) data and ML and AI algorithms (16140) are imported into an MM (16135). The MM identifies probable future disease progression scenarios (16145). The patient is monitored for evidence of the likelihood of onset of symptoms (16150). As these symptoms present, the model is updated.

FIG. 162 is a diagram showing an MM developing and testing therapeutic options after pre-diagnosis of probable disease progression. Patient biomarker data (16210), biomedical database data (16205) and ML and AI algorithms (16215) are input into an MM (16217). The MM probabilistically predicts a pre-diagnosis of a prospective disease (16220) based on an analysis of these data. The MM develops therapeutic options (16225), applies a drug (16240) and analytically predicts drug reactions (16230). The MM assesses therapy options (16245) and identifies scenarios of disease trajectories with different therapy options (16235). After feedback from evaluation of the drug application, there is a need to modify the drug application (16250). The process then repeats with the new drug of evaluating the new drug, predicting drug reactions and identifying scenarios of disease trajectories with the new therapy options.

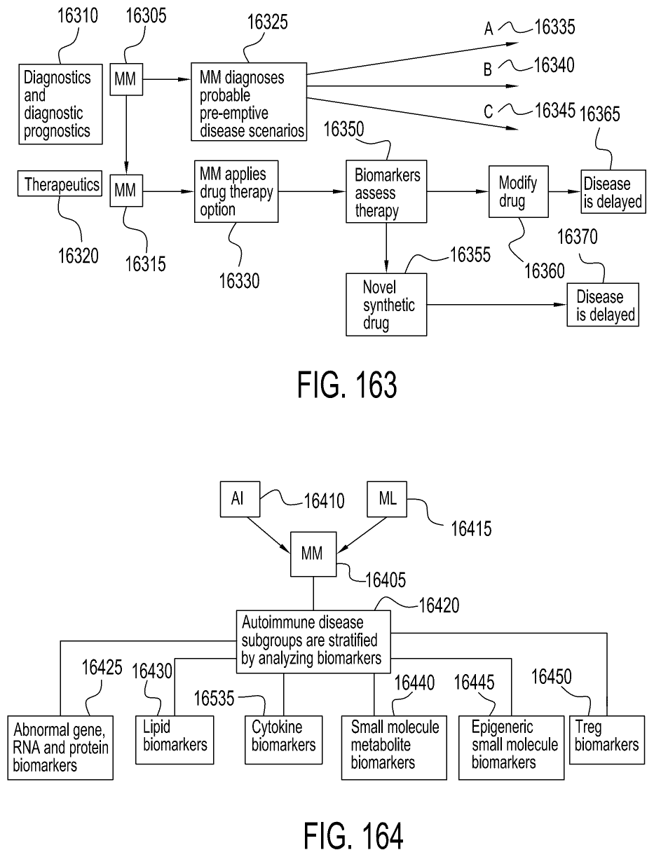

FIG. 163 is a diagram showing an MM applying, assessing and modifying drug therapy options in pre-emptive personalized medicine. An MM (16305) performs a diagnostics and diagnostic prognostics (16310) analysis in order to diagnose a probable pre-emptive disease scenario (16325). The MM identifies three scenarios A (16335), B (16340) and C (16345). An MM (16315) performs a therapeutics (16320) analysis in order to apply a drug therapy option (16330). Biomarkers assess the effectiveness of the therapy (16350), the drug is modified (16360) and the disease expression is delayed (16365). In another therapeutics scenario, the MM develops a novel synthetic drug (16355) and the disease expression is delayed (16370).

The KRAS gene has been shown to generate some types of cancers. In particular, specific mutations in the KRAS gene are traced to some cancers, particularly lung, CRC and pancreatic cancer. For example, a KRAS G12 variant may cause some forms of pancreatic cancer. In order to address this risk, KRAS inhibitors are applied. An mRNA vaccine may be configured to stimulate an immune response e.g., to attack a specific cancer mutation such as a KRAS variant. mRNA vaccines may, therefore, represent a novel therapeutic modality. Other therapeutic modalities may be applicable to blocking KRAS variants, including gene editing (CRISPR-Cas9), RNA-guided DNA editing tools (CRISPR-Cas12), RNA editing (CRISPR-Cas13) and mRNA therapies. In the case of programmable RNAi therapeutic modalities, a gene expression can be silenced. These novel therapeutic modalities are configured to prevent disease expression, particularly in patients with genetic mutations that are known to generate particular diseases. MMs are well suited to analyze patient KRAS variants and to develop personalized therapies to target these mutations.

A class of biomarker for pre-emptive medicine includes exosomes, which are 30-150 nanometer vesicles emitted by cells and which are comprised of protein, DNA, RNA, lipids and small molecules and typically enclosed in a lipid membrane. Though exosomes are involved in cellular networking, the contents of exosomes can reveal cellular disease characteristics. In a sense, exosomes supply an inter-cellular communications system. An exosome complex is also shown to contain proteins that damage RNA. Exosomes may represent a sort of cellular waste management system that illustrates the causes and effects of some chronic diseases.

Consequently, exosomes provide an excellent biomarker for understanding, describing and predicting disease states. Exosomes are implicated in diseases as varied as cancer, neurodegenerative disease and inflammatory disease. In particular, exosomes may influence the immune system. Exosome dysregulation may be a factor in some autoimmune diseases. Though exosomes are recognized as an important class of biomarker for diagnostics and diagnostic prognostics, they may also be represented as vehicles for therapies in their ability to carry protein or RNA solutions into cells by penetrating cellular membranes.

Exosomes may prove to be an important tool for predictive pre-emptive medicine by supplying biomarkers for predicting disease presence and disease state evolution. MMs are applied to identify exosomes and to describe their inter-cellular behaviors in the emergence of disease. Additionally, MMs may provide modeling solutions for therapeutic modalities involving exosomes as vehicles for transmission of novel therapies.

FIGS. 164 top 170 describe MMs applied to autoimmune disorders.

FIG. 164 is a diagram showing an MM stratify autoimmune disease subgroups by analyzing different classes of molecular biomarkers. AI (16410) and ML (16415) algorithms are applied in an MM (16405), which stratifies autoimmune disease subgroups by analyzing biomarkers (16420). The Subgroups include abnormal gene RNA and protein biomarkers (16425), lipid biomarkers (16430), cytokine biomarkers (16435), small molecule metabolite biomarkers (16440), epigenetic small molecule biomarkers (16445) and Treg biomarkers (16450).

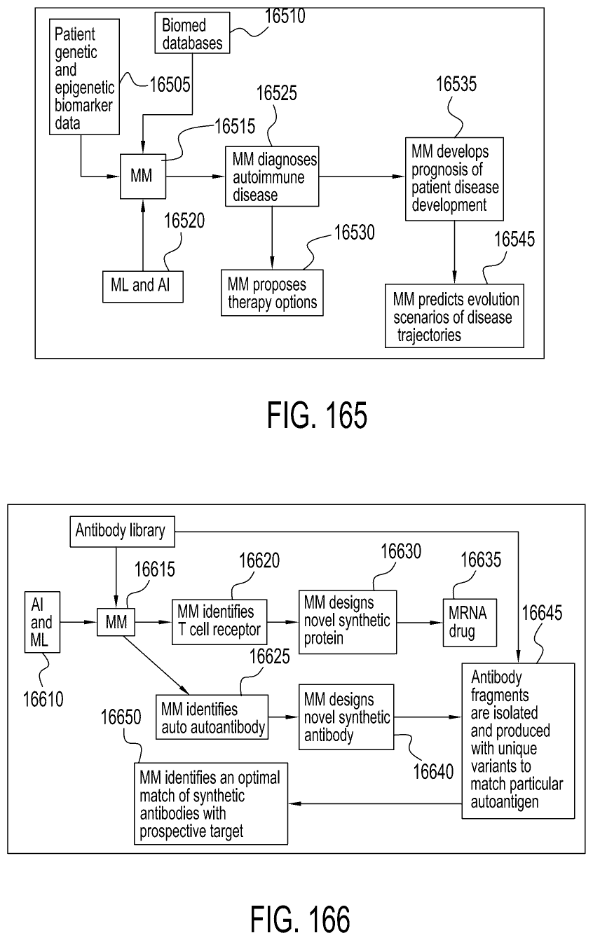

FIG. 165 is a diagram showing an MM developing a diagnosis and diagnostic prognosis of an autoimmune disease. Patient genetic and epigenetic biomarker data (16505) are input into an MM (16515) and ML and AI algorithms (16520) are applied in the MM to analyze the biomarker data. The MM diagnoses an autoimmune disease (16525) and the MM develops a prognosis of the patient disease development (16535). From the diagnostic prognostics analysis, the MM predicts the evolution scenarios of the disease trajectories (16540). From the diagnosis of the disease, the MM proposes therapy options (16530).

FIG. 166 is a diagram showing MMs building models that design novel synthetic proteins and novel synthetic antibodies. AI and ML algorithms (16610) are applied in an MM (16615) to analyze antibody data from an antibody library (16605). The MM identifies a T cell receptor (16620) and an autoantibody (16625). From the T cell receptor analysis, the MM designs a novel synthetic protein (16630) and an mRNA drug (16635) or vaccine. From the autoantibody analysis, the MM designs a novel synthetic antibody (16640). By applying data from the antibody library, the MM isolates and generates antibody fragments with unique variants to match particular autoantigens (16645). The MM identifies an optimal match of synthetic antibodies with prospective targets (16650).

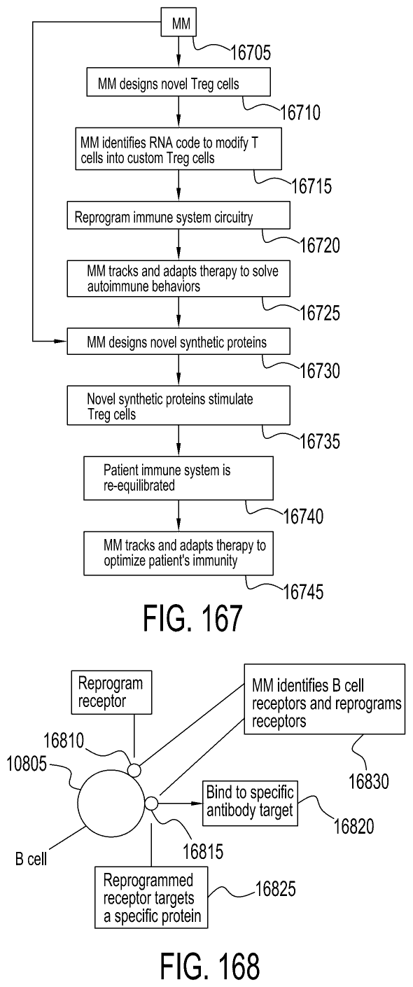

FIG. 167 is a flow chart showing an MM designing novel synthetic therapies to solve abnormal autoimmune behaviors and optimizing a patient's immunity. An MM (16705) is applied to design novel Treg cells (16710). The MM identifies an mRNA code to modify T cells into custom Treg cells (16715). By applying the mRNA code as a drug, the immune system circuitry is reprogrammed (16720) and the MM tracks and adapts the therapy to solve abnormal autoimmune behaviors (16725). The MM then designs novel synthetic proteins (16725) and the novel synthetic proteins stimulate Treg cells (16730). The patient immune system is re-equilibrated (16735) and the MM tracks and adapts therapy to optimize the patient's immunity (16740).

FIG. 168 is a diagram showing an MM identifying and reprogramming B cell receptors to bind an antibody to a specific antibody target. A B cell (16805) is shown with two receptor (16810 and 16815). An MM identifies B cell receptors and reprograms (16830) receptors (16810 and 16815). For example, an mRNA sequence can be configured to reprogram the receptors. The receptors are reprogrammed to target a specific protein (16825) and the reprogrammed receptors are configured to bind to a specific antibody target (16820).

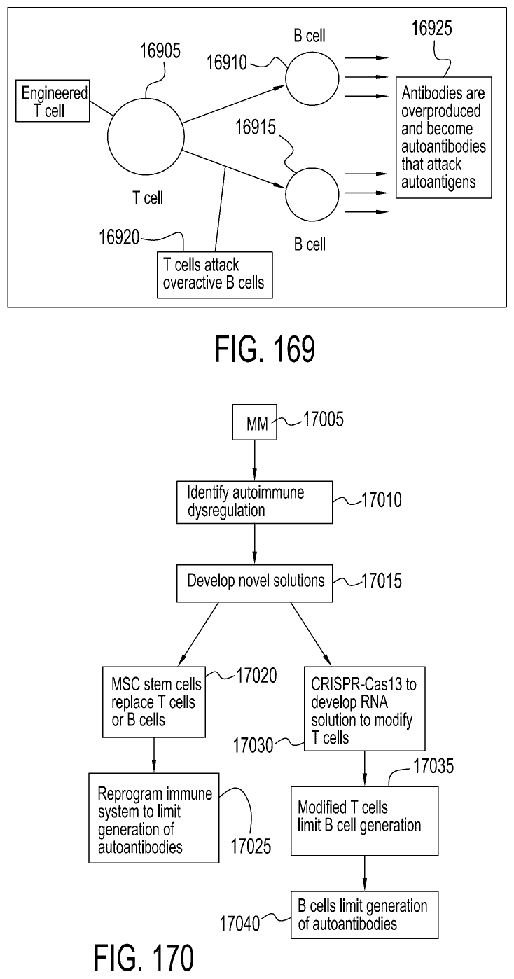

FIG. 169 is a diagram showing an MM designing T cells to attack over-active B cells that overproduce autoantibodies that attack autoantigens in CAAR T therapy. An engineered T cell (16905) is shown attacking (16920) overactive B cells (16910 and 16915). The antibodies are overproduced and become autoantibodies that attack autoantigens (16925).

FIG. 170 is a flow chart showing an MM applying therapeutic modalities of SMC stem cells and RNA editing to modify T cells and limit generation of autoantibodies. An MM (17005) identifies autoimmune dysregulation (17010) and develops novel solutions (17015). In one solution option, MSC stem cells are configured to replace T cells or B cells (17020) and the immune is reprogrammed to limit the generation of autoantibodies (17025). In another solution option, CRISPR-Cas13 is applied to develop an RNA solution to modify T cells (17030). In this option, modified T cells limit B cell generation (17035) and B cells limit generation of autoantibodies (17040).

MMs are applied to modeling complex dynamics involving the interaction of two or more autoimmune disorders. As an example, a patient may be diagnosed with both Hashimoto's disease (hypothyroidism) and psoriatic arthritis. About a third of psoriatic arthritis patients are also diagnosed with hypothyroid disease, suggesting a link. In some cases, these diseases represent genetic inheritances. Typically, each disease expresses with specific symptoms, such as arthritis inflammation of the joints.

However, MMs may analyze the dynamics between the two disorders. In the case of the thyroid, the degradation of the thyroid gland by the psoriatic arthritis may be responsible for the diminished thyroid output. A psoriatic arthritis “flare-up” may accelerate the thyroid gland degradation process. Consequently, the reduced thyroid adversely affects the psoriatic arthritis flare-up episodes. In some cases, a viral infection may stress the immune system, which stimulates the psoriatic arthritis episodes, putting a third variable in the mix of the dynamics of the auto-immune disorders. While monitoring and supplementation of the thyroid levels will stabilize the thyroid gland, the effect of thyroid supplementation may suppress the psoriatic arthritis flare-up episodes. In addition to thyroid supplementation to stabilize the thyroid levels, treatment of the psoriatic arthritis with biologics will stabilize this condition as well. The biologics will tune the immune system to minimize the autoantibodies and limit the psoriatic arthritis symptoms. An MM will assess periodic biomarkers of each disease over time and adjust the medications. Finally, in order to prevent or suppress a prospective virus that may stimulate the immune system and activate psoriatic arthritis symptoms, a vaccine regimen may be applied.

FIGS. 171 to 180 describe MMs applied metastatic cancer.

FIG. 171 is a list showing metastatic cascade stages. In stage 1, cancer cells separate from a primary tumor (17105). In stage 2, cancer cells invade adjacent tissue (17110). In stage 3, circulating tumor cells (CTCs) migrate to blood or lymphatic vessels (17115). In stage 4, CTCs exit blood lymphatic vessels at another organ (17120). In stage 5, several micrometastatic nodules form (17125). In stage 6, systematic metastasis is expressed (17130). In stage 7, drug resistance of tumor cells (17135) is presented. Unique differentiated biomarkers are identifiable at each stage. An MM is programmed to recognize each of these stages.

FIG. 172 is a diagram showing a modified therapeutic modality applied to treat a secondary tumor. In a primary tumor site (172050), a mutated gene profile in cancer cells (17210) is detected in biomarker 1. A circulating tumor cell (CTC) (17230) moves to a secondary tumor site (17215). At the secondary site, a modified gene profile is detected in biomarker 2. The secondary tumor is treated with a modified therapy (17225) from the therapy supplied to the primary tumor. MMs are applied to describe these metastases, to detail the diagnoses and to design two or more therapies to treat the cancer at each stage of development.

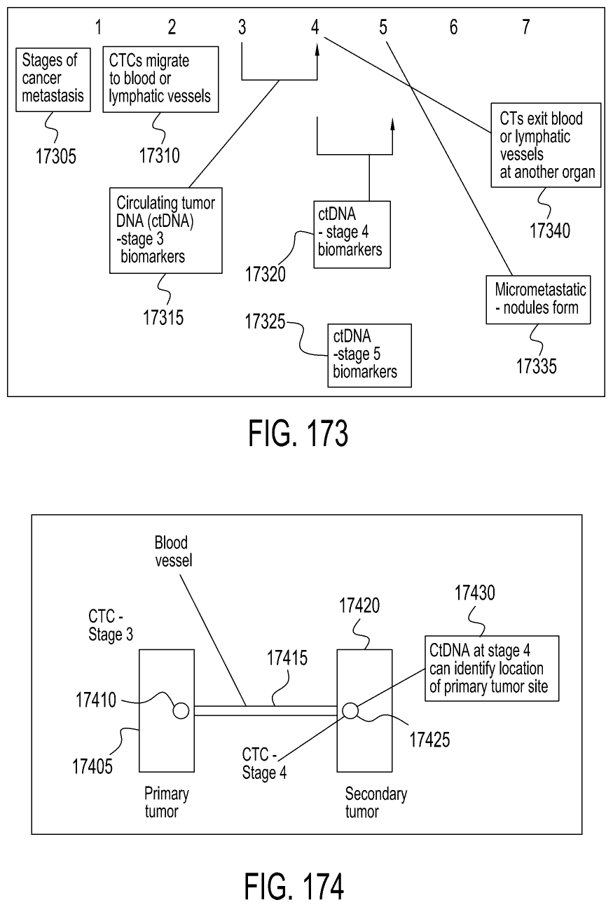

FIG. 173 is a diagram showing therapies applied to address CTCs at stages 3, 4 and 5 after unique stage biomarkers are identified. Seven stages of cancer metastasis are shown (1-7). At stage 1, the primary tumor develops and is detected. At stage three, CTCs migrate to blood or lymphatic vessels (17310). Between stages 3 and 4, circulating tumor DNA (ctDNA) biomarkers are detected (17315). At stage 4, CTCs exit blood or lymphatic vessels at another organ (17340). Between stages 4 and 5, circulating tumor DNA (ctDNA) stage four biomarkers are detected (17320). At stage 5, micrometastatic nodules form (17335) and ctDNA biomarkers (17325) are detected. At stage 6, tumors spread to multiple sites. At stage 7, the metastatic cancer process is maximized. MMs describe these metastatic cancer stages to provide diagnostics, diagnostic prognostics, therapeutics, and therapeutic prognostics analyses.

FIG. 174 is a diagram showing ctDNA of CTCs at secondary tumor site enabling the identification of a primary tumor site. A CTC at stage 3 (17410) in a primary tumor (17405) is transmitted in a blood vessel (17415) to a secondary site (17420) where it generates a secondary tumor (17420). Detection of a biomarker of the ctDNA at stage 4 in the secondary tumor site can identify the location of the primary tumor site (17430). MMs are configured to analyze these processes.

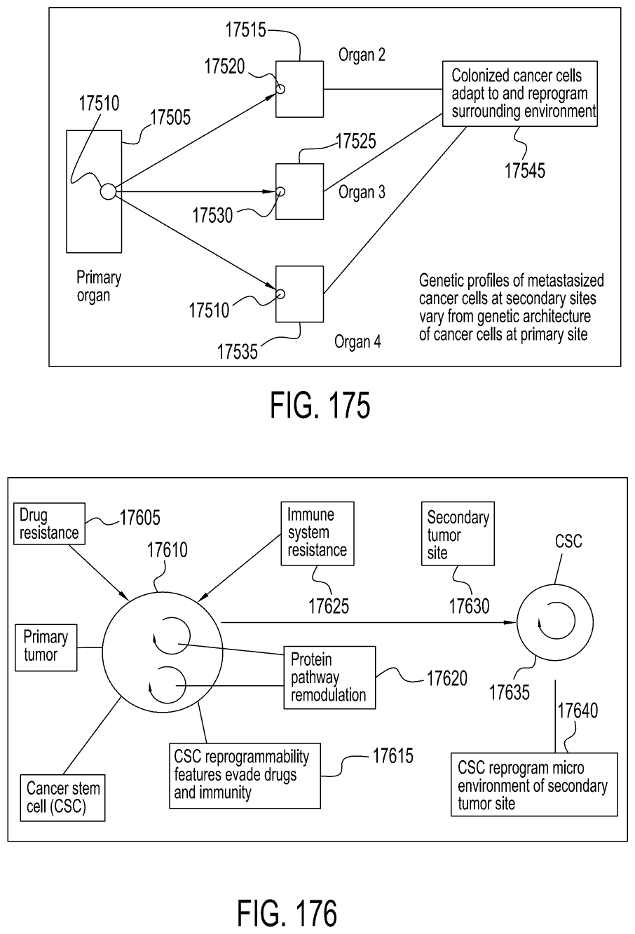

FIG. 175 is a diagram showing metastasized cancer cells, with modified genetic profiles, reprogrammed in their new tissues. After cancer cells (17510), 17520, 17530 and 17540) spread from a primary organ (175050) to other organs (2, 3 and 4) (17515, 17525 and 17535), the colonized cancer cells adapt to and reprogram their surrounding environment (17545). The genetic profiles of the metastasized cancer cells at secondary sites vary from genetic architecture of cancer cells at the primary site as revealed by analyses in MMs, as the cancer cells adapt to their new organs.

FIG. 176 is a diagram showing CSC's reprogramming protein pathways to resist drugs and immunity and reprogramming a secondary tumor site micro-environment. A cancer stem cell (CSC) in a primary tumor (17610) shows drug resistance (17615) and immune resistance (17625) as the CSC reprogrammability features evade drugs and immunity. The CSC shows protein pathway remodulation (17620). The CSC (17635) is transmitted to a secondary tumor site (17630) and the CSC reprograms the microenvironment of secondary tumor site (17640). MMs analyze these processes to develop diagnoses, prognoses and therapeutic modalities.

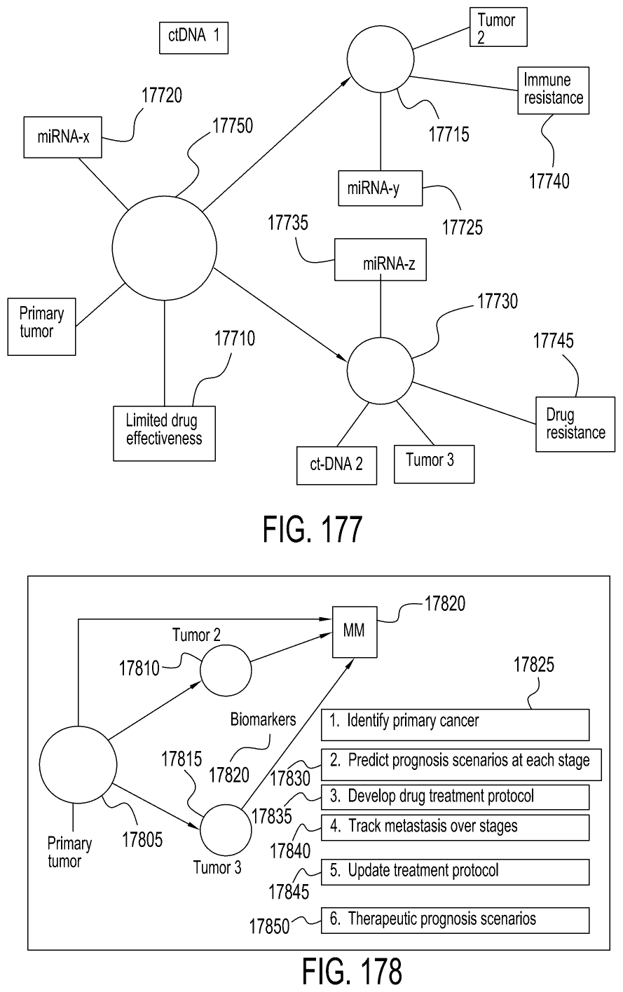

FIG. 177 is a diagram showing detection of mRNA and ctDNA biomarkers predicting drug and immunity resistance at secondary tumor sites. A miRNA-x (17720) is detected at a primary tumor (17705) the shows limited drug effectiveness (17710). A ctDNA1 is transmitted to tumor 2 (17715) and miRNA-y (17725) is shown to be immune resistant (17740). A ctDNA2 is transmitted to tumor 3 (17730) and miRNA-z (17735) is shown to be drug resistant (17745). MMs analyze these processes.

FIG. 178 is a diagram showing an MM analyzing biomarkers to diagnose, predict and treat cancer at each stage of development. Cancer cells spread from a primary tumor (17805) to tumor 2 (17810) and tumor 3 (17815). Biomarker data (17820) from the three tumors are input into an MM (17820). The MM identifies the primary cancer (17825), predicts prognosis scenarios at each stage (17830), develops drug treatment protocols (17835), tracks metastasis over stages (17840), updates the treatment protocol (17845) and develops therapeutic prognosis scenarios (17850).

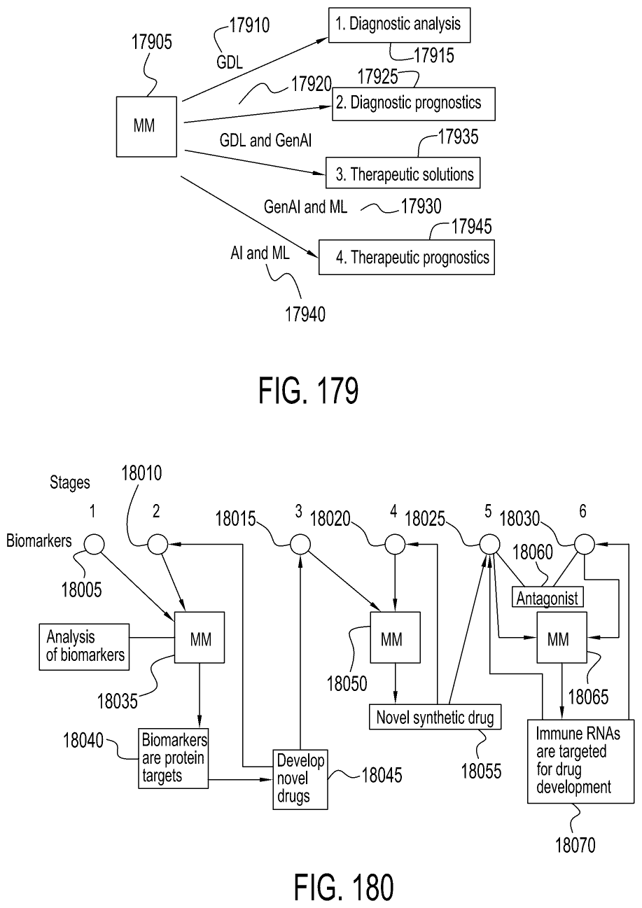

FIG. 179 is a diagram showing an MM applying different AI and ML techniques for cancer diagnostics, prognostics and therapeutics. An MM (17905) applies GDL algorithms (17910), including 2D and 3D GDL algorithms, for diagnostic analysis (17915). The MM applies GDL and GenAI algorithms (17920) to diagnostic prognostics ((17925). The MM applies GenAI and ML, including 2D and 3D GDL and GenAI, algorithms (17930) to therapeutic solutions (17935). The MM applies AI and ML algorithms (17940) to therapeutic prognostics (17945). These advanced ML, DL and AI algorithms solve descriptive and prescriptive problems associated with metastatic cancer.

FIG. 180 is a diagram showing MMs analyzing biomarkers at different stages of cancer metastasis, with MMs developing novel drug therapies at each state. Biomarkers (18005, 18010, 18015, 18020, 18025 and 18030) are input into an MM (18035) across six stages. The MM performs an analysis of the biomarkers. An MM analyzes the biomarkers as protein targets (18040) and develops novel drugs (18045), which are applied at stages 2 and 3. An MM (18050) evaluates the biomarkers at stages three and four and develops a novel synthetic drug (18055), which is applied at stages 4 and 5. Antagomirs (18060) are detected at stage 5 and analyzed by an MM (18065). The MM identifies RNAs to target for drug development (18070), which are applied at stage 6.

Although the present invention has been described in relation to particular embodiments thereof, many other variations and other uses will be apparent to those skilled in the art. It is preferred, therefore, that the present invention be limited not by the specific disclosure herein, but only by the gist and scope of the disclosure.

1-84. (canceled)

85. A system of individualized medical modeling for diagnosing a patient's disease, the system comprising:
a computer consisting of hardware logic, memory components and at least one database management system;
computer modeling software operable on the computer;
artificial intelligence (AI) or machine learning (ML) algorithms operable on the computer;
a reference biology database or large language model (LLM) with biomedical data on pathologies;
molecular biomarker data representing the patient's disease, the molecular biomarker data including gene, RNA and/or protein data;
the computer modeling software analyzing the molecular biomarker data and identifying dysfunctional patient genes, RNAs and/or proteins;
the AI or ML algorithms comparing the molecular biomarker data, including the identified dysfunctional patient genes, RNAs and/or proteins to the reference biology database to identify a specific disease; and
the computer modeling software generating or updating an individualized patient medical model to include the identified dysfunctional patient genes, RNAs and/or proteins and the identified specific disease.

86. The system of claim 85, wherein the patient's disease includes cardiovascular diseases, neurodegenerative diseases, cancer, autoimmune diseases and genetic diseases.

87. The system of claim 85, wherein the AI algorithms include GenAI algorithms, including at least one of generative adversarial networks (GANs), restricted Boltzmann Machines (RNBs), variational autoencoders (VAEs), natural language processing (NLP), large language models (LLMs) or diffusion models or generative pre-trained transformers (GPT).

88. The system of claim 85, wherein the AI algorithms include geometric deep learning (GDL) algorithms, including at least one of graph neural networks (GNNs), graph attention networks (GATs), graph convolutional neural networks (GCNs), manifold-valued neural networks (MVNs), spherical convolutional neural networks (SCNs), graphical autoencoders (GAEs) or graph of graphs neural networks (GoGNNs).

89. The system of claim 85, wherein the AI algorithms includes 3D geometric deep learning (3D GDL) algorithms, including at least one of 3D graph neural networks (3D GNNs), 3D graph attention networks (3D GATs), 3D graph convolutional neural networks (3D GCNs), 3D manifold-valued neural networks (3D MVNs), 3D spherical convolutional neural networks (3D SCNs), 3D graphical autoencoders (3D GAEs) or 3D graph of graphs neural networks (3D GoGNNs).

90. The system of claim 85, wherein the AI algorithms include generative 3D geometric deep learning (Gen 3D GDL) algorithms, including at least one of generative 3D graph neural networks (Gen 3D GNNs), generative 3D graph attention networks (Gen 3D GATs), generative 3D graph convolutional neural networks (Gen 3D GCNs), generative 3D manifold-valued neural networks (Gen 3D MVNs) or generative 3D graph of graphs neural networks (Gen 3D GoGNNs).

91. A system of individualized medical modeling for diagnosing a patient's disease, the system comprising:
at least one computer comprising hardware logic, memory components, software components and at least one database management system;
computer modeling software operable on the at least one computer;
at least one reference biology database storing gene, RNA, protein and other biological data;
at least one biology large language model (LLM), including at least one gene LLM, RNA LLM, protein LLM or antibody LLM;
artificial intelligence (AI), machine learning (ML) or deep learning (DL) software algorithms operable on the computer;
molecular biomarker data representing the patient's disease, the molecular biomarker data including gene, RNA and/or protein data;
the computer modeling software analyzing the molecular biomarker data and identifying dysfunctional patient genes, RNAs and/or proteins;
the AI or ML or DL software algorithms comparing the molecular biomarker data, including the identified dysfunctional patient genes, RNAs and/or proteins to the reference biology database or biology LLM to identify a specific patient disease; and
the computer modeling software generating or updating an individualized patient medical model to include the identified dysfunctional patient genes, RNAs and/or proteins and the identified specific patient disease.

92. The system of claim 91, wherein the computer is remotely accessed in a data center by software as a service (SaaS).

93. The system of claim 91, wherein the patient's disease includes cardiovascular, neurodegenerative, oncology, autoimmune and genetic diseases.

94. The system of claim 91, wherein the AI, ML or DL software algorithms conduct in silico experiments on patient biological data.

95. The system of claim 91, wherein the computer modeling software identifies a novel biomarker by analyzing patient biological data.

96. The system of claim 91, wherein the computer modeling software generates 4D simulations of abnormal protein pathways and abnormal protein interactions.

97. The system of claim 91, wherein the individualized patient medical model includes an individualized diagnostic prognostics model,
wherein the AI or ML or DL software algorithms further compares the molecular biomarker data, including the identified dysfunctional patient genes, RNAs and/or proteins to the reference biology database or biology LLM to generate a prediction of the progress of the patient's disease, and
wherein the computer modeling software updates the individualized diagnostic prognostics model to include the prediction of the progress of the patient's disease.

98. The system of claim 97, wherein the prediction of the progress of the patient's disease in the diagnostic prognostics model is used in personalized medicine for pre-emptive medicine.

99. A system of individualized medical modeling for medical diagnostics to diagnose a patient's disease, the system comprising:
at least one computer comprising hardware logic, memory components, software components and at least one database management system;
computer modeling software operable on the at least one computer;
at least one reference biology database storing gene, RNA, protein and other biological data;
at least one geometric deep learning algorithm operable on the at least one computer;
molecular biomarker data representing the patient's disease, the molecular biomarker data including gene, RNA and/or protein data;
the computer modeling software analyzing the molecular biomarker data and identifying dysfunctional patient genes, RNAs and/or proteins;
the at least one geometric deep learning algorithm comparing the molecular biomarker data, including the identified dysfunctional patient genes, RNAs and/or proteins, to the reference biology database to generate a diagnosis of the patient's disease; and
the computer modeling software generating or updating an individualized patient medical model to include the identified dysfunctional patient genes, RNAs and/or proteins and the diagnosis of the patient's disease.

100. The system of claim 99, wherein the individualized patient medical model includes an individualized diagnostic prognostics model,
wherein the at least one geometric deep learning algorithm further generates a prediction the progress of the patient's disease, and
wherein computer modeling software updates the individualized diagnostic prognostics model to include the prediction of the progress of the patient's disease.

101. The system of claim 99, wherein the at least one geometric deep learning algorithm is at least one generative 3D geometric deep learning algorithm.

102. The system of claim 99, wherein the at least one geometric deep learning algorithm is at least one 3D geometric deep learning algorithm.

103. The system of claim 102, wherein the individualized patient medical model includes an individualized diagnostic prognostics model,
wherein the at least one 3D geometric deep learning algorithm further generates a prediction of the progress of the patient's disease, and
wherein the computer modeling software further updates the individualized diagnostic prognostics model to include the prediction of the progress of the patient's disease.

104. The system of claim 103, wherein the at least one 3D geometric deep learning algorithm is at least one generative 3D geometric deep learning algorithm.

105. The system of claim 102, wherein the at least one 3D geometric deep learning algorithm further develops a solution to a biological pathology and generates a personalized therapy for the patient's disease, and
wherein the computer modeling software further updates the individualized patient medical model to include the personalized therapy for the patient's disease.

106. The system of claim 105, wherein the at least one 3D geometric deep learning algorithm is at least one generative 3D geometric deep learning algorithm.

107. A system of individualized medical modeling for predicting the progress of a patient's disease after a therapy is applied to the patient's disease, the system comprising:
at least one computer comprising hardware logic, memory components, software components and at least one database management system;
computer modeling software operable on the at least one computer;
at least one reference biology database storing gene, RNA, protein and other biological data;
at least one 3D geometric deep learning algorithm operable on the at least one computer;
artificial intelligence (AI) or machine learning (ML) algorithms operable on the at least one computer;
molecular biomarker data representing the patient's disease, the molecular biomarker data including gene, RNA and/or protein data;
the computer modeling software analyzing the molecular biomarker data and identifying dysfunctional patient genes, RNAs and/or proteins after the therapy is provided to target a specific disease;
the artificial intelligence (AI) or machine learning (ML) algorithms comparing the molecular biomarker data, including the identified dysfunctional patient genes, RNAs and/or proteins, to the reference biology database to identify the specific disease of the patient;
the at least one 3D geometric deep learning algorithm analyzing the molecular biomarker data, including the identified dysfunctional patient genes and analyzing the reference biology database and generating a prediction of the progress of the patient's disease after the therapy is provided; and
the computer modeling software generating or updating an individualized patient medical model to include the dysfunctional patient genes, RNAs and/or proteins, to include the identified target and to include the prediction of the progress of the patient's disease after the therapy has been applied to the patient.

108. The system of claim 107, wherein the at least one 3D geometric deep learning algorithm is at least one generative 3D geometric deep learning algorithm.

109. A system of individualized medical modeling for predicting the progress of a control arm patient's disease without therapeutic intervention in the control arm of a drug clinical trial, the system comprising:
at least one computer comprising hardware logic, memory components, software components and at least one database management system;
computer modeling software operable on the at least one computer;
at least one reference biology database storing gene, RNA, protein and other biological data;
at least one geometric deep learning or generative AI algorithm operable on the at least one computer;
molecular biomarker data representing the control arm patient's disease, the molecular biomarker data including gene, RNA and/or protein data;
the computer modeling software analyzing the molecular biomarker data of the control arm patient and identifying dysfunctional patient genes, RNAs and/or proteins;
the at least one geometric deep learning or generative AI algorithm generating a prediction of the progress of the control arm patient's disease by analyzing the molecular biomarker data, including the identified dysfunctional patient genes, RNAs and/or proteins and by analyzing the reference biology database; and
the computer modeling software generating or updating an individualized patient medical model for the control arm patient to include the identified dysfunctional patient genes, RNAs and/or proteins and the prediction of the progress of the control arm patient's disease without therapeutic intervention in the control arm of the drug clinical trial.

110. The system of claim 109 wherein the control arm includes virtual patients, wherein the virtual patients are emulated to represent an aggregation of patients with the disease, and wherein the virtual patients are analyzed to describe the progress of the disease without therapeutic intervention.

111. The system of claim 109, for predicting the progress of a disease of an active arm patient's disease with therapeutic intervention in an active arm of the drug clinical trial,
wherein the molecular biomarker data represents the active arm patient's disease,
wherein the computer modeling software analyzes the molecular biomarker data of the active arm patient in the active arm of the drug clinical trials after application of at least one therapy and identifies dysfunctional patient genes, RNAs and/or proteins,
wherein the at least one geometric deep learning or generative AI algorithm generates a prediction of the progress of the active arm patient's disease, and
wherein the computer modeling software generating or updating an individualized patient medical model for the active arm patient to include the identified dysfunctional patient genes, RNAs and/or proteins and the prediction of the progress of the active arm patient's disease with therapeutic intervention in the active arm of the drug clinical trial.

112. An integrated health record platform (IHRP) system to assist in the assessment or prediction of the progress of a patient's disease, the system comprising:
at least one computer comprising hardware logic, memory components, software components and at least one database management system;
at least one storage device operatively connected to the at least one computer;
computer modeling software operable on the at least one computer;
at least one reference biology database storing gene, RNA, protein and other biological data;
at least one geometric deep learning, machine learning or generative AI algorithm operable on the at least one computer;
biological data representing the patient's disease, including molecular biomarker data including gene, RNA, protein and/or multiomics data;
the computer modeling software analyzing the molecular biomarker data of the patient and identifying dysfunctional patient genes, RNAs and/or proteins;
the at least one geometric deep learning, machine learning or generative AI algorithm generating a prediction of the progress of the patient's disease;
the computer modeling software generating or updating an individualized patient medical model for the patient to include the identified dysfunctional patient genes, RNAs and/or proteins and the prediction of the progress of the patient's disease; and
wherein the individualized patient medical model is stored in the at least one storage device of the IHRP system.

113. The system of claim 112, further comprising medical security software operable on the at least one computer.

114. The system of claim 112, further comprising natural language processing software operable on the at least one computer, the natural language processing software surveying, translating, analyzing or summarizing medical articles or patient charts.

115. A personal health assistant (PHA) system of intelligent software agents for medical modeling to assist a physician in generating or updating an individualized patient medical model, the system comprising:
at least one computer comprising hardware logic, memory components, software components and at least one database management system;
computer modeling software operable on the at least one computer;
at least one reference biology database storing gene, RNA, protein and other biological data;
biological data representing the patient's disease, including molecular biomarker data including gene, RNA, protein and/or multiomics data;
a plurality of PHA intelligent agents operable on the at least one computer and including Artificial Intelligence (AI) algorithms, the plurality of PHA intelligent agents interfacing with the computer modeling software, the at least one reference biology database and the biological data representing the patient's disease;
the computer modeling software building or accessing an individualized medical model for the patient;
at least one of the plurality of PHA intelligent agents analyzing the molecular biomarker data of the patient and identifying dysfunctional patient genes, RNAs and/or proteins;
at least a second one of the plurality of PHA intelligent agents generating a prediction of the progress of the patient's disease;
the at least one and the at least second one of the plurality of PHA intelligent agents communicating their results to the computer modeling software; and
the computer modeling software generating or updating an individualized patient medical model for the patient to include the identified dysfunctional patient genes, RNAs and/or multiomics data and the prediction of the progress of the patient's disease.

116. The system of claim 115, further comprising:
a PHA typology that includes:
PHA-m, wherein the PHA-m is a model builder that perform tasks associated with building MMs, such as combining data into tables, graphs and models and representing data in models or simulations;
PHA-a, wherein the PHA-a is an analyzer that perform tasks involving analysis or synthesis of elements in MMs;
PHA-s, wherein the PHA-s is a searcher that seeks out data from databases;
PHA-c, wherein the PHA-c is a combiner that combines two or more AI techniques or algorithms into a hybrid synthesis for application to a particular issue involved in a MM;
PHA-i, PHA-i is an interrogator that actively interrogates data in order to build or optimize a model;
PHA-mes, wherein the PHA-m is a messenger or communicator that passes messages between models and other agents;
PHA-b, wherein the PHA-b is a broker that intermediates between MMs and LLMs or medical databases;
PHA-sec, wherein the PHA-sec is a security agent that enables different levels of security in MMs agents that enable different levels of security in MMs;
PHA-p, wherein the PHA-p is a predictor that forecasts or predicts event scenarios based on MM data; and
PHA-sims, wherein the PHA-sim is a simulator that constructs simulations from MMs.

117. A patient relationship management system for building or accessing an individualized medical model for a patient, the system comprising:
at least one computer comprising hardware logic, memory components, software components and at least one database management system;
computer modeling software operable on the at least one computer;
at least one reference biology database storing gene, RNA, protein and other biological data;
at least one geometric deep learning, machine learning or generative AI algorithm operable on the at least one computer;
biological data representing the patient's disease, including molecular biomarker data including gene, RNA, protein and/or multiomics data;
the computer modeling software interfacing with a medical modeling system; and
the computer modeling software interfacing between a patient or a doctor.

118. A system for medical modeling, the system comprising:
computer modeling hardware including at least one CPU or GPU logic circuit and at least one memory circuit;
computer modeling software operable on the computer modeling hardware;
a database storing data;
a database management system coupled to the computer modeling hardware and coupled to the database, the database management system storing and accessing data in the database;
a set of computer modeling levels contained in the database, the computer modeling levels configured to diagnose, predict or treat a patient medical condition, the levels including:
Level 1: General Patient Model;
Level 2: Diagnostics, Bioinformatics, Organ and Body System Analyses;
Level 3: Molecular and Cellular Description and Analysis;
Level 4: Structural Genetic Variant Combination Pathology Identification;
Level 5: Functional Molecular and Cellular Pathology Diagnosis;
Level 6: Diagnostic Prognosis Simulations;
Level 7: General Therapy Solutions;
Level 8: Unique Therapy Solution Genesis;
Level 9: Therapy Option Testing and Simulations;
Level 10: Therapy Prediction Scenarios;
Level 11: Unified Patient Model;
Level 12: Human Population Model; and
Level 13: Master Individualized Medical Model.

119. The system of claim 118 further including modular modeling layers on Level 1, the modular modeling layers comprising:
medical research and analysis models;
doctor observations and electronic medical records (EMR) data generated models;
electronic health records (EHR) data inputs, aggregation and analytics models;
patient history and hereditary data models;
patient blood, fluid and tissue test models; and
epigenetic models.

120. The system of claim 118 further including modular modeling layers on Level 2, the modular modeling layers comprising:
genomic, proteomic, multiomic, metabolic and cell biomarker models;
diagnostic imaging models;
body system models;
electrical system and medical device models;
organ models;
artificial organ models; and
surgical models.

121. The system of claim 118 further including modular modeling layers on Level 3, the modular modeling layers comprising:
DNA, chromosome, single nucleotide polymorphisms (SNPs), coding genes and non-coding gene models;
coding and non-coding RNA models;
protein and peptide models;
3D and 4D cell dynamics models;
multicellular network models; and
pathogen, vaccine, biologics and immune system models.

122. The system of claim 118 further including modular modeling layers on Level 4, the modular modeling layers comprising:
mutated gene models;
dysfunctional protein and peptide structure models;
cellular behaviors with dysfunctional DNA, RNA, proteins and peptides models;
in silico laboratory models for experiments of dysfunctional genes, RNA and proteins; and
epigenetics models of gene expression regulation.

123. The system of claim 118 further including modular modeling layers on Level 5, the modular modeling layers comprising:
functional models of dysfunctional structure of coding genes, non-coding genes, single nucleotide polymorphisms (SNPs), RNA and non-coding RNA;
dysfunctional protein and peptide functions models and dysfunctional protein function prediction models;
protein pathway mapping models;
protein-protein, protein-ligand and protein-ligand interaction models;
drug-target and drug-disease interaction prediction models;
cellular machinery dysfunction and dysfunctional intercellular models;
in silico experiments of dysfunctional genes, RNA and proteins models; and
auto-immune and Treg models.

124. The system of claim 118 further including modular modeling layers on Level 6, the modular modeling layers comprising:
general patient pathology progression models;
4D simulation scenario prediction of pathology evolution without therapy models;
biomarker models to identify novel biomarkers via analysis of precise phase of disease progress;
patient-environment interactions models and track patient-environment pathology progression models;
epigenetic models to analyze epigenetic patterns and networks to identify pathology characteristics and progression; and
pre-emptive medicine models for prediction and forecasting of future potential or probably pathology progression.

125. The system of claim 118 further including modular modeling layers on Level 7, the modular modeling layers comprising:
summarizing and analyzing medical research and clinical trial studies models;
rank and select exiting drug options models to fit medical diagnoses;
identification of existing drug(s) models for unique patient pathologies;
drug dose, side effect, toxicity and interactions evaluation and prediction models; and
drug delivery vehicles models such as nanoparticles, lipids and viruses.

126. The system of claim 118 further including modular modeling layers on Level 8, the modular modeling layers comprising:
identification models for gene or protein targets;
novel drug discovery models for, including experiments for novel drug discovery;
RNA, peptide and protein novel design models;
design of novel synthetic drugs models;
antibody-antigen models;
large and small molecule, antibody/ADC, radio conjugate and enzyme novel design for unique pathology models;
stem cell models;
gene, RNA, nc DNA and nc RNA editing models;
CRISPR-Cas9, CRISPR-Cas12, CRISPR-Cas13, siRNA and programmable RNA and DNA models;
cellular programming and reprogramming therapy models
immune system therapy models;
endocrine therapy models; and
CAR T cell therapy models.

127. The system of claim 118 further including modular modeling layers on Level 9, the modular modeling layers comprising:
RNA, peptide, protein, antibody and enzyme novel drug simulations models;
cellular mechanics, protein interactions and protein pathways models;
models for in silico experiments of optimal therapy options models;
drug-target and drug-disease interaction simulations models;
compare and predict models to compare or predict control group to pathology therapy group;
optimal probabilistic therapy selection models; and
precise therapy prediction and targeting models.

128. The system of claim 118 further including modular modeling layers on Level 10, the modular modeling layers comprising:
disease progression probabilities models with different drug therapy options;
drug-target interaction prediction scenarios models;
4D simulation scenarios models of disease progression with drug therapy option feedback;
drug reaction predictions models;
compare models to compare pathology diagnostic prognostic simulations to therapy option prognostic simulations;
patient cluster drug testing models;
prediction models to predict therapy responses from biomarkers;
multiomics models for drug prediction;
epigenetic biomarkers models to predict clinical response to medical interventions; and
prediction and forecasting models of probable pathology progression with therapy feedback.

129. The system of claim 118 further including modular modeling layers on Level 11, the modular modeling layers comprising:
patient models comprising a medical library of individual health events;
diagnostic integration models to integrate diagnostics model levels;
therapeutic integration models to integrate therapeutics model levels;
prognostics integration models to integrate prognostics model levels;
surgical elements integration models to integrate surgical elements; and
human longevity analyses models.

130. The system of claim 118 further including modular modeling layers on Level 12, the modular modeling layers comprising:
patient family and hereditary models;
infectious diseases and epidemiology clusters models;
public health models;
preventive medicine models;
large patient population classification models;
trauma medicine models;
medical devices-patient interactions models; and
hospital architecture, logistics and management models.

131. The system of claim 118 further including modular modeling layers on Level 13, the modular modeling layers comprising:
DNA, RNA and protein data aggregation and analysis models;
cell, organ, tissue and bio-system data models;
pathology diagnostics and prognostics models;
pathology therapeutics, prognostics and clinical testing models;
aggregate medical models; and
an atlas of human medical models.

132. A method of processing individualized medical models for diagnosing a patient's disease, the method operating on at least one computer comprising hardware logic, memory components, software components, at least one database management system, and computer modeling software, the method comprising:
executing at least one biology large language model (LLM), including at least one gene LLM, RNA LLM, protein LLM or antibody LLM;
executing at least one artificial intelligence (AI), machine learning (ML) or deep learning (DL) algorithm;
accessing a reference biology database;
accessing molecular biomarker data representing the patient's disease, the molecular biomarker data including gene, RNA and/or protein data;
analyzing, by the computer modeling software, the molecular biomarker data and identifying dysfunctional patient genes, RNAs and/or proteins;
comparing, by the at least one AI, ML or DL algorithm, the molecular biomarker data, including the identified dysfunctional patient genes, RNAs and/or proteins to the reference biology database and identifying a specific disease in the patient; and
generating or updating, by the computer modeling software, an individualized patient medical model for the patient to include the identified dysfunctional patient genes, RNAs and/or proteins and the identified specific disease.

133. The method of claim 132, wherein the patient's disease includes cardiovascular diseases, neurodegenerative diseases, cancer, autoimmune diseases and genetic diseases.

134. The method of claim 132, wherein the AI algorithm includes GenAI algorithms, including at least one of generative adversarial networks (GANs), restricted Boltzmann Machines (RNBs), variational autoencoders (VAEs), natural language processing (NLP), large language models (LLMs) or diffusion models or generative pre-trained transformers (GPT).

135. The method of claim 132, wherein the AI algorithm includes geometric deep learning (GDL) algorithms, including at least one of graph neural networks (GNNs), graph attention networks (GATs), graph convolutional neural networks (GCNs), manifold-valued neural networks (MVNs), spherical convolutional neural networks (SCNs), graphical autoencoders (GAEs) or graph of graphs neural networks (GoGNNs).

136. The method of claim 132, wherein the AI algorithm includes 3D geometric deep learning (3D GDL) algorithms, including at least one of 3D graph neural networks (3D GNNs), 3D graph attention networks (3D GATs), 3D graph convolutional neural networks (3D GCNs), 3D manifold-valued neural networks (3D MVNs), 3D spherical convolutional neural networks (3D SCNs), 3D graphical autoencoders (3D GAEs) or 3D graph of graphs neural networks (3D GoGNNs).

137. The method of claim 132, wherein the AI algorithm includes generative 3D geometric deep learning (Gen 3D GDL) algorithms, including at least one of generative 3D graph neural networks (Gen 3D GNNs), generative 3D graph attention networks (Gen 3D GATs), generative 3D graph convolutional neural networks (Gen 3D GCNs), generative 3D manifold-valued neural networks (Gen 3D MVNs) or generative 3D graph of graphs neural networks (Gen 3D GoGNNs).

138. A method of processing individualized medical models for diagnosing a patient's disease, the method operating on at least one computer comprising hardware logic, memory components, software components, at least one database management system, and computer modeling software, the method comprising:
executing at least one biology large language model (LLM), including at least one gene LLM, RNA LLM, protein LLM or antibody LLM;
executing at least one artificial intelligence (AI), machine learning (ML) or deep learning (DL) algorithm;
storing gene, RNA, protein and other biological data in at least one reference biology database;
identifying molecular biomarker data representing the patient's disease, the molecular biomarker data including gene, RNA and/or protein data;
analyzing, by the computer modeling software, the molecular biomarker data and identifying dysfunctional patient genes, RNAs and/or proteins;
comparing, by the at least one AI, ML or DL algorithms, the molecular biomarker data, including the identified dysfunctional patient genes, RNAs and/or proteins to the reference biology database and identifying a specific disease in the patient; and
generating or updating, by the computer modeling software, an individualized patient medical model for the patient to include the identified dysfunctional patient genes, RNAs and/or proteins and the identified specific disease.

139. The method of claim 138, further comprising:
accessing the computer remotely in a data center by software as a service (SaaS).

140. The method of claim 138, further comprising:
conducting in silico experiments on patient biological data.

141. The method of claim 138, further comprising:
identifying a novel biomarker by analyzing patient biological data.

142. The method of claim 138, further comprising:
generating 4D simulations of abnormal protein pathways and abnormal protein interactions.

143. The method of claim 138, further comprising:
comparing, by the AI or ML or DL software algorithms, the molecular biomarker data, including the identified dysfunctional patient genes, RNAs and/or proteins to the reference biology database or biology LLM to generate a prediction of the progress of the patient's disease; and
updating, by the computer modeling software, the individualized patient medical model to include the prediction of the progress of the patient's disease.

144. The method of claim 138, further comprising:
diagnosing, by the AI, ML or DL algorithms, the patient's disease;
developing, by the AI, ML or DL algorithms, a therapy for the patient's disease; and
updating, by the computer modeling software, the individualized patient medical model to include the therapy for the patient's disease.

145. The method of claim 138, further comprising:
diagnosing, by the AI, ML or DL algorithms, the patient's disease;
identifying, by the AI, ML or DL algorithms, at least one protein target;
generating, by the AI, ML or DL algorithms, a novel synthetic protein; and
developing, by the AI, ML or DL algorithms, a unique therapy to apply to the at least one protein target.

146. A method of processing individualized medical models for therapeutic prognostics to predict the progress of a patient's disease, the method operating on at least one computer comprising hardware logic, memory components, software components, at least one database management system, and computer modeling software, the method comprising:
accessing at least one biology large language model (LLM), including at least one gene LLM, RNA LLM, protein LLM or antibody LLM;
executing at least one artificial intelligence (AI), machine learning (ML) or deep learning (DL) algorithm;
storing gene, RNA, protein and/or other biological data in at least one reference biology database;
receiving molecular biomarker data representing the patient's disease, the molecular biomarker data including gene, RNA and/or protein data;
analyzing, by the computer modeling software, the molecular biomarker data after a therapy has been applied to the patient's disease and identifying dysfunctional patient genes, RNAs and/or proteins;
comparing, by the AI, ML, or DL algorithms, the molecular biomarker data, including the identified dysfunctional patient genes, RNAs and/or proteins to a reference biology database and identifying a specific disease in the patient after a therapy has been applied to the patient's disease;
identifying, by the AI, ML or DL algorithms, at least one protein target;
generating an assessment, by the AI, ML or DL algorithms, the progress of the therapy to the patient's disease; and
generating or updating, by the computer modeling software, an individualized patient medical model for the patient to include the identified dysfunctional patient genes, RNAs and/or proteins and the assessment of the progress of the therapy.

147. A method of processing individualized medical models for diagnosing a patient's disease, the method operating on at least one computer comprising hardware logic, memory components, program code, software components at least one database management system, and computer modeling software, the method comprising:
accessing at least one biology large language model (LLM), including at least one gene LLM, RNA LLM, protein LLM or antibody LLM;
executing at least one artificial intelligence (AI), machine learning (ML) or deep learning (DL) algorithm;
storing gene, RNA, protein and/or other biological data in at least one reference biology database;
receiving molecular biomarker data representing the patient's disease, the molecular biomarker data including gene, RNA and/or protein data;
analyzing, by the computer modeling software, the molecular biomarker data and identifying dysfunctional patient genes, RNAs and/or proteins;
comparing, by at least one geometric deep learning algorithm the molecular biomarker data, including the identified dysfunctional patient genes, to the reference biology database diagnose a patient's disease after a therapy has been applied to the patient's disease; and
generating or updating, by the computer modeling software, an individualized patient medical model for the patient to include the identified dysfunctional patient genes, RNAs and/or proteins and the diagnosis of the patient's disease after a therapy has been applied.

148. The method of claim 147, wherein the individualized patient medical model includes an individualized diagnostic prognostics model, the method further comprising:
generating, by the at least one geometric deep learning algorithm, a prediction the progress of the patient's disease; and
updating, by the computer modeling software the individualized diagnostic prognostics model to include the prediction of the progress of the patient's disease.

149. The method of claim 147, wherein the at least one geometric deep learning algorithm is at least one 3D geometric deep learning algorithm.

150. The method of claim 149, wherein the individualized patient medical model includes an individualized diagnostic prognostics model, the method further comprising:
generating, by the at least one 3D geometric deep learning algorithm, a prediction the progress of the patient's disease after a therapy has been applied to the patient's disease; and
updating, by the computer modeling software, the individualized diagnostic prognostics model to include the prediction of the progress of the patient's disease.

151. The method of claim 149, further comprising:
developing, by the at least one 3D geometric deep learning algorithm a solution to a biological pathology;
generating, by the at least one 3D geometric deep learning algorithm, a prediction of the progress of the patient's disease based on the solution; and
updating, by the computer modeling software, the individualized patient medical model to include the personalized therapy for the patient's disease.

152. The method of claim 149, wherein the individualized patient medical model includes an individualized diagnostic prognostics model, the method further comprising:
generating, by the at least one geometric deep learning algorithm, a prediction the progress of the patient's disease after a therapy has been applied; and
updating, by the computer modeling software, the individualized diagnostic prognostics model to include the prediction of the progress of the patient's disease.

153. The method of claim 149, wherein the at least one geometric deep learning algorithm is at least one generative 3D geometric deep learning algorithm.

154. The method of claim 153, further comprising:
developing, by the at least one generative 3D geometric deep learning algorithm a solution to a biological pathology;
generating, by the at least one generative 3D geometric deep learning algorithm, a personalized therapy for the patient's disease based on the solution; and
updating, by the computer modeling software, the individualized patient medical model to include the personalized therapy for the patient's disease.

155. The method of claim 153, wherein the individualized patient medical model includes an individualized diagnostic prognostics model, the method further comprising:
generating, by the at least one generative 3D geometric deep learning algorithm, a prediction the progress of the patient's disease after a therapy has been applied; and
updating, by the computer modeling software the individualized diagnostic prognostics model to include the prediction of the progress of the patient's disease.

156. The method of claim 155, further comprising:
applying the therapy to the patient.

157. A method for assessing the progress of a patient's disease in a control arm of a drug clinical trial, the method operating on at least one computer comprising hardware logic, memory components, software components, at least one database management system, and computer modeling software, the method comprising:
accessing at least one biology large language model (LLM), including at least one gene LLM, RNA LLM, protein LLM or antibody LLM;
executing at least one geometric deep learning or generative artificial intelligence algorithm;
storing gene, RNA, protein and/or other biological data in at least one reference biology database;
receiving molecular biomarker data representing the patient's disease, the molecular biomarker data including gene, RNA and/or protein data;
analyzing, by the computer modeling software, the molecular biomarker data and identifying dysfunctional patient genes, RNAs and/or proteins in the patient in the control arm of the drug clinical trial;
comparing, by the at least one geometric deep learning or generative artificial intelligence algorithms, the molecular biomarker data, including the identified dysfunctional patient genes, RNAs and/or proteins to the reference biology database and identifying a specific disease in the patient after a therapy is applied to the patient's disease;
generating, by the at least one geometric deep learning or generative artificial intelligence algorithm, a prediction of the progress of the patient's disease; and
generating or updating, by the computer modeling software, an individualized patient medical model for the patient to include the identified dysfunctional patient genes, RNAs and/or proteins and the prediction of the progress of the patient's disease without therapeutic intervention in the control arm of the drug clinical trials.

158. The method of claim 157, further comprising:
including virtual patients in the control arm;
emulating the virtual patients in the medical modeling system to represent an aggregation of patients with the disease;
analyzing the virtual patients to describe the progress of the disease without therapeutic intervention.

159. The method of claim 157, for predicting the progress of a disease of an active arm patient's disease with therapeutic intervention in an active arm of the drug clinical trial, wherein the molecular biomarker data represents the active arm patient's disease, the method further comprising:
analyzing, by the computer modeling software the molecular biomarker data of the active arm patient in the active arm of the drug clinical trial after application of at least one therapy and identifying dysfunctional patient genes, RNAs and/or proteins;
generating by the at least one geometric deep learning or generative AI algorithm a prediction of the progress of the active arm patient's disease, and
generating or updating by the computer modeling software, an individualized patient medical model for the active arm patient to include the identified dysfunctional patient genes, RNAs and/or proteins and the prediction of the progress of the active arm patient's disease with therapeutic intervention in the active arm of the drug clinical trial.

160. A method of processing individualized medical models in an integrated health record platform (IHRP) to assist in the assessment or prediction of the progress of a patient's disease, the method operating on at least one computer comprising logic hardware, memory components, software components, at least one database management system, and computer modeling software, the method comprising:
accessing at least one biology large language model (LLM), including at least one gene LLM, RNA LLM, protein LLM or antibody LLM;
executing at least one geometric deep learning or generative AI algorithm;
receiving, by the at least one computer, biological data representing a patient's disease, including molecular biomarker data including gene, RNA, protein and/or multiomics data;
accessing, by the at least one computer, a medical modeling system;
analyzing, by the medical modeling system, the molecular biomarker data of the patient and identifying dysfunctional patient genes, RNAs and/or proteins;
generating, by the at least one geometric deep learning, machine learning or generative AI algorithm, a prediction of the progress of the patient's disease;
generating or updating, by the computer modeling software, an individualized patient medical model to include the identified dysfunctional patient genes, RNAs and/or proteins and the prediction of the progress of the patient's disease; and
importing and storing, by the at least one computer, the individualized patient medical model.

161. The method of claim 160, further comprising:
executing, by the at least one computer, medical security software.

162. The method of claim 160, further comprising:
surveying, translating analyzing or summarizing, by natural language software, medical articles or patient charts.

163. A method of operating personal health assistant (PHA) software for medical modeling to assist a physician in generating or updating an individualized patient medical model, the PHA software operating on at least one computer comprising hardware logic, memory components, software components at least one database management system, and computer modeling software, the method comprising:
activating a plurality of PHA intelligent agents operable on the at least one computer and including Artificial Intelligence (AI) algorithms;
accessing, by at least one of the plurality of PHA intelligent agents at least one biology large language model (LLM), including at least one gene LLM, RNA LLM, protein LLM or antibody LLM;
receiving, by the at least one computer, biological data representing a patient's disease, including molecular biomarker data including gene, RNA, protein and/or multiomics data;
building or accessing, by the computer modeling software, an individualized medical model for the patient;
analyzing, by at least one of the PHA intelligent agents, the molecular biomarker data of the patient and identifying dysfunctional patient genes, RNAs and/or proteins;
generating, by at least a second one of the plurality of PHA intelligent agents, a prediction of the progress of the patient's disease;
generating or updating, by the computer modeling software, an individualized patient medical model to include the identified dysfunctional patient genes, RNAs and/or proteins and the prediction of the progress of the patient's disease; and
importing and storing, by the at least one computer, the individualized patient medical model into a database.

164. A method for operating patient relationship management software applied operable on at least one computer comprising hardware logic, memory components, software components, at least one database management system, and computer modeling software, the method comprising:
accessing at least one biology large language model (LLM), including at least one gene LLM, RNA LLM, protein LLM or antibody LLM;
receiving, by the patient relationship management software, biological data representing a patient's disease, including molecular biomarker data including gene, RNA, protein and/or multiomics data;
accessing, by the patient relationship management software, a medical modeling system;
analyzing, by the medical modeling system, the molecular biomarker data of the patient and identifying dysfunctional patient genes, RNAs and/or proteins;
generating, by at least one geometric deep learning, machine learning or generative AI algorithm, a prediction of the progress of the patient's disease;
generating or updating, by the medical modeling system, an individualized patient medical model to include the identified dysfunctional patient genes, RNAs and/or proteins and the prediction of the progress of the patient's disease;
interfacing the patient relationship management software between a patient or a doctor; and
importing and storing, by the patient relationship management software, the individualized patient medical model in a database.

165. The method of claim 164, further comprising:
executing, by the at least one computer, security software in connection with the patient relationship management software.
